(19) United States
(12) Patent Application Publication
(10) Pub. No.: US 2024/0010695 A1
(43) Pub. Date: Jan. 11, 2024

(54) FUSIONS OF MUTANT INTERLEUKIN-10 POLYPEPTIDES WITH ANTIGEN BINDING MOLECULES FOR MODULATING IMMUNE CELL FUNCTION

(71) Applicant: Asher Biotherapeutics, Inc., South San Francisco, CA (US)

(72) Inventors: Yik Andy YEUNG, South San Francisco, CA (US); David LIU, South San Francisco, CA (US); Renee GREER, South San Francisco, CA (US); Henry NGUYEN, South San Francisco, CA (US); Sindy Liao CHAN, South San Francisco, CA (US); Sherman Michael CHIN, South San Francisco, CA (US); Paul MESKO, South San Francisco, CA (US); Irene NI, South San Francisco, CA (US); Ivana DJURETIC, Pacifica, CA (US)

(21) Appl. No.: 18/039,608

(22) PCT Filed: Dec. 8, 2021
(86) PCT No.: PCT/US2021/062485

§ 371 Date: May 31, 2023

Related U.S. Application Data
(60) Provisional application No. 63/169,604, filed on Apr. 1, 2021.
(60) Provisional application No. 63/123,387, filed on Dec. 9, 2020.

(51) Int. Cl. C07K 14/54; C07K 16/28

(52) U.S. Cl. CPC C07K 14/5428; C07K 16/2815; C07K 2319/75; C07K 2317/92; A61K 38/00

(57) ABSTRACT

Provided herein are mutant interleukin-10 polypeptides, and fusion polypeptides comprising the mutant interleukin-10 polypeptides and antigen binding molecules. The present disclosure provides methods of modulating immune cell function by contacting the immune cell with fusion polypeptides of the present disclosure. In addition, the disclosure also provides polynucleotides encoding the disclosed fusion molecules, and vectors and host cells comprising such polynucleotides. The present disclosure further provides methods for producing the fusion molecules, pharmaceutical compositions comprising the same, and uses thereof.

### CROSS-REFERENCE TO RELATED APPLICATIONS

This application claims the priority benefit of U.S. Provisional Application Nos. 63/123,387, filed Dec. 9, 2020, and 63/169,604, filed Apr. 1, 2021, each of which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 182842000540SEQLIST.TXT, date recorded: Dec. 8, 2021, size: 565,560 bytes)

### FIELD

The present disclosure provides mutant interleukin-10 polypeptides, and fusion polypeptides comprising the mutant interleukin-10 polypeptides and antigen binding molecules. The present disclosure provides methods of modulating immune cell function by contacting the immune cell with fusion polypeptides of the present disclosure. In addition, the disclosure also provides polynucleotides encoding the disclosed fusion molecules, and vectors and host cells comprising such polynucleotides. The present disclosure further provides methods for producing the fusion molecules, pharmaceutical compositions comprising the same, and uses thereof.

### BACKGROUND

Interleukin-10 (IL-10) is a cytokine that regulates many immune cell subsets, some of which include monocytes, macrophages, dendritic cells, B cells, T cells, NK cells, and others. IL-10 binds to a heterodimeric receptor (IL-10 receptor, IL-10R) that consists of two subunits, IL-10RA, specific to IL-10 and expressed mostly on immune cells, and IL-10RB, shared with other cytokines and expressed more broadly. Binding of IL-10 to its receptor induces the phosphorylation of receptor-associated Janus kinase, JAKI, and Tyrosine kinase, TYK2, which promotes the phosphorylation of STAT3 transcription factor (pSTAT3) that regulates the transcription of many genes in lymphocytes.

IL-10 signaling induces diverse effects depending on the target cell (reviewed in Geginat et al, Cytokine Growth Factor Rev. 2016 August; 30:87-93). IL-10 is considered to be an immune suppressive cytokine, as its binding to antigen presenting cells, such as macrophages and dendritic cells, inhibits production of pro-inflammatory cytokines and capacity to stimulate T cells. For example, mice and patients with genetic defects in the IL-10/IL-10R pathway spontaneously develop colitis, suggesting that IL-10 is required to promote the homeostasis of intestinal immune cells and prevent autoimmunity. However, IL-10 has also been implicated in the development of autoimmune disease, such as systemic lupus erythematosus, through its action as a growth and differentiation factor for B-cells. Moreover, IL-10 can promote CD8+ T cell function, and this immune stimulatory activity of IL-10 (Chan et al, J Interferon Cytokine Res. 2015 December; 35(12):948-55; Nizzoli et al, Eur J Immunol. 2016 July; 46(7):1622-32) may be relevant for its ability to induce potent anti-tumor immune responses in mice (Mumm et al, Cancer Cell. 2011 Dec. 13; 20(6):781-96; Emmerich et al, Cancer Res. 2012 Jul. 15; 72(14):3570-81), and to activate CD8+ T cells in cancer patients (Naing et al, Cancer Cell. 2018 Nov. 12; 34(5):775-791).

Given its pleiotropic effects in regulating the immune response, IL-10 cytokine has been used as a therapeutic both in autoimmunity and cancer. However, despite its potent immune suppressive effects in preclinical models, the clinical benefit of IL-10 administration in Crohn's disease, psoriasis, and rheumatoid arthritis, was limited (O'Garra A, Immunol Rev. 2008; 223:114-131). Similarly, therapeutic effect of IL-10 was evaluated across multiple advanced solid tumors and, although the clinical activity was demonstrated, clinical benefit was modest and most promising in a small number of indications (Autio et al, Curr Oncol Rep. 2019 Feb. 21; 21(2):19).

These seemingly contradictory effects of IL-10 may be explained by the presence of its receptor on immune cells that can both suppress and activate the immune response in a given context. For example, in the context of cancer, stimulation of macrophages, dendritic cells, and regulatory T cells (Tregs) by IL-10 could result in immune suppression, and stimulation of CD8+ T cells by IL-10 could result in immune activation. This suggests that restricting the IL-10 activity to certain immune cell subsets could be beneficial for increasing its therapeutic effects in cancer. Furthermore, IL-10 therapy has been associated with severe anemia and hyper-ferritinemia that may require transfusion for certain patients (Tilg et al, J Immunol. 2002 Aug. 15; 169(4):2204-9). IL-10 was shown to directly stimulate ferritin translation in activated monocytic cells (Tilg et al, J Immunol. 2002 Aug. 15; 169(4):2204-9), which can lead to sequestration of iron needed for erythropoiesis. In addition to induction of ferritin in monocytes, IL-10 could also directly suppress erythropoiesis (Oehler et al, Exp Hematol. 1999 February; 27(2):217-23; Mullarky et al, Infect Immun. 2007 May; 75(5):2630-3).

CD8+ T cells have been shown to mediate efficacy of immunotherapeutic agents, including IL-10, in many preclinical cancer models (Mumm et al, Cancer Cell. 2011 Dec. 13; 20(6):781-96; Emmerich et al, Cancer Res. 2012 Jul. 15; 72(14):3570-81), and they have also been correlated with response to immunotherapies in patients (Sade-Feldman et al, Cell. 2018 Nov. 1; 175(4):998-1013). CD8+ T cells express CD8, which is a type I transmembrane glycoprotein found on the cell surface as a CD8 alpha (CD8a) homodimer and CD8 alpha-CD8 beta (CD8b) heterodimer. Alpha beta CD8+ T cells can express both CD8aa and CD8ab dimers, while CD8aa homodimers can also be expressed, albeit to a lower level, on some innate lymphocytes such as NK, NK T, and intraepithelial Tγδ cells (Baume et al, Cell Immunol. 1990 December; 131(2):352-65; Kadivar et al, J Immunol 2016; 197:4584-4592; Mayassi & Jabri, Mucosal Immunology 11, 1281-1289, 2018). CD8 dimers interact with the major histocompatibility (MHC) class I molecules on target cells and this interaction keeps the TCR closely engaged with MHC during CD8+ T cell activation. The cytoplasmic tail of CD8a contains binding sites for a T cell kinase (Lck) that initiates signal transduction downstream of the TCR during T cell activation, while CD8b is thought to increase the avidity of CD8 binding to MHC class I and influence specificity of the CD8/MHC/TCR interaction (Bosselut et al, Immunity. 2000 April; 12(4):409-18).

There is a need to reduce the toxicity of IL-10 and improve its efficacy by enhancing its activity on T cells, including CD8+ T cells, that have been associated with efficacy in preclinical cancer models and cancer patients and reducing its activity on other cells that have been associated with toxicity and undesired effects of IL-10, including monocytes, macrophages, dendritic cells, and Tregs.

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF SUMMARY

Provided herein are mutant IL-10 polypeptides comprising substitutions that enhance binding affinity to IL-10RB, reduce binding affinity to IL-10RA, and/or reduce binding to heparin. Further provided herein are fusion proteins containing such mutant IL-10 polypeptides. The present disclosure demonstrates significant advantages associated with certain fusion proteins, such as the ability to specifically target mutant IL-10 polypeptides to cell types of interest. For example, certain fusion proteins are demonstrated herein to preferentially activate CD8+ T cells over monocytes.

In some aspects, provided herein are mutant IL-10 polypeptides, wherein the mutant IL-10 polypeptides comprise an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the amino acid sequence of the wild-type mature IL-10 depicted in FIG. 1A, and wherein the mutant IL-10 polypeptides exhibit reduced binding affinity to IL-10RA polypeptide having an amino acid sequence depicted in FIG. 1B. Reduction in affinity to IL-10RA is obtained by introducing amino acid substitutions in the sequence of the wild-type IL-10 polypeptide to generate the mutant IL-10 polypeptides of the present disclosure, as depicted in FIG. 2 and FIG. 3. The mutant IL-10 polypeptides of the present disclosure have one or more amino acid substitutions relative to the amino acid sequence of the wild-type IL-10 polypeptide as depicted in FIG. 1A and selected from a group of: P20, L23, R24, R27, D28, K34, T35, Q38, M39, D41, L43, D44, N45, L46, K49, I87, V91, L94, L98, K138, S141, E142, D144, N148, E151, and I158. In some embodiments, the mutant IL-10 polypeptides of the present disclosure exhibit reduced binding affinity by 50% or more, 150% or more, two-fold or more, or ten-fold or more to IL-10RA polypeptide having an amino acid sequence depicted in FIG. 1B. In some embodiments, the one or more amino acid substitutions are at position(s) selected from the group consisting of: R24, R27, K34, Q38, D44, I87, K138, E142, D144, N148, and E151. In some embodiments, the one or more amino acid substitutions are selected from the group consisting of: R24A, R27A, K34A, K34D, K34E, K34S, K34P, K34G, K34T, K34H, K34L, K34N, K34F, K34R, K34Q, K34V, K34Y, Q38A, Q38D, Q38P, Q38G, Q38H, Q38I, Q38L, Q38R, Q38K, Q38N, Q38F, Q38T, Q38E, Q38S, Q38V, Q38Y, D44A, D44E, D44S, D44V, D44G, D44H, D44I, D44K, D44P, D44L, D44N, D44F, D44T, D44R, D44Q, I87A, K138A, E142A, E142G, E142N, E142L, E142F, E142I, E142V, E142K, E142R, E142P, E142Q, E142T, E142S, E142Y, D144A, D144E, D144G, D144H, D144R, D144I, D144K, D144N, D144Q, D144P, D144S, D144L, D144T, D144V, D144Y, N148G, N148P, N148S, N148D, N148T, N148K, N148V, N148I, N148E, N148F, E151A, E151G, E151H, E151I, E151N, E151F, E151L, E151V, E151R, E151K, E151P, E151Q, E151S, E151T, and E151Y. In some embodiments, the one or more amino acid substitutions are selected from the group consisting of: R24A, R27A, K34A, K34D, K34E, K34S, K34P, K34G, K34T, K34H, K34L, K34N, K34F, K34V, K34Y, Q38A, Q38D, Q38P, Q38G, Q38I, Q38L, Q38R, Q38K, Q38F, Q38T, Q38E, Q38S, Q38V, Q38Y, I87A, K138A, E142A, E142G, E142N, E142L, E142F, E142I, E142V, E142K, E142R, E142P, E142Q, E142T, E142S, E142Y, D144A, D144E, D144G, D144H, D144R, D144I, D144K, D144N, D144Q, D144P, D144S, D144L, D144T, D144V, D144Y, N148P, N148D, N148I, E151A, E151G, E151H, E151I, E151N, E151F, E151L, E151V, E151R, E151K, E151P, E151Q, E151S, E151T, and E151Y. In some embodiments, the mutant IL-10 polypeptide comprises an amino acid sequence selected from the group consisting of SEQ ID Nos: 87-89, 188-201, and 310-318. In some embodiments, the mutant IL-10 polypeptide comprises an amino acid sequence depicted in Table 4A, Table 8, Table 11, or Table 13.

In other embodiments, the mutant IL-10 polypeptides of the present disclosure may also: i) exhibit increased binding affinity to IL-10RB polypeptide having an amino acid sequence depicted in FIG. 1C; and ii) have one or more amino acid substitutions relative to the amino acid sequence of the wild-type mature IL-10 polypeptide as depicted in FIG. 1A and selected from a group of: N18, N21, M22, R24, D25, D28, S31, R32, D55, M68, I69, L73, E74, M77, P78, Q79, E81, N82, K88, A89, H90, N92, S93, G95, E96, N97, K99, T100, L101, L103, R104, R107, R110 and F111. In yet other embodiments, the mutant IL-10 polypeptides exhibit increased binding affinity by 150% or more to IL-IORB polypeptide having an amino acid sequence depicted in FIG. 1C. In some embodiments, the one or more amino acid substitutions are at position(s) selected from the group consisting of N18, D28, N92, K99, and L103, numbering according to SEQ ID NO:1. In some embodiments, the one or more amino acid substitutions are selected from the group consisting of: N18F, N18L, Ni8Y, D28Q, D28R, N92F, N92H, N92I, N92K, N92L, N92R, N92S, N92T, N92V, N92Y, K99N, L103N, and L103Q, numbering according to SEQ ID NO:1.

In some aspects, provided herein are mutant IL-10 polypeptides that exhibit increased binding affinity to an IL-10RB polypeptide, e.g., comprising the amino acid sequence of SEQ ID NO:3 or having an amino acid sequence depicted in FIG. 1C. In some embodiments, the mutant IL-10 polypeptides comprise one or more amino acid substitutions relative to the amino acid sequence of the wild-type mature IL-10 polypeptide according to SEQ ID NO:1, wherein the one or more amino acid substitutions are at position(s) selected from the group consisting of: N18, N21, M22, R24, D25, D28, S31, R32, D55, M68, I69, L73, E74, M77, P78, Q79, E81, N82, K88, A89, H90, N92, S93, G95, E96, N97, K99, T100, L101, L103, R104, R107, R110 and F111. In some embodiments, the one or more amino acid substitutions are at position(s) selected from the group consisting of N18, D28, N92, K99, and L103, numbering according to SEQ ID NO:1. In some embodiments, the one or more amino acid substitutions are selected from the group consisting of: NT8F, NT8L, NT8Y, D28Q, D28R, N92F, N92H, N92I, N92K, N92L, N92R, N92S, N92T, N92V, N92Y, K99N, L103N, and L103Q, numbering according to SEQ ID NO:1. In some embodiments, the mutant IL-10 polypeptide exhibits increased binding affinity by 50% or more, 100% or more, or 150% or more to an IL-10RB polypeptide, e.g., comprising the amino acid sequence of SEQ ID NO:3 or having an amino acid sequence depicted in FIG. 1C. In some embodiments, the mutant IL-10 polypeptide comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the amino acid sequence of the wild-type mature IL-10 depicted in FIG. 1A, and the mutant IL-10 polypeptides exhibit reduced binding affinity to IL-10RA polypeptide, e.g., comprising the amino acid sequence of SEQ ID NO:2 or having an amino acid sequence depicted in FIG. 1B. In some embodiments, the mutant IL-10 polypeptide further comprises one or more amino acid substitutions relative to the amino acid sequence of the wild-type IL-10 polypeptide according to SEQ ID NO:1, wherein the one or more amino acid substitutions are at position(s) selected from the group consisting of P20, L23, R24, R27, D28, K34, T35, Q38, M39, D41, L43, D44, N45, L46, K49, I87, V91, L94, L98, K138, S141, E142, D144, N148, E151, and I158. In some embodiments, the one or more amino acid substitutions are at position(s) selected from the group consisting of R24, R27, K34, Q38, D44, I87, K138, E142, D144, N148, and E151. In some embodiments, the one or more amino acid substitutions are selected from the group consisting of: R24A, R27A, K34A, K34D, K34E, K34S, K34P, K34G, K34T, K34H, K34L, K34N, K34F, K34R, K34Q, K34V, K34Y, Q38A, Q38D, Q38P, Q38G, Q38H, Q38I, Q38L, Q38R, Q38K, Q38N, Q38F, Q38T, Q38E, Q38S, Q38V, Q38Y, D44A, D44E, D44S, D44V, D44G, D44H, D44I, D44K, D44P, D44L, D44N, D44F, D44T, D44R, D44Q, I87A, K138A, E142A, E142G, E142N, E142L, E142F, E142I, E142V, E142K, E142R, E142P, E142Q, E142T, E142S, E142Y, D144A, D144E, D144G, D144H, D144R, D144I, D144K, D144N, D144Q, D144P, D144S, D144L, D144T, D144V, D144Y, N148G, N148P, N148S, N148D, N148T, N148K, N148V, N148I, N148E, N148F, E151A, E151G, E151H, E151I, E151N, E151F, E151L, E151V, E151R, E151K, E151P, E151Q, E151S, E151T, and E151Y. In some embodiments, the one or more amino acid substitutions are selected from the group consisting of: R24A, R27A, K34A, K34D, K34E, K34S, K34P, K34G, K34T, K34H, K34L, K34N, K34F, K34V, K34Y, Q38A, Q38D, Q38P, Q38G, Q38I, Q38L, Q38R, Q38K, Q38F, Q38T, Q38E, Q38S, Q38V, Q38Y, I87A, K138A, E142A, E142G, E142N, E142L, E142F, E142I, E142V, E142K, E142R, E142P, E142Q, E142T, E142S, E142Y, D144A, D144E, D144G, D144H, D144R, D144I, D144K, D144N, D144Q, D144P, D144S, D144L, D144T, D144V, D144Y, N148P, N148D, N148I, E151A, E151G, E151H, E151I, E151N, E151F, E151L, E151V, E151R, E151K, E151P, E151Q, E151S, E151T, and E151Y. In some embodiments, the mutant IL-10 polypeptide exhibits reduced binding affinity by 50% or more, 100% or more, 150% or more, two-fold or more, or ten-fold or more to an IL-10RA polypeptide, e.g., comprising the amino acid sequence of SEQ ID NO:2 or having an amino acid sequence depicted in FIG. 1B.

In some aspects, provided herein are mutant IL-10 polypeptides comprising an amino acid sequence having at least 80% amino acid, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the amino acid sequence of SEQ ID NO:1 with one or more amino acid substitutions relative to the amino acid sequence of SEQ ID NO:1, wherein the one or more amino acid substitutions are at position(s) selected from the group consisting of: N18, N21, M22, R24, D25, D28, S31, R32, D55, M68, I69, L73, E74, M77, P78, Q79, E81, N82, K88, A89, H90, N92, S93, G95, E96, N97, K99, T100, L101, L103, R104, R107, R110 and F111. In some embodiments, the one or more amino acid substitutions are at position(s) selected from the group consisting of: N18, D28, N92, K99, and L103, numbering according to SEQ ID NO:1. In some embodiments, the one or more amino acid substitutions are selected from the group consisting of: N18F, N18L, N18Y, D28Q, D28R, N92F, N92H, N92I, N92K, N92L, N92R, N92S, N92T, N92V, N92Y, K99N, L103N, and L103Q, numbering according to SEQ ID NO:1. In some embodiments, the mutant IL-10 polypeptide exhibits increased binding affinity to an IL-10RB polypeptide, e.g., comprising the amino acid sequence of SEQ ID NO:3 (for example, as compared to binding affinity of the wild-type mature IL-10 polypeptide comprising the amino acid sequence of SEQ ID NO:1 to the IL-10RB polypeptide comprising the amino acid sequence of SEQ ID NO:3). In some embodiments, the mutant IL-10 polypeptide exhibits increased binding affinity by 50% or more, by 100% or more, or by 150% or more to an IL-10RB polypeptide, e.g., comprising the amino acid sequence of SEQ ID NO:3 (for example, as compared to binding affinity of the wild-type mature IL-10 polypeptide comprising the amino acid sequence of SEQ ID NO:1 to the IL-10RB polypeptide comprising the amino acid sequence of SEQ ID NO:3). In some embodiments, the mutant IL-10 polypeptide comprises one or more further amino acid substitutions relative to the amino acid sequence of SEQ ID NO:1, wherein the one or more further amino acid substitutions are at position(s) selected from the group consisting of: P20, L23, R24, R27, D28, K34, T35, Q38, M39, D41, L43, D44, N45, L46, K49, I87, V91, L94, L98, K138, S141, E142, D144, N148, E151, and I158, numbering according to SEQ ID NO:1. In some embodiments, the mutant IL-10 polypeptide comprises one or more further amino acid substitutions relative to the amino acid sequence of SEQ ID NO:1, wherein the one or more further amino acid substitutions are at position(s) selected from the group consisting of: R24, R27, K34, Q38, D44, I87, K138, E142, D144, N148, and E151. In some embodiments, the one or more further amino acid substitutions are selected from the group consisting of: R24A, R27A, K34A, K34D, K34E, K34S, K34P, K34G, K34T, K34H, K34L, K34N, K34F, K34R, K34Q, K34V, K34Y, Q38A, Q38D, Q38P, Q38G, Q38H, Q38I, Q38L, Q38R, Q38K, Q38N, Q38F, Q38T, Q38E, Q38S, Q38V, Q38Y, D44A, D44E, D44S, D44V, D44G, D44H, D44I, D44K, D44P, D44L, D44N, D44F, D44T, D44R, D44Q, I87A, K138A, E142A, E142G, E142N, E142L, E142F, E142I, E142V, E142K, E142R, E142P, E142Q, E142T, E142S, E142Y, D144A, D144E, D144G, D144H, D144R, D144I, D144K, D144N, D144Q, D144P, D144S, D144L, D144T, D144V, D144Y, N148G, N148P, N148S, N148D, N148T, N148K, N148V, N148I, N148E, N148F, E151A, E151G, E151H, E151I, E151N, E151F, E151L, E151V, E151R, E151K, E151P, E151Q, E151S, E151T, and E151Y. In some embodiments, the one or more further amino acid substitutions are selected from the group consisting of: R24A, R27A, K34A, K34D, K34E, K34S, K34P, K34G, K34T, K34H, K34L, K34N, K34F, K34V, K34Y, Q38A, Q38D, Q38P, Q38G, Q38I, Q38L, Q38R, Q38K, Q38F, Q38T, Q38E, Q38S, Q38V, Q38Y, I87A, K138A, E142A, E142G, E142N, E142L, E142F, E142I, E142V, E142K, E142R, E142P, E142Q, E142T, E142S, E142Y, D144A, D144E, D144G, D144H, D144R, D144I, D144K, D144N, D144Q, D144P, D144S, D144L, D144T, D144V, D144Y, N148P, N148D, N148I, E151A, E151G, E151H, E151I, E151N, E151F, E151L, E151V, E151R, E151K, E151P, E151Q, E151S, E151T, and E151Y. In some embodiments, the mutant IL-10 polypeptide exhibits reduced binding affinity to an IL-10RA polypeptide, e.g., comprising the amino acid sequence of SEQ ID NO:2 (for example, as compared to binding affinity of the wild-type mature IL-10 polypeptide comprising the amino acid sequence of SEQ ID NO:1 to the IL-10RA polypeptide comprising the amino acid sequence of SEQ ID NO:2). In some embodiments, the mutant IL-10 polypeptide exhibits reduced binding affinity by 50% or more, by 100% or more, by 150% or more, by two-fold or more, or by 10-fold or more to an IL-10RA polypeptide, e.g., comprising the amino acid sequence of SEQ ID NO:2 (for example, as compared to binding affinity of the wild-type mature IL-10 polypeptide comprising the amino acid sequence of SEQ ID NO:1 to the IL-10RA polypeptide comprising the amino acid sequence of SEQ ID NO:2). In some embodiments according to any of the embodiments described herein, a mutant IL-10 polypeptide comprising one or more amino acid substitutions (e.g., that lead to increased binding affinity to an IL-10RB polypeptide, e.g., comprising the amino acid sequence of SEQ ID NO:3) comprises one, two, three, four, or more than four amino acid substitutions. In some embodiments according to any of the embodiments described herein, a mutant IL-10 polypeptide comprising one or more amino acid substitutions (e.g., that lead to reduced binding affinity to an IL-10RA polypeptide, e.g., comprising the amino acid sequence of SEQ ID NO:2) comprises one, two, three, four, or more than four amino acid substitutions. In some embodiments according to any of the embodiments described herein, a mutant IL-10 polypeptide comprises one or more amino acid substitutions (e.g., one or two amino acid substitutions) associated with increased binding affinity to an IL-10RB polypeptide and one or more amino acid substitutions (e.g., one or two amino acid substitutions) associated with reduced binding affinity to an IL-10RA polypeptide.

In some embodiments, a mutant IL-10 polypeptide comprises an amino acid substitution at position R24 relative to the amino acid sequence of SEQ ID NO:1, e.g., R24A. In some embodiments, a mutant IL-10 polypeptide comprises an amino acid substitution at position R27 relative to the amino acid sequence of SEQ ID NO:1, e.g., R27A. In some embodiments, a mutant IL-10 polypeptide comprises an amino acid substitution at position K34 relative to the amino acid sequence of SEQ ID NO:1, e.g., K34A, K34D, K34E, K34S, K34P, K34G, K34T, K34H, K34L, K34N, K34F, K34R, K34Q, K34V, or K34Y. In some embodiments, a mutant IL-10 polypeptide comprises an amino acid substitution at position Q38 relative to the amino acid sequence of SEQ ID NO:1, e.g., Q38A, Q38D, Q38P, Q38G, Q38H, Q38I, Q38L, Q38R, Q38K, Q38N, Q38F, Q38T, Q38E, Q38S, Q38V, or Q38Y. In some embodiments, a mutant IL-10 polypeptide comprises an amino acid substitution at position D44 relative to the amino acid sequence of SEQ ID NO:1, e.g., D44A, D44E, D44S, D44V, D44G, D44H, D44I, D44K, D44P, D44L, D44N, D44F, D44T, D44R, or D44Q. In some embodiments, a mutant IL-10 polypeptide comprises an amino acid substitution at position 187 relative to the amino acid sequence of SEQ ID NO:1, e.g., I87A. In some embodiments, a mutant IL-10 polypeptide comprises an amino acid substitution at position K138 relative to the amino acid sequence of SEQ ID NO:1, e.g., K138A. In some embodiments, a mutant IL-10 polypeptide comprises an amino acid substitution at position E142 relative to the amino acid sequence of SEQ ID NO:1, e.g., E142A, E142G, E142N, E142L, E142F, E142I, E142V, E142K, E142R, E142P, E142Q, E142T, E142S, or E142Y. In some embodiments, a mutant IL-10 polypeptide comprises an amino acid substitution at position D144 relative to the amino acid sequence of SEQ ID NO:1, e.g., D144A, D144E, D144G, D144H, D144R, D144I, D144K, D144N, D144Q, D144P, D144S, D144L, D144T, D144V, or D144Y. In some embodiments, a mutant IL-10 polypeptide comprises an amino acid substitution at position N148 relative to the amino acid sequence of SEQ ID NO:1, e.g., N148G, N148P, N148S, N148D, N148T, N148K, N148V, N148I, N148E, or N148F. In some embodiments, a mutant IL-10 polypeptide comprises an amino acid substitution at position E151 relative to the amino acid sequence of SEQ ID NO:1, e.g., E151A, E151G, E151H, E151I, E151N, E151F, E151L, E151V, E151R, E151K, E151P, E151Q, E151S, E151T, or E151Y. In some embodiments, a mutant IL-10 polypeptide comprises an amino acid substitution at position N18 relative to the amino acid sequence of SEQ ID NO:1, e.g., N18F, N18L, or N18Y. In some embodiments, a mutant IL-10 polypeptide comprises an amino acid substitution at position D28 relative to the amino acid sequence of SEQ ID NO:1, e.g., D28Q or D28R. In some embodiments, a mutant IL-10 polypeptide comprises an amino acid substitution at position N92 relative to the amino acid sequence of SEQ ID NO:1, e.g., N92F, N92H, N92I, N92K, N92L, N92R, N92S, N92T, N92V, or N92Y. In some embodiments, a mutant IL-10 polypeptide comprises an amino acid substitution at position K99 relative to the amino acid sequence of SEQ ID NO:1, e.g., K99N. In some embodiments, a mutant IL-10 polypeptide comprises an amino acid substitution at position L103 relative to the amino acid sequence of SEQ ID NO:1, e.g., L103N or L103Q.

In some aspects, provided herein are mutant IL-10 polypeptides comprising an amino acid sequence having at least 80% amino acid, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the amino acid sequence of SEQ ID NO:1 with one or more amino acid substitutions relative to the amino acid sequence of SEQ ID NO:1, wherein the one or more amino acid substitutions are at position(s) selected from the group consisting of: P20, L23, R24, R27, D28, K34, T35, Q38, M39, D41, L43, D44, N45, L46, K49, I87, V91, L94, L98, K138, S141, E142, D144, N148, E151, and I158, numbering according to SEQ ID NO:1. In some embodiments, the one or more amino acid substitutions are at position(s) selected from the group consisting of: R24, R27, K34, Q38, D44, I87, K138, E142, D144, N148, and E151, numbering according to SEQ ID NO:1. In some embodiments, the one or more amino acid substitutions are selected from the group consisting of: R24A, R27A, K34A, K34D, K34E, K34S, K34P, K34G, K34T, K34H, K34L, K34N, K34F, K34R, K34Q, K34V, K34Y, Q38A, Q38D, Q38P, Q38G, Q38H, Q38I, Q38L, Q38R, Q38K, Q38N, Q38F, Q38T, Q38E, Q38S, Q38V, Q38Y, D44A, D44E, D44S, D44V, D44G, D44H, D44I, D44K, D44P, D44L, D44N, D44F, D44T, D44R, D44Q, 187A, K138A, E142A, E142G, E142N, E142L, E142F, E142I, E142V, E142K, E142R, E142P, E142Q, E142T, E142S, E142Y, D144A, D144E, D144G, D144H, D144R, D144I, D144K, D144N, D144Q, D144P, D144S, D144L, D144T, D144V, D144Y, N148G, N148P, N148S, N148D, N148T, N148K, N148V, N148I, N148E, N148F, E151A, E151G, E151H, E151I, E151N, E151F, E151L, E151V, E151R, E151K, E151P, E151Q, E151S, E151T, and E151Y. In some embodiments, the one or more amino acid substitutions are selected from the group consisting of R24A, R27A, K34A, K34D, K34E, K34S, K34P, K34G, K34T, K34H, K34L, K34N, K34F, K34V, K34Y, Q38A, Q38D, Q38P, Q38G, Q38I, Q38L, Q38R, Q38K, Q38F, Q38T, Q38E, Q38S, Q38V, Q38Y, I87A, K138A, E142A, E142G, E142N, E142L, E142F, E142I, E142V, E142K, E142R, E142P, E142Q, E142T, E142S, E142Y, D144A, D144E, D144G, D144H, D144R, D144I, D144K, D144N, D144Q, D144P, D144S, D144L, D144T, D144V, D144Y, N148P, N148D, N148I, E151A, E151G, E151H, E151I, E151N, E151F, E151L, E151V, E151R, E151K, E151P, E151Q, E151S, E151T, and E151Y. In some embodiments, the one or more amino acid substitutions are selected from the group consisting of E151A and K138A, E151A and D144A, E151A and R27A, Q38A and R27A, R24A and Q38A, R24A and E151A, Q38A and E142A, E138A and E142A, R27A and K138A, R24A and K138A, and R24A and R27A. In some embodiments, the mutant IL-10 polypeptide exhibits reduced binding affinity to an IL-10RA polypeptide, e.g., comprising the amino acid sequence of SEQ ID NO:2 (for example, as compared to binding affinity of the wild-type mature IL-10 polypeptide comprising the amino acid sequence of SEQ ID NO:1 to the IL-10RA polypeptide comprising the amino acid sequence of SEQ ID NO:2). In some embodiments, the mutant IL-10 polypeptide exhibits reduced binding affinity by 50% or more, by 100% or more, by 150% or more, by 2-fold or more, or by 10-fold or more to an IL-10RA polypeptide, e.g., comprising the amino acid sequence of SEQ ID NO:2 (for example, as compared to binding affinity of the wild-type mature IL-10 polypeptide comprising the amino acid sequence of SEQ ID NO:1 to the IL-10RA polypeptide comprising the amino acid sequence of SEQ ID NO:2). In some embodiments, the mutant IL-10 polypeptide comprises one or more further amino acid substitutions relative to the amino acid sequence of SEQ ID NO:1, wherein the one or more further amino acid substitutions are at position(s) selected from the group consisting of N18, N21, M22, R24, D25, D28, S31, R32, D55, M68, I69, L73, E74, M77, P78, Q79, E81, N82, K88, A89, H90, N92, S93, G95, E96, N97, K99, T100, L101, L103, R104, R107, R110 and F111, numbering according to SEQ ID NO:1. In some embodiments, the one or more amino acid substitutions are at position(s) selected from the group consisting of: N18, D28, N92, K99, and L103, numbering according to SEQ ID NO:1. In some embodiments, the one or more amino acid substitutions are selected from the group consisting of: N18F, N18L, N18Y, D28Q, D28R, N92F, N92H, N92I, N92K, N92L, N92R, N92S, N92T, N92V, N92Y, K99N, L103N, and L103Q, numbering according to SEQ ID NO:1. In some embodiments, the mutant IL-10 polypeptide exhibits increased binding affinity to an IL-10RB polypeptide, e.g., comprising the amino acid sequence of SEQ ID NO:3 (for example, as compared to binding affinity of the wild-type mature IL-10 polypeptide comprising the amino acid sequence of SEQ ID NO:1 to the IL-10RB polypeptide comprising the amino acid sequence of SEQ ID NO:3). In some embodiments, the mutant IL-10 polypeptide exhibits increased binding affinity by 50% or more, by 100% or more, or by 150% or more to an IL-10RB polypeptide, e.g., comprising the amino acid sequence of SEQ ID NO:3 (for example, as compared to binding affinity of the wild-type mature IL-10 polypeptide comprising the amino acid sequence of SEQ ID NO:1 to the IL-10RB polypeptide comprising the amino acid sequence of SEQ ID NO:3). In some embodiments, the mutant IL-10 polypeptide comprises an amino acid sequence selected from the group consisting of SEQ ID Nos: 87-89, 188-201, and 310-318.

In some embodiments, the mutant IL-10 polypeptide further comprises an amino acid substitution relative to the amino acid sequence of SEQ ID NO:1 at position R107. In some embodiments, the mutant IL-10 polypeptide further comprises an R107A mutation, numbering according to SEQ ID NO:1. In some embodiments, the mutant IL-10 monomer polypeptide comprises a sequence selected from the group consisting of SEQ ID Nos:422-428. In some embodiments, the mutant IL-10 monomer polypeptide comprises the sequence of a mutant monomer IL-10 polypeptide shown in Table 11. In some embodiments, the mutant IL-10 monomer polypeptide comprises an amino acid sequence selected from the group consisting of SEQ ID Nos: 87-89, 188-201, 310-318, and 422-428.

In some embodiments, the mutant IL-10 polypeptide is a dimer, e.g., a homodimer or a heterodimer. In some embodiments, the mutant IL-10 polypeptide is a monomer, e.g., comprising an amino acid or peptide insertion between N116 and K117 (e.g., as depicted in FIG. 1D) to enable folding and expression as a monomer. In some embodiments, the insertion is 1-15 amino acids in length. In some embodiments, the insertion is 1, 2, 3, 4, 5, 6, 7, 8, 9, 10, 11, 12, 13, 14, or 15 amino acids in length. In certain embodiments, the insertion is 6 amino acids in length. In some embodiments, the mutant IL-10 monomer polypeptide comprises the amino acid sequence of SEQ ID NO:1 with an amino acid or peptide insertion of between 1 and 15 amino acids immediately following residue C114, E115, N116, K117, S118, K119, or A120, numbering based on SEQ ID NO:1. Examples of insertion can include, without limitation, G, GG, GGG, GGGG (SEQ ID NO:80), GGGSG (SEQ ID NO:81), GGGGG (SEQ ID NO:82), GGGGGG (SEQ ID NO:83), and GGGSGG (SEQ ID NO:84). In some embodiments, the mutant IL-10 monomer polypeptide comprises the amino acid sequence of SEQ ID NO:187. In some embodiments, the mutant monomer IL-10 polypeptides of the present disclosure have reduced binding affinity to IL-10RA polypeptide having an amino acid sequence depicted in FIG. 1B, and have amino acid substitutions selected from a group of: P20, L23, R24, R27, D28, K34, T35, Q38, M39, D41, L43, D44, N45, L46, K49, I87, V91, L94, L98, K138, S141, E142, D144, N148, E151, and 1158 (or selected from a group of: R24, R27, K34, Q38, D44, I87, K138, E142, D144, N148, and E151), where the amino acid numbering refers to the corresponding amino acids in the wild type IL-10 polypeptide without the 6 linker insertion. In some embodiments, the mutant monomer IL-10 polypeptides of the present disclosure also have increased binding affinity to IL-10RB polypeptide having an amino acid sequence depicted in FIG. 1C, and have amino acid substitutions selected from a group of: N18, N21, M22, R24, D25, D28, S31, R32, D55, M68, I69, L73, E74, M77, P78, Q79, E81, N82, K88, A89, H90, N92, S93, G95, E96, N97, K99, T100, L101, L103, R104, R107, RHO and F111 (or selected from a group of: N18, D28, N92, K99, and L103). In some embodiments, the mutant IL-10 monomer polypeptide comprises an amino acid substitution at position N92, numbering based on SEQ ID NO:1. In some embodiments, the mutant IL-10 monomer polypeptide comprises amino acid substitution N92I, N92A, N92V, N92L, N92M, N92Y, N92F, N92S, N92T, N92H, or N92Q. In some embodiments, the mutant IL-10 monomer polypeptide comprises amino acid substitution N92F, N92H, N92K, N92L, N92R, N92S, N92T, N92V, or N92Y. In some embodiments, the mutant IL-10 monomer polypeptide further comprises one or more of amino acid substitutions N18I, K99N and F111L, numbering based on SEQ ID NO:1. In some embodiments, the mutant IL-10 monomer polypeptide further comprises amino acid substitutions N18I, K99N and F111L, numbering based on SEQ ID NO:1. In some embodiments, the mutant IL-10 monomer polypeptide comprises amino acid substitutions N18I, N92I, K99N and F111L, numbering based on SEQ ID NO:1. In some embodiments, the mutant IL-10 monomer polypeptide comprises the sequence of SEQ ID NO:188. In some embodiments, the mutant IL-10 monomer polypeptide comprises amino acid substitutions N18I, N92I, K99N and F111 L and further comprises one or more further amino acid substitutions at position(s) R24, R27, Q38, I87, K138, E142, D144, and/or E151, numbering based on SEQ ID NO:1. In some embodiments, the mutant IL-10 monomer polypeptide comprises an amino acid sequence selected from the group consisting of SEQ ID Nos: 87-89, 188-201, 310-318, and 422-428.

Mutant IL-10 polypeptides disclosed herein, due to their decreased binding affinity for IL-10R complex, have decreased ability to stimulate IL-10R-expressing cells, including CD8+ T cells that have been shown to mediate beneficial effects of IL-10 in preclinical cancer models (Mumm et al, Cancer Cell. 2011 Dec. 13; 20(6):781-96; Emmerich et al, Cancer Res. 2012 Jul. 15; 72(14):3570-81). In order to turn mutant IL-10 polypeptides into therapeutics that could be both safer and more effective for the treatment of cancer and other immune-related diseases such as certain infectious diseases, fusion proteins comprising disclosed mutant IL-10 polypeptides and antigen binding molecules, such as antibodies, for antigens present on CD8+ T cells, such as CD8, were generated. Such fusion proteins comprising of mutant IL-10 polypeptides and antibodies binding specific antigens are also referred to as “targeted” fusion proteins as they bind to antigens recognized by the antigen binding molecules of the fusion. This distinguishes them from “untargeted” fusion proteins comprising mutant IL-10 polypeptides and control antibodies that do not bind to any particular antigens (i.e. Fc fusions or control antibody fusions with IL-10 polypeptides; Poutahidis et al, Carcinogenesis. 2007 December; 28(12):2614-23).

Without wishing to be bound to theory, FIG. 4A depicts the general mechanism for how antigen binding molecules binding to an antigen on CD8+ T cells could work to increase the binding and/or stimulation of CD8+ T cells by the mutant IL-10 polypeptides in the context of disclosed targeted fusion proteins containing said mutant IL-10 polypeptides. Certain antigen binding molecules, when fused to mutant IL-10 polypeptides, have the ability to substantially increase the binding and/or activity of mutant IL-10 polypeptides only on cells expressing the antigen for the antigen binding molecule of the fusion, resulting in preferential activation of antigen-expressing over antigen-non expressing cells (FIG. 4A). Unlike targeted fusion proteins, untargeted fusion proteins containing the same mutant IL-10 polypeptide, do not preferentially bind to and/or activate antigen-expressing cells (FIG. 4A). FIG. 4B depicts the general mechanism for a mutant monomer IL-10 polypeptide.

Without wishing to be bound to theory, it is thought that the difference in activation of antigen-expressing over antigen-non expressing cells by the targeted fusion protein, and the difference in activation of antigen-expressing cells by the targeted and the untargeted fusion protein are important for the effectiveness of the targeted fusion protein as a therapeutic. A fusion protein that is more selective for the cells that associate with efficacy, such as CD8+ T cells, over other cells that associate with toxicity or undesired effects on efficacy, may have greater therapeutic index.

In some embodiments, the fusion protein activates CD8+ T cells with 10-fold or greater potency, or 50-fold or greater potency, as compared to activation of monocytes. In some embodiments, said mutant IL-10 polypeptide comprises the sequence of SEQ ID NO:1 with one or more amino acid substitutions relative to SEQ ID NO:1, and wherein the substitutions are at positions of SEQ ID NO:1 selected from the group consisting of: P20, L23, R24, R27, D28, K34, T35, Q38, M39, D41, L43, D44, N45, L46, K49, I87, V91, L94, L98, K138, S141, E142, D144, N148, E151, and 1158 (or selected from the group consisting of: R24, R27, K34, Q38, D44, I87, K138, E142, D144, N148, and E151). In other embodiments, said mutant IL-10 polypeptide also contains one or more mutations at one or more positions of SEQ ID NO:1 selected from the group consisting of: N18, N21, M22, R24, D25, D28, S31, R32, D55, M68, I69, L73, E74, M77, P78, Q79, E81, N82, K88, A89, H90, N92, S93, G95, E96, N97, K99, T100, L101, L103, R104, R107, R110 and F111 (or selected from the group consisting of: N18, D28, N92, K99, and L103).

In some embodiments, the IL-10 fusion proteins disclosed herein activate antigen-expressing IL-10R+ cells, such as CD8+ T cells, over antigen-not expressing IL-10R+ cells, such as monocytes, by at least 5 fold, at least 10 fold, at least 50 fold, at least 100 fold, or at least 200 fold. In some embodiments, the fusion proteins activate antigen-expressing IL-10R+ cells at least 50 fold, at least 100 fold, or at least 200 fold, e.g., compared to a fusion molecule comprising the IL-10 mutant polypeptide and a control antibody not binding to any antigens expressed on said cells. In some embodiments, said cell activation by the IL-10 fusion protein is determined by measuring the expression of pSTAT3 in said cells following the treatment with said IL-10 fusion proteins.

In some aspects, the fusion proteins disclosed herein may reduce the pleiotropic effects of IL-10 on immune cell expressing the IL-10R complex down to a subset of effects by reducing the effects of IL-10 to certain immune cell subsets of interest, such as CD8+ T cells. Such reduction may increase the efficacy and reduce the toxicity of IL-10 polypeptides when administered as therapeutics by directing their action on subsets of T cells that contain tumor antigen-specific CD8+ T cells or viral antigen-specific CD8+ T cells thus sparring: 1) T cells that may not contribute to efficacy; or 2) systemically distributed myeloid cells that express IL-10R and may contribute to toxicity or act as a sink; 3) other immune cells that can negatively contribute to efficacy such as dendritic cells and Tregs.

In some embodiments according to any of the embodiments described herein, the T cells (e.g., CD8+ T cells) are human T cells. In some embodiments, the monocytes/other immune cells are human cells. In some embodiments, the fusion protein comprises the mutant IL-10 polypeptide according to any one of the above embodiments and an antigen binding molecule that binds to an antigen on T cells, e.g., CD8 (e.g., CD8ab, CD8a, or CD8aa; or CD8b and/or CD8ab), CD4, or PD-1. In some embodiments, a CD8 polypeptide, antigen, or dimer of the present disclosure (e.g., CD8a, CD8b, CD8aa, and/or CD8ab) is a human CD8 polypeptide, antigen, or dimer. In some embodiments, a CD4 polypeptide, antigen, or dimer of the present disclosure is a human CD4 polypeptide. In some embodiments, a PD-1 polypeptide, antigen, or dimer of the present disclosure is a human PD-1 polypeptide.

In some embodiments, the fusion protein comprises an antigen binding molecule that comprises a heavy chain variable (VH) domain and a light chain variable (VL) domain. In some embodiments, the VH domain comprises a CDR-H1 comprising the amino acid sequence of SEQ ID NO:110, a CDR-H2 comprising the amino acid sequence of SEQ ID NO:111, and a CDR-H3 comprising the amino acid sequence of SEQ ID NO:112; and wherein the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6. In some embodiments, the VH domain comprises a CDR-H1 comprising the amino acid sequence of SEQ ID NO:13, a CDR-H2 comprising the amino acid sequence of SEQ ID NO:14, and a CDR-H3 comprising the amino acid sequence of SEQ ID NO:15; and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:18. In some embodiments, the VH domain comprises a CDR-H1 comprising the amino acid sequence of SEQ ID NO:19, a CDR-H2 comprising the amino acid sequence of SEQ ID NO:20, and a CDR-H3 comprising the amino acid sequence of SEQ ID NO:21; and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24. In some embodiments, the VH domain comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; and wherein the VL domain comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30. In some embodiments, the VH domain comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; and wherein the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36. In some embodiments, the VH domain comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39; and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45; and wherein the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48. In some embodiments, the VH domain comprises a CDR-H1 comprising the amino acid sequence of SEQ ID NO:177, a CDR-H2 comprising the amino acid sequence of SEQ ID NO:178, and a CDR-H3 comprising the amino acid sequence of SEQ ID NO:179; and wherein the VL domain comprises a CDR-L1 comprising the amino acid sequence of SEQ ID NO:180, a CDR-L2 comprising the amino acid sequence of SEQ ID NO:181, and a CDR-L3 comprising the amino acid sequence of SEQ ID NO:182. In some embodiments, the VH domain comprises a CDR-H1 comprising the amino acid sequence of X₁X₂AIS, wherein X₁ is S, K, G, N, R, D, T, or G, and wherein X₂ is Y, L, H, or F (SEQ ID NO:259), a CDR-H2 comprising the amino acid sequence of X₁X₂X₃PX₄X₅X₆X₇X₈X₉YX₁₀QKFX₁₁G, wherein X₁ is G or H, X₂ is I or F, X₃ is I, N, or M, X₄ is G, N, H, S, R, I, or A, X₅ is A, N, H, S, T, F, or Y, X₆ is A, D, or G, X₇ is T, E, K, V, Q, or A, X₈ is A or T, X₉ is N or K, X₁₀ is A or N, and X₁₁ is Q or T (SEQ ID NO:260), and a CDR-H3 comprising the amino acid sequence of X₁X₂X₃GX₄X₅LFX₆X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO:261); and wherein the VL domain comprises a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO:262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO:263), and a CDR-L3 comprising the amino acid sequence of QX₁X₂X₃X₄X₅PWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO:264). In some embodiments, the VH domain comprises a CDR-H1 comprising the amino acid sequence of SEQ ID NO:225, a CDR-H2 comprising the amino acid sequence of SEQ ID NO:226, and a CDR-H3 comprising the amino acid sequence of SEQ ID NO:227; and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228. In some embodiments, the VH domain comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233; and wherein the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236. In some embodiments, the VH domain comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233; and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228. In some embodiments, the VH domain comprises a CDR-H1 comprising the amino acid sequence of X₁YX₂MS, wherein X₁ is S, D, E, A, or Q and X₂ is A, G, or T (SEQ ID NO:268), a CDR-H2 comprising the amino acid sequence of DIX₁X₂X₃GX₄X₅TX₆YADSVKG, wherein X₁ is T, N, S, Q, E, H, R, or A, X₂ is Y, W, F, or H, X₃ is A, S, Q, E, or T, X₄ is G or E, X₅ is S or I, and X₆ is A or G (SEQ ID NO:269), and a CDR-H3 comprising the amino acid sequence of X₁X₂X₃YX₄WX₅X₆AX₇DX₈, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO:270); and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the VH domain comprises a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231; and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the VH domain comprises a CDR-H1 comprising the amino acid sequence of SEQ ID NO:229, a CDR-H2 comprising the amino acid sequence of SEQ ID NO:237, and a CDR-H3 comprising the amino acid sequence of SEQ ID NO:231; and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the VH domain comprises a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:15; and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:18. In some embodiments, the VH domain comprises a CDR-H1 comprising the amino acid sequence of SEQ ID NO:53, a CDR-H2 comprising the amino acid sequence of SEQ ID NO:52, and a CDR-H3 comprising the amino acid sequence of SEQ ID NO:21; and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24. In some embodiments, the VH domain comprises a CDR-H1 comprising the amino acid sequence of SEQ ID NO:49, a CDR-H2 comprising the amino acid sequence of SEQ ID NO:52, and a CDR-H3 comprising the amino acid sequence of SEQ ID NO:27; and wherein the VL domain comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30. In some embodiments, the VH domain comprises a CDR-H1 comprising the amino acid sequence of SEQ ID NO:54, a CDR-H2 comprising the amino acid sequence of SEQ ID NO:52, and a CDR-H3 comprising the amino acid sequence of SEQ ID NO:33; and wherein the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36. In some embodiments, the VH domain comprises a CDR-H1 comprising the amino acid sequence of SEQ ID NO:55, a CDR-H2 comprising the amino acid sequence of SEQ ID NO:56, and a CDR-H3 comprising the amino acid sequence of SEQ ID NO:39; and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises a CDR-H1 comprising the amino acid sequence of SEQ ID NO:55, a CDR-H2 comprising the amino acid sequence of SEQ ID NO:57, and a CDR-H3 comprising the amino acid sequence of SEQ ID NO:45; and wherein the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48. In some embodiments, the VH domain comprises a CDR-H1 comprising the amino acid sequence of SEQ ID NO:49, a CDR-H2 comprising the amino acid sequence of SEQ ID NO:50, and a CDR-H3 comprising the amino acid sequence of SEQ ID NO:3; and wherein the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6. In some embodiments, the VH domain comprises a CDR-H1 comprising the amino acid sequence of SEQ ID NO:183, a CDR-H2 comprising the amino acid sequence of SEQ ID NO:184, and a CDR-H3 comprising the amino acid sequence of SEQ ID NO:179; and wherein the VL domain comprises a CDR-L1 comprising the amino acid sequence of SEQ ID NO:180, a CDR-L2 comprising the amino acid sequence of SEQ ID NO:181, and a CDR-L3 comprising the amino acid sequence of SEQ ID NO:182. In some embodiments, the VH domain comprises a CDR-H1 comprising the amino acid sequence of GX₁X₂FX₃X₄X₅, wherein X₁ is G, Y, S, or A, X₂ is T, S, G, R, N, or H, X₃ is S, T, R, H, Y, G, or P, X₄ is S, K, G, N, R, D, T, or G, and X₅ is Y, L, H, or F (SEQ ID NO:265), a CDR-H2 comprising the amino acid sequence of X₁PX₂X₃X₄X₅, wherein X₁ is I, N, or M, X₂ is G, N, H, S, R, I, or A, X₃ is A, N, H, S, T, F, or Y, X₄ is A, D, or G, and X₅ is T, E, K, V, Q, or A (SEQ ID NO:266), and a CDR-H3 comprising the amino acid sequence of X₁X₂X₃GX₄X₅LFX₆X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO:267); and wherein the VL domain comprises a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO:262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO:263), and a CDR-L3 comprising the amino acid sequence of QX₁X₂X₃X₄X₅PWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO:264). In some embodiments, the VH domain comprises a CDR-H1 comprising the amino acid sequence of SEQ ID NO:238, a CDR-H2 comprising the amino acid sequence of SEQ ID NO:239, and a CDR-H3 comprising the amino acid sequence of SEQ ID NO:233; and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228. In some embodiments, the VH domain comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233; and wherein the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236. In some embodiments, the VH domain comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233; and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228. In some embodiments, the VH domain comprises a CDR-H1 comprising the amino acid sequence of GFTFX₁X₂Y, wherein X₁ is S, D, E, Q, S, or A and X₂ is S, D, E, A, or Q (SEQ ID NO:271), a CDR-H2 comprising the amino acid sequence of X₁X₂X₃GX₄X₅, wherein X₁ is T, N, S, Q, E, H, R or A, X₂ is Y, W, F, or H, X₃ is A, S, Q, E, or T, X₄ is G or E, and X₅ is S or I (SEQ ID NO:272), and a CDR-H3 comprising the amino acid sequence of X₁X₂X₃YX₄WX₅X₆AX₇DX₈, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO:273); and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the VH domain comprises a CDR-H1 comprising the amino acid sequence of SEQ ID NO:240, a CDR-H2 comprising the amino acid sequence of SEQ ID NO:241, and a CDR-H3 comprising the amino acid sequence of SEQ ID NO:242; and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO: 42). In some embodiments, the VH domain comprises a CDR-H1 comprising the amino acid sequence of SEQ ID NO:240, a CDR-H2 comprising the amino acid sequence of SEQ ID NO:244, and a CDR-H3 comprising the amino acid sequence of SEQ ID NO:242; and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the VH domain comprises an amino acid sequence that is at least 90%, at least 95%, at least 99%, or 100% identical to the sequence of SEQ ID NO:62, and wherein the VL domain comprises an amino acid sequence that is at least 90%, at least 95%, at least 99%, or 100% identical to the sequence of SEQ ID NO:63. In some embodiments, the VH domain comprises the sequence of SEQ ID NO:62, and wherein the VL domain comprises the sequence of SEQ ID NO:63. In some embodiments, the VH domain comprises an amino acid sequence that is at least 90%, at least 95%, at least 99%, or 100% identical to the sequence of SEQ ID NO:64, and wherein the VL domain comprises an amino acid sequence that is at least 90%, at least 95%, at least 99%, or 100% identical to the sequence of SEQ ID NO:65. In some embodiments, the VH domain comprises the sequence of SEQ ID NO:64, and wherein the VL domain comprises the sequence of SEQ ID NO:65. In some embodiments, the VH domain comprises an amino acid sequence that is at least 90%, at least 95%, at least 99%, or 100% identical to the sequence of SEQ ID NO:66, and wherein the VL domain comprises an amino acid sequence that is at least 90%, at least 95%, at least 99%, or 100% identical to the sequence of SEQ ID NO:67. In some embodiments, the VH domain comprises the sequence of SEQ ID NO:66, and wherein the VL domain comprises the sequence of SEQ ID NO:67. In some embodiments, the VH domain comprises an amino acid sequence that is at least 90%, at least 95%, at least 99%, or 100% identical to the sequence of SEQ ID NO:68, and wherein the VL domain comprises an amino acid sequence that is at least 90%, at least 95%, at least 99%, or 100% identical to the sequence of SEQ ID NO:69. In some embodiments, the VH domain comprises the sequence of SEQ ID NO:68, and wherein the VL domain comprises the sequence of SEQ ID NO:69. In some embodiments, the VH domain comprises an amino acid sequence that is at least 90%, at least 95%, at least 99%, or 100% identical to the sequence of SEQ ID NO:70, and wherein the VL domain comprises an amino acid sequence that is at least 90%, at least 95%, at least 99%, or 100% identical to the sequence of SEQ ID NO:71. In some embodiments, the VH domain comprises the sequence of SEQ ID NO:70, and wherein the VL domain comprises the sequence of SEQ ID NO:71. In some embodiments, the VH domain comprises an amino acid sequence that is at least 90%, at least 95%, at least 99%, or 100% identical to the sequence of SEQ ID NO:72, and wherein the VL domain comprises an amino acid sequence that is at least 90%, at least 95%, at least 99%, or 100% identical to the sequence of SEQ ID NO:73. In some embodiments, the VH domain comprises the sequence of SEQ ID NO:72, and wherein the VL domain comprises the sequence of SEQ ID NO:73. In some embodiments, the VH domain comprises an amino acid sequence that is at least 90%, at least 95%, at least 99%, or 100% identical to the sequence of SEQ ID NO:245; and wherein the VL domain comprises an amino acid sequence that is at least 90%, at least 95%, at least 99%, or 100% identical to the sequence of SEQ ID NO:246. In some embodiments, the VH domain comprises the sequence of SEQ ID NO:245; and wherein the VL domain comprises the sequence of SEQ ID NO:246. In some embodiments, the VH domain comprises an amino acid sequence that is at least 90%, at least 95%, at least 99%, or 100% identical to the sequence of SEQ ID NO:251, and wherein the VL domain comprises an amino acid sequence that is at least 90%, at least 95%, at least 99%, or 100% identical to the sequence of SEQ ID NO:252. In some embodiments, the VH domain comprises the sequence of SEQ ID NO:251, and wherein the VL domain comprises the sequence of SEQ ID NO:252. In some embodiments, the VH domain comprises an amino acid sequence that is at least 90%, at least 95%, at least 99%, or 100% identical to the sequence of SEQ ID NO:253; and wherein the VL domain comprises an amino acid sequence that is at least 90%, at least 95%, at least 99%, or 100% identical to the sequence of SEQ ID NO:254. In some embodiments, the VH domain comprises the sequence of SEQ ID NO:253; and wherein the VL domain comprises the sequence of SEQ ID NO:254. In some embodiments, the VH domain comprises an amino acid sequence that is at least 90%, at least 95%, at least 99%, or 100% identical to the sequence of SEQ ID NO:247; and wherein the VL domain comprises an amino acid sequence that is at least 90%, at least 95%, at least 99%, or 100% identical to the sequence of SEQ ID NO:248. In some embodiments, the VH domain comprises the sequence of SEQ ID NO:247; and wherein the VL domain comprises the sequence of SEQ ID NO:248. In some embodiments, the VH domain comprises an amino acid sequence that is at least 90%, at least 95%, at least 99%, or 100% identical to the sequence of SEQ ID NO:249, and wherein the VL domain comprises an amino acid sequence that is at least 90%, at least 95%, at least 99%, or 100% identical to the sequence of SEQ ID NO:250. In some embodiments, the VH domain comprises the sequence of SEQ ID NO:249, and wherein the VL domain comprises the sequence of SEQ ID NO:250. In some embodiments, the VH domain comprises an amino acid sequence that is at least 90%, at least 95%, at least 99%, or 100% identical to the sequence of SEQ ID NO:255; and wherein the VL domain comprises an amino acid sequence that is at least 90%, at least 95%, at least 99%, or 100% identical to the sequence of SEQ ID NO:256. In some embodiments, the VH domain comprises the sequence of SEQ ID NO:255; and wherein the VL domain comprises the sequence of SEQ ID NO:256. In some embodiments, the VH domain comprises an amino acid sequence that is at least 90%, at least 95%, at least 99%, or 100% identical to the sequence of SEQ ID NO:257; and wherein the VL domain comprises an amino acid sequence that is at least 90%, at least 95%, at least 99%, or 100% identical to the sequence of SEQ ID NO:258. In some embodiments, the VH domain comprises the sequence of SEQ ID NO:257; and wherein the VL domain comprises the sequence of SEQ ID NO:258. In some embodiments, the VH domain comprises an amino acid sequence that is at least 90%, at least 95%, at least 99%, or 100% identical to the sequence of SEQ ID NO:58; and wherein the VL domain comprises an amino acid sequence that is at least 90%, at least 95%, at least 99%, or 100% identical to the sequence of SEQ ID NO:59. In some embodiments, the VH domain comprises the sequence of SEQ ID NO:58; and wherein the VL domain comprises the sequence of SEQ ID NO:59. In some embodiments, the VH domain comprises an amino acid sequence that is at least 90%, at least 95%, at least 99%, or 100% identical to the sequence of SEQ ID NO:185; and wherein the VL domain comprises an amino acid sequence that is at least 90%, at least 95%, at least 99%, or 100% identical to the sequence of SEQ ID NO: 186. In some embodiments, the VH domain comprises the sequence of SEQ ID NO: 185; and wherein the VL domain comprises the sequence of SEQ ID NO:186.

In some embodiments, the fusion protein comprises four polypeptide chains, wherein: (1) the first polypeptide chain comprises the amino acid sequence of SEQ ID NO:113, the second polypeptide chain comprises the amino acid sequence of SEQ ID NO:114, the third polypeptide chain comprises the amino acid sequence of SEQ ID NO:115, and the fourth polypeptide chain comprises the amino acid sequence of SEQ ID NO:113; (2) the first polypeptide chain comprises the amino acid sequence of SEQ ID NO:113, the second polypeptide chain comprises the amino acid sequence of SEQ ID NO:114, the third polypeptide chain comprises the amino acid sequence of SEQ ID NO:116, and the fourth polypeptide chain comprises the amino acid sequence of SEQ ID NO: 113; (3) the first polypeptide chain comprises the amino acid sequence of SEQ ID NO:117, the second polypeptide chain comprises the amino acid sequence of SEQ ID NO:118, the third polypeptide chain comprises the amino acid sequence of SEQ ID NO:119, and the fourth polypeptide chain comprises the amino acid sequence of SEQ ID NO: 117; (4) the first polypeptide chain comprises the amino acid sequence of SEQ ID NO: 117, the second polypeptide chain comprises the amino acid sequence of SEQ ID NO:118, the third polypeptide chain comprises the amino acid sequence of SEQ ID NO:120, and the fourth polypeptide chain comprises the amino acid sequence of SEQ ID NO: 117; (5) the first polypeptide chain comprises the amino acid sequence of SEQ ID NO:121, the second polypeptide chain comprises the amino acid sequence of SEQ ID NO:122, the third polypeptide chain comprises the amino acid sequence of SEQ ID NO:123, and the fourth polypeptide chain comprises the amino acid sequence of SEQ ID NO: 121; (6) the first polypeptide chain comprises the amino acid sequence of SEQ ID NO: 121, the second polypeptide chain comprises the amino acid sequence of SEQ ID NO:122, the third polypeptide chain comprises the amino acid sequence of SEQ ID NO:124, and the fourth polypeptide chain comprises the amino acid sequence of SEQ ID NO: 121; (7) the first polypeptide chain comprises the amino acid sequence of SEQ ID NO:125, the second polypeptide chain comprises the amino acid sequence of SEQ ID NO:126, the third polypeptide chain comprises the amino acid sequence of SEQ ID NO:127, and the fourth polypeptide chain comprises the amino acid sequence of SEQ ID NO: 125; (8) the first polypeptide chain comprises the amino acid sequence of SEQ ID NO: 125, the second polypeptide chain comprises the amino acid sequence of SEQ ID NO:126, the third polypeptide chain comprises the amino acid sequence of SEQ ID NO:128, and the fourth polypeptide chain comprises the amino acid sequence of SEQ ID NO: 125; (9) the first polypeptide chain comprises the amino acid sequence of SEQ ID NO:129, the second polypeptide chain comprises the amino acid sequence of SEQ ID NO:130, the third polypeptide chain comprises the amino acid sequence of SEQ ID NO:131, and the fourth polypeptide chain comprises the amino acid sequence of SEQ ID NO: 129; (10) the first polypeptide chain comprises the amino acid sequence of SEQ ID NO: 129, the second polypeptide chain comprises the amino acid sequence of SEQ ID NO:130, the third polypeptide chain comprises the amino acid sequence of SEQ ID NO:132, and the fourth polypeptide chain comprises the amino acid sequence of SEQ ID NO: 129; (11) the first polypeptide chain comprises the amino acid sequence of SEQ ID NO: 133, the second polypeptide chain comprises the amino acid sequence of SEQ ID NO:134, the third polypeptide chain comprises the amino acid sequence of SEQ ID NO: 135, and the fourth polypeptide chain comprises the amino acid sequence of SEQ ID NO: 133; (12) the first polypeptide chain comprises the amino acid sequence of SEQ ID NO: 133, the second polypeptide chain comprises the amino acid sequence of SEQ ID NO:134, the third polypeptide chain comprises the amino acid sequence of SEQ ID NO:136, and the fourth polypeptide chain comprises the amino acid sequence of SEQ ID NO: 133; (13) the first polypeptide chain comprises the amino acid sequence of SEQ ID NO:137, the second polypeptide chain comprises the amino acid sequence of SEQ ID NO:138, the third polypeptide chain comprises the amino acid sequence of SEQ ID NO:139, and the fourth polypeptide chain comprises the amino acid sequence of SEQ ID NO: 137; (14) the first polypeptide chain comprises the amino acid sequence of SEQ ID NO: 137, the second polypeptide chain comprises the amino acid sequence of SEQ ID NO:138, the third polypeptide chain comprises the amino acid sequence of SEQ ID NO:140, and the fourth polypeptide chain comprises the amino acid sequence of SEQ ID NO: 137; (15) the first polypeptide chain comprises the amino acid sequence of SEQ ID NO:141, the second polypeptide chain comprises the amino acid sequence of SEQ ID NO:142, the third polypeptide chain comprises the amino acid sequence of SEQ ID NO:143, and the fourth polypeptide chain comprises the amino acid sequence of SEQ ID NO: 141; (16) the first polypeptide chain comprises the amino acid sequence of SEQ ID NO: 141, the second polypeptide chain comprises the amino acid sequence of SEQ ID NO:142, the third polypeptide chain comprises the amino acid sequence of SEQ ID NO:144, and the fourth polypeptide chain comprises the amino acid sequence of SEQ ID NO: 141; (17) the first polypeptide chain comprises the amino acid sequence of SEQ ID NO:145, the second polypeptide chain comprises the amino acid sequence of SEQ ID NO:146, the third polypeptide chain comprises the amino acid sequence of SEQ ID NO:147, and the fourth polypeptide chain comprises the amino acid sequence of SEQ ID NO: 145; (18) the first polypeptide chain comprises the amino acid sequence of SEQ ID NO: 145, the second polypeptide chain comprises the amino acid sequence of SEQ ID NO:146, the third polypeptide chain comprises the amino acid sequence of SEQ ID NO:148, and the fourth polypeptide chain comprises the amino acid sequence of SEQ ID NO: 145; (19) the first polypeptide chain comprises the amino acid sequence of SEQ ID NO:149, the second polypeptide chain comprises the amino acid sequence of SEQ ID NO:150, the third polypeptide chain comprises the amino acid sequence of SEQ ID NO:151, and the fourth polypeptide chain comprises the amino acid sequence of SEQ ID NO: 149; (20) the first polypeptide chain comprises the amino acid sequence of SEQ ID NO: 149, the second polypeptide chain comprises the amino acid sequence of SEQ ID NO:150, the third polypeptide chain comprises the amino acid sequence of SEQ ID NO:152, and the fourth polypeptide chain comprises the amino acid sequence of SEQ ID NO: 149; (21) the first polypeptide chain comprises the amino acid sequence of SEQ ID NO:153, the second polypeptide chain comprises the amino acid sequence of SEQ ID NO:154, the third polypeptide chain comprises the amino acid sequence of SEQ ID NO:155, and the fourth polypeptide chain comprises the amino acid sequence of SEQ ID NO: 153; (22) the first polypeptide chain comprises the amino acid sequence of SEQ ID NO: 153, the second polypeptide chain comprises the amino acid sequence of SEQ ID NO:154, the third polypeptide chain comprises the amino acid sequence of SEQ ID NO:156, and the fourth polypeptide chain comprises the amino acid sequence of SEQ ID NO: 153; (23) the first polypeptide chain comprises the amino acid sequence of SEQ ID NO:157, the second polypeptide chain comprises the amino acid sequence of SEQ ID NO:158, the third polypeptide chain comprises the amino acid sequence of SEQ ID NO:159, and the fourth polypeptide chain comprises the amino acid sequence of SEQ ID NO: 157; or (24) the first polypeptide chain comprises the amino acid sequence of SEQ ID NO: 157, the second polypeptide chain comprises the amino acid sequence of SEQ ID NO:158, the third polypeptide chain comprises the amino acid sequence of SEQ ID NO:160, and the fourth polypeptide chain comprises the amino acid sequence of SEQ ID NO: 157.

In some embodiments, the fusion protein comprises a dimer of two mutant IL-10 polypeptides, and wherein one of the two mutant IL-10 polypeptides is fused to the antigen binding molecule. In some embodiments, the fusion protein comprises two polypeptides, each comprising an antigen binding site, and wherein one mutant IL-10 polypeptide is fused to each of the polypeptides. In some embodiments, the fusion protein comprises a mutant IL-10 monomer polypeptide, and wherein the mutant IL-10 monomer polypeptide is fused to the antigen binding molecule. In some embodiments, the mutant IL-10 polypeptide is fused to the antigen binding molecule directly or via linker.

In some embodiments, the antigen binding molecule comprises two antibody heavy chain polypeptides comprising a structure according to formula [I], from N-terminus to C-terminus:

VH-CH1-hinge-CH2-CH3 [I]

and two antibody light chain polypeptides comprising a structure according to formula [II], from N-terminus to C-terminus:

VL-CL [II]

wherein VH is an antibody heavy chain variable (VH) domain, wherein CH1 is an antibody CH1 domain, wherein hinge is an antibody hinge domain, wherein CH2 is an antibody CH2 domain, wherein CH3 is an antibody CH3 domain, wherein VL is an antibody light chain variable (VL) domain, wherein CL is an antibody constant light chain domain, and wherein VH/VL forms an antigen binding site. In some embodiments, the fusion protein comprises two mutant IL-10 polypeptides associated in a dimer; and wherein the N-terminus of one of the two mutant IL-10 polypeptides is fused to the C-terminus of one of the two CH3 domains directly or via linker. In some embodiments, the fusion protein comprises two mutant IL-10 polypeptides associated in a dimer; and wherein the N-terminus of a first of the two mutant IL-10 polypeptides is fused to the C-terminus of a first of the two CH3 domains directly or via linker, and the N-terminus of the second of the two mutant IL-10 polypeptides is fused to the C-terminus of the second of the two CH3 domains directly or via linker. In some embodiments, the fusion protein comprises one mutant IL-10 monomer polypeptide; and wherein the N-terminus of the mutant IL-10 monomer polypeptide is fused to the C-terminus of one of the two CH3 domains directly or via linker.

In some embodiments, the antigen binding molecule comprises a first antibody heavy chain polypeptide comprising a structure according to formula [I], from N-terminus to C-terminus:

VH-CH1-hinge-CH2-CH3 [I],

an antibody light chain polypeptide comprising a structure according to formula [II], from N-terminus to C-terminus:

VL-CL [II],

and a second antibody heavy chain polypeptide comprising a structure according to formula [III], fromN-terminus to C-terminus:

hinge-CH2-CH3 [III],

wherein VH is an antibody heavy chain variable (VH) domain, wherein CH1 is an antibody CH1 domain, wherein hinge is an antibody hinge domain, wherein CH2 is an antibody CH2 domain, wherein CH3 is an antibody CH3 domain, wherein VL is an antibody light chain variable (VL) domain, wherein CL is an antibody constant light chain domain, and wherein VH/VL forms an antigen binding site. In some embodiments, the fusion protein comprises two mutant IL-10 polypeptides associated in a dimer; and wherein the N-terminus of one of the two mutant IL-10 polypeptides is fused, directly or via linker, to one of the C-terminus of the CH3 domain of the second antibody heavy chain polypeptide or the C-terminus of the CH3 domain of the first antibody heavy chain polypeptide. In some embodiments, the fusion protein comprises two mutant IL-10 polypeptides associated in a dimer; and wherein the N-terminus of a first of the two mutant IL-10 polypeptides is fused to the C-terminus of the CH3 domain of the first antibody heavy chain polypeptide directly or via linker, and the N-terminus of the second of the two mutant IL-10 polypeptides is fused to the C-terminus of the CH3 domain of the second antibody heavy chain polypeptide directly or via linker. In some embodiments, the fusion protein comprises one mutant IL-10 monomer polypeptide; and wherein the N-terminus of the mutant IL-10 monomer polypeptide is fused, directly or via linker, to one of: the C-terminus of the CH3 domain of the second antibody heavy chain polypeptide or the C-terminus of the CH3 domain of the first antibody heavy chain polypeptide.

In some embodiments, one or both of the antibody heavy chain polypeptides comprise(s) the following amino acid substitutions: L234A, L235A, and G237A, numbering according to EU index. In some embodiments, a first of the two Fc domains comprises amino acid substitutions Y349C and T366W, and a second of the two Fc domain comprises amino acid substitutions S354C, T366S, L368A and Y407V, numbering according to EU index. In some embodiments, the linker comprises the sequence (GGGS)xGn (SEQ ID NO:74), (GGGGS)xGn (SEQ ID NO:75), (GGGGGS)xGn (SEQ ID NO:76), S(GGGS)xGn (SEQ ID NO:386), S(GGGGS)xGn (SEQ ID NO:387), or S(GGGGGS)xGn (SEQ ID NO:388), wherein x=1, 2, 3, 4, 5, 6, 7, 8, 9, 10, 11, or 12, and wherein n=0, 1, 2 or 3. In some embodiments, the linker comprises the sequence GGGGSGGGGSGGGGS (SEQ ID NO:79), SGGGGSGGGGSGGGGS (SEQ ID NO:77), or SGGGGSGGGGSGGGG (SEQ ID NO:78). In some embodiments, the antibody heavy chain polypeptide comprises a human IgG1 Fc region.

Further provided herein are one or more isolated polynucleotides encoding the mutant IL-10 polypeptide or fusion protein of any one of the above embodiments. Further provided herein are one or more vectors comprising the one or more polynucleotides of any one of the above embodiments. Further provided herein are host cells (e.g., isolated and/or recombinant host cells) comprising the one or more polynucleotides or vectors of any one of the above embodiments. Further provided herein are methods of producing a mutant IL-10 polypeptide or fusion protein, comprising culturing the host cell of any one of the above embodiments under conditions suitable for production of the polypeptide or fusion protein. In some embodiments, the methods further comprise recovering the polypeptide or fusion protein from the host cell.

Further provided herein are pharmaceutical compositions comprising the mutant IL-10 polypeptide or fusion protein of any one of the above embodiments and a pharmaceutically acceptable carrier.

Further provided herein are methods of treating cancer comprising administering to an individual with cancer an effective amount of the mutant IL-10 polypeptide, fusion protein, or composition thereof according to any one of the above embodiments. Further provided herein are the mutant IL-10 polypeptide, fusion protein, or composition thereof according to any one of the above embodiments for use as a medicament. Further provided herein are the mutant IL-10 polypeptide, fusion protein, or composition thereof according to any one of the above embodiments for use in a method of treating cancer in an individual in need thereof. Further provided herein are the mutant IL-10 polypeptide, fusion protein, or composition thereof according to any one of the above embodiments for use in manufacturing a medicament for treating cancer in an individual in need thereof. In some embodiments, the methods further comprise administering to the individual a T cell therapy, cancer vaccine, chemotherapeutic agent, IL-2 polypeptide, or immune checkpoint inhibitor (ICI). In some embodiments, the ICI is an inhibitor of PD-1, PD-L1, or CTLA-4. In some embodiments, the T cell therapy comprises a chimeric antigen receptor (CAR)-based T cell therapy, a tumor-infiltrating lymphocyte (TIL)-based therapy, or a therapy with T cells bearing a transduced TCR.

Further provided herein are methods of treating infection (e.g., chronic and/or viral infection) comprising administering to an individual in need thereof an effective amount of the mutant IL-10 polypeptide, fusion protein, or composition thereof according to any one of the above embodiments. Further provided herein are the mutant IL-10 polypeptide, fusion protein, or composition thereof according to any one of the above embodiments for use as a medicament. Further provided herein are the mutant IL-10 polypeptide, fusion protein, or composition thereof according to any one of the above embodiments for use in a method of treating infection (e.g., chronic and/or viral infection) in an individual in need thereof. Further provided herein are the mutant IL-10 polypeptide, fusion protein, or composition thereof according to any one of the above embodiments for use in manufacturing a medicament for treating infection (e.g., chronic and/or viral infection) in an individual in need thereof.

Further provided herein are methods of expanding T cells (e.g., ex vivo) comprising contacting one or more T cells (e.g., ex vivo) with an effective amount of the mutant IL-10 polypeptide, fusion protein, or composition thereof according to any one of the above embodiments. In some embodiments, the one or more T cells are tumor infiltrating lymphocytes (TILs).

It is to be understood that one, some, or all of the properties of the various embodiments described herein may be combined to form other embodiments of the present disclosure. These and other aspects of the disclosure will become apparent to one of skill in the art. These and other embodiments of the disclosure are further described by the detailed description that follows.

### BRIEF DESCRIPTION OF THE DRAWINGS

FIGS. 1A-1D show the amino acid sequences of the following polypeptides: mature IL-10 (FIG. 1A; SEQ ID NO:1), IL-10RA (FIG. 1B; SEQ ID NO:2), IL-10RB (FIG. 1C; SEQ ID NO:3), and mature monomer IL-10 (FIG. 1D; SEQ ID NO:187).

FIGS. 2A & 2B show the amino acid sequences of the wild-type mature IL-10 polypeptide (FIG. 2A; SEQ ID NO:1) and the mature monomer IL-10 (FIG. 2B; SEQ ID NO:187). “X” denotes the amino acid substituted in the sequence of wild-type IL-10 polypeptide for another amino acid to generate the mutant IL-10 polypeptides of the present disclosure.

FIGS. 3A & 3B show the amino acid sequences of the wild-type mature IL-10 polypeptide (FIG. 3A; SEQ ID NO:1) and the mature monomer IL-10 (FIG. 3B; SEQ ID NO:187). White boxes denote the residues that were substituted to modify IL-10 affinity to IL-10RA, grey shaded boxes denote the residues that were substituted to modify IL-10 affinity to IL-10RB. Amino acids that were substituted in place of wild-type residues for each position are shown.

FIG. 4A shows the general mechanism for how targeted fusions of mutant IL-10 polypeptides with CD8 antigen binding molecules and untargeted fusions with mutant IL-10 polypeptides work to stimulate cells expressing or not expressing CD8 antigens.

FIG. 4B shows the general mechanism for how targeted fusions of mutant monomer IL-10 polypeptides with CD8 antigen binding molecules and untargeted fusions with mutant monomer IL-10 polypeptides work to stimulate cells expressing or not expressing CD8 antigens.

FIGS. 5A & 5B show the activation of STAT3 by wild-type IL-10 dimer in human PBMCs (FIG. 5A) and human whole blood (FIG. 5B). STAT3 is shown for CD8+ T cells (filled squares) and monocytes (filled circles). STAT3 activation was measured by flow cytometry. STAT3 activation in both CD8 T cells and monocytes (gated as CD14+CD3−) using human PBMC was very similar to that using whole blood. Monocytes were found to be more sensitive to IL-10 than CD8+ T cells.

FIG. 6 depicts eight different fusion protein formats (formats A, B, C, D, E, F, G, and H), in accordance with some embodiments.

FIGS. 7A & 7B show STAT3 activation in human CD8+ T cells (filled squares) and monocytes (filled circles) by the fusion protein xmCD8a-IL10 wt of format A comprising the wild-type IL-10 polypeptide and a control antibody targeting mouse CD8 (xmCD8a-IL10 wt, FIG. 7A) or the fusion protein xhCD8a-IL10 wt of format A comprising the wild-type IL-10 polypeptide and an antibody targeting human CD8 (FIG. 7B). The anti-mouse CD8 antibody, xmCD8a, and the anti-human CD8 antibody, xhCD8a, were previously published (2.43 clone and OKT8 clone, respectively). The anti-mouse CD8 antibody (xmCD8a) does not bind human CD8 T cells and serves as a non-binding control. STAT3 activation in human PBMCs was measured by flow cytometry. IL-10 fusion protein of format A, xhCD8a-IL10 wt, comprising the antibody specifically binding to human CD8, preferentially activated CD8+ T cells over monocytes, while xmCD8a-IL10 wt, IL-10 fusion protein of format A, comprising the control antibody preferentially activated monocytes.

FIGS. 8A & 8B show STAT3 activation in human CD8+ T cells (filled squares) and monocytes (filled circles) by the fusion protein xmCD8a-IL10 wt in format C, comprising the wild-type IL-10 polypeptide and xmCD8a antibody (FIG. 8A), or xhCD8a-IL10 wt in format C, comprising the wild-type IL-10 polypeptide and xhCD8a antibody (FIG. 8B). Format C was not optimal for IL-10 fusion proteins comprising antibodies binding to human CD8, except at low concentrations (up to 0.01 nM), as higher concentrations did not fully activate STAT3 in CD8+ T cells (FIG. 8B).

FIGS. 9A & 9B show STAT3 activation in human CD8+ T cells (filled squares) and monocytes (filled circles) by the fusion protein xmCD8a-IL10 wt in format D, comprising the wild-type IL-10 polypeptide and xmCD8a antibody (FIG. 9A) or xhCD8a-IL10 wt in format D, comprising the wild-type IL-10 polypeptide and xhCD8a antibody (FIG. 9B). IL-10 fusion protein of format D, xhCD8a-IL10 wt, comprising the antibody specifically binding to human CD8, preferentially activated CD8+ T cells over monocytes, while xmCD8a-IL10 wt, IL-10 fusion protein of format D, comprising the control antibody, preferentially activated monocytes.

FIGS. 10A-10C show STAT3 activation in human CD8+ T cells (filled squares), monocytes (filled circles), and CD4+ T cells (filled triangles) by various fusion proteins. FIG. 10A shows STAT3 activation by xhCD8b-IL10mono in format F, comprising the mature monomer IL-10 polypeptide (SEQ ID NO:187) and xhCD8b antibody. FIG. 10B shows STAT3 activation by xhCD8b-IL10mono_RBenh in format F, comprising the monomer IL-10 polypeptide with amino acid substitutions for increased IL-10RB binding affinity and xhCD8b antibody. FIG. 10C shows STAT3 activation by xhCD8b-IL10mono_RBenh2 in format F, comprising the monomer IL-10 polypeptide with alternate amino acid substitutions for increased IL-10RB binding affinity and xhCD8b antibody. Wild-type monomer IL-10 fusion protein comprising antibodies to human CD8 preferentially activated CD8 T cells over monocytes and CD4 T cells at concentrations below 1 nM, but the degree of activation was not optimal. IL-10RB affinity-enhanced monomer IL-10 fusion protein comprising antibodies to human CD8 preferentially and effectively activated CD8 T cells over monocytes and CD4 T cells.

FIGS. 11A-11F show STAT3 activation by fusion proteins of xhCD8b antibody and various IL-10 polypeptides with amino acid substitutions for increased IL-10RB binding affinity in format F (xhCD8b-IL-10mono_RBenh #). FIGS. 11A and 11B show STAT3 activation in CD8+ T cells and monocytes for selected sets of muteins in human PBMCs. FIGS. 11C and 11D show STAT3 activation in CD8+ T cells and monocytes for additional sets of muteins in human PBMCs. FIGS. 11E and 11F show STAT3 activation in CD8+ T cells and monocytes for three sets of muteins in whole blood.

FIGS. 12A-12H show STAT3 activation by fusion proteins of xhCD8b antibody and various IL-10 polypeptides with amino acid substitutions for increased IL-10RB binding affinity and decreased IL-10RA binding affinity in format F. FIGS. 12A and 12B show STAT3 activation in CD8+ T cells and monocytes for five selected muteins in human PBMCs. FIGS. 12C and 12D show STAT3 activation in CD8+ T cells and monocytes for another five selected muteins in human PBMCs. FIGS. 12E and 12F show STAT3 activation in CD8+ T cells and monocytes for five selected muteins in human PBMCs. FIGS. 12G and 12H show STAT3 activation in CD8+ T cells and monocytes for six selected muteins in whole blood.

FIGS. 13A & 13B show STAT3 activation in CD8 T cells and monocytes, respectively, by fusion proteins of xhCD8b and either IL10mono_RBenh2 or IL10mono_RBenh2_m117. These STAT3 activation data are measured in human whole blood.

### DETAILED DESCRIPTION

### Definitions

As used in this specification and the appended claims, the singular forms “a”, “an” and “the” include plural referents unless the content clearly dictates otherwise. Thus, for example, reference to “a molecule” optionally includes a combination of two or more such molecules, and the like.

It is understood that aspects and embodiments of the present disclosure include “comprising,” “consisting,” and “consisting essentially of” aspects and embodiments.

The term “about” as used herein refers to the usual error range for the respective value readily known to the skilled person in this technical field. Reference to “about” a value or parameter herein includes (and describes) embodiments that are directed to that value or parameter per se.

“Immune cells” as used here are cells of the immune system that react to organisms or other entities that are deemed foreign to the immune system of the host. They protect the host against foreign pathogens, organisms and diseases. Immune cells, also called leukocytes, are involved in both innate and adaptive and immune responses to fight pathogens. Innate immune responses occur immediately upon exposure to pathogens without additional priming or learning processes. Adaptive immune processes require initial priming, and subsequently create memory, which in turn leads to enhanced responsiveness during subsequent encounters with the same pathogen. Innate immune cells include, but are not limited to monocytes, macrophages, dendritic cells, innate lymphoid cells (ILCs) including natural killer (NK) cells, neutrophils, megakaryocytes, eosinophils and basophils. Adaptive immune cells include B and T lymphocytes/cells. T cells subsets include, but are not limited to, alpha beta CD4+T (naïve CD4+, memory CD4+, effector memory CD4+, effector CD4+, regulatory CD4+), and alpha beta CD8+T (naïve CD8+, memory CD8+, effector memory CD8+, effector CD8+). B cell subsets include, but is not limited to, naïve B, memory B, and plasma cells. NK T cells and T gamma delta (Tγδ) cells exhibit properties of both innate and adaptive lymphocytes. In some embodiments, any of the immune cells herein are human cells.

“T cells” or “T lymphocytes” are immune cells that play a key role in the orchestration of immune responses in health and disease. Two major T cell subsets exist that have unique functions and properties: T cells that express the CD8 antigen (CD8⁺ T cells) are cytotoxic or killer T cells that can lyse target cells using the cytotoxic proteins such as granzymes and perforin; and T cells that express the CD4 antigen (CD4⁺ T cells) are helper T cells that are capable of regulating the function of many other immune cell types including that of CD8⁺ T cells, B cells, macrophages etc. Furthermore, CD4⁺ T cells are further subdivided into several subsets such as: T regulatory (Treg) cells that are capable of suppressing the immune response, and T helper 1 (Th1), T helper 2 (Th2), and T helper 17 (Th17) cells that regulate different types of immune responses by secreting immunomodulatory proteins such as cytokines. T cells recognize their targets via alpha beta T cell receptors that bind to unique antigen-specific motifs and this recognition mechanism is generally required in order to trigger their cytotoxic and cytokine-secreting functions. “Innate lymphocytes” can also exhibit properties of CD8⁺ and CD4⁺ T cells, such as the cytotoxic activity or the secretion of Th1, Th2, and Th17 cytokines. Some of these innate lymphocyte subsets include NK cells and ILC1, ILC2, and ILC3 cells; and innate-like T cells such as Tyb cells; and NK T cells. Typically, these cells can rapidly respond to inflammatory stimuli from infected or injured tissues, such as immunomodulatory cytokines, but unlike alpha beta T cells, they can respond without the need to recognize antigen-specific patterns.

“Cytokine” is a form of immunomodulatory polypeptide that mediates cross-talk between initiating/primary cells and target/effector cells. It can function as a soluble form or cell-surface associated to bind the “cytokine receptor” on target immune cells to activate signaling. “Cytokine receptor” (i.e. IL-10 receptor, IL-10R, composed of two subunits, IL-10RA and IL-10RB) as used here is the polypeptide on the cell surface that activates intracellular signaling upon binding the cytokine on the extracellular cell surface. Cytokines includes, but are not limited to, chemokines, interferons, interleukins, lymphokines, and tumor necrosis factors. Cytokines are produced by a wide range of cells, including immune cells, endothelial cells, fibroblasts, and stromal cells. A given cytokine may be produced by more than one cell type. Cytokine are pleiotropic; since the receptors are expressed on multiple immune cell subsets, one cytokine can activate the signaling pathway in multiple cells. However, depending on the cell type, the signaling events for a cytokine can result in different downstream cellular events such as activation, proliferation, survival, apoptosis, effector function and secretion of other immunomodulatory proteins.

“Amino acid” as used here refers to naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

“Polypeptide” or “protein” as used here refers to a molecule where monomers (amino acids) are linearly linked to one another by peptide bonds (also known as amide bonds). The term “polypeptide” refers to any chain of two or more amino acids and does not refer to a specific length of the product. Thus, peptides, dipeptides, tripeptides, oligopeptides, “protein”, “amino acid chain”, or any other term used to refer to a chain of two or more amino acids, are included within the definition of “polypeptide”, and the term “polypeptide” may be used instead of, or interchangeably with any of these terms. The term “polypeptide” is also intended to refer to the products of a polypeptide may be derived from a natural biological source or produced by recombinant technology but is not necessarily translated from a designated nucleic acid sequence. It may be generated in any manner, including by chemical synthesis. Polypeptides normally have a defined three-dimensional structure, but they do not necessarily have such structure. A polypeptide of the present disclosure may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides with a defined three-dimensional structure are referred to as folded, and polypeptides which do not possess a defined three-dimensional structure, but rather can adopt many different conformations and are referred to as unfolded. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. The terms “polypeptide” and “protein” also refer to modified polypeptides/proteins wherein the post-expression modification is affected including without limitation glycosylation, acetylation, phosphorylation, amidation, derivatization by known protecting/blocking groups, proteolytic cleavage, or modification by non-naturally occurring amino acids.

“Residue” as used herein is meant a position in a protein and its associated amino acid identity. For example, Leu 234 (also referred to as Leu234 or L234) is a residue at position 234 in the human antibody IgG1.

“Wild-type” herein means an amino acid sequence or a nucleotide sequence that is found in nature, including allelic variations. A wild-type protein has an amino acid sequence or a nucleotide sequence that has not been intentionally modified.

“Substitution” or “mutation” refers to a change to the polypeptide backbone wherein an amino acid occurring in the wild-type sequence of a polypeptide is substituted to another amino acid at the same position in the said polypeptide. In some embodiments, a mutation or mutations are introduced to modify polypeptide's affinity to its receptor thereby altering its activity such that it becomes different from the affinity and activity of the wild-type cognate polypeptide. Mutations can also improve polypeptide's biophysical properties. Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

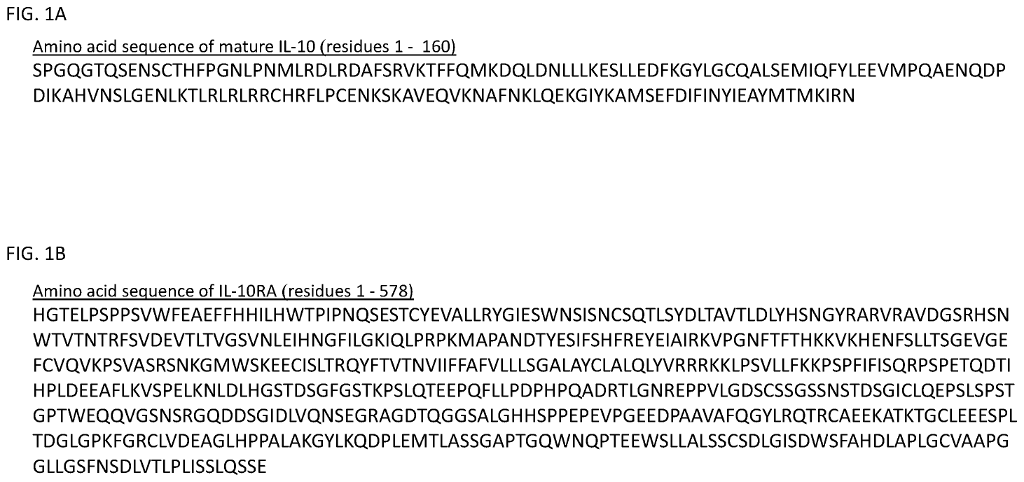

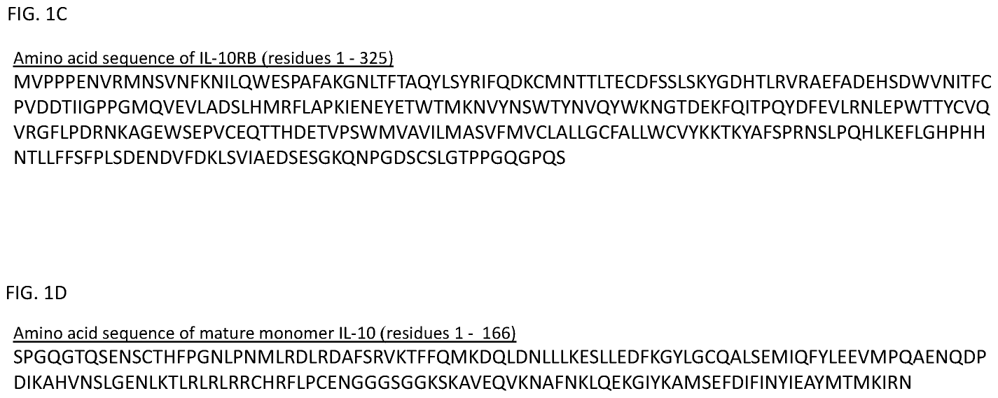

“Interleukin-10” or “IL-10” as used here refers to any native IL-10 from any vertebrate source, including mammals such as primates (e.g. humans) and rodents (e.g., mice and rats), unless otherwise indicated. IL-10 normally exist as a homodimer. “IL-10” encompasses unprocessed IL-10 as well as “mature IL-10” which is a form of IL-10 that results from processing in the cell. The sequence of “mature IL-10” is depicted in FIG. 1A. One exemplary form of unprocessed human IL-10 comprises of an additional N-terminal amino acid signal peptide attached to mature IL-10. “IL-10” also includes but is not limited to naturally occurring variants of IL-10, e.g. allelic or splice variants or variants. The amino acid sequence of an exemplary human IL-10 is described under UniProt P22301 (IL10_HUMAN).

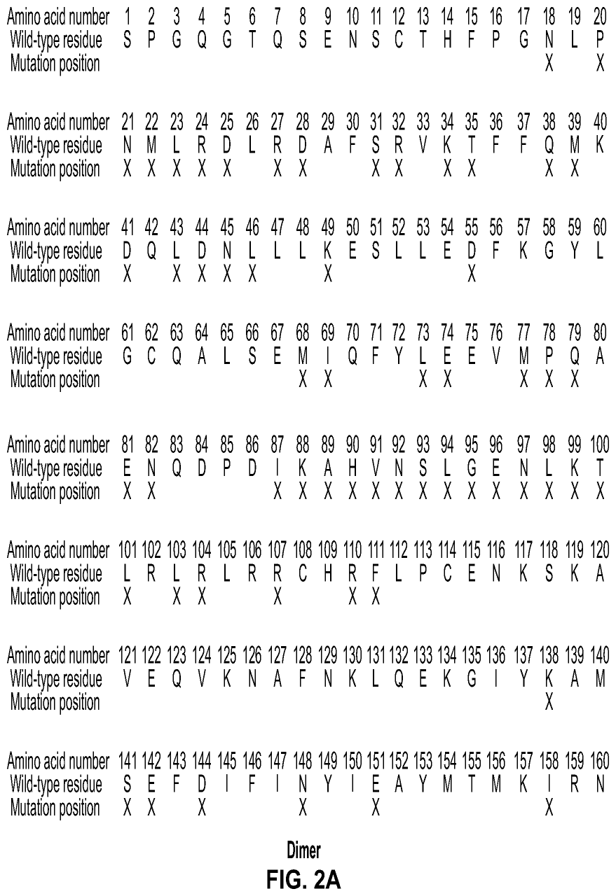

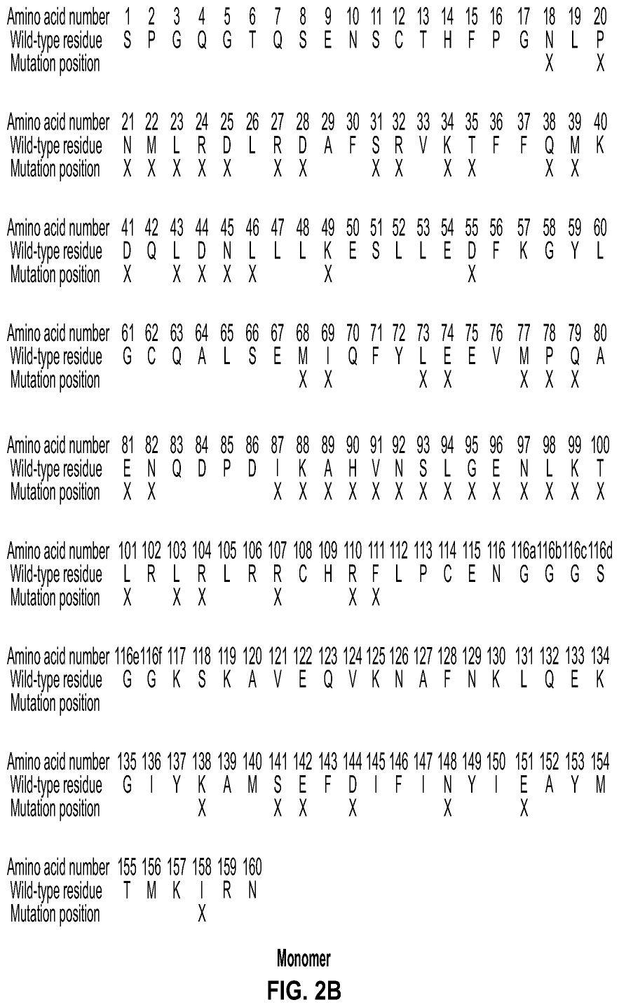

“IL-10 homodimer” or “IL-10 dimer” refers to a naturally symmetric homodimer form of wild-type IL-10 polypeptide that binds to a tetrameric IL-10 receptor (IL-10R) complex on the cell, consisting of 2 molecules of IL-10R α-chain (IL-10RA) and two molecules of the IL-10R β-chain (IL-10RB). The α-helices from each IL-10 polypeptide chain intertwine such that the first four helices of one chain (A-D) associate with the last two helices (E and F) of the other, hereby maintaining structural integrity of each domain when dimerized (Walter & Nagabhushan, Biochemistry. 1995 Sep. 26; 34(38):12118-25). “IL-10 monomer” refers to a monomeric form of IL-10 that can be generated by extending the loop that connects the swapped secondary structural elements. As described in Josephson et al, Biochemistry 1995 Sep. 26; 34(38):12118-25, insertion of 6 amino acids into the said loop was sufficient to prevent dimerization and induce IL-10 monomer formation. The resulting IL-10 monomer was biologically active and capable of binding to a single IL-10RA molecule and recruiting a single IL-10RB molecule into the signaling complex to elicit IL-10-mediated cellular responses. Therefore, inserting a short amino acid sequence or a short linker into the sequence of an IL-10 polypeptide (i.e. wild-type IL-10 or any mutant IL-10 polypeptide of the present disclosure) between loop D (ends with residue C114) and loop E (begins with residue V121) generates a “monomeric isomer” of said IL-10 polypeptide. This added amino acid sequence or linker can be inserted immediately after C114, E115, N116, K117, S118, K119, or A120. As described herein, the amino acid numbering for an IL-10 monomer polypeptide is based on the number of SEQ ID NO:1 (i.e., an IL-10 dimer polypeptide), such that the linker sequence/amino acid(s) are not counted. See, e.g., FIGS. 2B & 3B.

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity can generally be represented by the dissociation constant (K_{D}), which is the ratio of dissociation and association rate constants (koff and kon, respectively). Thus, equivalent affinities may comprise different rate constants, as long as the ratio of the rate constants remains the same. Affinity can be measured by common methods known in the art, such as enzyme-linked immunosorbent assay (ELISA), surface plasmon resonance (SPR) technologies (e.g. BIAcore), BioLayer Interferometry (BLI) technologies (e.g. Octet) and other traditional binding assays (Heeley, Endocr Res 28, 217-229 (2002).

“Binding” or “specific binding” as used here, refers the ability of a polypeptide or an antigen binding molecule to selectively interact with the receptor for the polypeptide or target antigen, respectively, and this specific interaction can be distinguished from non-targeted or undesired or non-specific interactions.

“Mutant IL-10 polypeptide” refers to an IL-10 polypeptide that has an amino acid sequence different from a wild type IL-10. For example, a mutant IL-10 polypeptide may have amino acid substitutions, deletions, and insertions. In some embodiments, a mutant IL-10 polypeptide has reduced affinity to its receptor wherein such decreased affinity results in reduced biological activity of the mutant. Reduction in affinity and thereby activity can be obtained by introducing a small number of amino acid mutations or substitutions. The mutant IL-10 polypeptides can also have other modifications to the peptide backbone, including but not limited to amino acid deletion, permutation, cyclization, disulfide bonds, or the post-translational modifications (e.g. glycosylation or altered carbohydrate) of a polypeptide, chemical or enzymatic modifications to the polypeptide (e.g. attaching PEG to the polypeptide backbone), addition of peptide tags or labels, or fusion to proteins or protein domains to generate a final construct with desired characteristics, such as reduced affinity to IL-10R. Desired activity may also include improved biophysical properties compared to the wild-type IL-10 polypeptide. Multiple modifications may be combined to achieve desired activity modification, such as reduction in affinity or improved biophysical properties. As a non-limiting example, amino acid sequences for consensus N-link glycosylation may be incorporated into the polypeptide to allow for glycosylation. Another non-limiting example is that a lysine may be incorporated onto the polypeptide to enable pegylation. In some embodiments, a mutation or mutations are introduced to the polypeptide to modify its activity by reducing its affinity to its receptor.

“Targeting moiety” and “antigen binding molecule” as used here refers in its broadest sense to a molecule that specifically binds an antigenic determinant. A targeting moiety or antigen binding molecule may be a protein, carbohydrate, lipid, or other chemical compound. It includes, but is not limited to, antibody, antibody fragments (Chames et al, 2009; Chan & Carter, 2010; Leavy, 2010; Holliger & Hudson, 2005), scaffold antigen binding proteins (Gebauer and Skerra, 2009; Stumpp et al, 2008), single domain antibodies (sdAb), minibodies (Tramontano et al, 1994), the variable domain of heavy chain antibodies (nanobody, VHH), the variable domain of the new antigen receptors (VNAR), carbohydrate binding domains (CBD) (Blake et al, 2006), collagen binding domain (Knight et al, 2000), lectin binding proteins (Tetranectin), collagen binding proteins, adnectin/fibronectin (Lipovsek, 2011), a serum transferrin (trans-body), Evibody, Protein A-derived molecule, such as Z-domain of Protein A (Affibody) (Nygren et al, 2008), an A-domain (Avimer/Maxibody), alphabodies (WO2010066740), Avimer/Maxibody, designed ankyrin-repeat domains (DARPins) (Stumpp et al, 2008), anticalins (Skerra et al, 2008), a human gamma-crystallin or ubiquitin (Affilin molecules), a kunitz type domain of human protease inhibitors, knottins (Kolmar et al, 2008), linear or constrained peptide with or without fusion to extend half-life e.g. (Fc fusion—Peptibody) (Rentero Rebollo & Heinis, 2013; EP 1144454 B2; Shimamoto et al, 2012; U.S. Pat. No. 7,205,275 B2), constrained bicyclic peptides (US 2018/0200378 A1), aptamer, engineered CH2 domains (nanoantibodies; Dimitrov, 2009)) and engineered CH3 domain “Fcab” domains (Wozniak-Knopp et al, 2010).

The terms “antibody” and “immunoglobulin” are used interchangeably and herein are used in the broadest sense and encompasses various antibody structures, including but not limited to monoclonal antibodies (e.g., full length or intact monoclonal antibodies), polyclonal antibodies, multispecific antibodies (e.g. bispecific antibodies), antibody fragments and single domain antibody (as described in greater detail herein), so long as they exhibit the desired antigen binding activity.

In some embodiments, antibodies (immunoglobulins) refer to a protein having a structure substantially similar to a native antibody structure. “Native antibodies” refer to naturally occurring immunoglobulin molecules with varying structures. For example, native immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The subunit structures and three-dimensional configurations of the different classes of immunoglobulins are well known and described generally, for example, in Abbas et al., 2000, Cellular and Mol, and Kindt et al., Kuby Immunology, 6th ed., W.H. Freeman and Co., page 91 (2007). Antibodies (immunoglobulins) are assigned to different classes, depending on the amino acid sequences of the heavy chain constant domains. There are five major classes of antibodies: α (IgA), δ (IgD), ϵ (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

“Fc” or “Fc region” or “Fc domain” as used herein refers to the C-terminal region of an antibody heavy chain that contains at least a portion of the constant region. The term includes native sequence Fc regions and variant Fc regions. An Fc can refer to the last two constant region immunoglobulin domains (e.g., CH2 and CH3) of IgA, IgD, and IgG, the last three constant region immunoglobulin domains of IgE and IgM, and optionally, all or a portion of the flexible hinge N-terminal to these domains. For IgA and IgM, Fc may include the J chain. An IgG Fc region comprises an IgG CH2 and an IgG CH3 domain and in some cases, inclusive of the hinge. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991. The “hinge” region usually extends from amino acid residue at about position 216 to amino acid residue at about position 230. The hinge region herein may be a native hinge domain or variant hinge domain. The “CH2 domain” of a human IgG Fc region usually extends from an amino acid residue at about position 231 to an amino acid residue at about position 340. The CH2 domain herein may be a native sequence CH2 domain or variant CH2 domain. The “CH3 domain” comprises the stretch of residues C-terminal to a CH2 domain in an Fc region, from an amino acid residue at about position 341 to an amino acid residue at about position 447 of an IgG. The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Thus, the definition of “Fc domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g. non-denaturing chromatography, size exclusion chromatography, etc.). Human IgG Fc domains are of particular use in the present disclosure, and can be the Fc domain from human IgG1, IgG2 or IgG4.

A “variant Fc domain” or “Fc variant” or “variant Fc” contains amino acid modifications (e.g. substitution, addition, and deletion) as compared to a parental Fc domain. The term also includes naturally occurring allelic variants of the Fc region of an immunoglobulin. In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. For example, one or more amino acids can be deleted from the N-terminus or C-terminus of the Fc region of an immunoglobulin without substantial loss of biological function. Additionally, as discussed herein, the variant Fc domains herein still retain the ability to form a dimer with another Fc domain as measured using known techniques as described herein, such as non-denaturing gel electrophoresis.

“Fc gamma receptor”, “FcγR” or “Fc gamma R” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “effector function” as used herein is meant a biochemical event that results from the interaction of an antibody Fc region with an Fc receptor or ligand, which vary with the antibody isotype. Effector functions include but are not limited to antibody-dependent cell-mediated cytotoxicity (ADCC), antibody-dependent cell-mediated phagocytosis (ADCP), complement-dependent cytotoxicity (CDC), cytokine secretion, immune complex-mediated antigen uptake by antigen presenting cells, down regulation of cell surface receptors (e.g. B cell receptor), and B cell activation. “Antibody-dependent cell-mediated cytotoxicity” or “ADCC” refer to a cell-mediated reaction in which nonspecific cytotoxic cells that express FcRs (such as Natural Killer (NK) cells, neutrophils, and macrophages) recognize bound antibody on a target cell and subsequently cause lysis of the target cell. ADCC is correlated with binding to FcγRIIIa; increased binding to FcγRIIIa leads to an increase in ADCC activity. To assess ADCC activity of a molecule of interest, an in vitro ADCC assay such as that described in U.S. Pat. No. 5,500,362 or 5,821,337 may be performed. “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

“Fc null” and “Fc null variant” are used interchangeably and used herein to describe a modified Fc which have reduced or abolished effector functions. Such Fc null or Fc null variant have reduced or abolished to FcγRs and/or complement receptors. In some embodiments, such Fc null or Fc null variant has abolished effector functions. Exemplary methods for the modification include but not limited to chemical alteration, amino acid residue substitution, insertion and deletions. Exemplary amino acid positions on Fc molecules where one or more modifications were introduced to decrease effector function of the resulting variant (numbering based on the EU numbering scheme) at position i) IgG1: C220, C226, C229, E233, L234, L235, G237, P238, S239 D265, S267, N297, L328, P331, K322, A327 and P329, ii) IgG2: V234, G237, D265, H268, N297, V309, A330, A331, K322 and iii) IgG4: L235, G237, D265 and E318. Exemplary Fc molecules having decreased effector function include those having one or more of the following substitutions: i) IgG1: N297A, N297Q, N297G, D265A/N297A, D265A/N297Q, C220S/C226S/C229S/P238S, S267E/L328F, C226S/C229S/E233P/L234V/L235A, L234F/L235E/P33IS, L234A/L235A, L234A/L235A/G237A, L234A/L235A/G237A/K322A, L234A/L235A/G237A/A330S/A331S, L234A/L235A/P329G, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, L234A/L235A/G237deleted; ii) IgG2: A330S/A331S, V234A/G237A, V234A/G237A/D265A, D265A/A330S/A331S, V234A/G237A/D265A/A330S/A331S, and H268Q/V309L/A330S/A331S; iii) IgG4: L235A/G237A/E318A, D265A, L235A/G237A/D265A and L235A/G237A/D265A/E318A.

“Epitope” as used herein refers to a determinant capable of specific binding to the variable region of an antibody molecule known as a paratope. Epitopes are groupings of molecules such as amino acids or sugar side chains and usually have specific structural characteristics, as well as specific charge characteristics. A single antigen may have more than one epitope. The epitope may comprise amino acid residues directly involved in the binding and other amino acid residues, which are not directly involved in the binding, such as amino acid residues which are effectively blocked by the antigen binding peptide (in other words, the amino acid residue is within the footprint of the antigen binding peptide). Epitopes may be either conformational or linear. An epitope typically includes at least 3, and more usually, at least 5 or 8-10 amino acids. Antibodies that recognize the same epitope can be verified in a simple immunoassay showing the ability of one antibody to block the binding of another antibody to a target antigen, for example “binning”.

“Linker” as used herein refers to a molecule that connect two polypeptide chains. Linker can be a polypeptide linker or a synthetic chemical linker (for example, see disclosed in Protein Engineering, 9(3), 299-305, 1996). The length and sequence of the polypeptide linkers is not particularly limited and can be selected according to the purpose by those skilled in the art. Polypeptide linker comprises one or more amino acids. In some embodiments, the polypeptide linker is a peptide with a length of at least 5 amino acids, in some embodiments with a length of 5 to 100, or 10 to 50 amino acids. In one embodiment, said peptide linker is G, S, GS, SG, SGG, GGS, and GSG (with G=glycine and S=serine). In another embodiment, said peptide linker is (GGGS)xGn (SEQ ID NO:74), (GGGGS)xGn (SEQ ID NO:75), (GGGGGS)xGn (SEQ ID NO:76), S(GGGS)xGn (SEQ ID NO:386), S(GGGGS)xGn (SEQ ID NO:387), or S(GGGGGS)xGn (SEQ ID NO:388), with x=1, 2, 3, 4, 5, 6, 7, 8, 9, 10, 11, or 12 and n=0, 1, 2 or 3. In some embodiments, said linker is (GGGGS)xGn with x=2, 3, or 4 and n=0 (SEQ ID NO: 85); in some embodiments the said linker is (GGGGS)xGn with x=3 and n=0 (SEQ ID NO:86). In some embodiments, the linker comprises the sequence GGGGSGGGGSGGGGS (SEQ ID NO:79), SGGGGSGGGGSGGGGS (SEQ ID NO:77), or SGGGGSGGGGSGGGG (SEQ ID NO:78). Synthetic chemical linkers include crosslinking agents that are routinely used to crosslink peptides, for example, N-hydroxy succinimide (NHS), disuccinimidyl suberate (DSS), bis(succinimidyl) suberate (BS3), dithiobis(succinimidyl propionate) (DSP), dithiobis(succinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis[2-(succinimidoxycarbonyloxy)ethyl]sulfone (BSOCOES), and bis[2-(succinimidoxycarbonyloxy)ethyl] sulfone (sulfo-BSOCOES).

“Percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs [0279] to [0280] of US Pub. No. 20160244525, hereby incorporated by reference.

The term “polynucleotide” refers to an isolated nucleic acid molecule or construct, e.g. messenger RNA (mRNA), virally-derived RNA, or plasmid DNA (pDNA) encoding the polypeptides of the present disclosure. A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide. In some aspects, one or more vectors (particularly expression vectors) comprising such nucleic acids are provided. In one aspect, a method for making a polypeptide of the present disclosure is provided, wherein the methods comprises culturing a host cell comprising a nucleic acid encoding the polypeptide under conditions suitable for expression of the polypeptide and recovering the polypeptide from the host cell. “Recombinant” means the proteins are generated using recombinant nucleic acid techniques in exogeneous host cells. Recombinantly produced proteins expressed in host cells are considered isolated for the purpose of the present disclosure, as are native or recombinant proteins which have been separated, fractionated, or partially or substantially purified by any suitable technique.

“Isolated,” when used to describe the various polypeptides disclosed herein, means a polypeptide that has been identified and separated and/or recovered from a cell or cell culture from which it was expressed. Typically, an isolated polypeptide will be purified by at least one purification step. There is no required level of purity; “purification” or “purified” refers to increase of the target protein concentration relative to the concentration of contaminants in a composition as compared to the starting material. An “isolated protein,” as used herein refers to a target protein which is substantially free of other proteins having different binding specificities.

The terms “cancer” refers the physiological condition in mammals that is typically characterized by unregulated and abnormal cell growth with the potential to invade or spread to other parts of the body. Examples of cancer include but are not limited to, carcinoma, lymphoma, blastoma, sarcoma, and leukemia. More particular examples of such cancers include lung cancer, small-cell lung cancer, non-small cell lung (NSCL) cancer, bronchioloalviolar cell lung cancer, squamous cell cancer, adenocarcinoma of the lung, squamous carcinoma of the lung, cancer of the peritoneum, head and neck cancer, bone cancer, pancreatic cancer, skin cancer, cancer of the head or neck, cutaneous or intraocular melanoma, thyroid cancer, uterine cancer, gastrointestinal cancer, ovarian cancer, rectal cancer, cancer of the anal region, stomach cancer, gastric cancer, colon cancer, breast cancer, endometrial carcinoma, uterine cancer, carcinoma of the fallopian tubes, carcinoma of the cervix, carcinoma of the vagina, vulval cancer, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, bladder cancer, liver cancer, hepatoma, hepatocellular cancer, cervical cancer, salivary gland carcinoma, biliay cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

### Mutant IL-10 Polypeptides

In some embodiments, the present disclosure relates to mutant IL-10 polypeptides, and fusion proteins thereof. In some embodiments, the mutant IL-10 polypeptides comprise one or more mutations (e.g., relative to SEQ ID NO:1) that increase binding affinity to an IL-10RB polypeptide (e.g., comprising the sequence of SEQ ID NO:3). In some embodiments, the mutant IL-10 polypeptides comprise one or more mutations (e.g., relative to SEQ ID NO:1) that decrease binding affinity to an IL-10RA polypeptide (e.g., comprising the sequence of SEQ ID NO:2). In some embodiments, the mutant IL-10 polypeptides comprise one or more mutations (e.g., relative to SEQ ID NO:1) that increase binding affinity to an IL-10RB polypeptide (e.g., comprising the sequence of SEQ ID NO:3) and comprise one or more mutations (e.g., relative to SEQ ID NO:1) that decrease binding affinity to an IL-10RA polypeptide (e.g., comprising the sequence of SEQ ID NO:2). In some embodiments, the mutant IL-10 polypeptide comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the amino acid sequence of either wild-type mature IL-10 depicted in FIG. 1A, or the mature monomer IL-10 depicted in FIG. 1D.

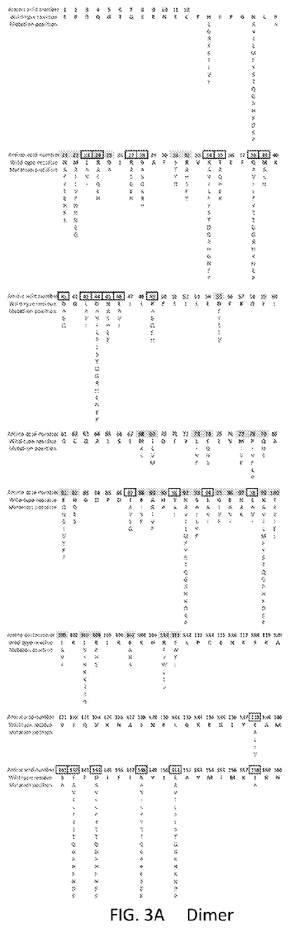

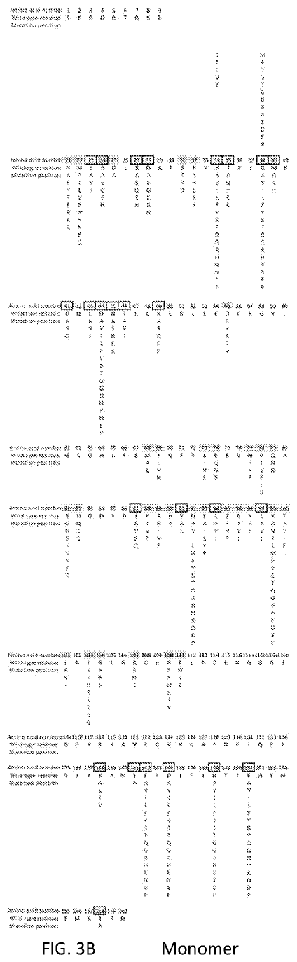

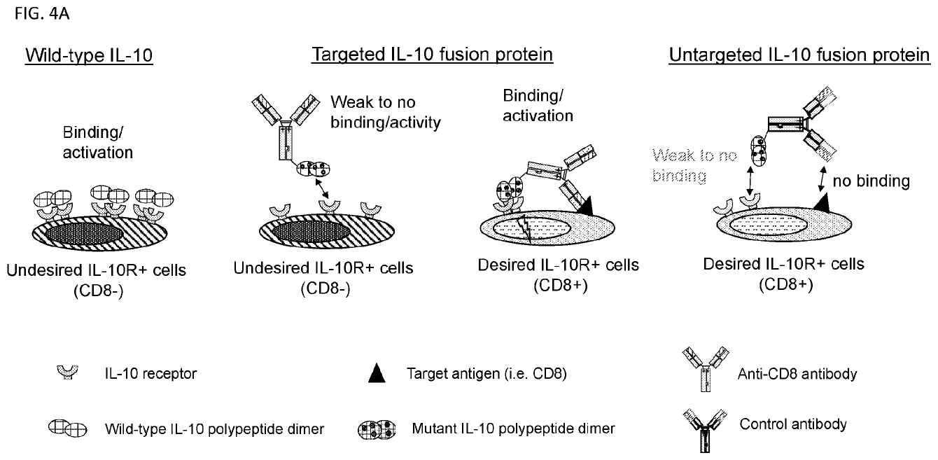

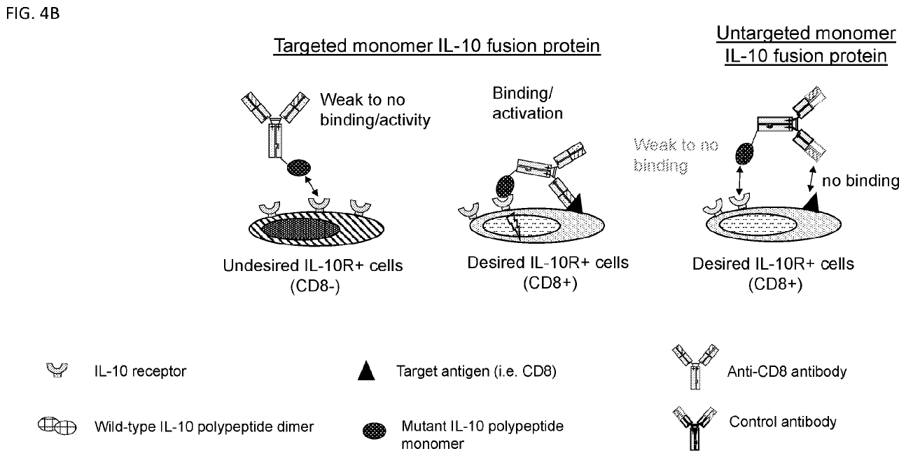

In some embodiments, the mutant IL-10 polypeptide comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the amino acid sequence of either wild-type mature IL-10 depicted in FIG. 1A, or the mature monomer IL-10 depicted in FIG. 1D. In some embodiments, the mutant IL-10 polypeptide: i) exhibits reduced binding affinity to IL-10RA polypeptide having an amino acid sequence depicted in FIG. 1B; and ii) has one or more amino acid substitutions relative to the amino acid sequence of the wild-type IL-10 polypeptide as depicted in FIG. 1A or the mature monomer IL-10 depicted in FIG. 1D and selected from a group of: P20, L23, R24, R27, D28, K34, T35, Q38, M39, D41, L43, D44, N45, L46, K49, I87, V91, L94, L98, K138, S141, E142, D144, N148, E151, and I158, as depicted in FIGS. 2A-3B. In some embodiments, the mutant IL-10 polypeptide of the present disclosure exhibits reduced binding affinity by 50% or more to IL-10RA polypeptide having an amino acid sequence depicted in FIG. 1B. Differences in binding affinity of the wild-type and mutant IL-10 polypeptides to IL-10RA are measured in standard SPR assays that measure affinity of protein-protein interactions familiar to those skilled in the art.

In some embodiments, the mutant IL-10 polypeptide comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the amino acid sequence of either wild-type mature IL-10 depicted in FIG. 1A, or the mature monomer IL-10 depicted in FIG. 1D. In some embodiments, the mutant IL-10 polypeptide: i) exhibits increased binding affinity to IL-10RB polypeptide having an amino acid sequence depicted in FIG. 1C; and ii) has one or more amino acid substitutions relative to the amino acid sequence of the wild-type mature IL-10 polypeptide as depicted in FIG. 1A and selected from a group of: N18, N21, M22, R24, D25, D28, S31, R32, D55, M68, I69, L73, E74, M77, P78, Q79, E81, N82, K88, A89, H90, N92, S93, G95, E96, N97, K99, T100, L101, L103, R104, R107, R110 and F111 (FIGS. 2A-3B). In yet other embodiments, the mutant IL-10 polypeptide exhibits increased binding affinity by 150% or more to IL-10RB polypeptide having an amino acid sequence depicted in FIG. 1C.

The location of possible amino acid substitutions in the sequence of the wild-type mature IL-10 polypeptide is depicted in FIG. 2. In some embodiments, denoted amino acids in the sequence of the wild-type mature IL-10 polypeptide were substituted for alanine or another amino acid, as depicted in FIG. 3.

In some embodiments, the mutant IL-10 polypeptides also contain other modifications, including but not limited to mutations and deletions, that provide additional advantages such as improved biophysical properties. Improved biophysical properties include but are not limited to improved thermostability, aggregation propensity, acid reversibility, viscosity, and production in a mammalian or bacterial or yeast cell.

In some embodiments, the mutant IL-10 polypeptide is a monomer, e.g., as described herein. For example, see SEQ ID NO:187 as shown in FIGS. 1D, 2B, & 3B. In some embodiments, the mutant IL-10 monomer polypeptide comprises amino acid substitutions N18I, N92I, K99N and F111L, e.g., as shown in SEQ ID NO:188 in Table 4A below. In some embodiments, the mutant IL-10 monomer polypeptide comprises an amino acid sequence listed in Table 4A. In some embodiments, the mutant IL-10 monomer polypeptide comprises an amino acid sequence selected from the group consisting of SEQ ID Nos:188-201. In some embodiments, the mutant IL-10 monomer polypeptide comprises amino acid substitutions N18I, N92I, K99N and F111L and one or more further amino acid substitutions. In some embodiments, the one or more further amino acid substitutions are at position(s) R24, R27, Q38, I87, K138, E142, D144, and/or E151. In some embodiments, the mutant IL-10 monomer polypeptide comprises amino acid substitutions N18I, Q38A, N92I, K99N and F111L. In some embodiments, the mutant IL-10 monomer polypeptide comprises the sequence of SEQ ID NO:189. In some embodiments, the mutant IL-10 monomer polypeptide comprises amino acid substitutions N18I, R24A, N92I, K99N and F111L. In some embodiments, the mutant IL-10 monomer polypeptide comprises the sequence of SEQ ID NO:190. In some embodiments, the mutant IL-10 monomer polypeptide comprises amino acid substitutions N18I, R24A, Q38A, N92I, K99N and F111L. In some embodiments, the mutant IL-10 monomer polypeptide comprises the sequence of SEQ ID NO:191. In some embodiments, the mutant IL-10 monomer polypeptide comprises amino acid substitutions N18I, N92I, K99N, F111L, and E151A. In some embodiments, the mutant IL-10 monomer polypeptide comprises the sequence of SEQ ID NO:192. In some embodiments, the mutant IL-10 monomer polypeptide comprises amino acid substitutions N18I, R24A, N92I, K99N, F111L, and E151A. In some embodiments, the mutant IL-10 monomer polypeptide comprises the sequence of SEQ ID NO:193. In some embodiments, the mutant IL-10 monomer polypeptide comprises amino acid substitutions N18I, I87A, N92I, K99N and F111L. In some embodiments, the mutant IL-10 monomer polypeptide comprises the sequence of SEQ ID NO:194. In some embodiments, the mutant IL-10 monomer polypeptide comprises amino acid substitutions N18I, N92I, K99N, F111L, and K138A. In some embodiments, the mutant IL-10 monomer polypeptide comprises the sequence of SEQ ID NO:195. In some embodiments, the mutant IL-10 monomer polypeptide comprises amino acid substitutions N18I, R27A, N92I, K99N and F111L. In some embodiments, the mutant IL-10 monomer polypeptide comprises the sequence of SEQ ID NO:196. In some embodiments, the mutant IL-10 monomer polypeptide comprises amino acid substitutions N18I, N92I, K99N, F111L, and E142A. In some embodiments, the mutant IL-10 monomer polypeptide comprises the sequence of SEQ ID NO:197. In some embodiments, the mutant IL-10 monomer polypeptide comprises amino acid substitutions N18I, N92I, K99N, F111L, and D144A. In some embodiments, the mutant IL-10 monomer polypeptide comprises the sequence of SEQ ID NO:198. In some embodiments, the mutant IL-10 monomer polypeptide comprises amino acid substitutions N18I, Q38A, N92I, K99N, F111L, and E142A. In some embodiments, the mutant IL-10 monomer polypeptide comprises the sequence of SEQ ID NO:199. In some embodiments, the mutant IL-10 monomer polypeptide comprises amino acid substitutions N18I, N92I, K99N, F111L, E142A, and K138A. In some embodiments, the mutant IL-10 monomer polypeptide comprises the sequence of SEQ ID NO:200. In some embodiments, the mutant IL-10 monomer polypeptide comprises amino acid substitution N92I. In some embodiments, the mutant IL-10 monomer polypeptide comprises the sequence of SEQ ID NO:201. In some embodiments, the mutant IL-10 monomer polypeptide comprises a sequence selected from the group consisting of SEQ ID Nos: 87-89 and 188-201. In some embodiments, the mutant IL-10 monomer polypeptide comprises the sequence of a mutant monomer IL-10 polypeptide shown in Table 4A. In some embodiments, the mutant IL-10 monomer polypeptide comprises an amino acid substitution and/or amino acid insertion sequence of a mutant monomer IL-10 polypeptide shown in Table 4A.

TABLE 4A
Amino acid sequences of exemplary mutant monomer IL-10 polypeptides.

| Construct | | |
|---|---|---|
| name | Amino Acid Sequence | SEQ ID NO. |
| IL10mono_ | SPGQGTQSENSCTHFPGILPNMLRDLRDAFSRVKTFFQMKDQLDNLL | 188 |
| Rbenh | LKESLLEDFKGYLGCQALSEMIQFYLEEVMPQAENQDPDIKAHVISLG | |
| | ENLNTLRLRLRRCHRLLPCENGGGSGGKSKAVEQVKNAFNKLQEKGIY | |
| | KAMSEFDIFINYIEAYMTMKIRN | |
| IL10mono_ | SPGQGTQSENSCTHFPGILPNMLRDLRDAFSRVKTFFAMKDQLDNLL | 189 |
| RBenh_ | LKESLLEDFKGYLGCQALSEMIQFYLEEVMPQAENQDPDIKAHVISLG | |
| m1 | ENLNTLRLRLRRCHRLLPCENGGGSGGKSKAVEQVKNAFNKLQEKGIY | |
| | KAMSEFDIFINYIEAYMTMKIRN | |
| IL10mono_ | SPGQGTQSENSCTHFPGILPNMLADLRDAFSRVKTFFQMKDQLDNLL | 190 |
| RBenh_ | LKESLLEDFKGYLGCQALSEMIQFYLEEVMPQAENQDPDIKAHVISLG | |
| m2 | ENLNTLRLRLRRCHRLLPCENGGGSGGKSKAVEQVKNAFNKLQEKGIY | |
| | KAMSEFDIFINYIEAYMTMKIRN | |
| IL10mono_ | SPGQGTQSENSCTHFPGILPNMLADLRDAFSRVKTFFAMKDQLDNLL | 191 |
| RBenh_ | LKESLLEDFKGYLGCQALSEMIQFYLEEVMPQAENQDPDIKAHVISLG | |
| m3 | ENLNTLRLRLRRCHRLLPCENGGGSGGKSKAVEQVKNAFNKLQEKGIY | |
| | KAMSEFDIFINYIEAYMTMKIRN | |
| IL10mono_ | SPGQGTQSENSCTHFPGILPNMLRDLRDAFSRVKTFFQMKDQLDNLL | 192 |
| RBenh_ | LKESLLEDFKGYLGCQALSEMIQFYLEEVMPQAENQDPDIKAHVISLG | |
| m4 | ENLNTLRLRLRRCHRLLPCENGGGSGGKSKAVEQVKNAFNKLQEKGIY | |
| | KAMSEFDIFINYIAAYMTMKIRN | |
| IL10mono_ | SPGQGTQSENSCTHFPGILPNMLADLRDAFSRVKTFFQMKDQLDNLL | 193 |
| RBenh_ | LKESLLEDFKGYLGCQALSEMIQFYLEEVMPQAENQDPDIKAHVISLG | |
| m5 | ENLNTLRLRLRRCHRLLPCENGGGSGGKSKAVEQVKNAFNKLQEKGIY | |
| | KAMSEFDIFINYIAAYMTMKIRN | |
| IL10mono_ | SPGQGTQSENSCTHFPGILPNMLRDLRDAFSRVKTFFQMKDQLDNLL | 194 |
| RBenh_m6 | LKESLLEDFKGYLGCQALSEMIQFYLEEVMPQAENQDPDAKAHVISLG | |
| | ENLNTLRLRLRRCHRLLPCENGGGSGGKSKAVEQVKNAFNKLQEKGIY | |
| | KAMSEFDIFINYIEAYMTMKIRN | |
| IL10mono_ | SPGQGTQSENSCTHFPGILPNMLRDLRDAFSRVKTFFQMKDQLDNLL | 195 |
| RBenh_ | LKESLLEDFKGYLGCQALSEMIQFYLEEVMPQAENQDPDIKAHVISLG | |
| m7 | ENLNTLRLRLRRCHRLLPCENGGGSGGKSKAVEQVKNAFNKLQEKGIY | |
| | AAMSEFDIFINYIEAYMTMKIRN | |
| IL10mono_ | SPGQGTQSENSCTHFPGILPNMLRDLADAFSRVKTFFQMKDQLDNLL | 196 |
| RBenh_m8 | LKESLLEDFKGYLGCQALSEMIQFYLEEVMPQAENQDPDIKAHVISLG | |
| | ENLNTLRLRLRRCHRLLPCENGGGSGGKSKAVEQVKNAFNKLQEKGIY | |
| | KAMSEFDIFINYIEAYMTMKIRN | |
| IL10mono_ | SPGQGTQSENSCTHFPGILPNMLRDLRDAFSRVKTFFQMKDQLDNLL | 197 |
| RBenh_ | LKESLLEDFKGYLGCQALSEMIQFYLEEVMPQAENQDPDIKAHVISLG | |
| m9 | ENLNTLRLRLRRCHRLLPCENGGGSGGKSKAVEQVKNAFNKLQEKGIY | |
| | KAMSAFDIFINYIEAYMTMKIRN | |
| IL10mono_ | SPGQGTQSENSCTHFPGILPNMLRDLRDAFSRVKTFFQMKDQLDNLL | 198 |
| RBenh_ | LKESLLEDFKGYLGCQALSEMIQFYLEEVMPQAENQDPDIKAHVISLG | |
| m10 | ENLNTLRLRLRRCHRLLPCENGGGSGGKSKAVEQVKNAFNKLQEKGIY | |
| | KAMSEFAIFINYIEAYMTMKIRN | |
| IL10mono_ | SPGQGTQSENSCTHFPGILPNMLRDLRDAFSRVKTFFAMKDQLDNLL | 199 |
| RBenh_m11 | LKESLLEDFKGYLGCQALSEMIQFYLEEVMPQAENQDPDIKAHVISLG | |
| | ENLNTLRLRLRRCHRLLPCENGGGSGGKSKAVEQVKNAFNKLQEKGIY | |
| | KAMSAFDIFINYIEAYMTMKIRN | |
| IL10mono_ | SPGQGTQSENSCTHFPGILPNMLRDLRDAFSRVKTFFQMKDQLDNLL | 200 |
| RBenh_m12 | LKESLLEDFKGYLGCQALSEMIQFYLEEVMPQAENQDPDIKAHVISLG | |
| | ENLNTLRLRLRRCHRLLPCENGGGSGGKSKAVEQVKNAFNKLQEKGIY | |
| | AAMSAFDIFINYIEAYMTMKIRN | |
| IL10mono_ | SPGQGTQSENSCTHFPGNLPNMLRDLRDAFSRVKTFFQMKDQLDNL | 201 |
| RBenh2 | LLKESLLEDFKGYLGCQALSEMIQFYLEEVMPQAENQDPDIKAHVISLG | |
| | ENLKTLRLRLRRCHRFLPCENGGGSGGKSKAVEQVKNAFNKLQEKGIY | |
| | KAMSEFDIFINYIEAYMTMKIRN | |
| IL10mono_ | SPGQGTQSENSCTHFPGNLPNMLRDLADAFSRVKTFFQMKDQLDNL | 87 |
| RBenh2_m13 | LLKESLLEDFKGYLGCQALSEMIQFYLEEVMPQAENQDPDIKAHVISLG | |
| | ENLKTLRLRLRRCHRFLPCENGGGSGGKSKAVEQVKNAFNKLQEKGIY | |
| | AAMSEFDIFINYIEAYMTMKIRN | |
| IL10mono_ | SPGQGTQSENSCTHFPGNLPNMLADLRDAFSRVKTFFQMKDQLDNL | 88 |
| RBenh2_m14 | LLKESLLEDFKGYLGCQALSEMIQFYLEEVMPQAENQDPDIKAHVISLG | |
| | ENLKTLRLRLRRCHRFLPCENGGGSGGKSKAVEQVKNAFNKLQEKGIY | |
| | AAMSEFDIFINYIEAYMTMKIRN | |
| IL10mono_ | SPGQGTQSENSCTHFPGNLPNMLADLADAFSRVKTFFQMKDQLDNL | 89 |
| RBenh2_m15 | LLKESLLEDFKGYLGCQALSEMIQFYLEEVMPQAENQDPDIKAHVISLG | |
| | ENLKTLRLRLRRCHRFLPCENGGGSGGKSKAVEQVKNAFNKLQEKGIY | |
| | KAMSEFDIFINYIEAYMTMKIRN | |
| IL10mono_ | SPGQGTQSENSCTHFPGNLPNMLADLADAFSRVKTFFQMKDQLDNL | 90 |
| RBenh2_m16 | LLKESLLEDFKGYLGCQALSEMIQFYLEEVMPQAENQDPDIKAHVISLG | |
| | ENLKTLRLRLRRCHRFLPCENGGGSGGKSKAVEQVKNAFNKLQEKGIY | |
| | AAMSEFDIFINYIEAYMTMKIRN | |

TABLE 8

| IL10 Mutein | Sequence | SEQ ID NO |
|---|---|---|
| IL10mono_RBenh2_ | SPGQGTQSENSCTHFPGNLPNMLRDLRDAFSRVKTFFQMKDQLDNLLLKE | 310 |
| m10 | SLLEDFKGYLGCQALSEMIQFYLEEVMPQAENQDPDIKAHVISLGENLKTLR | |
| | LRLRRCHRFLPCENGGGSGGKSKAVEQVKNAFNKLQEKGIYKAMSEFAIFIN | |
| | YIEAYMTMKIRN | |
| IL10mono_RBenh2_ | SPGQGTQSENSCTHFPGNLPNMLRDLRDAFSRVKTFFQMKDQLDNLLLKE | 311 |
| m12 | SLLEDFKGYLGCQALSEMIQFYLEEVMPQAENQDPDIKAHVISLGENLKTLR | |
| | LRLRRCHRFLPCENGGGSGGKSKAVEQVKNAFNKLQEKGIYAAMSAFDIFI | |
| | NYIEAYMTMKIRN | |
| IL10mono_RBenh7_ | SPGQGTQSENSCTHFPGNLPNMLRDLRDAFSRVKTFFQMKDQLDNLLLKE | 312 |
| m12 | SLLEDFKGYLGCQALSEMIQFYLEEVMPQAENQDPDIKAHVLSLGENLKTLR | |
| | LRLRRCHRFLPCENGGGSGGKSKAVEQVKNAFNKLQEKGIYAAMSAFDIFI | |
| | NYIEAYMTMKIRN | |
| IL10mono_RBenh7_ | SPGQGTQSENSCTHFPGNLPNMLRDLRDAFSRVKTFFQMKDQLDNLLLKE | 313 |
| m10 | SLLEDFKGYLGCQALSEMIQFYLEEVMPQAENQDPDIKAHVLSLGENLKTLR | |
| | LRLRRCHRFLPCENGGGSGGKSKAVEQVKNAFNKLQEKGIYKAMSEFAIFIN | |
| | YIEAYMTMKIRN | |
| IL10mono_RBenh6_ | SPGQGTQSENSCTHFPGNLPNMLRDLRDAFSRVKTFFQMKDQLDNLLLKE | 314 |
| m10 | SLLEDFKGYLGCQALSEMIQFYLEEVMPQAENQDPDIKAHVVSLGENLKTL | |
| | RLRLRRCHRFLPCENGGGSGGKSKAVEQVKNAFNKLQEKGIYKAMSEFAIFI | |
| | NYIEAYMTMKIRN | |
| IL10mono_RBenh8_ | SPGQGTQSENSCTHFPGNLPNMLRDLRDAFSRVKTFFQMKDQLDNLLLKE | 315 |
| m10 | SLLEDFKGYLGCQALSEMIQFYLEEVMPQAENQDPDIKAHVRSLGENLKTL | |
| | RLRLRRCHRFLPCENGGGSGGKSKAVEQVKNAFNKLQEKGIYKAMSEFAIFI | |
| | NYIEAYMTMKIRN | |
| IL10mono_RBenh2. | SPGQGTQSENSCTHFPGNLPNMLRDLRDAFSRVKTFFQMKD | 316 |
| 1-m10 | QLDNLLLKESLLEDFKGYLGCQALSEMIQFYLEEVMPQAEN | |
| | QDPDIKAHVISLGENLKTLRLRLRRCHRFLPCENKGGGSGGS | |
| | KAVEQVKNAFNKLQEKGIYKAMSEFAIFINYIEAYMTMKIRN | |
| IL10mono_RBenh7. | SPGQGTQSENSCTHFPGNLPNMLRDLRDAFSRVKTFFQMKD | 317 |
| 1-m10 | QLDNLLLKESLLEDFKGYLGCQALSEMIQFYLEEVMPQAEN | |
| | QDPDIKAHVLSLGENLKTLRLRLRRCHRFLPCENKGGGSGGS | |
| | KAVEQVKNAFNKLQEKGIYKAMSEFAIFINYIEAYMTMKIRN | |
| IL10mono_RBenh2. | SPGQGTQSENSCTHFPGNLPNMLADLADAFSRVKTFFQMKDQLDN | 318 |
| 1-m15 | LLLKESLLEDFKGYLGCQALSEMIQFYLEEVMPQAENQDPDIKAHVIS | |
| | LGENLKTLRLRLRRCHRFLPCENKGGGSGGSKAVEQVKNAFNKLQEK | |
| | GIYKAMSEFDIFINYIEAYMTMKIRN | |

In some embodiments, the mutant IL-10 monomer polypeptide comprises a sequence selected from the group consisting of SEQ ID Nos:310-318. In some embodiments, the mutant IL-10 monomer polypeptide comprises the sequence of a mutant monomer IL-10 polypeptide shown in Table 8. In some embodiments, the mutant IL-10 monomer polypeptide comprises an amino acid substitution and/or amino acid insertion sequence of a mutant monomer IL-10 polypeptide shown in Table 8.

TABLE 11

| | | SEQ ID |
|---|---|---|
| IL10 Mutein | Sequence | NO |
| IL10mono_RBenh2_ | SPGQGTQSENSCTHFPGNLPNMLRDLRDAFSRVKTFFQMKDQLDNLLLKES | 422 |
| m117 | LLEDFKGYLGCQALSEMIQFYLEEVMPQAENQDPDIKAHVISLGENLKTLRLR | |
| | LRACHRFLPCENGGGSGGKSKAVEQVKNAFNKLQEKGIYKAMSEFDIFINYIE | |
| | AYMTMKIRN | |
| IL-10mono_RBenh2- | SPGQGTQSENSCTHFPGNLPNMLRDLRDAFSRVKTFFQMKDQLDNL | 423 |
| m10 m117 | LLKESLLEDFKGYLGCQALSEMIQFYLEEVMPQAENQDPDIKAHVISLG | |
| | ENLKTLRLRLRACHRFLPCENGGGSGGKSKAVEQVKNAFNKLQEKGIY | |
| | KAMSEFAIFINYIEAYMTMKIRN | |
| IL-10mono_RBenh7- | SPGQGTQSENSCTHFPGNLPNMLRDLRDAFSRVKTFFQMKDQLDNL | 424 |
| m10 m117 | LLKESLLEDFKGYLGCQALSEMIQFYLEEVMPQAENQDPDIKAHVLSL | |
| | GENLKTLRLRLRACHRFLPCENGGGSGGKSKAVEQVKNAFNKLQEKG | |
| | IYKAMSEFAIFINYIEAYMTMKIRN | |
| IL-10mono_RBenh2- | SPGQGTQSENSCTHFPGNLPNMLADLADAFSRVKTFFQMKDQLDNL | 425 |
| m15 m117 | LLKESLLEDFKGYLGCQALSEMIQFYLEEVMPQAENQDPDIKAHVISLG | |
| | ENLKTLRLRLRACHRFLPCENGGGSGGKSKAVEQVKNAFNKLQEKGIY | |
| | KAMSEFDIFINYIEAYMTMKIRN | |
| IL-10mono_RBenh2.1- | SPGQGTQSENSCTHFPGNLPNMLRDLRDAFSRVKTFFQMKDQLDNL | 426 |
| m10 m117 | LLKESLLEDFKGYLGCQALSEMIQFYLEEVMPQAENQDPDIKAHVISLG | |
| | ENLKTLRLRLRACHRFLPCENKGGGSGGSKAVEQVKNAFNKLQEKGIY | |
| | KAMSEFAIFINYIEAYMTMKIRN | |
| IL-10mono_RBenh7.1- | SPGQGTQSENSCTHFPGNLPNMLRDLRDAFSRVKTFFQMKDQLDNL | 427 |
| m10 m117 | LLKESLLEDFKGYLGCQALSEMIQFYLEEVMPQAENQDPDIKAHVLSL | |
| | GENLKTLRLRLRACHRFLPCENKGGGSGGSKAVEQVKNAFNKLQEKG | |
| | IYKAMSEFAIFINYIEAYMTMKIRN | |
| IL-10mono_RBenh2.1- | SPGQGTQSENSCTHFPGNLPNMLADLADAFSRVKTFFQMKDQLDNL | 428 |
| m15 m117 | LLKESLLEDFKGYLGCQALSEMIQFYLEEVMPQAENQDPDIKAHVISLG | |
| | ENLKTLRLRLRACHRFLPCENKGGGSGGSKAVEQVKNAFNKLQEKGIY | |
| | KAMSEFDIFINYIEAYMTMKIRN | |

In some embodiments, the mutant IL-10 monomer polypeptide comprises a sequence selected from the group consisting of SEQ ID Nos:422-428. In some embodiments, the mutant IL-10 monomer polypeptide comprises the sequence of a mutant monomer IL-10 polypeptide shown in Table 11. In some embodiments, the mutant IL-10 monomer polypeptide comprises an amino acid substitution and/or amino acid insertion sequence of a mutant monomer IL-10 polypeptide shown in Table 11.

Table 4B depicts exemplary amino acid insertions and insertion positions for IL-10 monomer polypeptides of the present disclosure (insertions are underlined). In some embodiments, the mutant IL-10 monomer polypeptide comprises an amino acid sequence listed in Table 4B. In some embodiments, the mutant IL-10 monomer polypeptide comprises a sequence selected from the group consisting of SEQ ID Nos:91-101. In some embodiments, the mutant IL-10 monomer polypeptide comprises an amino acid insertion as listed in Table 4B and/or at a position as listed in Table 4B. In some embodiments, the insertion is 1, 2, 3, 4, 5, 6, 7, 8, 9, 10, 11, 12, 13, 14, or 15 amino acids in length. In some embodiments, the mutant IL-10 monomer polypeptide comprises the amino acid sequence of a mutant IL-10 monomer polypeptide of the present disclosure with an amino acid or peptide insertion of between 1 and 15 amino acids immediately following residue C114, E115, N116, K117, S118, K119, or A120, numbering based on SEQ ID NO:1. Examples of insertion can include, without limitation, G, GG, GGG, GGGG (SEQ ID NO:80), GGGSG (SEQ ID NO:81), GGGGG (SEQ ID NO:82), GGGGGG (SEQ ID NO:83), and GGGSGG (SEQ ID NO:84).

TABLE 4B
Exemplary insertions and insertion positions of mutant monomer IL-10
polypeptides

| Construct | | |
|---|---|---|
| name | Amino Acid Sequence | SEQ ID NO. |
| IL10mono_ | SPGQGTQSENSCTHFPGNLPNMLRDLRDAFSRVKTFFQMKDQLDNL | 91 |
| insertC114_E115 | LLKESLLEDFKGYLGCQALSEMIQFYLEEVMPQAENQDPDIKAHVNSL | |
| | GENLKTLRLRLRRCHRFLPCGGGSGGENKSKAVEQVKNAFNKLQEKGI | |
| | YKAMSEFDIFINYIEAYMTMKIRN | |
| IL10mono_ | SPGQGTQSENSCTHFPGNLPNMLRDLRDAFSRVKTFFQMKDQLDNL | 92 |
| insertE115_N116 | LLKESLLEDFKGYLGCQALSEMIQFYLEEVMPQAENQDPDIKAHVNSL | |
| | GENLKTLRLRLRRCHRFLPCEGGGSGGNKSKAVEQVKNAFNKLQEKGI | |
| | YKAMSEFDIFINYIEAYMTMKIRN | |
| IL10mono_ | SPGQGTQSENSCTHFPGNLPNMLRDLRDAFSRVKTFFQMKDQLDNL | 93 |
| insertK117_S118 | LLKESLLEDFKGYLGCQALSEMIQFYLEEVMPQAENQDPDIKAHVNSL | |
| | GENLKTLRLRLRRCHRFLPCENKGGGSGGSKAVEQVKNAFNKLQEKGI | |
| | YKAMSEFDIFINYIEAYMTMKIRN | |
| IL10mono_ | SPGQGTQSENSCTHFPGNLPNMLRDLRDAFSRVKTFFQMKDQLDNL | 94 |
| insertS118_K119 | LLKESLLEDFKGYLGCQALSEMIQFYLEEVMPQAENQDPDIKAHVNSL | |
| | GENLKTLRLRLRRCHRFLPCENKSGGGSGGKAVEQVKNAFNKLQEKGI | |
| | YKAMSEFDIFINYIEAYMTMKIRN | |
| IL10mono_ | SPGQGTQSENSCTHFPGNLPNMLRDLRDAFSRVKTFFQMKDQLDNL | 95 |
| insertK119_A120 | LLKESLLEDFKGYLGCQALSEMIQFYLEEVMPQAENQDPDIKAHVNSL | |
| | GENLKTLRLRLRRCHRFLPCENKSKGGGSGGAVEQVKNAFNKLQEKGI | |
| | YKAMSEFDIFINYIEAYMTMKIRN | |
| IL10mono_ | SPGQGTQSENSCTHFPGNLPNMLRDLRDAFSRVKTFFQMKDQLDNL | 96 |
| 1GinsertK117_ | LLKESLLEDFKGYLGCQALSEMIQFYLEEVMPQAENQDPDIKAHVNSL | |
| S118 | GENLKTLRLRLRRCHRFLPCENKGSKAVEQVKNAFNKLQEKGIYKAMS | |
| | EFDIFINYIEAYMTMKIRN | |
| IL10mono_ | SPGQGTQSENSCTHFPGNLPNMLRDLRDAFSRVKTFFQMKDQLDNL | 97 |
| 2GinsertK117_ | LLKESLLEDFKGYLGCQALSEMIQFYLEEVMPQAENQDPDIKAHVNSL | |
| S118 | GENLKTLRLRLRRCHRFLPCENKGGSKAVEQVKNAFNKLQEKGIYKA | |
| | MSEFDIFINYIEAYMTMKIRN | |
| IL10mono_ | SPGQGTQSENSCTHFPGNLPNMLRDLRDAFSRVKTFFQMKDQLDNL | 98 |
| 3GinsertK117_ | LLKESLLEDFKGYLGCQALSEMIQFYLEEVMPQAENQDPDIKAHVNSL | |
| S118 | GENLKTLRLRLRRCHRFLPCENKGGGSKAVEQVKNAFNKLQEKGIYKA | |
| | MSEFDIFINYIEAYMTMKIRN | |
| IL10mono_ | SPGQGTQSENSCTHFPGNLPNMLRDLRDAFSRVKTFFQMKDQLDNL | 99 |
| 4GinsertK117_ | LLKESLLEDFKGYLGCQALSEMIQFYLEEVMPQAENQDPDIKAHVNSL | |
| S118 | GENLKTLRLRLRRCHRFLPCENKGGGGSKAVEQVKNAFNKLQEKGIYK | |
| | AMSEFDIFINYIEAYMTMKIRN | |
| IL10mono_ | SPGQGTQSENSCTHFPGNLPNMLRDLRDAFSRVKTFFQMKDQLDNL | 100 |
| 5GinsertK117_ | LLKESLLEDFKGYLGCQALSEMIQFYLEEVMPQAENQDPDIKAHVNSL | |
| S118 | GENLKTLRLRLRRCHRFLPCENKGGGGGSKAVEQVKNAFNKLQEKGIY | |
| | KAMSEFDIFINYIEAYMTMKIRN | |
| IL10mono_ | SPGQGTQSENSCTHFPGNLPNMLRDLRDAFSRVKTFFQMKDQLDNL | 101 |
| 6GinsertK117_ | LLKESLLEDFKGYLGCQALSEMIQFYLEEVMPQAENQDPDIKAHVNSL | |
| S118 | GENLKTLRLRLRRCHRFLPCENKGGGGGGSKAVEQVKNAFNKLQEKG | |
| | IYKAMSEFDIFINYIEAYMTMKIRN | |

### Fusion Proteins

Further provided here are fusion proteins comprising any one of the mutant IL-10 polypeptides of the present disclosure and an antigen binding molecule binding to an antigen on T cells. In some embodiments, said fusion proteins preferentially stimulate T cells over monocytes. In some embodiments, the fusion proteins of the present disclosure comprise the mutant IL-10 polypeptides and antigen binding molecules binding to CD8+ T cells, wherein said fusion proteins preferentially stimulate CD8+ T cells over monocytes. In some embodiments, the antigen binding molecules bind to CD8 (e.g., CD8ab, CD8a, or CD8aa), CD4, or PD-1, e.g., human CD8 (e.g., human CD8ab, human CD8a, or human CD8aa), human CD4, or human PD-1. Human CD8, CD4, and PD-1 sequences are known in the art; see, e.g., NP_001139345 for human CD8a, NP_001171571 for human CD8b, NP_000607 for human CD4, and NP_005009 for human PD-1.

In other embodiments, the fusion proteins comprise the mutant IL-10 polypeptide and antigen binding molecules binding to the CD8ab and/or CD8a antigens, wherein said fusion proteins preferentially stimulate CD8+ T cells over monocytes.

Preferential activity of the targeted IL-10 fusion proteins of the present disclosure on antigen-expressing cells is demonstrated in assays that contain antigen-expressing and antigen-non expressing cells that also express the IL-10R. One such assay is an in vitro assay that measures STAT3 phosphorylation (pSTAT3) in human immune cells, such as human peripheral blood and/or tumor-infiltrating immune cells upon exposure to IL-10 polypeptides. In one format of the assay, the activity of the targeted IL-10 fusion protein is measured on antigen-expressing and antigen non-expressing cells to demonstrate the selectivity on antigen-expressing cells. In another format of the assay, the activity of the targeted IL-10 fusion protein comprising the mutant IL-10 polypeptide on antigen-expressing cells is compared to that of the untargeted IL-10 fusion protein comprising the same mutant IL-10 polypeptide and a control antibody not recognizing any antigens on antigen-expressing cells. to demonstrate the magnitude of rescue in signaling of the mutant IL-10 polypeptide when fused to an antigen binding molecule.

In some embodiments, the fusion protein of the present disclosure containing CD8ab antigen binding molecule activates CD8ab+IL-10R+ cells over CD8ab− IL-10R+ cells, by at least 5 fold, at least 10 fold, at least 50 fold, or at least 100 fold. In some embodiments, said fusion protein activates CD8ab+IL-10R+ cells more than 50 fold, or more desirably, at least 100 fold, or even more desirably, at least 200 fold compared to a fusion molecule comprising the said IL-10 mutant polypeptide and a control antibody not binding to any antigens expressed on said cells. Said cell activation by the IL-10 fusion protein is determined by measuring the expression of pSTAT3 in said cells following the treatment with said IL-10 fusion protein.

In some embodiments, the fusion protein of the present disclosure containing CD8a antigen binding molecule activates CD8a+IL-10R+ cells over CD8a−IL-10R+ cells by at least 5 fold, at least 10 fold, at least 50 fold, or at least 100 fold. In some embodiments, said fusion protein activates CD8a+IL-10R+ cells more than 50 fold, or more desirably, at least 100 fold, or even more desirably, at least 200 fold compared to a fusion molecule comprising the said IL-10 mutant polypeptide and a control antibody not binding to any antigens expressed on said cells. Said cell activation by the IL-10 fusion protein is determined by measuring the expression of pSTAT3 in said cells following the treatment with said IL-10 fusion protein.

### Fusion Protein Formats

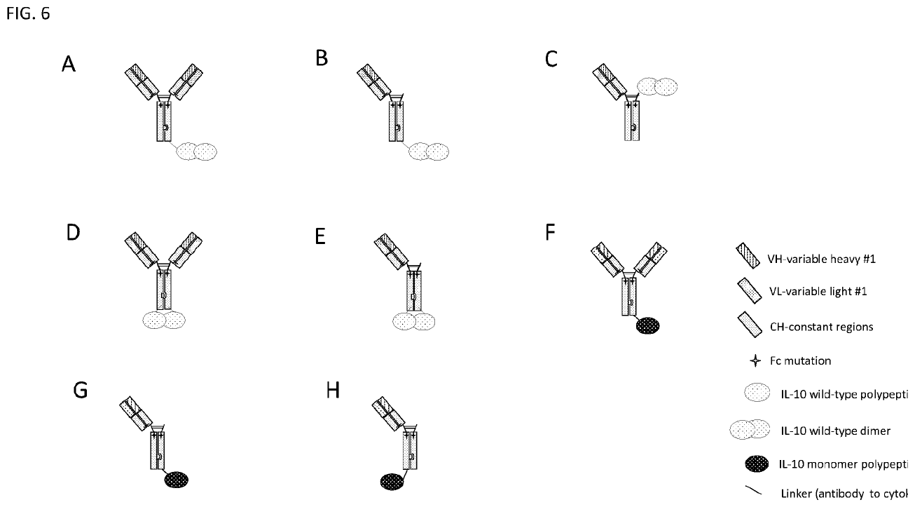

The present disclosure relates, inter alia, to fusion proteins comprising an antigen binding molecule (e.g., an antibody or other antigen binding protein) and a mutant IL-10 polypeptide of the present disclosure. In some embodiments, the IL-10 fusion proteins have different formats, e.g., as depicted in FIG. 6. In some embodiments, the fusion protein comprises a dimer of two mutant IL-10 polypeptides, and wherein one of the two mutant IL-10 polypeptides is fused to the antigen binding molecule (e.g., a dimer of IL-10 is fused to the antigen binding molecule via a single linkage). In some embodiments, the fusion protein comprises a single mutant monomer IL-10 polypeptide that is fused to the antigen binding molecule. In some embodiments, the fusion protein comprises two antigen binding molecules, wherein one mutant IL-10 polypeptide is fused to each of the two antigen binding molecules (e.g., the antigen binding molecule comprises two polypeptide chains, each fused to a single mutant IL-10 polypeptide, and the two mutant IL-10 polypeptides associate as a dimer upon assembly of the fusion protein). In some embodiments, the mutant IL-10 polypeptide and the antigen binding molecule are fused (e.g., covalently) via a linker.

In some embodiments, the fusion protein comprises an antigen binding molecule that comprises two antibody heavy chain polypeptides comprising a structure according to formula [I], from N-terminus to C-terminus:

VH-CH1-hinge-CH2-CH3 [I]

and two antibody light chain polypeptides comprising a structure according to formula [II], from N-terminus to C-terminus:

VL-CL [II]

wherein VH is an antibody heavy chain variable (VH) domain, wherein CH1 is an antibody CH1 domain, wherein hinge is an antibody hinge domain, wherein CH2 is an antibody CH2 domain, wherein CH3 is an antibody CH3 domain, wherein VL is an antibody light chain variable (VL) domain, and wherein CL is an antibody constant light chain domain. See, e.g., FIG. 6 at A, D, and F. In some embodiments, VH/VL form an antigen binding site.

In some embodiments, the fusion protein comprises two mutant IL-10 polypeptides associated in a dimer; and the N-terminus of one of the two mutant IL-10 polypeptides is fused to the C-terminus of one of the two CH3 domains directly or via linker, e.g., as depicted in FIG. 6 at A. In some embodiments, the fusion protein comprises two mutant IL-10 polypeptides associated in a dimer; and the N-terminus of a first of the two mutant IL-10 polypeptides is fused to the C-terminus of a first of the two CH3 domains directly or via linker, and the N-terminus of the second of the two mutant IL-10 polypeptides is fused to the C-terminus of the second of the two CH3 domains directly or via linker, e.g., as depicted in FIG. 6 at D. In some embodiments, the fusion protein comprises one mutant IL-10 monomer polypeptide; and the N-terminus of the mutant IL-10 monomer polypeptide is fused to the C-terminus of one of the two CH3 domains directly or via linker, e.g., as depicted in FIG. 6 at F.

In some embodiments, the fusion protein comprises an antigen binding molecule that comprises first antibody heavy chain polypeptide comprising a structure according to formula [I], from N-terminus to C-terminus:

VH-CH1-hinge-CH2-CH3 [I],

an antibody light chain polypeptide comprising a structure according to formula [II], from N-terminus to C-terminus:

VL-CL [II],

and a second antibody heavy chain polypeptide comprising a structure according to formula [III], fromN-terminus to C-terminus:

hinge-CH2-CH3 [III],

wherein VH is an antibody heavy chain variable (VH) domain, wherein CH1 is an antibody CH1 domain, wherein hinge is an antibody hinge domain, wherein CH2 is an antibody CH2 domain, wherein CH3 is an antibody CH3 domain, wherein VL is an antibody light chain variable (VL) domain, and wherein CL is an antibody constant light chain domain. See, e.g., FIG. 6 at B, C, E, G, and H. In some embodiments, VH/VL form an antigen binding site.

In some embodiments, the fusion protein comprises two mutant IL-10 polypeptides associated in a dimer; and the N-terminus of one of the two mutant IL-10 polypeptides is fused, directly or via linker, to one of: the C-terminus of the CH3 domain of the second antibody heavy chain polypeptide or the C-terminus of the CH3 domain of the first antibody heavy chain polypeptide, e.g., as depicted in FIG. 6 at B. In some embodiments, the fusion protein comprises two mutant IL-10 polypeptides associated in a dimer; and the N-terminus of one of the two mutant IL-10 polypeptides is fused, directly or via linker, to the N-terminus of the hinge region of the second antibody heavy chain polypeptide, e.g., as depicted in FIG. 6 at C. In some embodiments, the fusion protein comprises two mutant IL-10 polypeptides associated in a dimer; the N-terminus of a first of the two mutant IL-10 polypeptides is fused to the C-terminus of the CH3 domain of the first antibody heavy chain polypeptide directly or via linker, and the N-terminus of the second of the two mutant IL-10 polypeptides is fused to the C-terminus of the CH3 domain of the second antibody heavy chain polypeptide directly or via linker, e.g., as depicted in FIG. 6 at E. In some embodiments, the fusion protein comprises one mutant IL-10 monomer polypeptide; and the N-terminus of the mutant IL-10 monomer polypeptide is fused, directly or via linker, to one of: the C-terminus of the CH3 domain of the second antibody heavy chain polypeptide or the C-terminus of the CH3 domain of the first antibody heavy chain polypeptide, e.g., as depicted in FIG. 6 at G and H.

In some embodiments, the fusion protein is as depicted in FIG. 6 at A. For example, in some embodiments, the fusion protein comprises an antigen binding molecule that comprises two antibody heavy chain polypeptides comprising a structure according to formula [I], from N-terminus to C-terminus:

VH-CH1-hinge-CH2-CH3 [I]

and two antibody light chain polypeptides comprising a structure according to formula [II], from N-terminus to C-terminus:

VL-CL [II]

wherein VH is an antibody heavy chain variable (VH) domain, wherein CH1 is an antibody CH1 domain, wherein hinge is an antibody hinge domain, wherein CH2 is an antibody CH2 domain, wherein CH3 is an antibody CH3 domain, wherein VL is an antibody light chain variable (VL) domain, and wherein CL is an antibody constant light chain domain; wherein the fusion protein comprises two mutant IL-10 polypeptides associated in a dimer; and wherein the N-terminus of one of the two mutant IL-10 polypeptides is fused to the C-terminus of one of the two CH3 domains (e.g., covalently fused via a linker of the present disclosure). In some embodiments, each heavy chain is paired with a light chain. In some embodiments, the VH domain of each heavy chain forms an antigen binding site with the VL domain of the respectively paired light chain.

In some embodiments, the fusion protein is as depicted in FIG. 6 at B. For example, in some embodiments, the fusion protein comprises an antigen binding molecule that comprises a first antibody heavy chain polypeptide comprising a structure according to formula [I], from N-terminus to C-terminus:

VH-CH1-hinge-CH2-CH3 [I],

an antibody light chain polypeptide comprising a structure according to formula [II], from N-terminus to C-terminus:

VL-CL [II],

and a second antibody heavy chain polypeptide comprising a structure according to formula [III], from N-terminus to C-terminus:

hinge-CH2-CH3 [III],

wherein VH is an antibody heavy chain variable (VH) domain, wherein CH1 is an antibody CH1 domain, wherein hinge is an antibody hinge domain, wherein CH2 is an antibody CH2 domain, wherein CH3 is an antibody CH3 domain, wherein VL is an antibody light chain variable (VL) domain, and wherein CL is an antibody constant light chain domain; wherein the fusion protein comprises two mutant IL-10 polypeptides associated in a dimer. In some embodiments, the N-terminus of one of the two mutant IL-10 polypeptides is fused to the C-terminus of the CH3 domain of the second antibody heavy chain polypeptide (e.g., covalently fused via a linker of the present disclosure). In some embodiments, the N-terminus of one of the two mutant IL-10 polypeptides is fused to the C-terminus of the CH3 domain of the first antibody heavy chain polypeptide (e.g., covalently fused via a linker of the present disclosure). In some embodiments, the first heavy chain is paired with the light chain. In some embodiments, the VH domain of the first heavy chain forms an antigen binding site with the VL domain of the light chain.

In some embodiments, the fusion protein is as depicted in FIG. 6 at D. For example, in some embodiments, the fusion protein comprises an antigen binding molecule that comprises two antibody heavy chain polypeptides comprising a structure according to formula [I], from N-terminus to C-terminus:

VH-CH1-hinge-CH2-CH3 [I]

and two antibody light chain polypeptides comprising a structure according to formula [II], from N-terminus to C-terminus:

VL-CL [II]

wherein VH is an antibody heavy chain variable (VH) domain, wherein CH1 is an antibody CH1 domain, wherein hinge is an antibody hinge domain, wherein CH2-CH3 is an antibody Fc domain, wherein VL is an antibody light chain variable (VL) domain, and wherein CL is an antibody constant light chain domain; wherein the fusion protein comprises two mutant IL-10 polypeptides associated in a dimer; and wherein the N-terminus of a first of the two mutant IL-10 polypeptides is fused to the C-terminus of a first of the two CH3 domains, and the N-terminus of the second of the two mutant IL-10 polypeptides is fused to the C-terminus of the second of the two CH3 domains (e.g., covalently fused via a linker of the present disclosure). In some embodiments, each heavy chain is paired with a light chain. In some embodiments, the VH domain of each heavy chain forms an antigen binding site with the VL domain of the respectively paired light chain.

In some embodiments, the fusion protein is as depicted in FIG. 6 at E. For example, in some embodiments, the fusion protein comprises an antigen binding molecule that comprises a first antibody heavy chain polypeptide comprising a structure according to formula [I], from N-terminus to C-terminus:

VH-CH1-hinge-CH2-CH3 [I],

an antibody light chain polypeptide comprising a structure according to formula [II], from N-terminus to C-terminus:

VL-CL [II],

and a second antibody heavy chain polypeptide comprising a structure according to formula [III], from N-terminus to C-terminus:

hinge-CH2-CH3 [III],

wherein VH is an antibody heavy chain variable (VH) domain, wherein CH1 is an antibody CH1 domain, wherein hinge is an antibody hinge domain, wherein CH2 is an antibody CH2 domain, wherein CH3 is an antibody CH3 domain, wherein VL is an antibody light chain variable (VL) domain, and wherein CL is an antibody constant light chain domain; wherein the fusion protein comprises two mutant IL-10 polypeptides associated in a dimer; and wherein the N-terminus of a first of the two mutant IL-10 polypeptides is fused to the C-terminus of the CH3 domain of the first antibody heavy chain polypeptide, and the N-terminus of the second of the two mutant IL-10 polypeptides is fused to the C-terminus of the CH3 domain of the second antibody heavy chain polypeptide (e.g., covalently fused via a linker of the present disclosure). In some embodiments, the first heavy chain is paired with the light chain. In some embodiments, the VH domain of the first heavy chain forms an antigen binding site with the VL domain of the light chain.

In some embodiments, the fusion protein is as depicted in FIG. 6 at F. For example, in some embodiments, the fusion protein comprises an antigen binding molecule that comprises two antibody heavy chain polypeptides comprising a structure according to formula [I], from N-terminus to C-terminus:

VH-CH1-hinge-CH2-CH3 [I]

and two antibody light chain polypeptides comprising a structure according to formula [II], from N-terminus to C-terminus:

VL-CL [II]

wherein VH is an antibody heavy chain variable (VH) domain, wherein CH1 is an antibody CH1 domain, wherein hinge is an antibody hinge domain, wherein CH2 is an antibody CH2 domain, wherein CH3 is an antibody CH3 domain, wherein VL is an antibody light chain variable (VL) domain, and wherein CL is an antibody constant light chain domain; wherein the fusion protein comprises one mutant monomer IL-10 polypeptide; and wherein the N-terminus of the mutant monomer IL-10 polypeptide is fused to the C-terminus of one of the two CH3 domains (e.g., covalently fused via a linker of the present disclosure). In some embodiments, each heavy chain is paired with a light chain. In some embodiments, the VH domain of each heavy chain forms an antigen binding site with the VL domain of the respectively paired light chain.

In some embodiments, said first and second Fc domains of the fusion protein contain one or more of the following Fc mutations to decrease effector function according to EU numbering: L234A, L235A, G237A, and K322A. In some embodiments, said first and second Fc domains of the fusion protein contain the following Fc mutations to decrease effector function according to EU numbering: L234A, L235A, and G237A. In some embodiments, said first and second Fc domains of the fusion protein contain the following Fc mutations to decrease effector function according to EU numbering: L234A, L235A, G237A, and K322A. In some embodiments, said first and second Fc domains of the fusion protein contain the following amino acid substitutions to facilitate heterodimeric formation: Y349C/T366W (knob) and S354C, T366S, L368A and Y407V (hole). In some embodiments, one or both of the antibody Fc domains do not have a C-terminal lysine. In some embodiments, the first and second Fc domains are human IgG1 Fc domains.

In some embodiments, bispecific antibody can be generated by post-production assembly from half-antibodies, thereby solving the issues of heavy and light chain mispairing. These antibodies often contain modification to favor heterodimerization of half-antibodies. Exemplary systems include but not limited to the knob-into-hole, IgG1 (EEE-RRR), IgG2 (EEE-RRRR) (Strop et al. J Mol Biol (2012)) and DuoBody (F405L-K409R). In such case, half-antibody is individually produced in separate cell line and purified. The purified antibodies were then subjected to mild reduction to obtain half-antibodies, which were then assembled into bispecific antibodies. Heterodimeric bispecific antibody was then purified from the mixture using conventional purifications methods.

In some embodiments, strategies on bispecific antibody generation that do not rely on the preferential chain pairing can also be employed. These strategies typically involve introducing genetic modification on the antibody in such a manner that the heterodimer will have distinct biochemical or biophysical properties from the homodimers; thus the post-assembled or expressed heterodimer can be selectively purified from the homodimers. One example was to introduce H435R/Y436F in IgG1 CH3 domain to abolish the Fc binding to protein A resin and then co-express the H435R/Y436F variant with a wildtype Fc. The resulting homodimeric antibodies containing two copies of H435R/Y436F cannot bind to the Protein A column, while heterodimeric antibody comprising one copy of H435R/Y436F mutation will have a decreased affinity for protein A as compared to the strong interaction from homodimeric wildtype antibody (Tustian et al Mabs 2016). Other examples include kappa/lambda antibody (Fischer et al., Nature Communication 2015) and introduction of differential charges (E357Q, S267K or N208D/Q295E/N384D/Q418E/N421D) on the respective chains (US 2018/0142040 A1; (Strop et al. J Mol Biol (2012)).

In some embodiments, bispecific antibody can be generated via fusion of an additional binding site to either the heavy or light chain of an immunoglobulin. Examples of the additional binding site include but not limited to variable regions, scFv, Fab, VHH, and peptide.

In some embodiments, the heterodimeric mutations and/or mutations to modify Fc gamma receptor binding resulted in reduction of Fc stability. Therefore, additional mutation(s) was added to the Fc region to increase its stability. For example, one or more pairs of disulfide bonds such as A287C and L306C, V259C and L306C, R292C and V302C, and V323C and I332C are introduced into the Fc region. Another example is to introduce S228P to IgG4 based bispecific antibodies to stabilize the hinge disulfide. Additional example includes introducing K338I, A339K, and K340S mutations to enhance Fc stability and aggregation resistance (Gao et al, 2019 Mol Pharm. 2019; 16:3647).

In some embodiments, a fusion protein of the present disclosure comprises a linker. In some embodiments, the linker is a chemical linker (for example, see disclosed in Protein Engineering, 9(3), 299-305, 1996). Synthetic chemical linkers include crosslinking agents that are routinely used to crosslink peptides, for example, N-hydroxy succinimide (NHS), disuccinimidyl suberate (DSS), bis(succinimidyl) suberate (BS3), dithiobis(succinimidyl propionate) (DSP), dithiobis(succinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis[2-(succinimidoxycarbonyloxy)ethyl] sulfone (BSOCOES), and bis[2-(succinimidoxycarbonyloxy)ethyl] sulfone (sulfo-BSOCOES).

In some embodiments, the linker is an amino acid- or peptide-based linker. In some embodiments, the polypeptide linker is a peptide with a length of at least 5 amino acids, or with a length of 5 to 100, or of 10 to 50 amino acids. In one embodiment, said peptide linker is G, S, GS, SG, SGG, GGS, and GSG (with G=glycine and S=serine). In some embodiments, the linker comprises the sequence (GGGS)xGn (SEQ ID NO:74), (GGGGS)xGn (SEQ ID NO:75), (GGGGGS)xGn (SEQ ID NO:76), S(GGGS)xGn (SEQ ID NO:386), S(GGGGS)xGn (SEQ ID NO:387), or S(GGGGGS)xGn (SEQ ID NO:388), wherein x=1, 2, 3, 4, 5, 6, 7, 8, 9, 10, 11, or 12, and wherein n=0, 1, 2 or 3. In some embodiments, the linker comprises the sequence GGGGSGGGGSGGGGS (SEQ ID NO:79), SGGGGSGGGGSGGGGS (SEQ ID NO:77), or SGGGGSGGGGSGGGG (SEQ ID NO:78).

### Antigen Binding Molecules

In some embodiments, the antigen binding molecules of the present disclosure bind to an epitope on CD8a wherein the binding of the antigen binding molecule to CD8a does not block the interaction of CD8aa or CD8ab with MHC class I molecules on target cells or antigen presenting cells. In some embodiments, the antigen binding molecule of the present disclosure binds to an epitope on CD8b wherein the binding of the antigen binding molecule to CD8b does not block the interaction of CD8ab with MHC class I molecules on target cells or antigen presenting cells.

In some embodiments, the fusion protein binds human CD8, and the binding of the fusion protein to CD8 does not block the interaction of CD8 with MHC class I. In some embodiments, the antigen binding molecule of the present disclosure binds to an epitope on CD8ab wherein the binding of the antigen binding molecule to CD8ab does not block the interaction of CD8aa or CD8ab with MHC class I molecules on target cells or antigen presenting cells. In some embodiments, the antigen binding molecule of the present disclosure binds to an epitope on CD8a wherein the binding of the antigen binding molecule to CD8a does not block the interaction of CD8ab with MHC class I molecules on target cells or antigen presenting cells.

In some embodiments, the fusion protein binds human CD8, and the binding of the fusion protein to CD8 does not block the interaction of CD8 with MHC class I. In some embodiments, the antigen binding molecule of the present disclosure binds to an epitope on CD8α wherein the binding of the antigen binding molecule to CD8α does not block the interaction of CD8αα or CD8αβ with MHC class I molecules on target cells or antigen presenting cells. In some embodiments, the antigen binding molecule of the present disclosure binds to an epitope on CD8β wherein the binding of the antigen binding molecule to CD8β does not block the interaction of CD8αβ with MHC class I molecules on target cells or antigen presenting cells. In some embodiments, whether an anti-CD8 antibody or fusion protein of the present disclosure blocks the interaction of CD8 with MHC class I can be assayed, e.g., by assaying activation status of CD8+ T cells (e.g., upon antigen stimulation) in the presence or absence of the anti-CD8 antibody or fusion protein.

In some embodiments, an anti-CD8 antibody or fusion protein of the present disclosure comprises a VH domain comprising the sequence of EVQLVESGGGLVQPGRSLKLSCAASGFTFSNYYMAWVRQAPTKGLEWVAYINTGG GTTYYRDSVKGRFTISRDDAKSTLYLQMDSLRSEDTATYYCTTAIGYYFDYWGQGV MVTVSS (SEQ ID NO:102) and a VL domain comprising the sequence of DIQLTQSPASLSASLGETVSIECLASEDIYSYLAWYQQKPGKSPQVLIYAANRLQDGV PSRFSGSGSGTQYSLKISGMQPEDEGDYFCLQGSKFPYTFGAGTKLELK (SEQ ID NO:103). In some embodiments, an anti-CD8 antibody or fusion protein of the present disclosure comprises a VH domain comprising the sequence of EVKLQESGPSLVQPSQTLSLTCSVSGFSLISDSVHWVRQPPGKGLEWMGGIWADGST DYNSALKSRLSISRDTSKSQGFLKMNSLQTDDTAIYFCTSNRESYYFDYWGQGTMVT VSS (SEQ ID NO:104) and a VL domain comprising the sequence of DIQMTQSPASLSASLGDKVTITCQASQNIDKYIAWYQQKPGKAPRQLIHYTSTLVSGT PSRFSGSGSGRDYSFSISSVESEDIASYYCLQYDTLYTFGAGTKLELK (SEQ ID NO:105). In some embodiments, an anti-CD8 antibody or fusion protein of the present disclosure comprises a VH domain comprising the sequence of EVKLQESGPSLVQPSQTLSLTCSVSGFSLISDSVHWVRQPPGKGLEWMGGIWADGST DYNSALKSRLSISRDTSKSQGFLKMNSLQTDDTAIYFCTSARESYYFDYWGQGTMVT VSS (SEQ ID NO:106) and a VL domain comprising the sequence of DIQMTQSPASLSASLGDKVTITCQASQNIDKYIAWYQQKPGKAPRQLIHYTSTLVSGT PSRFSGSGSGRDYSFSISSVESEDIASYYCLQYATLYTFGAGTKLELK (SEQ ID NO:107). In some embodiments, an anti-CD8 antibody or fusion protein of the present disclosure comprises a VH domain comprising the sequence of EVQLVESGGALVQPGRSLKLSCAASGLTFSDCYMAWVRQTPTKGLEWVSYISSDGG STYYGDSVKGRFTISRDNAKSTLYLQMNSLRSEDMATYYCACATDLSSYWSFDFWG PGTMVTVSS (SEQ ID NO:108) and a VL domain comprising the sequence of

(SEQ ID NO: 109)
DIQMTQSPSSLPVSLGERVTISCRASQGISNNLNWYQQKPDGTIKPLIY
HTSNLQSGVPSRFSGSGSGTDYSLTISSLEPEDFAMYYCQQDATFPLTF
GSGTKLEIK.

In some embodiments, an anti-CD8 antibody or fusion protein of the present disclosure comprises a VH domain comprising the sequence of SEQ ID NO:58 and a VL domain comprising the sequence of SEQ ID NO:59. In some embodiments, an anti-CD8 antibody or fusion protein of the present disclosure comprises a VH domain comprising the sequence of SEQ ID NO:62 and a VL domain comprising the sequence of SEQ ID NO:63. In some embodiments, an anti-CD8 antibody or fusion protein of the present disclosure comprises a VH domain comprising the sequence of SEQ ID NO:64 and a VL domain comprising the sequence of SEQ ID NO:65. In some embodiments, an anti-CD8 antibody or fusion protein of the present disclosure comprises a VH domain comprising the sequence of SEQ ID NO:66 and a VL domain comprising the sequence of SEQ ID NO:67. In some embodiments, an anti-CD8 antibody or fusion protein of the present disclosure comprises a VH domain comprising the sequence of SEQ ID NO:68 and a VL domain comprising the sequence of SEQ ID NO:69. In some embodiments, an anti-CD8 antibody or fusion protein of the present disclosure comprises a VH domain comprising the sequence of SEQ ID NO:70 and a VL domain comprising the sequence of SEQ ID NO:71. In some embodiments, an anti-CD8 antibody or fusion protein of the present disclosure comprises a VH domain comprising the sequence of SEQ ID NO:72 and a VL domain comprising the sequence of SEQ ID NO:73. In some embodiments, an anti-CD8 antibody or fusion protein of the present disclosure comprises a VH domain comprising the sequence of SEQ ID NO:185 and a VL domain comprising the sequence of SEQ ID NO: 186. In some embodiments, an anti-CD8 antibody or fusion protein of the present disclosure comprises a VH domain comprising the sequence of SEQ ID NO:245 and a VL domain comprising the sequence of SEQ ID NO:246. In some embodiments, an anti-CD8 antibody or fusion protein of the present disclosure comprises a VH domain comprising the sequence of SEQ ID NO:247 and a VL domain comprising the sequence of SEQ ID NO:248. In some embodiments, an anti-CD8 antibody or fusion protein of the present disclosure comprises a VH domain comprising the sequence of SEQ ID NO:249 and a VL domain comprising the sequence of SEQ ID NO:250. In some embodiments, an anti-CD8 antibody or fusion protein of the present disclosure comprises a VH domain comprising the sequence of SEQ ID NO:251 and a VL domain comprising the sequence of SEQ ID NO:252. In some embodiments, an anti-CD8 antibody or fusion protein of the present disclosure comprises a VH domain comprising the sequence of SEQ ID NO:253 and a VL domain comprising the sequence of SEQ ID NO:254. In some embodiments, an anti-CD8 antibody or fusion protein of the present disclosure comprises a VH domain comprising the sequence of SEQ ID NO:255 and a VL domain comprising the sequence of SEQ ID NO:256. In some embodiments, an anti-CD8 antibody or fusion protein of the present disclosure comprises a VH domain comprising the sequence of SEQ ID NO:257 and a VL domain comprising the sequence of SEQ ID NO:258.

In some embodiments, the antigen binding molecules (and fusion proteins) of the present disclosure specifically bind human CD8b and/or human CD8ab.

In some embodiments, the anti-CD8 antibody of the present disclosure is a human antibody or antibody fragment. In some embodiments, the anti-CD8 antibody of the present disclosure is a humanized antibody or antibody fragment.

In some embodiments, the anti-CD8 antibody of the present disclosure specifically binds human CD8b and/or human CD8ab with at least 10-fold, at least 20-fold, at least 30-fold, at least 40-fold, at least 50-fold, at least 60-fold, at least 70-fold, at least 80-fold, at least 90-fold, at least 100-fold, or at least 200-fold higher affinity than its binding to human CD8a and/or human CD8aa, e.g., as expressed on natural killer (NK) cells (e.g., human NK cells). In some embodiments, the anti-CD8 antibody of the present disclosure specifically binds human CD8b and/or human CD8ab with at least 10-fold higher affinity than its binding to human CD8a and/or human CD8aa, e.g., as expressed on natural killer (NK) cells. In some embodiments, the human CD8b and/or human CD8ab are expressed on the surface of a human cell, e.g., a human T cell.

In some embodiments, the anti-CD8 antibody of the present disclosure specifically binds to a cell expressing a human CD8ab heterodimer on its surface (e.g., a human T cell) with an EC50 that is less than 1000 nM. In some embodiments, the anti-CD8 antibody of the present disclosure specifically binds to human CD8+ T cells.

In some embodiments, an anti-CD8 antibody of the present disclosure comprises a VH domain comprising a CDR-H1 comprising the amino acid sequence of SEQ ID NO:110, a CDR-H2 comprising the amino acid sequence of SEQ ID NO:111, and a CDR-H3 comprising the amino acid sequence of SEQ ID NO:112 and a VL domain comprising a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6. In some embodiments, an anti-CD8 antibody of the present disclosure comprises 1, 2, or 3 heavy chain CDRs of antibody xhCD8v1 (e.g., as shown in Tables 1-3) and/or 1, 2, or 3 light chain CDRs of antibody xhCD8v1 (e.g., as shown in Tables 1-3). In some embodiments, the antibody is humanized.

In some embodiments, an anti-CD8 antibody of the present disclosure comprises a VH domain comprising a CDR-H1 comprising the amino acid sequence of SEQ ID NO:177, a CDR-H2 comprising the amino acid sequence of SEQ ID NO:178, and a CDR-H3 comprising the amino acid sequence of SEQ ID NO:179 and a VL domain comprising a CDR-L1 comprising the amino acid sequence of SEQ ID NO:180, a CDR-L2 comprising the amino acid sequence of SEQ ID NO:181, and a CDR-L3 comprising the amino acid sequence of SEQ ID NO: 182. In some embodiments, an anti-CD8 antibody of the present disclosure comprises 1, 2, or 3 heavy chain CDRs of antibody xhCD8v8 (e.g., as shown in Tables 1-3) and/or 1, 2, or 3 light chain CDRs of antibody xhCD8v8 (e.g., as shown in Tables 1-3). In some embodiments, the antibody is humanized.

In some embodiments, an anti-CD8 antibody of the present disclosure comprises a VH domain comprising a CDR-H1 comprising the amino acid sequence of SEQ ID NO:13, a CDR-H2 comprising the amino acid sequence of SEQ ID NO:14, and a CDR-H3 comprising the amino acid sequence of SEQ ID NO:15 and a VL domain comprising a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:18. In some embodiments, the VH domain comprises an amino acid sequence that is at least 90%, at least 95%, at least 99%, or 100% identical to the sequence of SEQ ID NO:62 and/or the VL domain comprises an amino acid sequence that is at least 90%, at least 95%, at least 99%, or 100% identical to the sequence of SEQ ID NO:63. In some embodiments, an anti-CD8 antibody of the present disclosure comprises 1, 2, or 3 heavy chain CDRs of antibody xhCD8v2 (e.g., as shown in Tables 1-3) and/or 1, 2, or 3 light chain CDRs of antibody xhCD8v2 (e.g., as shown in Tables 1-3). In some embodiments, the antibody is humanized.

In some embodiments, an anti-CD8 antibody of the present disclosure comprises a VH domain comprising a CDR-H1 comprising the amino acid sequence of SEQ ID NO:19, a CDR-H2 comprising the amino acid sequence of SEQ ID NO:20, and a CDR-H3 comprising the amino acid sequence of SEQ ID NO:21 and a VL domain comprising a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24. In some embodiments, the VH domain comprises an amino acid sequence that is at least 90%, at least 95%, at least 99%, or 100% identical to the sequence of SEQ ID NO:64 and/or the VL domain comprises an amino acid sequence that is at least 90%, at least 95%, at least 99%, or 100% identical to the sequence of SEQ ID NO:65. In some embodiments, an anti-CD8 antibody of the present disclosure comprises 1, 2, or 3 heavy chain CDRs of antibody xhCD8v3 (e.g., as shown in Tables 1-3) and/or 1, 2, or 3 light chain CDRs of antibody xhCD8v3 (e.g., as shown in Tables 1-3). In some embodiments, the antibody is humanized.

In some embodiments, an anti-CD8 antibody of the present disclosure comprises a VH domain comprising a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27 and a VL domain comprising a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30. In some embodiments, the VH domain comprises an amino acid sequence that is at least 90%, at least 95%, at least 99%, or 100% identical to the sequence of SEQ ID NO:66 and/or the VL domain comprises an amino acid sequence that is at least 90%, at least 95%, at least 99%, or 100% identical to the sequence of SEQ ID NO:67. In some embodiments, an anti-CD8 antibody of the present disclosure comprises 1, 2, or 3 heavy chain CDRs of antibody xhCD8v4 (e.g., as shown in Tables 1-3) and/or 1, 2, or 3 light chain CDRs of antibody xhCD8v4 (e.g., as shown in Tables 1-3). In some embodiments, the antibody is humanized.

In some embodiments, an anti-CD8 antibody of the present disclosure comprises a VH domain comprising a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33 and a VL domain comprising a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36. In some embodiments, the VH domain comprises an amino acid sequence that is at least 90%, at least 95%, at least 99%, or 100% identical to the sequence of SEQ ID NO:68 and/or the VL domain comprises an amino acid sequence that is at least 90%, at least 95%, at least 99%, or 100% identical to the sequence of SEQ ID NO:69. In some embodiments, an anti-CD8 antibody of the present disclosure comprises 1, 2, or 3 heavy chain CDRs of antibody xhCD8v5 (e.g., as shown in Tables 1-3) and/or 1, 2, or 3 light chain CDRs of antibody xhCD8v5 (e.g., as shown in Tables 1-3). In some embodiments, the antibody is humanized.

In some embodiments, an anti-CD8 antibody of the present disclosure comprises a VH domain comprising a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:70 and/or the VL domain comprises an amino acid sequence that is at least 90%, at least 95%, at least 99%, or 100% identical to the sequence of SEQ ID NO:71. In some embodiments, an anti-CD8 antibody of the present disclosure comprises 1, 2, or 3 heavy chain CDRs of antibody xhCD8v6 (e.g., as shown in Tables 1-3) and/or 1, 2, or 3 light chain CDRs of antibody xhCD8v6 (e.g., as shown in Tables 1-3). In some embodiments, the antibody is human.

In some embodiments, an anti-CD8 antibody of the present disclosure comprises a VH domain comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45 and a VL domain comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48. In some embodiments, the VH domain comprises an amino acid sequence that is at least 90%, at least 95%, at least 99%, or 100% identical to the sequence of SEQ ID NO:72 and/or the VL domain comprises an amino acid sequence that is at least 90%, at least 95%, at least 99%, or 100% identical to the sequence of SEQ ID NO:73. In some embodiments, an anti-CD8 antibody of the present disclosure comprises 1, 2, or 3 heavy chain CDRs of antibody xhCD8v7 (e.g., as shown in Tables 1-3) and/or 1, 2, or 3 light chain CDRs of antibody xhCD8v7 (e.g., as shown in Tables 1-3). In some embodiments, the antibody is human.

In some embodiments, an anti-CD8 antibody of the present disclosure comprises a VH domain comprising a CDR-H1 comprising the amino acid sequence of X₁X₂AIS, wherein X₁ is S, K, G, N, R, D, T, or G, and wherein X₂ is Y, L, H, or F (SEQ ID NO:259), a CDR-H2 comprising the amino acid sequence of X₁X₂X₃PX₄X₅X₆X₇X₈X₉YX₁₀QKFX₁₁G, wherein X₁ is G or H, X₂ is I or F, X₃ is I, N, or M, X₄ is G, N, H, S, R, I, or A, X₅ is A, N, H, S, T, F, or Y, X₆ is A, D, or G, X₇ is T, E, K, V, Q, or A, X₈ is A or T, X₉ is N or K, X₁₀ is A or N, and X₁₁ is Q or T (SEQ ID NO:260), and a CDR-H3 comprising the amino acid sequence of X₁X₂X₃GX₄X₅LFX₆X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO:261) and a VL domain comprising a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO:262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO:263), and a CDR-L3 comprising the amino acid sequence of QX₁X₂X₃X₄X₅PWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO:264). In some embodiments, the VH domain further comprises a FW-1 comprising the sequence QVQLVQSGAEVKKPGSSVKVSCKASGGTFS (SEQ ID NO:274), a FW-2 comprising the sequence WVRQAPGQGLEWMG (SEQ ID NO:275), a FW-3 comprising the sequence RVTITADESTSTAYMELSSLRSEDTAVYYCAR (SEQ ID NO:276), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:225, a CDR-H2 comprising the amino acid sequence of SEQ ID NO:226, and a CDR-H3 comprising the amino acid sequence of SEQ ID NO:227 and a VL domain comprising a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228. In some embodiments, the VH domain comprises an amino acid sequence that is at least 90%, at least 95%, at least 99%, or 100% identical to the sequence of SEQ ID NO:245 and/or the VL domain comprises an amino acid sequence that is at least 90%, at least 95%, at least 99%, or 100% identical to the sequence of SEQ ID NO:246. In some embodiments, an anti-CD8 antibody of the present disclosure comprises 1, 2, or 3 heavy chain CDRs of antibody xhCD8v9 (e.g., as shown in Tables 1-3) and/or 1, 2, or 3 light chain CDRs of antibody xhCD8v9 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:245 and a VL domain comprising a CDR-L1, CDR-L2, and CDR-L3 from the sequence of SEQ ID NO:246. In some embodiments, the VH domain further comprises a FW-1 comprising the sequence QVQLVQSGAEVKKPGSSVKVSCKASGGTFS (SEQ ID NO:274), a FW-2 comprising the sequence WVRQAPGQGLEWMG (SEQ ID NO:275), a FW-3 comprising the sequence RVTITADESTSTAYMELSSLRSEDTAVYYCAR (SEQ ID NO:276), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233 and a VL domain comprising a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236. In some embodiments, the VH domain comprises an amino acid sequence that is at least 90%, at least 95%, at least 99%, or 100% identical to the sequence of SEQ ID NO:251 and/or the VL domain comprises an amino acid sequence that is at least 90%, at least 95%, at least 99%, or 100% identical to the sequence of SEQ ID NO:252. In some embodiments, the VH domain comprises the amino acid sequence of SEQ ID NO:251; and the VL domain comprises the amino acid sequence of SEQ ID NO:252. In some embodiments, an anti-CD8 antibody of the present disclosure comprises 1, 2, or 3 heavy chain CDRs of antibody xhCD8v12 (e.g., as shown in Tables 1-3) and/or 1, 2, or 3 light chain CDRs of antibody xhCD8v12 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:251 and a VL domain comprising a CDR-L1, CDR-L2, and CDR-L3 from the sequence of SEQ ID NO:252. In some embodiments, the VH domain further comprises a FW-1 comprising the sequence QVQLVQSGAEVKKPGSSVKVSCKASGGTFS (SEQ ID NO:274), a FW-2 comprising the sequence WVRQAPGQGLEWMG (SEQ ID NO:275), a FW-3 comprising the sequence RVTITADESTSTAYMELSSLRSEDTAVYYCAR (SEQ ID NO:276), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233 and a VL domain comprising a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228. In some embodiments, the VH domain comprises an amino acid sequence that is at least 90%, at least 95%, at least 99%, or 100% identical to the sequence of SEQ ID NO:253 and/or the VL domain comprises an amino acid sequence that is at least 90%, at least 95%, at least 99%, or 100% identical to the sequence of SEQ ID NO:254. In some embodiments, an anti-CD8 antibody of the present disclosure comprises 1, 2, or 3 heavy chain CDRs of antibody xhCD8v13 (e.g., as shown in Tables 1-3) and/or 1, 2, or 3 light chain CDRs of antibody xhCD8v13 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:253 and a VL domain comprising a CDR-L1, CDR-L2, and CDR-L3 from the sequence of SEQ ID NO:254. In some embodiments, the VH domain further comprises a FW-1 comprising the sequence QVQLVQSGAEVKKPGSSVKVSCKASGGTFS (SEQ ID NO:274), a FW-2 comprising the sequence WVRQAPGQGLEWMG (SEQ ID NO:275), a FW-3 comprising the sequence RVTITADESTSTAYMELSSLRSEDTAVYYCAR (SEQ ID NO:276), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of X₁YX2MS, wherein X₁ is S, D, E, A, or Q and X₂ is A, G, or T (SEQ ID NO:268), a CDR-H2 comprising the amino acid sequence of DIX₁X₂X₃GX₄X₅TX₆YADSVKG, wherein X₁ is T, N, S, Q, E, H, R, or A, X₂ is Y, W, F, or H, X₃ is A, S, Q, E, or T, X₄ is G or E, X₅ is S or I, and X₆ is A or G (SEQ ID NO:269), and a CDR-H3 comprising the amino acid sequence of X₁X₂X₃YX₄WX₅X₆AX₇DX₈, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO:270) and a VL domain comprising a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the VH domain further comprises a FW-1 comprising the sequence EVQLVESGGGLVQPGGSLRLSCAASGFTFS (SEQ ID NO:281), a FW-2 comprising the sequence WVRQAPGKGLEWVS (SEQ ID NO:282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:247 and/or the VL domain comprises an amino acid sequence that is at least 90%, at least 95%, at least 99%, or 100% identical to the sequence of SEQ ID NO:248. In some embodiments, an anti-CD8 antibody of the present disclosure comprises 1, 2, or 3 heavy chain CDRs of antibody xhCD8v10 (e.g., as shown in Tables 1-3) and/or 1, 2, or 3 light chain CDRs of antibody xhCD8v10 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:247 and a VL domain comprising a CDR-L1, CDR-L2, and CDR-L3 from the sequence of SEQ ID NO:248. In some embodiments, the VH domain further comprises a FW-1 comprising the sequence EVQLVESGGGLVQPGGSLRLSCAASGFTFS (SEQ ID NO:281), a FW-2 comprising the sequence WVRQAPGKGLEWVS (SEQ ID NO:282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:249 and/or the VL domain comprises an amino acid sequence that is at least 90%, at least 95%, at least 99%, or 100% identical to the sequence of SEQ ID NO:250. In some embodiments, the VH domain comprises the amino acid sequence of SEQ ID NO:249; and the VL domain comprises the amino acid sequence of SEQ ID NO:250. In some embodiments, an anti-CD8 antibody of the present disclosure comprises 1, 2, or 3 heavy chain CDRs of antibody xhCD8v11 (e.g., as shown in Tables 1-3) and/or 1, 2, or 3 light chain CDRs of antibody xhCD8v11 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:249 and a VL domain comprising a CDR-L1, CDR-L2, and CDR-L3 from the sequence of SEQ ID NO:250. In some embodiments, the VH domain further comprises a FW-1 comprising the sequence EVQLVESGGGLVQPGGSLRLSCAASGFTFS (SEQ ID NO:281), a FW-2 comprising the sequence WVRQAPGKGLEWVS (SEQ ID NO:282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:229, a CDR-H2 comprising the amino acid sequence of SEQ ID NO:237, and a CDR-H3 comprising the amino acid sequence of SEQ ID NO:231 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:255 and/or the VL domain comprises an amino acid sequence that is at least 90%, at least 95%, at least 99%, or 100% identical to the sequence of SEQ ID NO:256. In some embodiments, an anti-CD8 antibody of the present disclosure comprises 1, 2, or 3 heavy chain CDRs of antibody xhCD8v14 (e.g., as shown in Tables 1-3) and/or 1, 2, or 3 light chain CDRs of antibody xhCD8v14 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:255 and a VL domain comprising a CDR-L1, CDR-L2, and CDR-L3 from the sequence of SEQ ID NO:256. In some embodiments, the VH domain further comprises a FW-1 comprising the sequence EVQLVESGGGLVQPGGSLRLSCAASGFTFS (SEQ ID NO:281), a FW-2 comprising the sequence WVRQAPGKGLEWVS (SEQ ID NO:282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:229, a CDR-H2 comprising the amino acid sequence of SEQ ID NO:237, and a CDR-H3 comprising the amino acid sequence of SEQ ID NO:231 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:257 and/or the VL domain comprises an amino acid sequence that is at least 90%, at least 95%, at least 99%, or 100% identical to the sequence of SEQ ID NO:258. In some embodiments, an anti-CD8 antibody of the present disclosure comprises 1, 2, or 3 heavy chain CDRs of antibody xhCD8v15 (e.g., as shown in Tables 1-3) and/or 1, 2, or 3 light chain CDRs of antibody xhCD8v15 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:257 and a VL domain comprising a CDR-L1, CDR-L2, and CDR-L3 from the sequence of SEQ ID NO:258. In some embodiments, the VH domain further comprises a FW-1 comprising the sequence EVQLVESGGGLVQPGGSLRLSCAASGFTFS (SEQ ID NO:281), a FW-2 comprising the sequence WVRQAPGKGLEWVS (SEQ ID NO:282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

Multiple definitions for the CDR sequences of antibody variable domains are known in the art. Unless otherwise specified, CDR sequences are described herein according to the definition of Kabat (see, e.g., Kabat et al., Sequences of Proteins of Immunological Interest, Fifth Edition, NIH Publication 91-3242, Bethesda MD (1991), vols. 1-3). However, other definitions are known and contemplated for use. For example, in some embodiments, CDR sequences can be described by the definition of Chothia (see, e.g., Chothia and Lesk, J. Mol. Biol. 196:901-917 (1987).

In some embodiments, an anti-CD8 antibody of the present disclosure comprises a VH domain comprising a CDR-H1 comprising the amino acid sequence of SEQ ID NO:49, a CDR-H2 comprising the amino acid sequence of SEQ ID NO:50, and a CDR-H3 comprising the amino acid sequence of SEQ ID NO:3 and a VL domain comprising a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6. In some embodiments, an anti-CD8 antibody of the present disclosure comprises a VH domain comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:15 and a VL domain comprising a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:18. In some embodiments, an anti-CD8 antibody of the present disclosure comprises a VH domain comprising a CDR-H1 comprising the amino acid sequence of SEQ ID NO:53, a CDR-H2 comprising the amino acid sequence of SEQ ID NO:52, and a CDR-H3 comprising the amino acid sequence of SEQ ID NO:21 and a VL domain comprising a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24. In some embodiments, an anti-CD8 antibody of the present disclosure comprises a VH domain comprising a CDR-H1 comprising the amino acid sequence of SEQ ID NO:49, a CDR-H2 comprising the amino acid sequence of SEQ ID NO:52, and a CDR-H3 comprising the amino acid sequence of SEQ ID NO:27 and a VL domain comprising a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30. In some embodiments, an anti-CD8 antibody of the present disclosure comprises a VH domain comprising a CDR-H1 comprising the amino acid sequence of SEQ ID NO:54, a CDR-H2 comprising the amino acid sequence of SEQ ID NO:52, and a CDR-H3 comprising the amino acid sequence of SEQ ID NO:33 and a VL domain comprising a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36. In some embodiments, an anti-CD8 antibody of the present disclosure comprises a VH domain comprising a CDR-H1 comprising the amino acid sequence of SEQ ID NO:55, a CDR-H2 comprising the amino acid sequence of SEQ ID NO:56, and a CDR-H3 comprising the amino acid sequence of SEQ ID NO:39 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, an anti-CD8 antibody of the present disclosure comprises a VH domain comprising a CDR-H1 comprising the amino acid sequence of SEQ ID NO:55, a CDR-H2 comprising the amino acid sequence of SEQ ID NO:57, and a CDR-H3 comprising the amino acid sequence of SEQ ID NO:45 and a VL domain comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48. In some embodiments, an anti-CD8 antibody of the present disclosure comprises a VH domain comprising a CDR-H1 comprising the amino acid sequence of SEQ ID NO:183, a CDR-H2 comprising the amino acid sequence of SEQ ID NO:184, and a CDR-H3 comprising the amino acid sequence of SEQ ID NO:179 and a VL domain comprising a CDR-L1 comprising the amino acid sequence of SEQ ID NO:180, a CDR-L2 comprising the amino acid sequence of SEQ ID NO:181, and a CDR-L3 comprising the amino acid sequence of SEQ ID NO:182.

In some embodiments, an anti-CD8 antibody of the present disclosure comprises a VH domain comprising a CDR-H1 comprising the amino acid sequence of GX₁X₂FX₃X₄X₅, wherein X₁ is G, Y, S, or A, X₂ is T, S, G, R, N, or H, X₃ is S, T, R, H, Y, G, or P, X₄ is S, K, G, N, R, D, T, or G, and X₅ is Y, L, H, or F (SEQ ID NO:265), a CDR-H2 comprising the amino acid sequence of X₁PX₂X₃X₄X₅, wherein X₁ is I, N, or M, X₂ is G, N, H, S, R, I, or A, X₃ is A, N, H, S, T, F, or Y, X₄ is A, D, or G, and X₅ is T, E, K, V, Q, or A (SEQ ID NO:266), and a CDR-H3 comprising the amino acid sequence of X₁X₂X₃GX₄X₅LFX₆X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO:267) and a VL domain comprising a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO:262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO:263), and a CDR-L3 comprising the amino acid sequence of QX₁X₂X₃X₄X₅PWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO:264). In some embodiments, the VH domain further comprises a FW-1 comprising the sequence QVQLVQSGAEVKKPGSSVKVSCKAS (SEQ ID NO:278), a FW-2 comprising the sequence AISWVRQAPGQGLEWMGGI (SEQ ID NO:279), a FW-3 comprising the sequence ANYAQKFQGRVTITADESTSTAYMELSSLRSEDTAVYYCAR (SEQ ID NO:280), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:238, a CDR-H2 comprising the amino acid sequence of SEQ ID NO:239, and a CDR-H3 comprising the amino acid sequence of SEQ ID NO:233 and a VL domain comprising a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228. In some embodiments, the VH domain further comprises a FW-1 comprising the sequence QVQLVQSGAEVKKPGSSVKVSCKAS (SEQ ID NO:278), a FW-2 comprising the sequence AISWVRQAPGQGLEWMGGI (SEQ ID NO:279), a FW-3 comprising the sequence ANYAQKFQGRVTITADESTSTAYMELSSLRSEDTAVYYCAR (SEQ ID NO:280), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233 and a VL domain comprising a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236. In some embodiments, the VH domain further comprises a FW-1 comprising the sequence QVQLVQSGAEVKKPGSSVKVSCKAS (SEQ ID NO:278), a FW-2 comprising the sequence AISWVRQAPGQGLEWMGGI (SEQ ID NO:279), a FW-3 comprising the sequence ANYAQKFQGRVTITADESTSTAYMELSSLRSEDTAVYYCAR (SEQ ID NO:280), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233 and a VL domain comprising a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228. In some embodiments, the VH domain further comprises a FW-1 comprising the sequence QVQLVQSGAEVKKPGSSVKVSCKAS (SEQ ID NO:278), a FW-2 comprising the sequence AISWVRQAPGQGLEWMGGI (SEQ ID NO:279), a FW-3 comprising the sequence ANYAQKFQGRVTITADESTSTAYMELSSLRSEDTAVYYCAR (SEQ ID NO:280), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of GFTFX₁X₂Y, wherein X₁ is S, D, E, Q, S, or A and X₂ is S, D, E, A, or Q (SEQ ID NO:271), a CDR-H2 comprising the amino acid sequence of X₁X₂X₃GX₄X₅, wherein X₁ is T, N, S, Q, E, H, R or A, X₂ is Y, W, F, or H, X₃ is A, S, Q, E, or T, X₄ is G or E, and X₅ is S or I (SEQ ID NO:272), and a CDR-H3 comprising the amino acid sequence of X₁X₂X₃YX₄WX₅X₆AX₇DX₈, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO:273) and a VL domain comprising a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the VH domain further comprises a FW-1 comprising the sequence EVQLVESGGGLVQPGGSLRLSCAAS (SEQ ID NO:286), a FW-2 comprising the sequence AMSWVRQAPGKGLEWVSDI (SEQ ID NO:287), a FW-3 comprising the sequence TAYADSVKGRFTISRDNAKNSLYLQMNSLRAEDTAVYYCAR (SEQ ID NO:288), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:240, a CDR-H2 comprising the amino acid sequence of SEQ ID NO:241, and a CDR-H3 comprising the amino acid sequence of SEQ ID NO:242 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain further comprises a FW-1 comprising the sequence EVQLVESGGGLVQPGGSLRLSCAAS (SEQ ID NO:286), a FW-2 comprising the sequence AMSWVRQAPGKGLEWVSDI (SEQ ID NO:287), a FW-3 comprising the sequence TAYADSVKGRFTISRDNAKNSLYLQMNSLRAEDTAVYYCAR (SEQ ID NO:288), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:240, a CDR-H2 comprising the amino acid sequence of SEQ ID NO:244, and a CDR-H3 comprising the amino acid sequence of SEQ ID NO:242 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain further comprises a FW-1 comprising the sequence EVQLVESGGGLVQPGGSLRLSCAAS (SEQ ID NO:286), a FW-2 comprising the sequence AMSWVRQAPGKGLEWVSDI (SEQ ID NO:287), a FW-3 comprising the sequence TAYADSVKGRFTISRDNAKNSLYLQMNSLRAEDTAVYYCAR (SEQ ID NO:288), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296). In some embodiments, the present disclosure provides an anti-CD8 antibody comprising a VH domain comprising CDR-H1, CDR-H2, and CDR-H3 sequences of a single antibody listed in Table 1 and a VL domain comprising CDR-L1, CDR-L2, and CDR-L3 sequences of the single antibody listed in Table 1. For example, the anti-CD8 antibody comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 1. In some embodiments, the present disclosure provides an anti-CD8 antibody comprising a VH domain comprising CDR-H1, CDR-H2, and CDR-H3 sequences of a single antibody listed in Table 2 and a VL domain comprising CDR-L1, CDR-L2, and CDR-L3 sequences of the single antibody listed in Table 2. For example, the anti-CD8 antibody comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 2. In some embodiments, the present disclosure provides a fusion protein comprising an anti-CD8 antibody comprising a VH domain comprising CDR-H1, CDR-H2, and CDR-H3 sequences of the single antibody listed in Table 1 and a VL domain comprising CDR-L1, CDR-L2, and CDR-L3 sequences of a single antibody listed in Table 1. For example, the anti-CD8 antibody of the fusion protein comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 1. In some embodiments, the present disclosure provides a fusion protein comprising an anti-CD8 antibody comprising a VH domain comprising CDR-H1, CDR-H2, and CDR-H3 sequences of a single antibody listed in Table 2 and a VL domain comprising CDR-L1, CDR-L2, and CDR-L3 sequences of the single antibody listed in Table 2. For example, the anti-CD8 antibody of the fusion protein comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 2. In some embodiments, the present disclosure provides an anti-CD8 antibody comprising a VH domain comprising CDR-H1, CDR-H2, and CDR-H3 sequences of a VH domain listed in Table 3 and a VL domain comprising CDR-L1, CDR-L2, and CDR-L3 sequences of a VL domain listed in Table 3 (in some embodiments, the VH and VL domains are from the same single antibody listed in Table 3). For example, the anti-CD8 antibody comprises the VH and VL of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, or xhCD8v15 shown in Table 3. In some embodiments, the present disclosure provides a fusion protein comprising an anti-CD8 antibody comprising a VH domain comprising CDR-H1, CDR-H2, and CDR-H3 sequences of a VH domain listed in Table 3 and a VL domain comprising CDR-L1, CDR-L2, and CDR-L3 sequences of a VL domain listed in Table 3 (in some embodiments, the VH and VL domains are from the same single antibody listed in Table 3). In some embodiments, the present disclosure provides an anti-CD8 antibody comprising a VH domain sequence and a VL domain sequence for a single antibody as listed in Table 3. In some embodiments, the present disclosure provides a fusion protein comprising an anti-CD8 antibody comprising a VH domain sequence and a VL domain sequence for a single antibody as listed in Table 3. For example, the anti-CD8 antibody of the fusion protein comprises the VH and VL of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, or xhCD8v15 shown in Table 3.

TABLE 1
Anti-CD8 antibody CDRs (Kabat)

| Name | CDR-H1 | CDR-H2 | CDR-H3 | CDR-L1 | CDR-L2 | CDR-L3 |
|---|---|---|---|---|---|---|
| xhCD8v1 | KYTMH | HFNPNND | DGLGLRL | GASENIY | GATNLAD | QNILDTP |
| | (SEQ ID | ETKYNQK | FAD | GALN | (SEQ ID | WT |
| | NO: 110) | FTG | (SEQ ID | (SEQ ID | NO: 5) | (SEQ ID |
| | | (SEQ ID | NO: 112) | NO: 4) | | NO: 6) |
| | | NO: 111) | | | | |
| xhCD8v1.1 | KYAIS | HFNPNND | DGLGLRL | RASENIY | GATNLAD | QNILDTP |
| | (SEQ ID | ETKYNQK | FAD | GALN | (SEQ ID | WT |
| | NO: 7) | FQG | (SEQ ID | (SEQ ID | NO: 11) | (SEQ ID |
| | | (SEQ ID | NO: 9) | NO: 10) | | NO: 12) |
| | | NO: 8) | | | | |
| xhCD8v2 | NFAIS | GIIPGHAK | DGLGIRL | RASQEIY | GATNLQS | QDIYDAP |
| | (SEQ ID | ANYAQK | FAD | GALN | (SEQ ID | WT |
| | NO: 13) | FQG | (SEQ ID | (SEQ ID | NO: 17) | (SEQ ID |
| | | (SEQ ID | NO: 15) | NO: 16) | | NO: 18) |
| | | NO: 14) | | | | |
| xhCD8v3 | KFAIS | GIIPGHAK | DGLGIRL | RASQEIY | GATNLQS | QDIYDAP |
| | (SEQ ID | ANYAQK | FAD | GALN | (SEQ ID | WT |
| | NO: 19) | FQG | (SEQ ID | (SEQ ID | NO: 23) | (SEQ ID |
| | | (SEQ ID | NO: 21) | NO: 22) | | NO: 24) |
| | | NO: 20) | | | | |
| xhCD8v4 | KYAIS | GIIPGHAK | DGLGIRL | RASQKIY | GATNLQS | QNTYDTP |
| | (SEQ ID | ANYAQK | FAD | GALN | (SEQ ID | WT |
| | NO: 25) | FQG | (SEQ ID | (SEQ ID | NO: 29) | (SEQ ID |
| | | (SEQ ID | NO: 27) | NO: 28) | | NO: 30) |
| | | NO: 26) | | | | |
| xhCD8v5 | GHAIS | GIIPGHAK | DGLGIRL | RASQKIY | GATNLQS | QNTYDTP |
| | (SEQ ID | ANYAQK | FAD | GALN | (SEQ ID | WT |
| | NO: 31) | FQG | (SEQ ID | (SEQ ID | NO: 35) | (SEQ ID |
| | | (SEQ ID | NO: 33) | NO: 34) | | NO: 36) |
| | | NO: 32) | | | | |
| xhCD8v6 | DYGMS | DINWSGE | SNSYRW | RASQSVS | GASSRAT | QQYGSSP |
| | (SEQ ID | ITAYADS | DDALDI | SNLA | (SEQ ID | PVT |
| | NO: 37) | VKG | (SEQ ID | (SEQ ID | NO: 41) | (SEQ ID |
| | | (SEQ ID | NO: 39) | NO: 40) | | NO: 42) |
| | | NO: 38) | | | | |
| xhCD8v7 | DYAMH | VISYDGS | DRIGWYD | RASHSVG | DASNRAT | QQRSNW |
| | (SEQ ID | NKYYAD | YDAFDI | SNLA | (SEQ ID | PPT |
| | NO: 43) | SVKG | (SEQ ID | (SEQ ID | NO: 47) | (SEQ ID |
| | | (SEQ ID | NO: 45) | NO: 46) | | NO: 48) |
| | | NO: 44) | | | | |
| xhCD8v8 | SYWMN | QIYPGDG | SGAAFSS | RASENIY | AATNLAD | QHFWGTP |
| | (SEQ ID | DTNYNG | YYAMDY | SNLA | (SEQ ID | WT (SEQ |
| | NO: 177) | KFKG | (SEQ ID | (SEQ ID | NO: 181) | ID NO: 182) |
| | | (SEQ ID | NO: 179) | NO: 180) | | |
| | | NO: 178) | | | | |
| xhCD8v9 | SYAIS | GIIPGAAT | DAAGIRL | RASQEIY | GATNLQS | QSTYDAP |
| | (SEQ ID | ANYAQK | FAD | GALN | (SEQ ID | WT (SEQ |
| | NO: 225) | FQG | (SEQ ID | (SEQ ID | NO: 17) | ID NO: 228) |
| | | (SEQ ID | NO: 227) | NO: 16) | | |
| | | NO: 226) | | | | |
| xhCD8v10 | SYAMS | DITYAGG | SNAYAW | RASQSVS | GASSRAT | QQYGSSP |
| | (SEQ ID | STAYADS | DDALDI | SNLA | (SEQ ID | PVT |
| | NO: 229) | VKG (SEQ | (SEQ ID | (SEQ ID | NO: 41) | (SEQ ID |
| | | ID NO: 230) | NO: 231) | NO: 40) | | NO: 42) |
| xhCD8v11 | SYAMS | DITYAGG | SNAYAW | RASQSVS | GASSRAT | QQYGSSP |
| | (SEQ ID | STAYADS | DDALDI | SNLA | (SEQ ID | PVT |
| | NO: 229) | VKG (SEQ | (SEQ ID | (SEQ ID | NO: 41) | (SEQ ID |
| | | ID NO: 230) | NO: 231) | NO: 40) | | NO: 42) |
| xhCD8v12 | SYAIS | GIIPGYAT | DAAGIRL | RASQSIY | GASNLQS | QSTYTAP |
| | (SEQ ID | ANYAQK | FAD (SEQ | GALN | (SEQ ID | WT (SEQ |
| | NO: 225) | FQG (SEQ | ID NO: 233) | (SEQ ID | NO: 235) | ID NO: 236) |
| | | ID NO: 232) | | NO: 234) | | |
| xhCD8v13 | SYAIS | GIIPGYAT | DAAGIRL | RASQEIY | GATNLQS | QSTYDAP |
| | (SEQ ID | ANYAQK | FAD (SEQ | GALN | (SEQ ID | WT (SEQ |
| | NO: 225) | FQG (SEQ | ID NO: 233) | (SEQ ID | NO: 17) | ID NO: 228) |
| | | ID NO: 232) | | NO: 16) | | |
| xhCD8v14 | SYAMS | DISYAGG | SNAYAW | RASQSVS | GASSRAT | QQYGSSP |
| | (SEQ ID | STAYADS | DDALDI | SNLA | (SEQ ID | PVT |
| | NO: 229) | VKG (SEQ | (SEQ ID | (SEQ ID | NO: 41) | (SEQ ID |
| | | ID NO: 237) | NO: 231) | NO: 40) | | NO: 42) |
| xhCD8v15 | SYAMS | DISYAGG | SNAYAW | RASQSVS | GASSRAT | QQYGSSP |
| | (SEQ ID | STAYADS | DDALDI | SNLA | (SEQ ID | PVT |
| | NO: 229) | VKG (SEQ | (SEQ ID | (SEQ ID | NO: 41) | (SEQ ID |
| | | ID NO: 237) | NO: 231) | NO: 40) | | NO: 42) |
| V9 family | X₁X₂AIS | X₁X₂X₃PX₄ | X₁X₂X₃GX₄ | X₁X₂SX₃X₄ | GX₁X₂X₃L | QX₁X₂X₃X |
| | X₁ is S, K, | X₅X₆X₇X₈ | X₅LFX₆X₇ | IX₅GX₆LN | X₄X₅ | ₄X₅PWT |
| | G, N, R, D, | X₉YX₁₀QK | X₁ is D or | X₁ is R or | X₁ is A or | X₁ is S, N, |
| | T, or G | FX₁₁G | A, | G, | S, X₂ is T, | D, Q, A, or |
| | X₂ is Y, L, | X₁ is G or | X₂ is A, G, | X₂ is A or | S, E, Q, or | E, |
| | H, or F | H, X₂ is I | E, R, Y, K, | T, | D, X₃ is N, | X₂ is T, I, |
| | (SEQ ID | or F, | N, Q, L, or | X₃ is Q or | R, A, E, or | or S, |
| | NO: 259) | X₃ is I, N, | F, | E, | H, X₄ is Q | X₃ is Y, L, |
| | | or M, | X₃ is A, L, | X₄ is E, N, | or A, | or F, |
| | | X₄ is G, N, | P, or Y, | T, S, A, K, | X₅ is S or D | X₄ is D, G, |
| | | H, S, R, I, | X₄ is I or L, | D, G, R, or | (SEQ ID | T, E, Q, A, |
| | | or A, X₅ is | X₅ is R, A, | Q, | NO: 263) | or Y, |
| | | A, N, H, S, | Q, or S, | X₅ is Y or | | X₅ is A, T, |
| | | T, F, or Y, | X₆ is A or | S, X₆ is A | | R, S, K, or |
| | | X₆ is A, D, | D, | or V (SEQ | | Y |
| | | or G, | X₇ is D, E, | ID NO: 262) | | (SEQ ID |
| | | X₇ is T, E, | A, or S | | | NO: 264) |
| | | K, V, Q, or | (SEQ ID | | | |
| | | A, | NO: 261) | | | |
| | | X₈ is A or | | | | |
| | | T, X₉ is N | | | | |
| | | or K, X₁₀ is | | | | |
| | | A or N, X₁₁ | | | | |
| | | is Q or T | | | | |
| | | (SEQ ID | | | | |
| | | NO: 260) | | | | |
| V11 family | X₁YX₂MS | DIX₁X₂X₃ | X₁X₂X₃YX₄ | RASQSVS | GASSRAT | QQYGSSP |
| | X₁ is S, D, | GX₄X₅TX₆ | WX₅X₆AX₇ | SNLA | (SEQ ID | PVT |
| | E, A, or Q | YADSVK | DX₈ | (SEQ ID | NO: 41) | (SEQ ID |
| | X₂ is A, G, | G | X₁ is S or | NO: 40) | | NO: 42) |
| | or T (SEQ | X₁ is T, N, | A, X₂ is N, | | | |
| | ID NO: 268) | S, Q, E, H, | H, A, D, L, | | | |
| | | R, or A, | Q, Y, or R, | | | |
| | | X₂ is Y, W, | X₃ is A, N, | | | |
| | | F, or H, | S, or G, | | | |
| | | X₃ is A, S, | X₄ is A, V, | | | |
| | | Q, E, or T, | R, E, or S, | | | |
| | | X₄ is G or | X₅ is D or | | | |
| | | E, X₅ is S | S, X₆ is D, | | | |
| | | or I, | N, Q, E, S, | | | |
| | | X₆ is A or | T, or L, | | | |
| | | G (SEQ ID | X₇ is L, F, | | | |
| | | NO: 269) | or M, | | | |
| | | | X₈ is I, Y, | | | |
| | | | or V | | | |
| | | | (SEQ ID | | | |
| | | | NO: 270) | | | |

TABLE 2
Anti-CD8 antibody CDRs (Chothia)

| Name | CDR-H1 | CDR-H2 | CDR-H3 | CDR-L1 | CDR-L2 | CDR-L3 |
|---|---|---|---|---|---|---|
| xhCD8v1 | GYTFTKY | NPNNDE | DGLGLRL | GASENIY | GATNLAD | QNILDTP |
| | (SEQ ID | (SEQ ID | FAD | GALN | (SEQ ID | WT |
| | NO: 49) | NO: 50) | (SEQ ID | (SEQ ID | NO: 5) | (SEQ ID |
| | | | NO: 3) | NO: 4) | | NO: 6) |
| xhCD8v1.1 | GYTFTKY | NPNNDE | DGLGLRL | RASENIY | GATNLAD | QNILDTP |
| | (SEQ ID | (SEQ ID | FAD | GALN | (SEQ ID | WT |
| | NO: 49) | NO: 50) | (SEQ ID | (SEQ ID | NO: 11) | (SEQ ID |
| | | | NO: 9) | NO: 10) | | NO: 12) |
| xhCD8v2 | GYRFHNF | IPGHAK | DGLGIRL | RASQEIY | GATNLQS | QDIYDAP |
| | (SEQ ID | (SEQ ID | FAD | GALN | (SEQ ID | WT |
| | NO: 51) | NO: 52) | (SEQ ID | (SEQ ID | NO: 17) | (SEQ ID |
| | | | NO: 15) | NO: 16) | | NO: 18) |
| xhCD8v3 | GSRFYKF | IPGHAK | DGLGIRL | RASQEIY | GATNLQS | QDIYDAP |
| | (SEQ ID | (SEQ ID | FAD | GALN | (SEQ ID | WT |
| | NO: 53) | NO: 52) | (SEQ ID | (SEQ ID | NO: 23) | (SEQ ID |
| | | | NO: 21) | NO: 22) | | NO: 24) |
| xhCD8v4 | GYTFTKY | IPGHAK | DGLGIRL | RASQKIY | GATNLQS | QNTYDTP |
| | (SEQ ID | (SEQ ID | FAD | GALN | (SEQ ID | WT |
| | NO: 49) | NO: 52) | (SEQ ID | (SEQ ID | NO: 29) | (SEQ ID |
| | | | NO: 27) | NO: 28) | | NO: 30) |
| xhCD8v5 | GSGFRGH | IPGHAK | DGLGIRL | RASQKIY | GATNLQS | QNTYDTP |
| | (SEQ ID | (SEQ ID | FAD | GALN | (SEQ ID | WT |
| | NO: 54) | NO: 52) | (SEQ ID | (SEQ ID | NO: 35) | (SEQ ID |
| | | | NO: 33) | NO: 34) | | NO: 36) |
| xhCD8v6 | GFTFDDY | NWSGEI | SNSYRW | RASQSVS | GASSRAT | QQYGSSP |
| | (SEQ ID | (SEQ ID | DDALDI | SNLA | (SEQ ID | PVT |
| | NO: 55) | NO: 56) | (SEQ ID | (SEQ ID | NO: 41) | (SEQ ID |
| | | | NO: 39) | NO: 40) | | NO: 42) |
| xhCD8v7 | GFTFDDY | SYDGSN | DRIGWYD | RASHSVG | DASNRAT | QQRSNW |
| | (SEQ ID | (SEQ ID | YDAFDI | SNLA | (SEQ ID | PPT |
| | NO: 55) | NO: 57) | (SEQ ID | (SEQ ID | NO: 47) | (SEQ ID |
| | | | NO: 45) | NO: 46) | | NO: 48) |
| xhCD8v8 | GYAFSSY | YPGDGD | SGAAFSS | RASENIY | AATNLAD | QHFWGTP |
| | (SEQ ID | (SEQ ID | YYAMDY | SNLA | (SEQ ID | WT (SEQ |
| | NO: 183) | NO: 184) | (SEQ ID | (SEQ ID | NO: 181) | ID NO: 182) |
| | | | NO: 179) | NO: 180) | | |
| xhCD8v9 | GGTFSSY | IPGAAT | DAAGIRL | RASQEIY | GATNLQS | QSTYDAP |
| | (SEQ ID | (SEQ ID | FAD (SEQ | GALN | (SEQ ID | WT (SEQ |
| | NO: 238) | NO: 239) | ID NO: 233) | (SEQ ID | NO: 17) | ID NO: 228) |
| | | | | NO: 16) | | |
| xhCD8v10 | GFTFSSY | TYAGGS | SNAYAW | RASQSVS | GASSRAT | QQYGSSP |
| | (SEQ ID | (SEQ ID | DDALDI | SNLA | (SEQ ID | PVT |
| | NO: 240) | NO: 241) | (SEQ ID | (SEQ ID | NO: 41) | (SEQ ID |
| | | | NO: 242) | NO: 40) | | NO: 42) |
| xhCD8v11 | GFTFSSY | TYAGGS | SNAYAW | RASQSVS | GASSRAT | QQYGSSP |
| | (SEQ ID | (SEQ ID | DDALDI | SNLA | (SEQ ID | PVT |
| | NO: 240) | NO: 241) | (SEQ ID | (SEQ ID | NO: 41) | (SEQ ID |
| | | | NO: 242) | NO: 40) | | NO: 42) |
| xhCD8v12 | GGTFSSY | IPGYAT | DAAGIRL | RASQSIY | GASNLQS | QSTYTAP |
| | (SEQ ID | (SEQ ID | FAD (SEQ | GALN | (SEQ ID | WT (SEQ |
| | NO: 238) | NO: 243) | ID NO: 233) | (SEQ ID | NO: 235) | ID NO: 236) |
| | | | | NO: 234) | | |
| xhCD8v13 | GGTFSSY | IPGYAT | DAAGIRL | RASQEIY | GATNLQS | QSTYDAP |
| | (SEQ ID | (SEQ ID | FAD (SEQ | GALN | (SEQ ID | WT (SEQ |
| | NO: 238) | NO: 243) | ID NO: 233) | (SEQ ID | NO: 17) | ID NO: 228) |
| | | | | NO: 16) | | |
| xhCD8v14 | GFTFSSY | SYAGGS | SNAYAW | RASQSVS | GASSRAT | QQYGSSP |
| | (SEQ ID | (SEQ ID | DDALDI | SNLA | (SEQ ID | PVT |
| | NO: 240) | NO: 244) | (SEQ ID | (SEQ ID | NO: 41) | (SEQ ID |
| | | | NO: 242) | NO: 40) | | NO: 42) |
| xhCD8v15 | GFTFSSY | SYAGGS | SNAYAW | RASQSVS | GASSRAT | QQYGSSP |
| | (SEQ ID | (SEQ ID | DDALDI | SNLA | (SEQ ID | PVT |
| | NO: 240) | NO: 244) | (SEQ ID | (SEQ ID | NO: 41) | (SEQ ID |
| | | | NO: 242) | NO: 40) | | NO: 42) |
| V9 family | GX₁X₂FX₃ | X₁PX₂X₃X₄ | X₁X₂X₃GX₄ | X₁X₂SX₃X₄ | GX₁X₂X₃L | QX₁X₂X₃X |
| | X₄X₅ | X₅ | X₅LFX₆X₇ | IX₅GX₆LN | X₄X₅ | ₄X₅PWT |
| | X₁ is G, Y, | X₁ is I, N, | X₁ is D or | X₁ is R or | X₁ is A or | X₁ is S, N, |
| | S, or A, | or M, | A, | G, | S, X₂ is T, | D, Q, A, or |
| | X₂ is T, S, | X₂ is G, N, | X₂ is A, G, | X₂ is A or | S, E, Q, or | E, |
| | G, R, N, or | H, S, R, I, | E, R, Y, K, | T, | D, X₃ is N, | X₂ is T, I, |
| | H, | or A, | N, Q, L, or | X₃ is Q or | R, A, E, or | or S, |
| | X₃ is S, T, | X₃ is A, N, | F, | E, | H, X₄ is Q | X₃ is Y, L, |
| | R, H, Y, G, | H, S, T, F, | X₃ is A, L, | X₄ is E, N, | or A, | or F, |
| | or P, | or Y, | P, or Y, | T, S, A, K, | X₅ is S or D | X₄ is D, G, |
| | X₄ is S, K, | X₄ is A, D, | X₄ is I | D, G, R, | (SEQ ID | T, E, Q, A, |
| | G, N, R, D, | or G, | or L, | or Q, | NO: 263) | or Y, |
| | T, or G, | X₅ is T, E, | X₅ is R, A, | X₅ is Y or | | X₅ is A, T, |
| | X₅ is Y, L, | K, V, Q, or | Q, or S, | S, X₆ is A | | R, S, K, or |
| | H, or F | A | X₆ is A or | or V (SEQ | | Y |
| | (SEQ ID | (SEQ ID | D, | ID NO: 262) | | (SEQ ID |
| | NO: 265) | NO: 266) | X₇ is D, E, | | | NO: 264) |
| | | | A, or S | | | |
| | | | (SEQ ID | | | |
| | | | NO: 267) | | | |
| V11 family | GFTFX₁X₂ | X₁X₂X₃GX | X₁X₂X₃YX₄ | RASQSVS | GASSRAT | QQYGSSP |
| | Y | ₄X₅ | WX₅X₆AX₇ | SNLA | (SEQ ID | PVT |
| | X₁ is S, D, | X₁ is T, N, | DX₈ | (SEQ ID | NO: 41) | (SEQ ID |
| | E, Q, S, or | S, Q, E, H, | X₁ is S or | NO: 40) | | NO: 42) |
| | A | R or A, | A, X₂ is N, | | | |
| | X₂ is S, D, | X₂ is Y, W, | H, A, D, L, | | | |
| | E, A, or Q | F, or H, | Q, Y, or R, | | | |
| | (SEQ ID | X₃ is A, S, | X₃ is A, N, | | | |
| | NO: 271) | Q, E, or T, | S, or G, | | | |
| | | X₄ is G or | X₄ is A, V, | | | |
| | | E, | R, E, or S, | | | |
| | | X₅ is S or | X₅ is D or | | | |
| | | I | S, X₆ is D, | | | |
| | | (SEQ ID | N, Q, E, S, | | | |
| | | NO: 272) | T, or L, | | | |
| | | | X₇ is L, F, | | | |
| | | | or M, | | | |
| | | | X₈ is I, Y, | | | |
| | | | or V | | | |
| | | | (SEQ ID | | | |
| | | | NO: 273) | | | |

TABLE 3
Anti-CD8 antibody variable domain sequences

| Name | VH | VL |
|---|---|---|
| xhCD8v1 | QVHLQQSGPELVKPGASVKMSCKT | DIQMTQSPASLSASVGETVTITCGA |
| | SGYTFTKYTMHWVKQGHEESLEWI | SENIYGALNWYQRKQGKSPQLLIFG |
| | GHFNPNNDETKYNQKFTGKATLTV | ATNLADGVSSRFSGSGSDRQYSLKI |
| | DKSSTTAYMELRSLTSDDSALYYC | SSLHPDDVATYYCQNILDTPWTFG |
| | ARDGLGLRLFADWGQGTLITVSA | GGTKLEIK |
| | (SEQ ID NO: 58) | (SEQ ID NO: 59) |
| xhCD8v1.1 | QVQLVQSGAEVKKPGSSVKVSCKA | DIQMTQSPSSLSASVGDRVTITCRA |
| | SGYTFTKYAISWVRQAPGQGLEW | SENIYGALNWYQQKPGKAPKLLIY |
| | MGHFNPNNDETKYNQKFQGRVTIT | GATNLADGVPSRFSGSGSGTDFTLT |
| | ADESTSTAYMELSSLRSEDTAVYY | ISSLQPEDFATYYCQNILDTPWTFG |
| | CARDGLGLRLFADWGQGT | GGTKLEIK |
| | LVTVSS | (SEQ ID NO: 61) |
| | (SEQ ID NO: 60) | |
| xhCD8v2 | QVQLVQSGAEVKKPGSSVKVSCKA | DIQMTQSPSSLSASVGDRVTITCRA |
| | SGYRFHNFAISWVRQAPGQGLEW | SQEIYGALNWYQQKPGKAPKLLIY |
| | MGGIIPGHAKANYAQKFQGRVTIT | GATNLQSGVPSRFSGSGSGTDFTLTI |
| | ADESTSTAYMELSSLRSEDTAVYY | SSLQPEDFATYYCQDIYDAPWTFG |
| | CARDGLGIRLFADWGQGTLVTVSS | GGTKVEIK |
| | (SEQ ID NO: 62) | (SEQ ID NO: 63) |
| xhCD8v3 | QVQLVQSGAEVKKPGSSVKVSCKA | DIQMTQSPSSLSASVGDRVTITCRA |
| | SGSRFYKFAISWVRQAPGQGLEWM | SQEIYGALNWYQQKPGKAPKLLIY |
| | GGIIPGHAKANYAQKFQGRVTITAD | GATNLQSGVPSRFSGSGSGTDFTLTI |
| | ESTSTAYMELSSLRSEDTAVYYCAR | SSLQPEDFATYYCQDIYDAPWTFG |
| | DGLGIRLFADWGQGTLVTVSS | GGTKVEIK |
| | (SEQ ID NO: 64) | (SEQ ID NO: 65) |
| xhCD8v4 | QVQLVQSGAEVKKPGSSVKVSCKA | DIQMTQSPSSLSASVGDRVTITCRA |
| | SGYTFTKYAISWVRQAPGQGLEW | SQKIYGALNWYQQKPGKAPKLLIY |
| | MGGIIPGHAKANYAQKFQGRVTIT | GATNLQSGVPSRFSGSGSGTDFTLTI |
| | ADESTSTAYMELSSLRSEDTAVYY | SSLQPEDFATYYCQNTYDTPWTFG |
| | CARDGLGIRLFADWGQGTLVTVSS | GGTKVEIK |
| | (SEQ ID NO: 66) | (SEQ ID NO: 67) |
| xhCD8v5 | QVQLVQSGAEVKKPGSSVKVSCKA | DIQMTQSPSSLSASVGDRVTITCRA |
| | SGSGFRGHAISWVRQAPGQGLEW | SQKIYGALNWYQQKPGKAPKLLIY |
| | MGGIIPGHAKANYAQKFQGRVTIT | GATNLQSGVPSRFSGSGSGTDFTLTI |
| | ADESTSTAYMELSSLRSEDTAVYY | SSLQPEDFATYYCQNTYDTPWTFG |
| | CARDGLGIRLFADWGQGTLVTVSS | GGTKVEIK |
| | (SEQ ID NO: 68) | (SEQ ID NO: 69) |
| xhCD8v6 | EVQLVESGGGAVRPGGSLRLSCAA | EIVLTQSPATLSVSPGERATLSCRAS |
| | SGFTFDDYGMSWVRQAPGKGLEW | QSVSSNLAWYQQKPGQAPRLLIYG |
| | VSDINWSGEITAYADSVKGRFTISR | ASSRATGIPDRFSGSGSGTDFTLTIS |
| | DNAKNSLYLQMNSLRAEDTAVYY | RLEPEDFAVYYCQQYGSSPPVTFGQ |
| | CARSNSYRWDDALDIWGQGTMVT | GTKVEIK |
| | VSS | (SEQ ID NO: 71) |
| | (SEQ ID NO: 70) | |
| xhCD8v7 | EVQLVESGGGLVQPGRSLRLSCAA | EIVLTQSPATLSVTPGEGATLSCRAS |
| | SGFTFDDYAMHWVRQAPGKGLEW | HSVGSNLAWYQQKPGQAPRLLIYD |
| | VAVISYDGSNKYYADSVKGRFTISR | ASNRATGIPARFSGSGSGTDFTLTIS |
| | DNSKNTLYLQMNSLRAEDTAVYY | SLEPEDLAVYYCQQRSNWPPTFGQ |
| | CAKDRIGWYDYDAFDIWGQGTMV | GTRLEIK |
| | TVSS | (SEQ ID NO: 73) |
| | (SEQ ID NO: 72) | |
| xhCD8v8 | QVQLQQSGAELVRPGSSVKISCKAS | DIQMTQSPASLSVSVGETVTITCRA |
| | GYAFSSYWMNWVKQRPGQGLEWI | SENIYSNLAWYQQKQGKSPQLLVY |
| | GQIYPGDGDTNYNGKFKGKATLTA | AATNLADGVPSRFSGSGSGTQYSL |
| | DKSSSTAYMQLSSLTSEDSAVYFCA | KINSLQSEDFGSYYCQHFWGTPWT |
| | RSGAAFSSYYAMDYWGQGTSVTV | FGGGTKLEIK (SEQ ID NO: 186) |
| | SS (SEQ ID NO: 185) | |
| xhCD8v9 | QVQLVQSGAEVKKPGSSVKVSCKA | DIQMTQSPSSLSASVGDRVTITCRA |
| | SGGTFSSYAISWVRQAPGQGLEWM | SQEIYGALNWYQQKPGKAPKLLIY |
| | GGIIPGAATANYAQKFQGRVTITAD | GATNLQSGVPSRFSGSGSGTDFTLTI |
| | ESTSTAYMELSSLRSEDTAVYYCAR | SSLQPEDFATYYCQSTYDAPWTFG |
| | DAAGIRLFADWGQGTLVTVSS | GGTKVEIK (SEQ ID NO: 246) |
| | (SEQ ID NO: 245) | |
| xhCD8v10 | EVQLVESGGGLVQPGGSLRLSCAA | EIVLTQSPGTLSLSPGERATLSCRAS |
| | SGFTFSSYAMSWVRQAPGKGLEW | QSVSSNLAWYQQKPGQAPRLLIYG |
| | VSDITYAGGSTAYADSVKGRFTISR | ASSRATGIPDRFSGSGSGTDFTLTIS |
| | DNAKNSLYLQMNSLRAEDTAVYY | RLEPEDFAVYYCQQYGSSPPVTFGQ |
| | CARSNAYAWDDALDIWGQGTMVT | GTKVEIK (SEQ ID NO: 248) |
| | VSS (SEQ ID NO: 247) | |
| xhCD8v11 | EVQLVESGGGLVQPGGSLRLSCAA | EIVLTQSPGTLSLSPGERATLSCRAS |
| | SGFTFSSYAMSWVRQAPGKGLEW | QSVSSNLAWYQQKPGQAPRLLIYG |
| | VSDITYAGGSTAYADSVKGRFTISR | ASSRATGIPDRFSGSGSGTDFTLTIS |
| | DNAKNSLYLQMNSLRAEDTAVYY | RLEPEDFAVYYCQQYGSSPPVTFGQ |
| | CARSNAYAWDDALDIWGQGTLVT | GTKVEIK (SEQ ID NO: 250) |
| | VSS (SEQ ID NO: 249) | |
| xhCD8v12 | QVQLVQSGAEVKKPGSSVKVSCKA | DIQMTQSPSSLSASVGDRVTITCRA |
| | SGGTFSSYAISWVRQAPGQGLEWM | SQSIYGALNWYQQKPGKAPKLLIY |
| | GGIIPGYATANYAQKFQGRVTITAD | GASNLQSGVPSRFSGSGSGTDFTLTI |
| | ESTSTAYMELSSLRSEDTAVYYCAR | SSLQPEDFATYYCQSTYTAPWTFG |
| | DAAGIRLFADWGQGTLVTVSS | GGTKVEIK (SEQ ID NO: 252) |
| | (SEQ ID NO: 251) | |
| xhCD8v13 | QVQLVQSGAEVKKPGSSVKVSCKA | DIQMTQSPSSLSASVGDRVTITCRA |
| | SGGTFSSYAISWVRQAPGQGLEWM | SQEIYGALNWYQQKPGKAPKLLIY |
| | GGIIPGYATANYAQKFQGRVTITAD | GATNLQSGVPSRFSGSGSGTDFTLTI |
| | ESTSTAYMELSSLRSEDTAVYYCAR | SSLQPEDFATYYCQSTYDAPWTFG |
| | DAAGIRLFADWGQGTLVTVSS | GGTKVEIK (SEQ ID NO: 254) |
| | (SEQ ID NO: 253) | |
| xhCD8v14 | EVQLVESGGGLVQPGGSLRLSCAA | EIVLTQSPGTLSLSPGERATLSCRAS |
| | SGFTFSSYAMSWVRQAPGKGLEW | QSVSSNLAWYQQKPGQAPRLLIYG |
| | VSDISYAGGSTAYADSVKGRFTISR | ASSRATGIPDRFSGSGSGTDFTLTIS |
| | DNAKNSLYLQMNSLRAEDTAVYY | RLEPEDFAVYYCQQYGSSPPVTFGQ |
| | CARSNAYAWDDALDIWGQGTMVT | GTKVEIK (SEQ ID NO: 256) |
| | VSS (SEQ ID NO: 255) | |
| xhCD8v15 | EVQLVESGGGLVQPGGSLRLSCAA | EIVLTQSPGTLSLSPGERATLSCRAS |
| | SGFTFSSYAMSWVRQAPGKGLEW | QSVSSNLAWYQQKPGQAPRLLIYG |
| | VSDISYAGGSTAYADSVKGRFTISR | ASSRATGIPDRFSGSGSGTDFTLTIS |
| | DNAKNSLYLQMNSLRAEDTAVYY | RLEPEDFAVYYCQQYGSSPPVTFGQ |
| | CARSNAYAWDDALDIWGQGTLVT | GTKVEIK (SEQ ID NO: 258) |
| | VSS (SEQ ID NO: 257) | |

Further provided herein are fusion proteins comprising any one of the anti-CD8 antibodies, or antigen binding domains, or antibody fragments disclosed herein. In some embodiments, a fusion protein of the present disclosure comprises

In some embodiments, the present disclosure provides a fusion protein comprising two heavy chain sequences and two light chain sequences of a single fusion protein listed in Table 13, wherein one of the heavy chain sequences has an IL-10 fusion and the other heavy chain sequence is without an IL-10 fusion, and wherein the two light chain sequences are identical. In some embodiments, the heavy chain sequence without an IL-10 fusion comprises a lysine at the C terminus. In some embodiments, the fusion protein is of format F shown in FIG. 6. For example, in some embodiments, the fusion protein comprises four polypeptide chains, wherein (1) the first polypeptide chain comprises the amino acid sequence of SEQ ID NO:113, the second polypeptide chain comprises the amino acid sequence of SEQ ID NO:114, the third polypeptide chain comprises the amino acid sequence of SEQ ID NO:115, and the fourth polypeptide chain comprises the amino acid sequence of SEQ ID NO:113; (2) the first polypeptide chain comprises the amino acid sequence of SEQ ID NO:113, the second polypeptide chain comprises the amino acid sequence of SEQ ID NO:114, the third polypeptide chain comprises the amino acid sequence of SEQ ID NO:116, and the fourth polypeptide chain comprises the amino acid sequence of SEQ ID NO:113; (3) the first polypeptide chain comprises the amino acid sequence of SEQ ID NO:117, the second polypeptide chain comprises the amino acid sequence of SEQ ID NO:118, the third polypeptide chain comprises the amino acid sequence of SEQ ID NO:119, and the fourth polypeptide chain comprises the amino acid sequence of SEQ ID NO: 117; (4) the first polypeptide chain comprises the amino acid sequence of SEQ ID NO: 117, the second polypeptide chain comprises the amino acid sequence of SEQ ID NO:118, the third polypeptide chain comprises the amino acid sequence of SEQ ID NO:120, and the fourth polypeptide chain comprises the amino acid sequence of SEQ ID NO: 117; (5) the first polypeptide chain comprises the amino acid sequence of SEQ ID NO:121, the second polypeptide chain comprises the amino acid sequence of SEQ ID NO:122, the third polypeptide chain comprises the amino acid sequence of SEQ ID NO:123, and the fourth polypeptide chain comprises the amino acid sequence of SEQ ID NO: 121; (6) the first polypeptide chain comprises the amino acid sequence of SEQ ID NO: 121, the second polypeptide chain comprises the amino acid sequence of SEQ ID NO:122, the third polypeptide chain comprises the amino acid sequence of SEQ ID NO:124, and the fourth polypeptide chain comprises the amino acid sequence of SEQ ID NO: 121; (7) the first polypeptide chain comprises the amino acid sequence of SEQ ID NO: 125, the second polypeptide chain comprises the amino acid sequence of SEQ ID NO:126, the third polypeptide chain comprises the amino acid sequence of SEQ ID NO:127, and the fourth polypeptide chain comprises the amino acid sequence of SEQ ID NO: 125; (8) the first polypeptide chain comprises the amino acid sequence of SEQ ID NO: 125, the second polypeptide chain comprises the amino acid sequence of SEQ ID NO:126, the third polypeptide chain comprises the amino acid sequence of SEQ ID NO:128, and the fourth polypeptide chain comprises the amino acid sequence of SEQ ID NO: 125; (9) the first polypeptide chain comprises the amino acid sequence of SEQ ID NO:129, the second polypeptide chain comprises the amino acid sequence of SEQ ID NO:130, the third polypeptide chain comprises the amino acid sequence of SEQ ID NO:131, and the fourth polypeptide chain comprises the amino acid sequence of SEQ ID NO: 129; (10) the first polypeptide chain comprises the amino acid sequence of SEQ ID NO: 129, the second polypeptide chain comprises the amino acid sequence of SEQ ID NO:130, the third polypeptide chain comprises the amino acid sequence of SEQ ID NO:132, and the fourth polypeptide chain comprises the amino acid sequence of SEQ ID NO: 129; (11) the first polypeptide chain comprises the amino acid sequence of SEQ ID NO: 133, the second polypeptide chain comprises the amino acid sequence of SEQ ID NO:134, the third polypeptide chain comprises the amino acid sequence of SEQ ID NO:135, and the fourth polypeptide chain comprises the amino acid sequence of SEQ ID NO: 133; (12) the first polypeptide chain comprises the amino acid sequence of SEQ ID NO: 133, the second polypeptide chain comprises the amino acid sequence of SEQ ID NO:134, the third polypeptide chain comprises the amino acid sequence of SEQ ID NO:136, and the fourth polypeptide chain comprises the amino acid sequence of SEQ ID NO: 133; (13) the first polypeptide chain comprises the amino acid sequence of SEQ ID NO: 137, the second polypeptide chain comprises the amino acid sequence of SEQ ID NO:138, the third polypeptide chain comprises the amino acid sequence of SEQ ID NO:139, and the fourth polypeptide chain comprises the amino acid sequence of SEQ ID NO: 137; (14) the first polypeptide chain comprises the amino acid sequence of SEQ ID NO: 137, the second polypeptide chain comprises the amino acid sequence of SEQ ID NO:138, the third polypeptide chain comprises the amino acid sequence of SEQ ID NO:140, and the fourth polypeptide chain comprises the amino acid sequence of SEQ ID NO: 137; (15) the first polypeptide chain comprises the amino acid sequence of SEQ ID NO:141, the second polypeptide chain comprises the amino acid sequence of SEQ ID NO:142, the third polypeptide chain comprises the amino acid sequence of SEQ ID NO:143, and the fourth polypeptide chain comprises the amino acid sequence of SEQ ID NO: 141; (16) the first polypeptide chain comprises the amino acid sequence of SEQ ID NO: 141, the second polypeptide chain comprises the amino acid sequence of SEQ ID NO:142, the third polypeptide chain comprises the amino acid sequence of SEQ ID NO:144, and the fourth polypeptide chain comprises the amino acid sequence of SEQ ID NO: 141; (17) the first polypeptide chain comprises the amino acid sequence of SEQ ID NO:145, the second polypeptide chain comprises the amino acid sequence of SEQ ID NO:146, the third polypeptide chain comprises the amino acid sequence of SEQ ID NO:147, and the fourth polypeptide chain comprises the amino acid sequence of SEQ ID NO: 145; (18) the first polypeptide chain comprises the amino acid sequence of SEQ ID NO: 145, the second polypeptide chain comprises the amino acid sequence of SEQ ID NO:146, the third polypeptide chain comprises the amino acid sequence of SEQ ID NO:148, and the fourth polypeptide chain comprises the amino acid sequence of SEQ ID NO: 145; (19) the first polypeptide chain comprises the amino acid sequence of SEQ ID NO:149, the second polypeptide chain comprises the amino acid sequence of SEQ ID NO:150, the third polypeptide chain comprises the amino acid sequence of SEQ ID NO:151, and the fourth polypeptide chain comprises the amino acid sequence of SEQ ID NO: 149; (20) the first polypeptide chain comprises the amino acid sequence of SEQ ID NO: 149, the second polypeptide chain comprises the amino acid sequence of SEQ ID NO:150, the third polypeptide chain comprises the amino acid sequence of SEQ ID NO:152, and the fourth polypeptide chain comprises the amino acid sequence of SEQ ID NO: 149; (21) the first polypeptide chain comprises the amino acid sequence of SEQ ID NO:153, the second polypeptide chain comprises the amino acid sequence of SEQ ID NO:154, the third polypeptide chain comprises the amino acid sequence of SEQ ID NO:155, and the fourth polypeptide chain comprises the amino acid sequence of SEQ ID NO: 153; (22) the first polypeptide chain comprises the amino acid sequence of SEQ ID NO: 153, the second polypeptide chain comprises the amino acid sequence of SEQ ID NO:154, the third polypeptide chain comprises the amino acid sequence of SEQ ID NO:156, and the fourth polypeptide chain comprises the amino acid sequence of SEQ ID NO: 153; (23) the first polypeptide chain comprises the amino acid sequence of SEQ ID NO:157, the second polypeptide chain comprises the amino acid sequence of SEQ ID NO:158, the third polypeptide chain comprises the amino acid sequence of SEQ ID NO:159, and the fourth polypeptide chain comprises the amino acid sequence of SEQ ID NO: 157; or (24) the first polypeptide chain comprises the amino acid sequence of SEQ ID NO: 157, the second polypeptide chain comprises the amino acid sequence of SEQ ID NO: 158, the third polypeptide chain comprises the amino acid sequence of SEQ ID NO: 160, and the fourth polypeptide chain comprises the amino acid sequence of SEQ ID NO: 157.

TABLE 13

| | | | | Heavy chain |
|---|---|---|---|---|
| | | | Heavy chain | sequence |
| | | | sequence | (without IL10 |
| | Light chain | Heavy chain sequence | (without | fusion) plus C- |
| Name | sequence | (with IL10 fusion) | IL10 fusion) | term lysine |
| xhCD8v12- | DIQMTQSPSS | QVQLVQSGAEVKKP | QVQLVQSGAEV | QVQLVQSGAE |
| IL- | LSASVGDRV | GSSVKVSCKASGGTF | KKPGSSVKVSCK | VKKPGSSVKV |
| 10mono_ | TITCRASQSI | SSYAISWVRQAPGQG | ASGGTFSSYAIS | SCKASGGTFSS |
| RBenh2-m10 | YGALNWYQ | LEWMGGIIPGYATAN | WVRQAPGQGLE | YAISWVRQAP |
| | QKPGKAPKL | YAQKFQGRVTITADE | WMGGIIPGYATA | GQGLEWMGGI |
| | LIYGASNLQS | STSTAYMELSSLRSE | NYAQKFQGRVTI | IPGYATANYA |
| | GVPSRFSGSG | DTAVYYCARDAAGI | TADESTSTAYME | QKFQGRVTITA |
| | SGTDFTLTIS | RLFADWGQGTLVTV | LSSLRSEDTAVY | DESTSTAYMEL |
| | SLQPEDFAT | SSASTKGPSVFPLAPS | YCARDAAGIRLF | SSLRSEDTAVY |
| | YYCQSTYTA | SKSTSGGTAALGCLV | ADWGQGTLVTV | YCARDAAGIR |
| | PWTFGGGTK | KDYFPEPVTVSWNSG | SSASTKGPSVFPL | LFADWGQGTL |
| | VEIKRTVAA | ALTSGVHTFPAVLQS | APSSKSTSGGTA | VTVSSASTKGP |
| | PSVFIFPPSDE | SGLYSLSSVVTVPSSS | ALGCLVKDYFPE | SVFPLAPSSKS |
| | QLKSGTASV | LGTQTYICNVNHKPS | PVTVSWNSGAL | TSGGTAALGC |
| | VCLLNNFYP | NTKVDKKVEPKSCD | TSGVHTFPAVLQ | LVKDYFPEPVT |
| | REAKVQWK | KTHTCPPCPAPEAAG | SSGLYSLSSVVT | VSWNSGALTS |
| | VDNALQSGN | APSVFLFPPKPKDTL | VPSSSLGTQTYIC | GVHTFPAVLQS |
| | SQESVTEQD | MISRTPEVTCVVVDV | NVNHKPSNTKV | SGLYSLSSVVT |
| | SKDSTYSLSS | SHEDPEVKFNWYVD | DKKVEPKSCDK | VPSSSLGTQTY |
| | TLTLSKADY | GVEVHNAKTKPREE | THTCPPCPAPEA | ICNVNHKPSNT |
| | EKHKVYACE | QYNSTYRVVSVLTVL | AGAPSVFLFPPK | KVDKKVEPKS |
| | VTHQGLSSP | HQDWLNGKEYKCKV | PKDTLMISRTPE | CDKTHTCPPCP |
| | VTKSFNRGE | SNKALPAPIEKTISKA | VTCVVVDVSHE | APEAAGAPSVF |
| | C | KGQPREPQVYTLPPC | DPEVKFNWYVD | LFPPKPKDTLM |
| | (SEQ ID | REEMTKNQVSLSCA | GVEVHNAKTKP | ISRTPEVTCVV |
| | NO: 113) | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSSPGQGTQSE | NQVSLWCLVKG | SNKALPAPIEK |
| | | NSCTHFPGNLPNMLR | FYPSDIAVEWES | TISKAKGQPRE |
| | | DLRDAFSRVKTFFQM | NGQPENNYKTTP | PQVCTLPPSRE |
| | | KDQLDNLLLKESLLE | PVLDSDGSFFLY | EMTKNQVSLW |
| | | DFKGYLGCQALSEMI | SKLTVDKSRWQ | CLVKGFYPSDI |
| | | QFYLEEVMPQAENQ | QGNVFSCSVMH | AVEWESNGQP |
| | | DPDIKAHVISLGENL | EALHNHYTQKS | ENNYKTTPPVL |
| | | KTLRLRLRRCHRFLP | LSLSPG | DSDGSFFLYSK |
| | | CENGGGSGGKSKAV | SEQ ID NO: 115) | LTVDKSRWQQ |
| | | EQVKNAFNKLQEKGI | | GNVFSCSVMH |
| | | YKAMSEFAIFINYIEA | | EALHNHYTQK |
| | | YMTMKIRN | | SLSLSPGK |
| | | (SEQ ID NO: 114) | | (SEQ ID |
| | | | | NO: 116) |
| xhCD8v12- | DIQMTQSPSS | QVQLVQSGAEVKKP | QVQLVQSGAEV | QVQLVQSGAE |
| IL- | LSASVGDRV | GSSVKVSCKASGGTF | KKPGSSVKVSCK | VKKPGSSVKV |
| 10mono_ | TITCRASQSI | SSYAISWVRQAPGQG | ASGGTFSSY AIS | SCKASGGTFSS |
| RBenh7-m10 | YGALNWYQ | LEWMGGIIPGYATAN | WVRQAPGQGLE | YAISWVRQAP |
| | QKPGKAPKL | YAQKFQGRVTITADE | WMGGIIPGYATA | GQGLEWMGGI |
| | LIYGASNLQS | STSTAYMELSSLRSE | NYAQKFQGRVTI | IPGYATANYA |
| | GVPSRFSGSG | DTAVYYCARDAAGI | TADESTSTAYME | QKFQGRVTITA |
| | SGTDFTLTIS | RLFADWGQGTLVTV | LSSLRSEDTAVY | DESTSTAYMEL |
| | SLQPEDFAT | SSASTKGPSVFPLAPS | YCARDAAGIRLF | SSLRSEDTAVY |
| | YYCQSTYTA | SKSTSGGTAALGCLV | ADWGQGTLVTV | YCARDAAGIR |
| | PWTFGGGTK | KDYFPEPVTVSWNSG | SSASTKGPSVFPL | LFADWGQGTL |
| | VEIKRTVAA | ALTSGVHTFPAVLQS | APSSKSTSGGTA | VTVSSASTKGP |
| | PSVFIFPPSDE | SGLYSLSSVVTVPSSS | ALGCLVKDYFPE | SVFPLAPSSKS |
| | QLKSGTASV | LGTQTYICNVNHKPS | PVTVSWNSGAL | TSGGTAALGC |
| | VCLLNNFYP | NTKVDKKVEPKSCD | TSGVHTFPAVLQ | LVKDYFPEPVT |
| | REAKVQWK | KTHTCPPCPAPEAAG | SSGLYSLSSVVT | VSWNSGALTS |
| | VDNALQSGN | APSVFLFPPKPKDTL | VPSSSLGTQTYIC | GVHTFPAVLQS |
| | SQESVTEQD | MISRTPEVTCVVVDV | NVNHKPSNTKV | SGLYSLSSVVT |
| | SKDSTYSLSS | SHEDPEVKFNWYVD | DKKVEPKSCDK | VPSSSLGTQTY |
| | TLTLSKADY | GVEVHNAKTKPREE | THTCPPCPAPEA | ICNVNHKPSNT |
| | EKHKVYACE | QYNSTYRVVSVLTVL | AGAPSVFLFPPK | KVDKKVEPKS |
| | VTHQGLSSP | HQDWLNGKEYKCKV | PKDTLMISRTPE | CDKTHTCPPCP |
| | VTKSFNRGE | SNKALPAPIEKTISKA | VTCVVVDVSHE | APEAAGAPSVF |
| | C | KGQPREPQVYTLPPC | DPEVKFNWYVD | LFPPKPKDTLM |
| | (SEQ ID | REEMTKNQVSLSCA | GVEVHNAKTKP | ISRTPEVTCVV |
| | NO: 117) | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSSPGQGTQSE | NQVSLWCLVKG | SNKALPAPIEK |
| | | NSCTHFPGNLPNMLR | FYPSDIAVEWES | TISKAKGQPRE |
| | | DLRDAFSRVKTFFQM | NGQPENNYKTTP | PQVCTLPPSRE |
| | | KDQLDNLLLKESLLE | PVLDSDGSFFLY | EMTKNQVSLW |
| | | DFKGYLGCQALSEMI | SKLTVDKSRWQ | CLVKGFYPSDI |
| | | QFYLEEVMPQAENQ | QGNVFSCSVMH | AVEWESNGQP |
| | | DPDIKAHVLSLGENL | EALHNHYTQKS | ENNYKTTPPVL |
| | | KTLRLRLRRCHRFLP | LSLSPG | DSDGSFFLYSK |
| | | CENGGGSGGKSKAV | (SEQ ID NO: 119) | LTVDKSRWQQ |
| | | EQVKNAFNKLQEKGI | | GNVFSCSVMH |
| | | YKAMSEFAIFINYIEA | | EALHNHYTQK |
| | | YMTMKIRN | | SLSLSPGK |
| | | (SEQ ID NO: 118) | | (SEQ ID |
| | | | | NO: 120) |
| xhCD8v12- | DIQMTQSPSS | QVQLVQSGAEVKKP | QVQLVQSGAEV | QVQLVQSGAE |
| IL- | LSASVGDRV | GSSVKVSCKASGGTF | KKPGSSVKVSCK | VKKPGSSVKV |
| 10mono_ | TITCRASQSI | SSYAISWVRQAPGQG | ASGGTFSSYAIS | SCKASGGTFSS |
| RBenh2-m15 | YGALNWYQ | LEWMGGIIPGYATAN | WVRQAPGQGLE | YAISWVRQAP |
| | QKPGKAPKL | YAQKFQGRVTITADE | WMGGIIPGYATA | GQGLEWMGGI |
| | LIYGASNLQS | STSTAYMELSSLRSE | NYAQKFQGRVTI | IPGYATANYA |
| | GVPSRFSGSG | DTAVYYCARDAAGI | TADESTSTAYME | QKFQGRVTITA |
| | SGTDFTLTIS | RLFADWGQGTLVTV | LSSLRSEDTAVY | DESTSTAYMEL |
| | SLQPEDFAT | SSASTKGPSVFPLAPS | YCARDAAGIRLF | SSLRSEDTAVY |
| | YYCQSTYTA | SKSTSGGTAALGCLV | ADWGQGTLVTV | YCARDAAGIR |
| | PWTFGGGTK | KDYFPEPVTVSWNSG | SSASTKGPSVFPL | LFADWGQGTL |
| | VEIKRTVAA | ALTSGVHTFPAVLQS | APSSKSTSGGTA | VTVSSASTKGP |
| | PSVFIFPPSDE | SGLYSLSSVVTVPSSS | ALGCLVKDYFPE | SVFPLAPSSKS |
| | QLKSGTASV | LGTQTYICNVNHKPS | PVTVSWNSGAL | TSGGTAALGC |
| | VCLLNNFYP | NTKVDKKVEPKSCD | TSGVHTFPAVLQ | LVKDYFPEPVT |
| | REAKVQWK | KTHTCPPCPAPEAAG | SSGLYSLSSVVT | VSWNSGALTS |
| | VDNALQSGN | APSVFLFPPKPKDTL | VPSSSLGTQTYIC | GVHTFPAVLQS |
| | SQESVTEQD | MISRTPEVTCVVVDV | NVNHKPSNTKV | SGLYSLSSVVT |
| | SKDSTYSLSS | SHEDPEVKFNWYVD | DKKVEPKSCDK | VPSSSLGTQTY |
| | TLTLSKADY | GVEVHNAKTKPREE | THTCPPCPAPEA | ICNVNHKPSNT |
| | EKHKVYACE | QYNSTYRVVSVLTVL | AGAPSVFLFPPK | KVDKKVEPKS |
| | VTHQGLSSP | HQDWLNGKEYKCKV | PKDTLMISRTPE | CDKTHTCPPCP |
| | VTKSFNRGE | SNKALPAPIEKTISKA | VTCVVVDVSHE | APEAAGAPSVF |
| | C | KGQPREPQVYTLPPC | DPEVKFNWYVD | LFPPKPKDTLM |
| | (SEQ ID | REEMTKNQVSLSCA | GVEVHNAKTKP | ISRTPEVTCVV |
| | NO: 121) | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSSPGQGTQSE | NQVSLWCLVKG | SNKALPAPIEK |
| | | NSCTHFPGNLPNMLA | FYPSDIAVEWES | TISKAKGQPRE |
| | | DLADAFSRVKTFFQM | NGQPENNYKTTP | PQVCTLPPSRE |
| | | KDQLDNLLLKESLLE | PVLDSDGSFFLY | EMTKNQVSLW |
| | | DFKGYLGCQALSEMI | SKLTVDKSRWQ | CLVKGFYPSDI |
| | | QFYLEEVMPQAENQ | QGNVFSCSVMH | AVEWESNGQP |
| | | DPDIKAHVISLGENL | EALHNHYTQKS | ENNYKTTPPVL |
| | | KTLRLRLRRCHRFLP | LSLSPG | DSDGSFFLYSK |
| | | CENGGGSGGKSKAV | (SEQ ID NO: 123) | LTVDKSRWQQ |
| | | EQVKNAFNKLQEKGI | | GNVFSCSVMH |
| | | YKAMSEFDIFINYIEA | | EALHNHYTQK |
| | | YMTMKIRN (SEQ ID | | SLSLSPGK |
| | | NO: 122) | | (SEQ ID |
| | | | | NO: 124) |
| xhCD8v12- | DIQMTQSPSS | QVQLVQSGAEVKKP | QVQLVQSGAEV | QVQLVQSGAE |
| IL- | LSASVGDRV | GSSVKVSCKASGGTF | KKPGSSVKVSCK | VKKPGSSVKV |
| 10mono_ | TITCRASQSI | SSYAISWVRQAPGQG | ASGGTFSSYAIS | SCKASGGTFSS |
| RBenh2-m10 | YGALNWYQ | LEWMGGIIPGYATAN | WVRQAPGQGLE | YAISWVRQAP |
| m117 | QKPGKAPKL | YAQKFQGRVTITADE | WMGGIIPGYATA | GQGLEWMGGI |
| | LIYGASNLQS | STSTAYMELSSLRSE | NYAQKFQGRVTI | IPGYATANYA |
| | GVPSRFSGSG | DTAVYYCARDAAGI | TADESTSTAYME | QKFQGRVTITA |
| | SGTDFTLTIS | RLFADWGQGTLVTV | LSSLRSEDTAVY | DESTSTAYMEL |
| | SLQPEDFAT | SSASTKGPSVFPLAPS | YCARDAAGIRLF | SSLRSEDTAVY |
| | YYCQSTYTA | SKSTSGGTAALGCLV | ADWGQGTLVTV | YCARDAAGIR |
| | PWTFGGGTK | KDYFPEPVTVSWNSG | SSASTKGPSVFPL | LFADWGQGTL |
| | VEIKRTVAA | ALTSGVHTFPAVLQS | APSSKSTSGGTA | VTVSSASTKGP |
| | PSVFIFPPSDE | SGLYSLSSVVTVPSSS | ALGCLVKDYFPE | SVFPLAPSSKS |
| | QLKSGTASV | LGTQTYICNVNHKPS | PVTVSWNSGAL | TSGGTAALGC |
| | VCLLNNFYP | NTKVDKKVEPKSCD | TSGVHTFPAVLQ | LVKDYFPEPVT |
| | REAKVQWK | KTHTCPPCPAPEAAG | SSGLYSLSSVVT | VSWNSGALTS |
| | VDNALQSGN | APSVFLFPPKPKDTL | VPSSSLGTQTYIC | GVHTFPAVLQS |
| | SQESVTEQD | MISRTPEVTCVVVDV | NVNHKPSNTKV | SGLYSLSSVVT |
| | SKDSTYSLSS | SHEDPEVKFNWYVD | DKKVEPKSCDK | VPSSSLGTQTY |
| | TLTLSKADY | GVEVHNAKTKPREE | THTCPPCPAPEA | ICNVNHKPSNT |
| | EKHKVYACE | QYNSTYRVVSVLTVL | AGAPSVFLFPPK | KVDKKVEPKS |
| | VTHQGLSSP | HQDWLNGKEYKCKV | PKDTLMISRTPE | CDKTHTCPPCP |
| | VTKSFNRGE | SNKALPAPIEKTISKA | VTCVVVDVSHE | APEAAGAPSVF |
| | C | KGQPREPQVYTLPPC | DPEVKFNWYVD | LFPPKPKDTLM |
| | (SEQ ID | REEMTKNQVSLSCA | GVEVHNAKTKP | ISRTPEVTCVV |
| | NO: 125) | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSSPGQGTQSE | NQVSLWCLVKG | SNKALPAPIEK |
| | | NSCTHFPGNLPNMLR | FYPSDIAVEWES | TISKAKGQPRE |
| | | DLRDAFSRVKTFFQM | NGQPENNYKTTP | PQVCTLPPSRE |
| | | KDQLDNLLLKESLLE | PVLDSDGSFFLY | EMTKNQVSLW |
| | | DFKGYLGCQALSEMI | SKLTVDKSRWQ | CLVKGFYPSDI |
| | | QFYLEEVMPQAENQ | QGNVFSCSVMH | AVEWESNGQP |
| | | DPDIKAHVISLGENL | EALHNHYTQKS | ENNYKTTPPVL |
| | | KTLRLRLRACHRFLP | LSLSPG | DSDGSFFLYSK |
| | | CENGGGSGGKSKAV | (SEQ ID NO: 127) | LTVDKSRWQQ |
| | | EQVKNAFNKLQEKGI | | GNVFSCSVMH |
| | | YKAMSEFAIFINYIEA | | EALHNHYTQK |
| | | YMTMKIRN (SEQ ID | | SLSLSPGK |
| | | NO: 126) | | (SEQ ID |
| | | | | NO: 128) |
| xhCD8v12- | DIQMTQSPSS | QVQLVQSGAEVKKP | QVQLVQSGAEV | QVQLVQSGAE |
| IL- | LSASVGDRV | GSSVKVSCKASGGTF | KKPGSSVKVSCK | VKKPGSSVKV |
| 10mono_ | TITCRASQSI | SSYAISWVRQAPGQG | ASGGTFSSYAIS | SCKASGGTFSS |
| RBenh7-m10 | YGALNWYQ | LEWMGGIIPGYATAN | WVRQAPGQGLE | YAISWVRQAP |
| m117 | QKPGKAPKL | YAQKFQGRVTITADE | WMGGIIPGYATA | GQGLEWMGGI |
| | LIYGASNLQS | STSTAYMELSSLRSE | NYAQKFQGRVTI | IPGYATANYA |
| | GVPSRFSGSG | DTAVYYCARDAAGI | TADESTSTAYME | QKFQGRVTITA |
| | SGTDFTLTIS | RLFADWGQGTLVTV | LSSLRSEDTAVY | DESTSTAYMEL |
| | SLQPEDFAT | SSASTKGPSVFPLAPS | YCARDAAGIRLF | SSLRSEDTAVY |
| | YYCQSTYTA | SKSTSGGTAALGCLV | ADWGQGTLVTV | YCARDAAGIR |
| | PWTFGGGTK | KDYFPEPVTVSWNSG | SSASTKGPSVFPL | LFADWGQGTL |
| | VEIKRTVAA | ALTSGVHTFPAVLQS | APSSKSTSGGTA | VTVSSASTKGP |
| | PSVFIFPPSDE | SGLYSLSSVVTVPSSS | ALGCLVKDYFPE | SVFPLAPSSKS |
| | QLKSGTASV | LGTQTYICNVNHKPS | PVTVSWNSGAL | TSGGTAALGC |
| | VCLLNNFYP | NTKVDKKVEPKSCD | TSGVHTFPAVLQ | LVKDYFPEPVT |
| | REAKVQWK | KTHTCPPCPAPEAAG | SSGLYSLSSVVT | VSWNSGALTS |
| | VDNALQSGN | APSVFLFPPKPKDTL | VPSSSLGTQTYIC | GVHTFPAVLQS |
| | SQESVTEQD | MISRTPEVTCVVVDV | NVNHKPSNTKV | SGLYSLSSVVT |
| | SKDSTYSLSS | SHEDPEVKFNWYVD | DKKVEPKSCDK | VPSSSLGTQTY |
| | TLTLSKADY | GVEVHNAKTKPREE | THTCPPCPAPEA | ICNVNHKPSNT |
| | EKHKVYACE | QYNSTYRVVSVLTVL | AGAPSVFLFPPK | KVDKKVEPKS |
| | VTHQGLSSP | HQDWLNGKEYKCKV | PKDTLMISRTPE | CDKTHTCPPCP |
| | VTKSFNRGE | SNKALPAPIEKTISKA | VTCVVVDVSHE | APEAAGAPSVF |
| | C | KGQPREPQVYTLPPC | DPEVKFNWYVD | LFPPKPKDTLM |
| | (SEQ ID | REEMTKNQVSLSCA | GVEVHNAKTKP | ISRTPEVTCVV |
| | NO: 129) | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSSPGQGTQSE | NQVSLWCLVKG | SNKALPAPIEK |
| | | NSCTHFPGNLPNMLR | FYPSDIAVEWES | TISKAKGQPRE |
| | | DLRDAFSRVKTFFQM | NGQPENNYKTTP | PQVCTLPPSRE |
| | | KDQLDNLLLKESLLE | PVLDSDGSFFLY | EMTKNQVSLW |
| | | DFKGYLGCQALSEMI | SKLTVDKSRWQ | CLVKGFYPSDI |
| | | QFYLEEVMPQAENQ | QGNVFSCSVMH | AVEWESNGQP |
| | | DPDIKAHVLSLGENL | EALHNHYTQKS | ENNYKTTPPVL |
| | | KTLRLRLRACHRFLP | LSLSPG | DSDGSFFLYSK |
| | | CENGGGSGGKSKAV | (SEQ ID NO: 131) | LTVDKSRWQQ |
| | | EQVKNAFNKLQEKGI | | GNVFSCSVMH |
| | | YKAMSEFAIFINYIEA | | EALHNHYTQK |
| | | YMTMKIRN (SEQ ID | | SLSLSPGK |
| | | NO: 130) | | (SEQ ID |
| | | | | NO: 132) |
| xhCD8v12- | DIQMTQSPSS | QVQLVQSGAEVKKP | QVQLVQSGAEV | QVQLVQSGAE |
| IL- | LSASVGDRV | GSSVKVSCKASGGTF | KKPGSSVKVSCK | VKKPGSSVKV |
| 10mono_ | TITCRASQSI | SSYAISWVRQAPGQG | ASGGTFSSYAIS | SCKASGGTFSS |
| RBenh2-m15 | YGALNWYQ | LEWMGGIIPGYATAN | WVRQAPGQGLE | YAISWVRQAP |
| m117 | QKPGKAPKL | YAQKFQGRVTITADE | WMGGIIPGYATA | GQGLEWMGGI |
| | LIYGASNLQS | STSTAYMELSSLRSE | NYAQKFQGRVTI | IPGYATANYA |
| | GVPSRFSGSG | DTAVYYCARDAAGI | TADESTSTAYME | QKFQGRVTITA |
| | SGTDFTLTIS | RLFADWGQGTLVTV | LSSLRSEDTAVY | DESTSTAYMEL |
| | SLQPEDFAT | SSASTKGPSVFPLAPS | YCARDAAGIRLF | SSLRSEDTAVY |
| | YYCQSTYTA | SKSTSGGTAALGCLV | ADWGQGTLVTV | YCARDAAGIR |
| | PWTFGGGTK | KDYFPEPVTVSWNSG | SSASTKGPSVFPL | LFADWGQGTL |
| | VEIKRTVAA | ALTSGVHTFPAVLQS | APSSKSTSGGTA | VTVSSASTKGP |
| | PSVFIFPPSDE | SGLYSLSSVVTVPSSS | ALGCLVKDYFPE | SVFPLAPSSKS |
| | QLKSGTASV | LGTQTYICNVNHKPS | PVTVSWNSGAL | TSGGTAALGC |
| | VCLLNNFYP | NTKVDKKVEPKSCD | TSGVHTFPAVLQ | LVKDYFPEPVT |
| | REAKVQWK | KTHTCPPCPAPEAAG | SSGLYSLSSVVT | VSWNSGALTS |
| | VDNALQSGN | APSVFLFPPKPKDTL | VPSSSLGTQTYIC | GVHTFPAVLQS |
| | SQESVTEQD | MISRTPEVTCVVVDV | NVNHKPSNTKV | SGLYSLSSVVT |
| | SKDSTYSLSS | SHEDPEVKFNWYVD | DKKVEPKSCDK | VPSSSLGTQTY |
| | TLTLSKADY | GVEVHNAKTKPREE | THTCPPCPAPEA | ICNVNHKPSNT |
| | EKHKVYACE | QYNSTYRVVSVLTVL | AGAPSVFLFPPK | KVDKKVEPKS |
| | VTHQGLSSP | HQDWLNGKEYKCKV | PKDTLMISRTPE | CDKTHTCPPCP |
| | VTKSFNRGE | SNKALPAPIEKTISKA | VTCVVVDVSHE | APEAAGAPSVF |
| | C | KGQPREPQVYTLPPC | DPEVKFNWYVD | LFPPKPKDTLM |
| | (SEQ ID | REEMTKNQVSLSCA | GVEVHNAKTKP | ISRTPEVTCVV |
| | NO: 133) | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSSPGQGTQSE | NQVSLWCLVKG | SNKALPAPIEK |
| | | NSCTHFPGNLPNMLA | FYPSDIAVEWES | TISKAKGQPRE |
| | | DLADAFSRVKTFFQM | NGQPENNYKTTP | PQVCTLPPSRE |
| | | KDQLDNLLLKESLLE | PVLDSDGSFFLY | EMTKNQVSLW |
| | | DFKGYLGCQALSEMI | SKLTVDKSRWQ | CLVKGFYPSDI |
| | | QFYLEEVMPQAENQ | QGNVFSCSVMH | AVEWESNGQP |
| | | DPDIKAHVISLGENL | EALHNHYTQKS | ENNYKTTPPVL |
| | | KTLRLRLRACHRFLP | LSLSPG | DSDGSFFLYSK |
| | | CENGGGSGGKSKAV | (SEQ ID NO: 135) | LTVDKSRWQQ |
| | | EQVKNAFNKLQEKGI | | GNVFSCSVMH |
| | | YKAMSEFDIFINYIEA | | EALHNHYTQK |
| | | YMTMKIRN (SEQ ID | | SLSLSPGK |
| | | NO: 134) | | (SEQ ID |
| | | | | NO: 136) |
| xhCD8v12- | DIQMTQSPSS | QVQLVQSGAEVKKP | QVQLVQSGAEV | QVQLVQSGAE |
| IL- | LSASVGDRV | GSSVKVSCKASGGTF | KKPGSSVKVSCK | VKKPGSSVKV |
| 10mono_ | TITCRASQSI | SSYAISWVRQAPGQG | ASGGTFSSYAIS | SCKASGGTFSS |
| RBenh2.1- | YGALNWYQ | LEWMGGIIPGYATAN | WVRQAPGQGLE | YAISWVRQAP |
| m10 | QKPGKAPKL | YAQKFQGRVTITADE | WMGGIIPGYATA | GQGLEWMGGI |
| | LIYGASNLQS | STSTAYMELSSLRSE | NYAQKFQGRVTI | IPGYATANYA |
| | GVPSRFSGSG | DTAVYYCARDAAGI | TADESTSTAYME | QKFQGRVTITA |
| | SGTDFTLTIS | RLFADWGQGTLVTV | LSSLRSEDTAVY | DESTSTAYMEL |
| | SLQPEDFAT | SSASTKGPSVFPLAPS | YCARDAAGIRLF | SSLRSEDTAVY |
| | YYCQSTYTA | SKSTSGGTAALGCLV | ADWGQGTLVTV | YCARDAAGIR |
| | PWTFGGGTK | KDYFPEPVTVSWNSG | SSASTKGPSVFPL | LFADWGQGTL |
| | VEIKRTVAA | ALTSGVHTFPAVLQS | APSSKSTSGGTA | VTVSSASTKGP |
| | PSVFIFPPSDE | SGLYSLSSVVTVPSSS | ALGCLVKDYFPE | SVFPLAPSSKS |
| | QLKSGTASV | LGTQTYICNVNHKPS | PVTVSWNSGAL | TSGGTAALGC |
| | VCLLNNFYP | NTKVDKKVEPKSCD | TSGVHTFPAVLQ | LVKDYFPEPVT |
| | REAKVQWK | KTHTCPPCPAPEAAG | SSGLYSLSSVVT | VSWNSGALTS |
| | VDNALQSGN | APSVFLFPPKPKDTL | VPSSSLGTQTYIC | GVHTFPAVLQS |
| | SQESVTEQD | MISRTPEVTCVVVDV | NVNHKPSNTKV | SGLYSLSSVVT |
| | SKDSTYSLSS | SHEDPEVKFNWYVD | DKKVEPKSCDK | VPSSSLGTQTY |
| | TLTLSKADY | GVEVHNAKTKPREE | THTCPPCPAPEA | ICNVNHKPSNT |
| | EKHKVYACE | QYNSTYRVVSVLTVL | AGAPSVFLFPPK | KVDKKVEPKS |
| | VTHQGLSSP | HQDWLNGKEYKCKV | PKDTLMISRTPE | CDKTHTCPPCP |
| | VTKSFNRGE | SNKALPAPIEKTISKA | VTCVVVDVSHE | APEAAGAPSVF |
| | C | KGQPREPQVYTLPPC | DPEVKFNWYVD | LFPPKPKDTLM |
| | (SEQ ID | REEMTKNQVSLSCA | GVEVHNAKTKP | ISRTPEVTCVV |
| | NO: 137) | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSSPGQGTQSE | NQVSLWCLVKG | SNKALPAPIEK |
| | | NSCTHFPGNLPNMLR | FYPSDIAVEWES | TISKAKGQPRE |
| | | DLRDAFSRVKTFFQM | NGQPENNYKTTP | PQVCTLPPSRE |
| | | KDQLDNLLLKESLLE | PVLDSDGSFFLY | EMTKNQVSLW |
| | | DFKGYLGCQALSEMI | SKLTVDKSRWQ | CLVKGFYPSDI |
| | | QFYLEEVMPQAENQ | QGNVFSCSVMH | AVEWESNGQP |
| | | DPDIKAHVISLGENL | EALHNHYTQKS | ENNYKTTPPVL |
| | | KTLRLRLRRCHRFLP | LSLSPG | DSDGSFFLYSK |
| | | CENKGGGSGGSKAV | (SEQ ID NO: 139) | LTVDKSRWQQ |
| | | EQVKNAFNKLQEKGI | | GNVFSCSVMH |
| | | YKAMSEFAIFINYIEA | | EALHNHYTQK |
| | | YMTMKIRN | | SLSLSPGK |
| | | (SEQ ID NO: 138) | | (SEQ ID |
| | | | | NO: 140) |
| xhCD8v12- | DIQMTQSPSS | QVQLVQSGAEVKKP | QVQLVQSGAEV | QVQLVQSGAE |
| IL- | LSASVGDRV | GSSVKVSCKASGGTF | KKPGSSVKVSCK | VKKPGSSVKV |
| 10mono_ | TITCRASQSI | SSYAISWVRQAPGQG | ASGGTFSSYAIS | SCKASGGTFSS |
| RBenh7.1- | YGALNWYQ | LEWMGGIIPGYATAN | WVRQAPGQGLE | YAISWVRQAP |
| m10 | QKPGKAPKL | YAQKFQGRVTITADE | WMGGIIPGYATA | GQGLEWMGGI |
| | LIYGASNLQS | STSTAYMELSSLRSE | NYAQKFQGRVTI | IPGYATANYA |
| | GVPSRFSGSG | DTAVYYCARDAAGI | TADESTSTAYME | QKFQGRVTITA |
| | SGTDFTLTIS | RLFADWGQGTLVTV | LSSLRSEDTAVY | DESTSTAYMEL |
| | SLQPEDFAT | SSASTKGPSVFPLAPS | YCARDAAGIRLF | SSLRSEDTAVY |
| | YYCQSTYTA | SKSTSGGTAALGCLV | ADWGQGTLVTV | YCARDAAGIR |
| | PWTFGGGTK | KDYFPEPVTVSWNSG | SSASTKGPSVFPL | LFADWGQGTL |
| | VEIKRTVAA | ALTSGVHTFPAVLQS | APSSKSTSGGTA | VTVSSASTKGP |
| | PSVFIFPPSDE | SGLYSLSSVVTVPSSS | ALGCLVKDYFPE | SVFPLAPSSKS |
| | QLKSGTASV | LGTQTYICNVNHKPS | PVTVSWNSGAL | TSGGTAALGC |
| | VCLLNNFYP | NTKVDKKVEPKSCD | TSGVHTFPAVLQ | LVKDYFPEPVT |
| | REAKVQWK | KTHTCPPCPAPEAAG | SSGLYSLSSVVT | VSWNSGALTS |
| | VDNALQSGN | APSVFLFPPKPKDTL | VPSSSLGTQTYIC | GVHTFPAVLQS |
| | SQESVTEQD | MISRTPEVTCVVVDV | NVNHKPSNTKV | SGLYSLSSVVT |
| | SKDSTYSLSS | SHEDPEVKFNWYVD | DKKVEPKSCDK | VPSSSLGTQTY |
| | TLTLSKADY | GVEVHNAKTKPREE | THTCPPCPAPEA | ICNVNHKPSNT |
| | EKHKVYACE | QYNSTYRVVSVLTVL | AGAPSVFLFPPK | KVDKKVEPKS |
| | VTHQGLSSP | HQDWLNGKEYKCKV | PKDTLMISRTPE | CDKTHTCPPCP |
| | VTKSFNRGE | SNKALPAPIEKTISKA | VTCVVVDVSHE | APEAAGAPSVF |
| | C | KGQPREPQVYTLPPC | DPEVKFNWYVD | LFPPKPKDTLM |
| | (SEQ ID | REEMTKNQVSLSCA | GVEVHNAKTKP | ISRTPEVTCVV |
| | NO: 141) | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSSPGQGTQSE | NQVSLWCLVKG | SNKALPAPIEK |
| | | NSCTHFPGNLPNMLR | FYPSDIAVEWES | TISKAKGQPRE |
| | | DLRDAFSRVKTFFQM | NGQPENNYKTTP | PQVCTLPPSRE |
| | | KDQLDNLLLKESLLE | PVLDSDGSFFLY | EMTKNQVSLW |
| | | DFKGYLGCQALSEMI | SKLTVDKSRWQ | CLVKGFYPSDI |
| | | QFYLEEVMPQAENQ | QGNVFSCSVMH | AVEWESNGQP |
| | | DPDIKAHVLSLGENL | EALHNHYTQKS | ENNYKTTPPVL |
| | | KTLRLRLRRCHRFLP | LSLSPG | DSDGSFFLYSK |
| | | CENKGGGSGGSKAV | (SEQ ID NO: 143) | LTVDKSRWQQ |
| | | EQVKNAFNKLQEKGI | | GNVFSCSVMH |
| | | YKAMSEFAIFINYIEA | | EALHNHYTQK |
| | | YMTMKIRN | | SLSLSPGK |
| | | (SEQ ID NO: 142) | | (SEQ ID |
| | | | | NO: 144) |
| xhCD8v12- | DIQMTQSPSS | QVQLVQSGAEVKKP | QVQLVQSGAEV | QVQLVQSGAE |
| IL- | LSASVGDRV | GSSVKVSCKASGGTF | KKPGSSVKVSCK | VKKPGSSVKV |
| 10mono RBe | TITCRASQSI | SSYAISWVRQAPGQG | ASGGTFSSY AIS | SCKASGGTFSS |
| nh2.1-m15 | YGALNWYQ | LEWMGGIIPGYATAN | WVRQAPGQGLE | YAISWVRQAP |
| | QKPGKAPKL | YAQKFQGRVTITADE | WMGGIIPGYATA | GQGLEWMGGI |
| | LIYGASNLQS | STSTAYMELSSLRSE | NYAQKFQGRVTI | IPGYATANYA |
| | GVPSRFSGSG | DTAVYYCARDAAGI | TADESTSTAYME | QKFQGRVTITA |
| | SGTDFTLTIS | RLFADWGQGTLVTV | LSSLRSEDTAVY | DESTSTAYMEL |
| | SLQPEDFAT | SSASTKGPSVFPLAPS | YCARDAAGIRLF | SSLRSEDTAVY |
| | YYCQSTYTA | SKSTSGGTAALGCLV | ADWGQGTLVTV | YCARDAAGIR |
| | PWTFGGGTK | KDYFPEPVTVSWNSG | SSASTKGPSVFPL | LFADWGQGTL |
| | VEIKRTVAA | ALTSGVHTFPAVLQS | APSSKSTSGGTA | VTVSSASTKGP |
| | PSVFIFPPSDE | SGLYSLSSVVTVPSSS | ALGCLVKDYFPE | SVFPLAPSSKS |
| | QLKSGTASV | LGTQTYICNVNHKPS | PVTVSWNSGAL | TSGGTAALGC |
| | VCLLNNFYP | NTKVDKKVEPKSCD | TSGVHTFPAVLQ | LVKDYFPEPVT |
| | REAKVQWK | KTHTCPPCPAPEAAG | SSGLYSLSSVVT | VSWNSGALTS |
| | VDNALQSGN | APSVFLFPPKPKDTL | VPSSSLGTQTYIC | GVHTFPAVLQS |
| | SQESVTEQD | MISRTPEVTCVVVDV | NVNHKPSNTKV | SGLYSLSSVVT |
| | SKDSTYSLSS | SHEDPEVKFNWYVD | DKKVEPKSCDK | VPSSSLGTQTY |
| | TLTLSKADY | GVEVHNAKTKPREE | THTCPPCPAPEA | ICNVNHKPSNT |
| | EKHKVYACE | QYNSTYRVVSVLTVL | AGAPSVFLFPPK | KVDKKVEPKS |
| | VTHQGLSSP | HQDWLNGKEYKCKV | PKDTLMISRTPE | CDKTHTCPPCP |
| | VTKSFNRGE | SNKALPAPIEKTISKA | VTCVVVDVSHE | APEAAGAPSVF |
| | C | KGQPREPQVYTLPPC | DPEVKFNWYVD | LFPPKPKDTLM |
| | (SEQ ID | REEMTKNQVSLSCA | GVEVHNAKTKP | ISRTPEVTCVV |
| | NO: 145) | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSSPGQGTQSE | NQVSLWCLVKG | SNKALPAPIEK |
| | | NSCTHFPGNLPNMLA | FYPSDIAVEWES | TISKAKGQPRE |
| | | DLADAFSRVKTFFQM | NGQPENNYKTTP | PQVCTLPPSRE |
| | | KDQLDNLLLKESLLE | PVLDSDGSFFLY | EMTKNQVSLW |
| | | DFKGYLGCQALSEMI | SKLTVDKSRWQ | CLVKGFYPSDI |
| | | QFYLEEVMPQAENQ | QGNVFSCSVMH | AVEWESNGQP |
| | | DPDIKAHVISLGENL | EALHNHYTQKS | ENNYKTTPPVL |
| | | KTLRLRLRRCHRFLP | LSLSPG | DSDGSFFLYSK |
| | | CENKGGGSGGSKAV | (SEQ ID NO: 147) | LTVDKSRWQQ |
| | | EQVKNAFNKLQEKGI | | GNVFSCSVMH |
| | | YKAMSEFDIFINYIEA | | EALHNHYTQK |
| | | YMTMKIRN (SEQ ID | | SLSLSPGK |
| | | NO: 146) | | (SEQ ID |
| | | | | NO: 148) |
| xhCD8v12- | DIQMTQSPSS | QVQLVQSGAEVKKP | QVQLVQSGAEV | QVQLVQSGAE |
| IL- | LSASVGDRV | GSSVKVSCKASGGTF | KKPGSSVKVSCK | VKKPGSSVKV |
| 10mono_RBe | TITCRASQSI | SSYAISWVRQAPGQG | ASGGTFSSYAIS | SCKASGGTFSS |
| nh2.1-m10 | YGALNWYQ | LEWMGGIIPGYATAN | WVRQAPGQGLE | YAISWVRQAP |
| m117 | QKPGKAPKL | YAQKFQGRVTITADE | WMGGIIPGYATA | GQGLEWMGGI |
| | LIYGASNLQS | STSTAYMELSSLRSE | NYAQKFQGRVTI | IPGYATANYA |
| | GVPSRFSGSG | DTAVYYCARDAAGI | TADESTSTAYME | QKFQGRVTITA |
| | SGTDFTLTIS | RLFADWGQGTLVTV | LSSLRSEDTAVY | DESTSTAYMEL |
| | SLQPEDFAT | SSASTKGPSVFPLAPS | YCARDAAGIRLF | SSLRSEDTAVY |
| | YYCQSTYTA | SKSTSGGTAALGCLV | ADWGQGTLVTV | YCARDAAGIR |
| | PWTFGGGTK | KDYFPEPVTVSWNSG | SSASTKGPSVFPL | LFADWGQGTL |
| | VEIKRTVAA | ALTSGVHTFPAVLQS | APSSKSTSGGTA | VTVSSASTKGP |
| | PSVFIFPPSDE | SGLYSLSSVVTVPSSS | ALGCLVKDYFPE | SVFPLAPSSKS |
| | QLKSGTASV | LGTQTYICNVNHKPS | PVTVSWNSGAL | TSGGTAALGC |
| | VCLLNNFYP | NTKVDKKVEPKSCD | TSGVHTFPAVLQ | LVKDYFPEPVT |
| | REAKVQWK | KTHTCPPCPAPEAAG | SSGLYSLSSVVT | VSWNSGALTS |
| | VDNALQSGN | APSVFLFPPKPKDTL | VPSSSLGTQTYIC | GVHTFPAVLQS |
| | SQESVTEQD | MISRTPEVTCVVVDV | NVNHKPSNTKV | SGLYSLSSVVT |
| | SKDSTYSLSS | SHEDPEVKFNWYVD | DKKVEPKSCDK | VPSSSLGTQTY |
| | TLTLSKADY | GVEVHNAKTKPREE | THTCPPCPAPEA | ICNVNHKPSNT |
| | EKHKVYACE | QYNSTYRVVSVLTVL | AGAPSVFLFPPK | KVDKKVEPKS |
| | VTHQGLSSP | HQDWLNGKEYKCKV | PKDTLMISRTPE | CDKTHTCPPCP |
| | VTKSFNRGE | SNKALPAPIEKTISKA | VTCVVVDVSHE | APEAAGAPSVF |
| | C | KGQPREPQVYTLPPC | DPEVKFNWYVD | LFPPKPKDTLM |
| | (SEQ ID | REEMTKNQVSLSCA | GVEVHNAKTKP | ISRTPEVTCVV |
| | NO: 149) | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSSPGQGTQSE | NQVSLWCLVKG | SNKALPAPIEK |
| | | NSCTHFPGNLPNMLR | FYPSDIAVEWES | TISKAKGQPRE |
| | | DLRDAFSRVKTFFQM | NGQPENNYKTTP | PQVCTLPPSRE |
| | | KDQLDNLLLKESLLE | PVLDSDGSFFLY | EMTKNQVSLW |
| | | DFKGYLGCQALSEMI | SKLTVDKSRWQ | CLVKGFYPSDI |
| | | QFYLEEVMPQAENQ | QGNVFSCSVMH | AVEWESNGQP |
| | | DPDIKAHVISLGENL | EALHNHYTQKS | ENNYKTTPPVL |
| | | KTLRLRLRACHRFLP | LSLSPG | DSDGSFFLYSK |
| | | CENKGGGSGGSKAV | (SEQ ID NO: 151) | LTVDKSRWQQ |
| | | EQVKNAFNKLQEKGI | | GNVFSCSVMH |
| | | YKAMSEFAIFINYIEA | | EALHNHYTQK |
| | | YMTMKIRN (SEQ ID | | SLSLSPGK |
| | | NO: 150) | | (SEQ ID |
| | | | | NO: 152) |
| xhCD8v12- | DIQMTQSPSS | QVQLVQSGAEVKKP | QVQLVQSGAEV | QVQLVQSGAE |
| IL- | LSASVGDRV | GSSVKVSCKASGGTF | KKPGSSVKVSCK | VKKPGSSVKV |
| 10mono_ | TITCRASQSI | SSYAISWVRQAPGQG | ASGGTFSSYAIS | SCKASGGTFSS |
| RBenh7.1- | YGALNWYQ | LEWMGGIIPGYATAN | WVRQAPGQGLE | YAISWVRQAP |
| m10 m117 | QKPGKAPKL | YAQKFQGRVTITADE | WMGGIIPGYATA | GQGLEWMGGI |
| | LIYGASNLQS | STSTAYMELSSLRSE | NYAQKFQGRVTI | IPGYATANYA |
| | GVPSRFSGSG | DTAVYYCARDAAGI | TADESTSTAYME | QKFQGRVTITA |
| | SGTDFTLTIS | RLFADWGQGTLVTV | LSSLRSEDTAVY | DESTSTAYMEL |
| | SLQPEDFAT | SSASTKGPSVFPLAPS | YCARDAAGIRLF | SSLRSEDTAVY |
| | YYCQSTYTA | SKSTSGGTAALGCLV | ADWGQGTLVTV | YCARDAAGIR |
| | PWTFGGGTK | KDYFPEPVTVSWNSG | SSASTKGPSVFPL | LFADWGQGTL |
| | VEIKRTVAA | ALTSGVHTFPAVLQS | APSSKSTSGGTA | VTVSSASTKGP |
| | PSVFIFPPSDE | SGLYSLSSVVTVPSSS | ALGCLVKDYFPE | SVFPLAPSSKS |
| | QLKSGTASV | LGTQTYICNVNHKPS | PVTVSWNSGAL | TSGGTAALGC |
| | VCLLNNFYP | NTKVDKKVEPKSCD | TSGVHTFPAVLQ | LVKDYFPEPVT |
| | REAKVQWK | KTHTCPPCPAPEAAG | SSGLYSLSSVVT | VSWNSGALTS |
| | VDNALQSGN | APSVFLFPPKPKDTL | VPSSSLGTQTYIC | GVHTFPAVLQS |
| | SQESVTEQD | MISRTPEVTCVVVDV | NVNHKPSNTKV | SGLYSLSSVVT |
| | SKDSTYSLSS | SHEDPEVKFNWYVD | DKKVEPKSCDK | VPSSSLGTQTY |
| | TLTLSKADY | GVEVHNAKTKPREE | THTCPPCPAPEA | ICNVNHKPSNT |
| | EKHKVYACE | QYNSTYRVVSVLTVL | AGAPSVFLFPPK | KVDKKVEPKS |
| | VTHQGLSSP | HQDWLNGKEYKCKV | PKDTLMISRTPE | CDKTHTCPPCP |
| | VTKSFNRGE | SNKALPAPIEKTISKA | VTCVVVDVSHE | APEAAGAPSVF |
| | C | KGQPREPQVYTLPPC | DPEVKFNWYVD | LFPPKPKDTLM |
| | (SEQ ID | REEMTKNQVSLSCA | GVEVHNAKTKP | ISRTPEVTCVV |
| | NO: 153) | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSSPGQGTQSE | NQVSLWCLVKG | SNKALPAPIEK |
| | | NSCTHFPGNLPNMLR | FYPSDIAVEWES | TISKAKGQPRE |
| | | DLRDAFSRVKTFFQM | NGQPENNYKTTP | PQVCTLPPSRE |
| | | KDQLDNLLLKESLLE | PVLDSDGSFFLY | EMTKNQVSLW |
| | | DFKGYLGCQALSEMI | SKLTVDKSRWQ | CLVKGFYPSDI |
| | | QFYLEEVMPQAENQ | QGNVFSCSVMH | AVEWESNGQP |
| | | DPDIKAHVLSLGENL | EALHNHYTQKS | ENNYKTTPPVL |
| | | KTLRLRLRACHRFLP | LSLSPG | DSDGSFFLYSK |
| | | CENKGGGSGGSKAV | (SEQ ID NO: 155) | LTVDKSRWQQ |
| | | EQVKNAFNKLQEKGI | | GNVFSCSVMH |
| | | YKAMSEFAIFINYIEA | | EALHNHYTQK |
| | | YMTMKIRN (SEQ ID | | SLSLSPGK |
| | | NO: 154) | | (SEQ ID |
| | | | | NO: 156) |
| xhCD8v12- | DIQMTQSPSS | QVQLVQSGAEVKKP | QVQLVQSGAEV | QVQLVQSGAE |
| IL- | LSASVGDRV | GSSVKVSCKASGGTF | KKPGSSVKVSCK | VKKPGSSVKV |
| 10mono_ | TITCRASQSI | SSYAISWVRQAPGQG | ASGGTFSSYAIS | SCKASGGTFSS |
| RBenh2.1- | YGALNWYQ | LEWMGGIIPGYATAN | WVRQAPGQGLE | YAISWVRQAP |
| m15 m117 | QKPGKAPKL | YAQKFQGRVTITADE | WMGGIIPGYATA | GQGLEWMGGI |
| | LIYGASNLQS | STSTAYMELSSLRSE | NYAQKFQGRVTI | IPGYATANYA |
| | GVPSRFSGSG | DTAVYYCARDAAGI | TADESTSTAYME | QKFQGRVTITA |
| | SGTDFTLTIS | RLFADWGQGTLVTV | LSSLRSEDTAVY | DESTSTAYMEL |
| | SLQPEDFAT | SSASTKGPSVFPLAPS | YCARDAAGIRLF | SSLRSEDTAVY |
| | YYCQSTYTA | SKSTSGGTAALGCLV | ADWGQGTLVTV | YCARDAAGIR |
| | PWTFGGGTK | KDYFPEPVTVSWNSG | SSASTKGPSVFPL | LFADWGQGTL |
| | VEIKRTVAA | ALTSGVHTFPAVLQS | APSSKSTSGGTA | VTVSSASTKGP |
| | PSVFIFPPSDE | SGLYSLSSVVTVPSSS | ALGCLVKDYFPE | SVFPLAPSSKS |
| | QLKSGTASV | LGTQTYICNVNHKPS | PVTVSWNSGAL | TSGGTAALGC |
| | VCLLNNFYP | NTKVDKKVEPKSCD | TSGVHTFPAVLQ | LVKDYFPEPVT |
| | REAKVQWK | KTHTCPPCPAPEAAG | SSGLYSLSSVVT | VSWNSGALTS |
| | VDNALQSGN | APSVFLFPPKPKDTL | VPSSSLGTQTYIC | GVHTFPAVLQS |
| | SQESVTEQD | MISRTPEVTCVVVDV | NVNHKPSNTKV | SGLYSLSSVVT |
| | SKDSTYSLSS | SHEDPEVKFNWYVD | DKKVEPKSCDK | VPSSSLGTQTY |
| | TLTLSKADY | GVEVHNAKTKPREE | THTCPPCPAPEA | ICNVNHKPSNT |
| | EKHKVYACE | QYNSTYRVVSVLTVL | AGAPSVFLFPPK | KVDKKVEPKS |
| | VTHQGLSSP | HQDWLNGKEYKCKV | PKDTLMISRTPE | CDKTHTCPPCP |
| | VTKSFNRGE | SNKALPAPIEKTISKA | VTCVVVDVSHE | APEAAGAPSVF |
| | C | KGQPREPQVYTLPPC | DPEVKFNWYVD | LFPPKPKDTLM |
| | (SEQ ID | REEMTKNQVSLSCA | GVEVHNAKTKP | ISRTPEVTCVV |
| | NO: 157) | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSSPGQGTQSE | NQVSLWCLVKG | SNKALPAPIEK |
| | | NSCTHFPGNLPNMLA | FYPSDIAVEWES | TISKAKGQPRE |
| | | DLADAFSRVKTFFQM | NGQPENNYKTTP | PQVCTLPPSRE |
| | | KDQLDNLLLKESLLE | PVLDSDGSFFLY | EMTKNQVSLW |
| | | DFKGYLGCQALSEMI | SKLTVDKSRWQ | CLVKGFYPSDI |
| | | QFYLEEVMPQAENQ | QGNVFSCSVMH | AVEWESNGQP |
| | | DPDIKAHVISLGENL | EALHNHYTQKS | ENNYKTTPPVL |
| | | KTLRLRLRACHRFLP | LSLSPG | DSDGSFFLYSK |
| | | CENKGGGSGGSKAV | (SEQ ID NO: 159) | LTVDKSRWQQ |
| | | EQVKNAFNKLQEKGI | | GNVFSCSVMH |
| | | YKAMSEFDIFINYIEA | | EALHNHYTQK |
| | | YMTMKIRN (SEQ ID | | SLSLSPGK |
| | | NO: 158) | | (SEQ ID |
| | | | | NO: 160) |

Further provided herein are polynucleotides (e.g., isolated polynucleotides) encoding any of the antibodies, antibody fragments, and fusion proteins described herein. Further provided herein are vectors (e.g., expression vectors) encoding any of the antibodies, antibody fragments, and fusion proteins described herein.

Further provided herein are host cells (e.g., isolated host cells or host cell lines) comprising any of the polynucleotides or vectors described herein.

Further provided herein are methods of producing any of the antibodies, antibody fragments, and fusion proteins described herein. In some embodiments, the methods comprise culturing a host cell of the present disclosure under conditions suitable for production of the antibody, antibody fragment, or fusion protein. In some embodiments, the methods further comprise recovering the antibody, antibody fragment, or fusion protein.

Antibodies, antibody fragments, and fusion proteins may be produced using recombinant methods, e.g., as exemplified infra. In some embodiments, nucleic acid encoding the antibody/fusion protein can be isolated and inserted into a replicable vector for further cloning or for expression. DNA encoding the antibody/fusion protein may be readily isolated and sequenced using conventional procedures (e.g., via oligonucleotide probes capable of binding specifically to genes encoding the heavy and light chains of the antibody/fragment). Many vectors are known in the art; vector components generally include, but are not limited to, one or more of the following: a signal sequence, an origin of replication, one or more marker genes, an enhancer element, a promoter, and a transcription termination sequence. Suitable host cells for cloning or expressing the DNA in the vectors herein are the prokaryote, yeast, or higher eukaryote cells. When using recombinant techniques, the antibody/fusion protein can be produced intracellularly, in the periplasmic space, or directly secreted into the medium. If the antibody/fragment is produced intracellularly, the particulate debris, either host cells or lysed fragments, are removed, for example, by centrifugation or ultrafiltration. Where the antibody/fusion protein is secreted into the medium, supernatants from such expression systems are generally first concentrated using a commercially available protein concentration filter.

In some embodiments, a fusion protein of the present disclosure is part of a pharmaceutical composition, e.g., including the fusion protein and one or more pharmaceutically acceptable carriers. Pharmaceutical compositions and formulations as described herein can be prepared by mixing the active ingredients (such as a fusion protein) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives; low molecular weight (less than about 10 residues) polypeptides; proteins, such as serum albumin, gelatin, or immunoglobulins; hydrophilic polymers such as polyvinylpyrrolidone; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). In some embodiments, a fusion protein of the present disclosure is lyophilized.

Certain aspects of the present disclosure relate to methods of treating cancer or chronic infection. In some embodiments, the methods comprise administering an effective amount of a fusion protein or antibody, or a pharmaceutical composition comprising the fusion protein or antibody and a pharmaceutically acceptable carrier, to a patient. In some embodiments, the patient in need of said treatment has been diagnosed with cancer.

In some embodiments, the fusion protein or composition is administered in combination with a T cell therapy, cancer vaccine, chemotherapeutic agent, or immune checkpoint inhibitor (ICI). In some embodiments, the chemotherapeutic agent is a kinase inhibitor, antimetabolite, cytotoxin or cytostatic agent, anti-hormonal agent, platinum-based chemotherapeutic agent, methyltransferase inhibitor, antibody, or anti-cancer peptide. In some embodiments, the immune checkpoint inhibitor targets PD-L1, PD-1, CTLA-4, CEACAM, LAIR1, CD160, 2B4, CD80, CD86, CD276, VTCN1, HVEM, KIR, A2AR, MHC class I, MHC class II, GALS, adenosine, TGFR, OX40, CD137, CD40, CD47, TREM1, TREM2, HLA-G, CCR4, CCR8, CD39, CD73, IDO, CSF1R, TIM-3, BTLA, VISTA, LAG-3, TIGIT, IDO, MICA/B, LILRB4, SIGLEC-15, or arginase, including without limitation an inhibitor of PD-1 (e.g., an anti-PD-1 antibody), PD-L1 (e.g., an anti-PD-L1 antibody), or CTLA-4 (e.g., an anti-CTLA-4 antibody). In some embodiments, the fusion protein or composition is administered in combination with an IL-2 polypeptide (including muteins or variants thereof), or a fusion protein comprising an IL-2 polypeptide (including muteins or variants thereof), including but not limited to antibody:IL-2 fusion proteins (e.g., anti-CD8:IL-2 fusion proteins).

Examples of anti-PD-1 antibodies include, without limitation, pembrolizumab, nivolumab, cemiplimab, zimberelimab (Arcus), sasanlimab (Pfizer), JTX-4014, spartalizumab (PDR001; Novartis), camrelizumab (SHR1210; Jiangsu HengRui Medicine), sintilimab (1B1308; Innovent and Eli Lilly), tislelizumab (BGB-A317), toripalimab (JS 001), dostarlimab (TSR-042, WBP-285), INCMGA00012 (MGA012), AMP-224, and AMP-514 (MEDI0680). Examples of anti-PD-L1 antibodies include, without limitation, atezolizumab, avelumab, durvalumab, KN035, and CK-301 (Checkpoint Therapeutics). Examples of PD-L1 inhibitors (non-antibody based) include, without limitation, AUNP12, CA-170, and BMS-986189. Examples of anti-CTLA-4 antibodies include, without limitation, ipilimumab, tremelimumab, BMS-986218, BMS-986249, BMS-986288, HBM4003, ONC-392, KN044, ADG116, ADU-1604, AGEN1181, AGEN1884, MK-1308, and REGN4659.

Examples of T cell therapies include, without limitation, CD4+ or CD8+ T cell-based therapies, adoptive T cell therapies, chimeric antigen receptor (CAR)-based T cell therapies, tumor-infiltrating lymphocyte (TIL)-based therapies, autologous T cell therapies, allogeneic T cell therapies, and therapies with T cells bearing a transduced TCR. Exemplary cancer vaccines include, without limitation, dendritic cell vaccines, vaccines comprising one or more polynucleotides encoding one or more cancer antigens, and vaccines comprising one or more cancer antigenic peptides.

Certain aspects of the present disclosure relate to methods of expanding T cells, e.g., ex vivo. In some embodiments, the methods comprise contacting one or more T cells, e.g., ex vivo with an effective amount of the antibody or fusion protein of the present disclosure. In some embodiments, the one or more T cells are tumor infiltrating lymphocytes (TILs). In some embodiments, the methods further comprise isolating tumor infiltrating lymphocytes (TILs) from a tumor or tumor specimen. In some embodiments, the methods comprise contacting one or more T cells, e.g., ex vivo with an effective amount of the antibody or fusion protein of the present disclosure in combination with an IL-2 polypeptide (including muteins or variants thereof), or a fusion protein comprising an IL-2 polypeptide (including muteins or variants thereof), including but not limited to antibody:IL-2 fusion proteins (e.g., anti-CD8:IL-2 fusion proteins).

### EXAMPLES

### Example 1: Preparation of IL-10 Fusion Proteins and Ability of IL-10 to Activate STAT3 by Phosphorylation of STAT3

Materials and Methods

### Recombinant DNA Techniques

Techniques involving recombinant DNA manipulation were previously described in Sambrook et al., Molecular cloning: A laboratory manual; Cold Spring Harbor Laboratory Press, Cold Spring Harbor, N.Y., 1989. All reagents were used according to the manufacturer's instructions. DNA sequences were determined by double strand sequencing.

### Gene Synthesis

Desired gene segments were either generated by PCR using appropriate templates or synthesized at Genewiz (South Plainfield, NJ), Integrated DNA Technologies (Coralville, IA) or GeneScript (Piscataway, NJ) from synthetic oligonucleotides. The gene segments were cloned into the expression vectors using either Gibson assembly@ method or using restriction digest followed by ligation. DNA was purified from transformed bacteria and concentration was determined by UV visible spectroscopy. DNA sequencing was used to confirm the DNA sequences of the subcloned gene fragments.

### Isolation of Antibody Genes

Antibodies binding to CD8 antigens were generated using either humanization of mouse antibodies or in vitro phage display system.

For humanization, complementarity-determining regions (CDRs) of mouse residues were grafted into selected human framework(s) which exhibit close sequence similarity to the parental mouse framework and good stability. The resulting CDR-grafted antibodies were further humanized to remove any unnecessary non-human mutations.

For in vitro display method, a non-immune human single chain Fv phage library generated from naïve B cells was panned for 5 to 6 rounds to isolate antibodies against the CD8 antigens. After the panning, individual phage clones that exhibited specific binding to target antigen over non-specific antigens in ELISA were identified. DNA fragments of heavy and light chain V-domain of the specific binders were subsequently cloned and sequenced.

### Cloning of Antibody Constructs

General information regarding the nucleotide sequences of human immunoglobulins light and heavy chains is given in: IMGT® (the international ImMunoGeneTics information System®) from Lefranc et al. IMGT®, the international ImMunoGeneTics information System® 25 years on. Nucleic Acids Res. 2015 January; 43. The DNA fragments of heavy and light chain V-domains were inserted in frame into the human IgG1 and CK containing mammalian expression vector.

### Cloning of Fusion Constructs

The IL-10 portions of the constructs were cloned in frame with the heavy chain using a glycine-serine based linker between the C-terminus of the IgG heavy chain and the N-terminus of IL-10. The C-terminal lysine residue of the IgG heavy chain was eliminated after fusing the IL-10 portion. To generate the constructs with asymmetric geometry, knob-into-hole modification was introduced into the CH3 domains of the Fc region to facilitate heterodimerization. Specifically, the “hole” domain carried the Y349C, T366S, L368A and Y407V mutations in the CH3 domain, whereas the “knob” domain carried the S354C and T366W mutations in the CH3 domain (EU numbering). To abolish FcγR binding/effector function and prevent FcR co-activation, mutations L234A/L235A/G237A (EU numbering) were introduced into the CH2 domain of each of the IgG heavy chains or the Fc region. The expression of the antibody-IL-10 fusion constructs was driven by an CMV promoter and transcription terminated by a synthetic polyA signal sequence located downstream of the coding sequence.

Purification of Fusion Proteins with IL-10 Polypeptides

Constructs encoding fusion proteins with IL-10 polypeptides as used in the examples were produced by co-transfecting exponentially growing Expi293 cells with the mammalian expression vectors using polyethylenimine (PEI). Supernatants were collected after 4-5 days of culture. IL-10 fusion constructs were first purified by affinity chromatography using a protein A matrix. The protein A column was equilibrated and washed in phosphate-buffered saline (PBS). The fusion constructs were eluted with 20 mM sodium citrate, 50 mM sodium chloride, pH 3.6. The eluted fractions were pooled and dialyzed into 10 mM MES, 25 mM sodium chloride pH 6. The proteins were further purified using ion-exchange chromatograph (Mono-S, GE Healthcare) to purify the heterodimers over the homodimers. After loading the protein, the column was washed with 10 mM MES 25 mM sodium chloride pH 6. The protein was then eluted with increasing gradient of sodium chloride from 25 mM up to 500 mM in 10 mM MES pH 6 buffer. The major eluent peak corresponding to the heterodimer was collected and concentrated. The purified protein was then polished by size exclusion chromatography (Superdex 200, GE Healthcare) in PBS.

The protein concentration of purified IL-10 fusion constructs was determined by measuring the optical density (OD) at 280 nm, using the molar extinction coefficient calculated on the basis of the amino acid sequence. Purity, integrity and monomeric state of the fusion constructs were analyzed by SDS-PAGE in the presence and absence of a reducing agent (5 mM 1,4-dithiothreitol) and stained with Coomassie blue (SimpleBlue™ SafeStain, Invitrogen). The NuPAGE® Pre-Cast gel system (Invitrogen) was used according to the manufacturer's instructions (4-20% Tris-glycine gels or 3-12% Bis-Tris). The aggregate content of immunoconjugate samples was analyzed using a Superdex 200 10/300 GL analytical size-exclusion column (GE Healthcare).

### Binding Affinity Determination by Surface Plasmon Resonance (SPR) for CD8, IL-10RA and IL-10RB

Kinetic rate constants (kon and koff) as well as affinity (K_{D}) of IL-10 fusion proteins for human and cynomolgus CD8, IL-10RA and IL-10RB were measured by surface plasmon resonance (SPR) using a BIAcore® T200 (Cytiva) at 37° C. Briefly, to determine the affinities towards human and cyno CD8, antibody or fusion proteins were captured onto the CM4 sensor chip via their Fc by a covalently immobilized anti-human Fc capture antibody prepared using the Human Antibody Capture Kit (Cytiva). Protein was not captured on flow cell 1 to serve as a reference surface. Soluble antigen, diluted in HBS-EP+ buffer at four or more concentrations spanning 0.1× to 10× of the KD, was flowed over the surface-captured antibody/fusion protein for 1-2 minutes. Dissociation was monitored for 5-10 minutes, and the anti-hIgG-Fc surface was regenerated with 3M MgCl2 before recapturing antibody/fusion protein in each subsequent cycle. Binding data were analyzed by Biacore® Evaluation Software version 3.2 using a 1:1 Langmuir with mass transport model.

To determine the affinities with IL-10RA, antibody or fusion proteins were captured onto the CM4 sensor chip via their Fc by covalently anti-human Fc antibody (Southern Biotech, Catalog No. 2081-01). Protein was not captured on flow cell 1 to serve as a reference surface. In-house generated human IL-10RA ECD, diluted in HBS-P+ buffer supplemented with 1 g/L BSA, at concentrations of 8, 40, 200, 1000, and 5000 nM, or buffer was flowed over the surface-captured fusion protein for 2 minutes at 30 μL/min. Dissociation was monitored for 4-5 minutes, and the anti-hIgG-Fc surface was regenerated with three 30-seconds injections of 75 mM phosphoric acid between analysis cycles. Binding data were analyzed by Biacore® Evaluation Software version 3.2 using a 1:1 Langmuir with mass transport model or by steady-state affinity analysis.

To determine binding with IL-10RB, in-house generated biotinylated human IL-10RA were captured onto the chip with biotin CAPture reagent (Cytiva) that was first immobilized onto a CAP sensor chip (Cytiva) following the manufacturer instructions. Surfaces were blocked with 20 μM amine-PEG2-biotin (ThermoFisher Scientific) for 60 sec and then IL-10 fusion proteins were then injected for 3 min at 10 μL/min. An IL-10 fusion protein was not captured on flow cell 1 to serve as a reference surface. In-house generated human IL-10RB ECD, diluted in HBS-EP+ buffer supplemented with 1 g/L BSA, at concentrations of 0.2, 1, 5, 10, and 20 μM, or buffer was flowed as analyte for 2 minutes at 30 μL/min and allowed to dissociate for 4 minutes. The CAP sensor chip surfaces were regenerated with a 2-min injection of a mixture of 3 parts of 8M guanidine-HCl with 1 part of 1M NaOH between analysis cycles. Sensorgrams were double-referenced. To rank binding, the capture response units of the IL-10 fusion proteins were normalized, and then, the binding response units of IL-10RB, at the highest concentration and 5-sec before the end of the association step, were recorded using Biacore Evaluation Software version 3.2.

### STAT3 Phosphorylation Assay in Primary Human Cells

Ability of IL-10 to activate various immune cell subsets was determined in an assay measuring the phosphorylation of STAT3 by flow cytometry in either human PBMCs or human whole blood.

PBMCs were isolated from blood of healthy donors using Ficoll-Paque Plus (GE Healthcare) and red blood cells were lysed using ACK lysis buffer (Gibco) according to manufacturer's instructions. Typically, PBMCs were resuspended in serum-free RPMI1640 media at 20×10⁶ cells/ml and aliquoted into 96-well U-bottom plates (50 μl per well). IL-10 fusion proteins and control proteins, such as wild-type IL-10 dimer and control fusion proteins, were diluted to desired concentrations and added to wells (50 μl added as 2× stimulus). Incubation was typically performed for 30 min at 37° C. To stain with CD8 antibodies, such as CD8a (SKI, Biolegend; RPA-T8, Biolegend), antibodies were added directly to the wells and incubated on ice for 10 min. Staining was stopped with 100 μl ice cold 8% PFA (4% final) for 10 min on ice. Cells were washed 3× with wash buffer (2% FBS in PBS). Cells were permeabilized in 100 ml pre-chilled Phosflow Perm buffer III (BD Biosciences) according to manufacturer's protocol and stored at −20° C. overnight. The next day cells were washed 2× with wash buffer and stained for 30-45 min at 4° C. with antibodies against: CD3 (UCHT1, BD Biosciences), CD4 (RPA-T4, Biolegend), CD14 (M5E2, Biolegend) CD25 (M-A251, Biolegend), CD56 (HCD56, Biolegend) and/or perform (clone 6G9, BD Biosciences), Foxp3 (259D, Biolegend), pSTAT3 [pY705] (clone 4, BD Biosciences). Cells were then analyzed on a flow cytometer. Data were expressed as percent pSTAT3 positive, and in some cases as pSTAT3 mean fluorescence intensity (MFI), and imported into GraphPad Prism.

For assays in human whole blood, 90 μL human blood was used per well in a 1 mL 96 well deep plate well and prewarmed for 10 minutes at 37° C. IL-10 fusion proteins and control proteins, such as wild-type IL-10 dimer and control fusion proteins were prepared and pre-warmed to 37° C. at 10× strength. 10 μL of prewarmed 10× stimuli was added to each well, creating 100 μL total volume at 1× stimuli concentration. Incubation was typically performed for 25 min at 37° C. The stimulation was quenched by adding pre-fix antibody staining cocktail, vortexing briefly and incubating on ice for 10 minutes in the dark. The pre-fix staining cocktail contained TruStain FcX (Biolgened) and antibodies against: CD4 (RPA-T4, Biolegend), CD19 (HIB19, BD), CD56 (NCAM16.2, BD), CD16 (3G8, Biolegend), and CD8 (SKI, Biolegend). 900 μL pre-warmed Lyse Fix (BD) was added to the sample wells and incubated at 37° C. for 10 minutes. Cells were washed in pre-chilled wash buffer containing PBS+0.5% bovine serum albumin and 2 mM EDTA. Pre-chilled Perm Buffer III (BD) was added and incubated for 60 minutes at −20° C., followed by two washes in wash buffer and one wash in TFP Perm/Wash (BD). Cells were resuspended in 25 μL “post-methanol” staining cocktail prepared in TFP Perm/Wash containing antibodies against: CD3 (UCHT, BD), CD14 (MΦP9, BD), CD1 Ic (B-ly6, BD), HLADR (L243, Biolegend), and pSTAT3 pY705 (4/P-STAT3, BD). Cells were incubated for 30 min at 4° C. in the dark, then washed in TFP Perm/Wash buffer, followed by fixation in 100 μL 4% PFA for 10 minutes at room temperature. Cells were washed twice in wash buffer and analyzed on a flow cytometer. Data were expressed as percent pSTAT3 positive and imported into GraphPad Prism.

### Polyreactivity Assessment by ELISA

In order to measure polyreactivity of candidate fusion proteins, an ELISA assay was used to check for binding to a panel of irrelevant antigens. The following were used as antigens and purchased from Sigma: dsDNA salmon sperm, human serum albumin, keyhole limpet hemocyanin, lipopolysaccharide, insulin, and heparin biotin sodium salt.

Antigens were diluted in PBS to a concentration ranging from 0.3-10 μg/mL and coated onto a 384-well Nunc MaxiSorp plate (Thermo Fisher Scientific) at a volume of 25 μL per well. As a no-antigen control, 25 μL of PBS only was used. The plates were incubated overnight at 4° C. The antigens were removed, and the plate was washed with milli-Q water (Millipore). Wells were filled with PBS supplemented with 0.05% Tween and 1 mM EDTA (assay buffer) and then incubated at room temperature for 1 hour. The assay buffer was removed, and the wells washed with milli-Q water. 25 μL of 10 μg/mL of fusion proteins or bococizumab, a positive control for polyreactivity, diluted in assay buffer were added and incubated at room temperature for 1 hour. Samples were removed and the plate was washed with milli-Q water. 25 μl of the detection antibody, 1:25000 diluted horseradish peroxidase conjugated goat anti-human IgG (Jackson ImmunoResearch), was added and allowed to incubate for 1 hour at room temperature. The reagent was removed, and wells were washed with milli-Q water. Wells were developed using 25 μL of KPL SureBlue TMB Microwell Substrate (SeraCare) for 5-7 mins and quenched with 25 μL of 0.1 M HCl. The absorbance at 450 nm was recorded on a SpectraMax iD5 plate reader (Molecular Devices) and normalized against the no-antigen control well.

Results

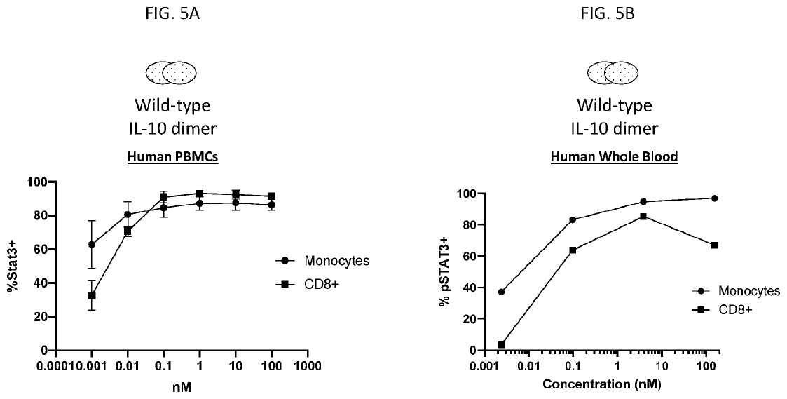

Ability of wild-type IL-10 dimer to activate STAT3 in monocytes and CD8+ T cells is depicted in in FIGS. 5A & 5B. PBMCs from two healthy donors were used and representative data are shown in FIG. 5A. Whole blood from two separate healthy donors was used, and representative data are shown in FIG. 5B. The degree of activation and EC50 of activation in each cell type are comparable across both assays. In both assays, monocytes (gated as CD14+CD3−) were found to be more sensitive to IL-10 than CD8+ T cells.

### Example 2: Preferential Activation of STAT3 in CD8+ T Cells by Fusion Proteins Containing IL-10 Dimers

Fusion proteins comprising the CD8 antibodies and IL-10 dimer polypeptides were made in one of five dimeric formats (A, B, C, D and E shown in FIG. 6).

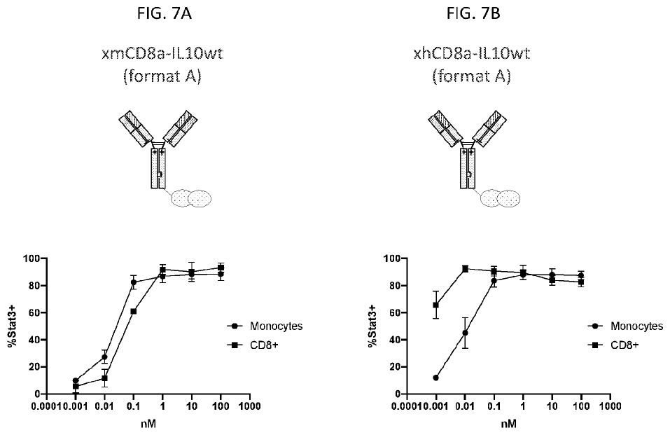

Selectivity and potency of STAT3 activation in hPBMCs by IL-10 fusion proteins in format A are shown in FIG. 7. Fusion proteins tested included xmCD8a-IL10 wt in format A, comprising the wild-type IL-10 polypeptide and a control antibody targeting mouse CD8 (FIG. 7A), and xhCD8a-IL10 wt in format A, comprising the wild-type IL-10 polypeptide and an antibody targeting human CD8 (FIG. 7B). Antibody xmCD8a was a previously published anti-mouse CD8 antibody (2.43 clone), and xhCD8a was a previously published anti-human CD8 antibody (OKT8). STAT3 activation in human PBMCs was measured as described in Example 1. IL-10 fusion protein of format A, xhCD8a-IL10 wt, comprising the antibody specifically binding to human CD8, preferentially activated CD8+ T cells over monocytes, while xmCD8a-IL10 wt, IL-10 fusion protein of format A, comprising the control antibody, preferentially activated monocytes.

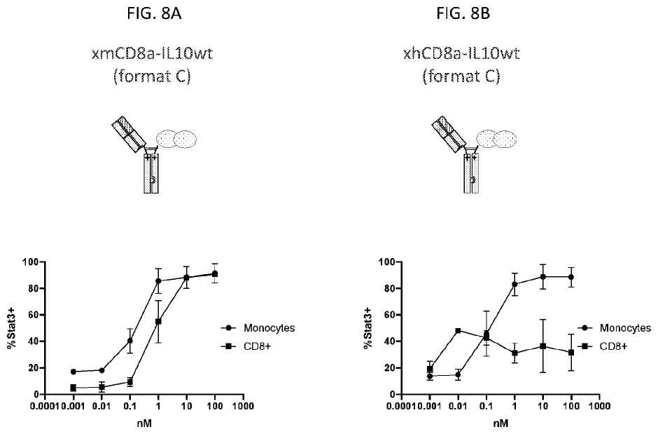

IL-10 fusion proteins xmCD8a-IL10 wt and xhCD8a-IL10 wt were made in format C and their ability to activate STAT3 on human PBMCs was assessed. The results for xmCD8a-IL10 wt in format C are shown in FIG. 8A and for xhCD8a-IL10 wt in format C in FIG. 8B. Fusion protein xmCD8a-IL10 wt in format C was ˜10× less potent than xmCD8a-IL10 wt in format A (compare FIG. 7A and FIG. 8A), suggesting that fusing IL-10 at the N-terminus of human Fc decreased its activity compared to that when IL-10 was fused at the C-terminus of human Fc. Furthermore, format C was not optimal for IL-10 fusion proteins comprising antibodies binding to human CD8, except at low concentrations (up to 0.01 nM). Higher concentrations of xhCD8a-IL10 wt in format C did not fully activate STAT3 in CD8+ T cells and did not preferentially activate CD8+ T cells over monocytes (FIG. 8B).

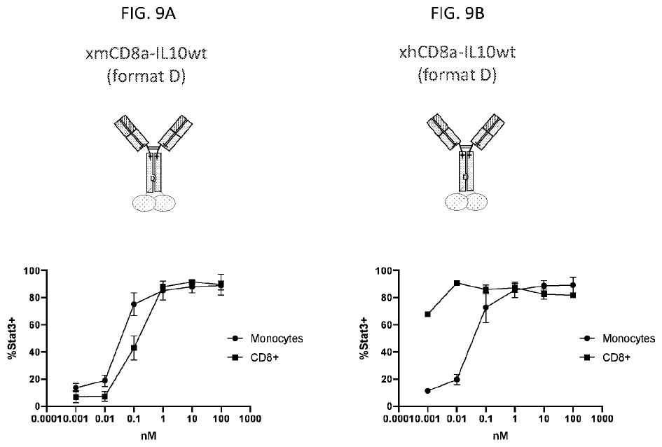

Selectivity and potency of of STAT3 activation in hPBMCs by IL-10 fusion proteins in format D are shown in FIG. 9. The results for xmCD8a-IL10 wt in format D are shown in FIG. 9A and for xhCD8a-IL10 wt in format D in FIG. 9B. IL-10 fusion protein of format D, xhCD8a-IL10 wt, comprising the antibody specifically binding to human CD8, preferentially activated CD8+ T cells over monocytes, while xmCD8a-IL10 wt, IL-10 fusion protein of format D, comprising the control antibody, preferentially activated monocytes.

### Example 3: Preferential Activation of STAT3 in CD8+ T Cells by Fusion Proteins Containing IL-10 Monomers

Fusion proteins comprising CD8 antibodies and IL-10 monomer polypeptides were made according to format F as shown in FIG. 6. The amino acid sequences of the IL-10 monomer polypeptides that were constructed as part of fusion proteins are shown in FIG. 1D in the case of the unmodified monomer IL-10 polypeptide, termed IL10mono, or in Table 4A as shown above in the case of mutant monomer IL-10 polypeptides. IL10mono_RBenh is a mutant monomer IL-10 polypeptide described previously (Gorby et al. Sci Signal. 2020 Sep. 15; 13(649):eabc0653). It contains the amino acid substitutions N18I, N92I, K99N and F111L on the IL10mono background, and has increased binding affinity to IL-10RB. IL10mono_RBenh2 is a mutant monomer IL-10 polypeptide that contains the single amino acid substitution N92I on the IL10 mono background.

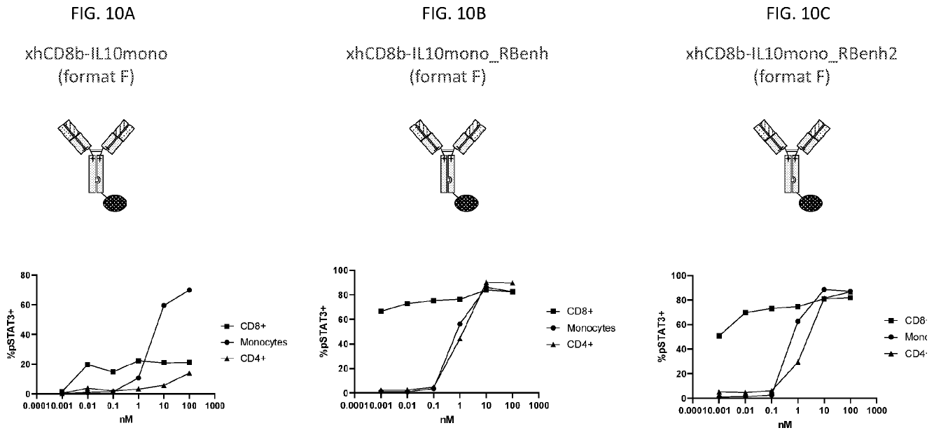

Selectivity and potency of STAT3 activation in hPBMCs by IL-10 monomer fusion proteins are shown in FIGS. 10A-10C. Fusion proteins tested included xhCD8b-IL10mono in format F, comprising the IL10mono polypeptide described above and an antibody targeting human CD8 (FIG. 10A); xhCD8b-IL10mono_RBenh in format F, comprising the IL10mono_RBenh mutant monomer IL-10 polypeptide described above and an antibody targeting human CD8 (FIG. 10B); and xhCD8b-IL10mono_RBenh2 in format F, comprising the IL10mono_RBenh2 mutant monomer IL-10 polypeptide described above and an antibody targeting human CD8 (FIG. 10C). Antibody xhCD8b was an antibody with specificity to human CD8b. STAT3 activation in human PBMCs was measured as described in Example 2.

The fusion protein xhCD8b-IL10mono preferentially activated CD8+ T cells over monocytes and CD4+ T cells at concentrations of 1 nM and below, though the degree of activation was relatively low (FIG. 10A). IL-10RB affinity-enhanced mutant monomer fusion protein comprising the antibody specifically binding to human CD8, xhCD8b-IL10mono_RBenh, also preferentially activated CD8+ T cells over monocytes and CD4+ T cells (FIG. 10B). The difference in activation potency of CD8+ T cells over monocytes/CD4 T cells by xhCD8b-IL10mono_RBenh was significantly higher than the difference in activation potency of CD8+ T cells over monocytes/CD4 T cells by xhCD8b-IL10mono. Additionally, the potency of activation of CD8+ T cells by xhCD8b-IL10mono_RBenh was much higher than by xhCD8b-IL10mono and comparable to that of fusion proteins of wild type dimer IL-10. This illustrates that increased binding affinity to IL-10RB is preferred over unmodified IL-10RB for both potency of activation of CD8+ T cells as well as selectivity of activation in CD8+ T cells over monocytes and CD4+ T cells. The fusion protein xhCD8b-IL10mono_RBenh2, which contains only the single N92I substitution on the IL10mono background, also exhibited increased potency and selective activation of CD8+ T cells over monocytes and CD4+ T cells (FIG. 10C).

### Example 4: Attenuation of Binding Affinity to IL-10RA

As shown in Example 3 and in FIG. 10, enhancement of binding to IL-10RB was able to enhance STAT3 activation by monomer IL-10. To this end, putative mutations for enhancement of IL-10RB binding on the IL-10mono background were identified using a combination of existing structures of IL-10 and its receptors (eg. PDB IDs 1J7V, 6X93, 3LQM) and homology modeling to similar cytokine/receptor complexes (eg. PDB IDs 5T5W, 4DOH, 1Y6K). Amino acid sequences of representative mutant monomer IL-10 polypeptides that were evaluated are shown in Table 5.

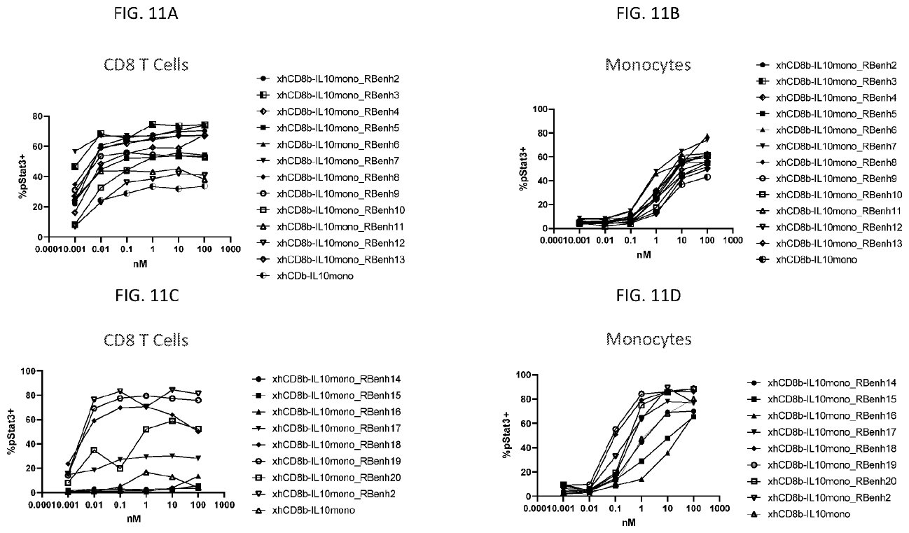

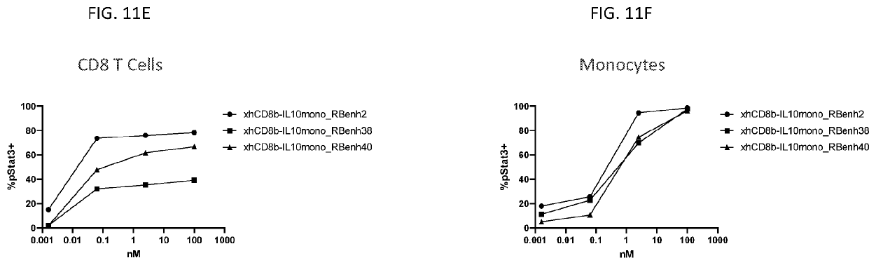

STAT3 activation in hPBMCs by fusion proteins of xhCD8b antibody to a panel of IL-10RB enhanced IL-10 monomers, IL10mono_RBenh3 through IL10mono_RBenh20, are shown in FIGS. 11A through 11D. All fusion proteins were constructed in format F, and STAT3 activation in human PBMCs was measured as described in Example 1. STAT3 activation of fusion proteins of IL10mono_RBenh3 through IL10mono_RBenh13 are shown for CD8 T cells in FIG. 11A, and for monocytes in FIG. 11B. STAT3 activation of fusion proteins of IL10mono_RBenh14 through IL10mono_RBenh20 are shown for CD8 T cells in FIG. 11C, and for monocytes in FIG. 11D. In all figures, STAT3 activation is compared to that of xhCDb-IL10mono_RBenh2. From this panel, all fusions of CD8b-IL10mono_RBenh3 to CD8b-IL10mono_RBenh13, and CD8b-IL10mono_RBenh17 to CD8b-IL10mono_RBenh20 showed enhanced STAT3 activation in CD8 T cells and CD8 T cell selectivity over monocytes compared to xCD8b-IL10mono. Also, Fusion proteins of IL10mono_RBenh3, IL10mono_RBenh4, IL10mono_RBenh6, IL10mono_RBenh7, IL10mono_RBenh8, IL10mono_RBenh18, and IL10mono_RBenh19 show comparable potency and CD8 selectivity of STAT3 activation to IL10mono_RBenh2 fusion protein.

Additional putative mutations for IL-10RB enhancement were constructed and screened using a BIAcore-based assay as described in Example 1. Due to the low affinity of binding to IL-10RB, precise kinetics could not be determined. Instead, the binding response to 20 μM IL-10RB was measured and normalized to the capture level of IL10mono fusion protein in order to rank the relative binding affinities of the IL10mono muteins to IL-10RB. Normalized binding response to IL-10RB for fusion proteins of IL10mono_RBenh21 through IL10mono_RBenh60, along with several controls, are shown in Table 6. In particular, IL10mono_RBenh38, IL10mono_RBenh40, and IL10mono_RBenh60 show enhanced binding over IL10mono.

STAT3 activation in human whole blood was evaluated for selected fusion proteins identified by the BIAcore-based screen. STAT3 activation is shown for CD8 T cells in FIG. 11E and for monocytes in FIG. 11F. Here, the fusion protein of IL10mono_RBenh38 shows STAT3 activation in CD8 T cells that is slightly lower but comparable to that of IL10mono_RBenh2.

TABLE 5

| IL10 Mutein | Sequence | SEQ ID NO |
|---|---|---|
| IL10mono_RBenh3 | SPGQGTQSENSCTHFPGYLPNMLRDLRDAFSRVKTFFQMKDQL | 161 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh4 | SPGQGTQSENSCTHFPGNLPNMLRDLRDAFSRVKTFFQMKDQL | 162 |
| | DNLLLKESLLEDFKGYLGCQALSEMIQFYLEEVMPQAENQDPDIK | |
| | AHVNSLGENLNTLRLRLRRCHRFLPCENGGGSGGKSKAVEQVKN | |
| | AFNKLQEKGIYKAMSEFDIFINYIEAYMTMKIRN | |
| IL10mono_RBenh5 | SPGQGTQSENSCTHFPGNLPNMLRDLRDAFSRVKTFFQMKDQL | 163 |
| | DNLLLKESLLEDFKGYLGCQALSEMIQFYLEEVMPQAENQDPDIK | |
| | AHVSSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh6 | SPGQGTQSENSCTHFPGNLPNMLRDLRDAFSRVKTFFQMKDQL | 164 |
| | DNLLLKESLLEDFKGYLGCQALSEMIQFYLEEVMPQAENQDPDIK | |
| | AHVVSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh7 | SPGQGTQSENSCTHFPGNLPNMLRDLRDAFSRVKTFFQMKDQL | 165 |
| | DNLLLKESLLEDFKGYLGCQALSEMIQFYLEEVMPQAENQDPDIK | |
| | AHVLSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh8 | SPGQGTQSENSCTHFPGNLPNMLRDLRDAFSRVKTFFQMKDQL | 166 |
| | DNLLLKESLLEDFKGYLGCQALSEMIQFYLEEVMPQAENQDPDIK | |
| | AHVRSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh9 | SPGQGTQSENSCTHFPGNLPNMLRDLRDAFSRVKTFFQMKDQL | 167 |
| | DNLLLKESLLEDFKGYLGCQALSEMIQFYLEEVMPQAENQDPDIK | |
| | AHVFSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh10 | SPGQGTQSENSCTHFPGNLPNMLRDLRDAFSRVKTFFQMKDQL | 168 |
| | DNLLLKESLLEDFKGYLGCQALSEMIQFYLEEVMPQAENQDPDIK | |
| | AHVHSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh11 | SPGQGTQSENSCTHFPGNLPNMLRDLRDAFSRVKTFFQMKDQL | 169 |
| | DNLLLKESLLEDFKGYLGCQALSEMIQFYLEEVMPQAENQDPDIK | |
| | AHVYSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh12 | SPGQGTQSENSCTHFPGNLPNMLRDLRDAFSRVKTFFQMKDQL | 170 |
| | DNLLLKESLLEDFKGYLGCQALSEMIQFYLEEVMPQAENQDPDIK | |
| | AHVKSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh13 | SPGQGTQSENSCTHFPGNLPNMLRDLRDAFSRVKTFFQMKDQL | 171 |
| | DNLLLKESLLEDFKGYLGCQALSEMIQFYLEEVMPQAENQDPDIK | |
| | AHVTSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh14 | SPGQGTQSENSCTHFPGNLPNMLRDLRDAFSKVKTFFQMKDQL | 172 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh15 | SPGQGTQSENSCTHFPGNLPNMLRDLRDAFSQVKTFFQMKDQL | 173 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh16 | SPGQGTQSENSCTHFPGNLPNMLRDLRDAFSRVKTFFQMKDQL | 174 |
| | DNLLLKESLLEDFKGYLGCQALSEMIQFYLEEVMPQAQNQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh17 | SPGQGTQSENSCTHFPGNLPNMLRDLRDAFSRVKTFFQMKDQL | 175 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh18 | SPGQGTQSEQSCTHFPGNLPNMLRDLRDAFSRVKTFFQMKDQL | 176 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh19 | SPGQGTQSENSCTHFPGNLPNMLRDLRRAFSRVKTFFQMKDQL | 202 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh20 | SPGQGTQSENSCTHFPGNLPNMLRDLRQAFSRVKTFFQMKDQL | 203 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh21 | SPGQGTQSENSCTHFPGNLPNMLRDLRDAFSHVKTFFQMKDQL | 204 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh22 | SPGQGTQSENSCTHFPGNLPNMLRDLRDAFSYVKTFFQMKDQL | 205 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh23 | SPGQGTQSENSCTHFPGNLPNMLRDLRDAFSRVKTFFQMKDQL | 206 |
| | DNLLLKESLLEDFKGYLGCQALSEMIQFYLEEVMPQAYNQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh24 | SPGQGTQSENSCTHFPGNLPNMLRDLRDAFSRVKTFFQMKDQL | 207 |
| | DNLLLKESLLEDFKGYLGCQALSEMIQFYLEEVMPQANNQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh25 | SPGQGTQSENSCTHFPGNLPNMLRDLRDAFSRVKTFFQMKDQL | 208 |
| | DNLLLKESLLEDFKGYLGCQALSEMIQFYLEEVMPQASNQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh26 | SPGQGTQSENSCTHFPGNLPNMLRDLRDAFSRVKTFFQMKDQL | 209 |
| | DNLLLKESLLEDFKGYLGCQALSEMIQFYLEEVMPQAINQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh27 | SPGQGTQSENSCTHFPGNLPNMLRDLRDAFSRVKTFFQMKDQL | 210 |
| | DNLLLKESLLEDFKGYLGCQALSEMIQFYLEEVMPQAVNQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh28 | SPGQGTQSENSCTHFPGNLPNMLRDLRDAFSRVKTFFQMKDQL | 211 |
| | DNLLLKESLLEDFKGYLGCQALSEMIQFYLEEVMPQAENQDPDIK | |
| | AHVNSLGENLKTLRHRLRRCHRFLPCENGGGSGGKSKAVEQVKN | |
| | AFNKLQEKGIYKAMSEFDIFINYIEAYMTMKIRN | |
| IL10mono_RBenh29 | SPGQGTQSENSCTHFPGNLPNMLRDLRDAFSRVKTFFQMKDQL | 212 |
| | DNLLLKESLLEDFKGYLGCQALSEMIQFYLEEVMPQAENQDPDIK | |
| | AHVNSLGENLKTLRRRLRRCHRFLPCENGGGSGGKSKAVEQVKN | |
| | AFNKLQEKGIYKAMSEFDIFINYIEAYMTMKIRN | |
| IL10mono_RBenh30 | SPGQGTQSENSCTHFPGNLPNMLRDLRDAFSRVKTFFQMKDQL | 213 |
| | DNLLLKESLLEDFKGYLGCQALSEMIQFYLEEVMPQAENQDPDIK | |
| | AHVNSLGENLKTLRKRLRRCHRFLPCENGGGSGGKSKAVEQVKN | |
| | AFNKLQEKGIYKAMSEFDIFINYIEAYMTMKIRN | |
| IL10mono_RBenh31 | SPGQGTQSENSCTHFPGNLPNMLRDLRDAFSRVKTFFQMKDQL | 214 |
| | DNLLLKESLLEDFKGYLGCQALSEMIQFYLEEVMPQAENQDPDIK | |
| | AHVNSLGENLKTLRTRLRRCHRFLPCENGGGSGGKSKAVEQVKN | |
| | AFNKLQEKGIYKAMSEFDIFINYIEAYMTMKIRN | |
| IL10mono_RBenh32 | SPGQGTQSENSCTHFPGNLPNMLRDLRDAFSRVKTFFQMKDQL | 215 |
| | DNLLLKESLLEDFKGYLGCQALSEMIQFYLEEVMPQAENQDPDIK | |
| | AHVNSLGENLKTLRSRLRRCHRFLPCENGGGSGGKSKAVEQVKN | |
| | AFNKLQEKGIYKAMSEFDIFINYIEAYMTMKIRN | |
| IL10mono_RBenh33 | SPGQGTQSENSCTHFPGNLPNMLRDLRKAFSRVKTFFQMKDQL | 216 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh34 | SPGQGTQSENSCTHFPGNLPNMLRDLRHAFSRVKTFFQMKDQL | 217 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh35 | SPGQGTQSENSCTHFPGELPNMLRDLRDAFSRVKTFFQMKDQL | 218 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh36 | SPGQGTQSENSCTHFPGSLPNMLRDLRDAFSRVKTFFQMKDQL | 219 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh37 | SPGQGTQSENSCTHFPGTLPNMLRDLRDAFSRVKTFFQMKDQL | 220 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh38 | SPGQGTQSENSCTHFPGLLPNMLRDLRDAFSRVKTFFQMKDQL | 221 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh39 | SPGQGTQSENSCTHFPGVLPNMLRDLRDAFSRVKTFFQMKDQL | 222 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh40 | SPGQGTQSENSCTHFPGFLPNMLRDLRDAFSRVKTFFQMKDQL | 223 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh41 | SPGQGTQSENSCTEFPGNLPNMLRDLRDAFSRVKTFFQMKDQL | 224 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh42 | SPGQGTQSENSCTQFPGNLPNMLRDLRDAFSRVKTFFQMKDQL | 274 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh43 | SPGQGTQSENSCTRFPGNLPNMLRDLRDAFSRVKTFFQMKDQL | 275 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh44 | SPGQGTQSENSCTKFPGNLPNMLRDLRDAFSRVKTFFQMKDQL | 276 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh45 | SPGQGTQSENSCTSFPGNLPNMLRDLRDAFSRVKTFFQMKDQL | 281 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh46 | SPGQGTQSENSCTTFPGNLPNMLRDLRDAFSRVKTFFQMKDQL | 282 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh47 | SPGQGTQSENSCTIFPGNLPNMLRDLRDAFSRVKTFFQMKDQL | 283 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh48 | SPGQGTQSENSCTVFPGNLPNMLRDLRDAFSRVKTFFQMKDQL | 297 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh49 | SPGQGTQSENSCTYFPGNLPNMLRDLRDAFSRVKTFFQMKDQL | 298 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh50 | SPGQGTQSENSCTHFPGNLPEMLRDLRDAFSRVKTFFQMKDQL | 299 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh51 | SPGQGTQSENSCTHFPGNLPRMLRDLRDAFSRVKTFFQMKDQL | 300 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh52 | SPGQGTQSENSCTHFPGNLPKMLRDLRDAFSRVKTFFQMKDQL | 301 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh53 | SPGQGTQSENSCTHFPGNLPLMLRDLRDAFSRVKTFFQMKDQL | 302 |
| | DNLLLKESLLEDFKGYLGCQALSEMIQFYLEEVMPQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh54 | SPGQGTQSENSCTHFPGNLPNMLRDLRDAFSRVKTFFQMKDQL | 303 |
| | DNLLLKESLLEDFKGYLGCQALSEMIQFYLEEVMLQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh55 | SPGQGTQSENSCTHFPGNLPNMLRDLRDAFSRVKTFFQMKDQL | 304 |
| | DNLLLKESLLEDFKGYLGCQALSEMIQFYLEEVMIQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh56 | SPGQGTQSENSCTHFPGNLPNMLRDLRDAFSRVKTFFQMKDQL | 305 |
| | DNLLLKESLLEDFKGYLGCQALSEMIQFYLEEVMFQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh57 | SPGQGTQSENSCTHFPGNLPNMLRDLRDAFSRVKTFFQMKDQL | 306 |
| | DNLLLKESLLEDFKGYLGCQALSEMIQFYLEEVMRQAENQDPDIK | |
| | AHVNSLGENLKTLRLRLRRCHRFLPCENGGGSGGKSKAVEQVKN | |
| | AFNKLQEKGIYKAMSEFDIFINYIEAYMTMKIRN | |
| IL10mono_RBenh58 | SPGQGTQSENSCTHFPGNLPNMLRDLRDAFSRVKTFFQMKDQL | 307 |
| | DNLLLKESLLEDFKGYLGCQALSEMIQFYLEEVMPQAENQDPDIK | |
| | AHVNSLGENLKELRLRLRRCHRFLPCENGGGSGGKSKAVEQVKN | |
| | AFNKLQEKGIYKAMSEFDIFINYIEAYMTMKIRN | |
| IL10mono_RBenh59 | SPGQGTQSENSCTHFPGNLPNMLRDLRDAFSRVKTFFQMKDQL | 308 |
| | DNLLLKESLLEDFKGYLGCQALSEMIQFYLEEVMPQAENQDPDIK | |
| | AHVNSLGENLKLLRLRLRRCHRFLPCENGGGSGGKSKAVEQVKN | |
| | AFNKLQEKGIYKAMSEFDIFINYIEAYMTMKIRN | |
| IL10mono_RBenh60 | SPGQGTQSENSCTHFPGNLPNMLRDLRDAFSRVKTFFQMKDQL | 309 |
| | DNLLLKESLLEDFKGYLGCQALSEMIQFYLEEVMPQAENQDPDIK | |
| | AHVNSLGENLKILRLRLRRCHRFLPCENGGGSGGKSKAVEQVKN | |
| | AFNKLQEKGIYKAMSEFDIFINYIEAYMTMKIRN | |

TABLE 6

| | | | | IL10-RB | |
|---|---|---|---|---|---|
| | | | | Binding | |
| | | Mutein | | Response | |
| | | capture | | (Normalized | |
| | Mutein | level | IL10RB (20 | to capture | Fold |
| | Ligand | (Normalized | μM) Binding | level of WT) | difference |
| IL-10 mutein | Level (RU) | to WT) (RU) | Response (RU) | (RU) | over WT |
| IL10mono | 1197 | 1.00 | 4.8 | 4.8 | 1.0 |
| IL10mono_RBenh | 1026 | 0.86 | 141.5 | 121.3 | 25.3 |
| IL10mono_RBenh2 | 1046 | 0.87 | 18.3 | 16.0 | 3.3 |
| IL10mono_RBenh6 | 1078 | 0.90 | 11.1 | 10.0 | 2.1 |
| IL10mono_RBenh19 | 1025 | 0.86 | 5.2 | 4.5 | 0.9 |
| IL10mono_RBenh21 | 842 | 0.70 | 2.3 | 1.6 | 0.3 |
| IL10mono_RBenh22 | 1020 | 0.85 | 1.9 | 1.6 | 0.3 |
| IL10mono_RBenh23 | 1116 | 0.93 | 1.2 | 1.1 | 0.2 |
| IL10mono_RBenh24 | 1067 | 0.89 | 1.4 | 1.2 | 0.3 |
| IL10mono_RBenh25 | 1010 | 0.84 | 1.8 | 1.5 | 0.3 |
| IL10mono_RBenh26 | 1012 | 0.85 | 1.1 | 0.9 | 0.2 |
| IL10mono_RBenh27 | 940 | 0.79 | 1.8 | 1.4 | 0.3 |
| IL10mono_RBenh28 | 1411 | 1.18 | 2.3 | 2.7 | 0.6 |
| IL10mono_RBenh29 | 1260 | 1.05 | 1.5 | 1.6 | 0.3 |
| IL10mono_RBenh30 | 986 | 0.82 | 2.4 | 2.0 | 0.4 |
| IL10mono_RBenh31 | 949 | 0.79 | 3.7 | 2.9 | 0.6 |
| IL10mono_RBenh32 | 1208 | 1.01 | 3.5 | 3.5 | 0.7 |
| IL10mono_RBenh33 | 1113 | 0.93 | 3.5 | 3.3 | 0.7 |
| IL10mono_RBenh34 | 1170 | 0.98 | 4.1 | 4.0 | 0.8 |
| IL10mono_RBenh35 | 900 | 0.75 | 4 | 3.0 | 0.6 |
| IL10mono_RBenh36 | 982 | 0.82 | 3.2 | 2.6 | 0.5 |
| IL10mono_RBenh37 | 1032 | 0.86 | 3.5 | 3.0 | 0.6 |
| IL10mono_RBenh38 | 1118 | 0.93 | 6.8 | 6.4 | 1.3 |
| IL10mono_RBenh39 | 1038 | 0.87 | 5.2 | 4.5 | 0.9 |
| IL10mono_RBenh40 | 1141 | 0.95 | 14 | 13.4 | 2.8 |
| IL10mono_RBenh41 | 1091 | 0.91 | 2.4 | 2.2 | 0.5 |
| IL10mono_RBenh42 | 1076 | 0.90 | 2.9 | 2.6 | 0.5 |
| IL10mono_RBenh43 | 1197 | 1.00 | 3.1 | 3.1 | 0.6 |
| IL10mono_RBenh44 | 1228 | 1.03 | 2.3 | 2.4 | 0.5 |
| IL10mono_RBenh45 | 1082 | 0.90 | 2.7 | 2.4 | 0.5 |
| IL10mono_RBenh46 | 1111 | 0.93 | 2.9 | 2.7 | 0.6 |
| IL10mono_RBenh47 | 1087 | 0.91 | 2.4 | 2.2 | 0.5 |
| IL10mono_RBenh48 | 987 | 0.82 | 2.8 | 2.3 | 0.5 |
| IL10mono_RBenh49 | 1058 | 0.88 | 3.2 | 2.8 | 0.6 |
| IL10mono_RBenh50 | 1130 | 0.94 | 1.5 | 1.4 | 0.3 |
| IL10mono_RBenh51 | 1297 | 1.08 | 2.5 | 2.7 | 0.6 |
| IL10mono_RBenh52 | 1349 | 1.13 | 2.1 | 2.4 | 0.5 |
| IL10mono_RBenh53 | 1183 | 0.99 | 3.3 | 3.3 | 0.7 |
| IL10mono_RBenh54 | 941 | 0.79 | 2.3 | 1.8 | 0.4 |
| IL10mono_RBenh55 | 1013 | 0.85 | 2.3 | 1.9 | 0.4 |
| IL10mono_RBenh56 | 997 | 0.83 | 2.8 | 2.3 | 0.5 |
| IL10mono_RBenh57 | 880 | 0.74 | 3.1 | 2.3 | 0.5 |
| IL10mono_RBenh58 | 1090 | 0.91 | 3.7 | 3.4 | 0.7 |
| IL10mono_RBenh59 | 1177 | 0.98 | 3.8 | 3.7 | 0.8 |
| IL10mono_RBenh60 | 1120 | 0.94 | 7.2 | 6.7 | 1.4 |

### Example 5: Attenuation of Binding Affinity to IL-10RA

As shown in Example 3, fusion proteins comprising CD8 antibodies and IL-10 monomer polypeptides selectively activated CD8+ T cells over monocytes and CD4+ T cells. In order to further reduce activity on non-specific cells, a panel of amino acid substitutions were designed to reduce binding affinity to IL-10RA on the background of IL-10RB-enhanced polypeptides, IL10mono_RBenh or IL10mono_RBenh2. Amino acid substitutions and the amino acid sequences of representative mutant monomer IL-10 polypeptides that were evaluated are shown in Table 4A. The binding affinity of these constructs to IL-10RA was measured by BIAcore as described in Example 1, and these data are summarized in Table 7. In all cases, the IL-10 polypeptide was expressed as a fusion protein, with the antibody binding domain specified in column 2, and the format shown in parentheses, based on the schematics depicted in FIG. 6. The Kd of binding is listed in column 3, where “ND” indicates that binding was detected, but the affinity was too low for a reliable calculation of Kd. “NT” indicates that the mutein was not evaluated by BIAcore.

In all cases, the mutations reduced binding affinity of the IL-10 polypeptides, indicating that there is utility of these amino acid substitutions to reduce activity on monocytes and other non-specific cell types.

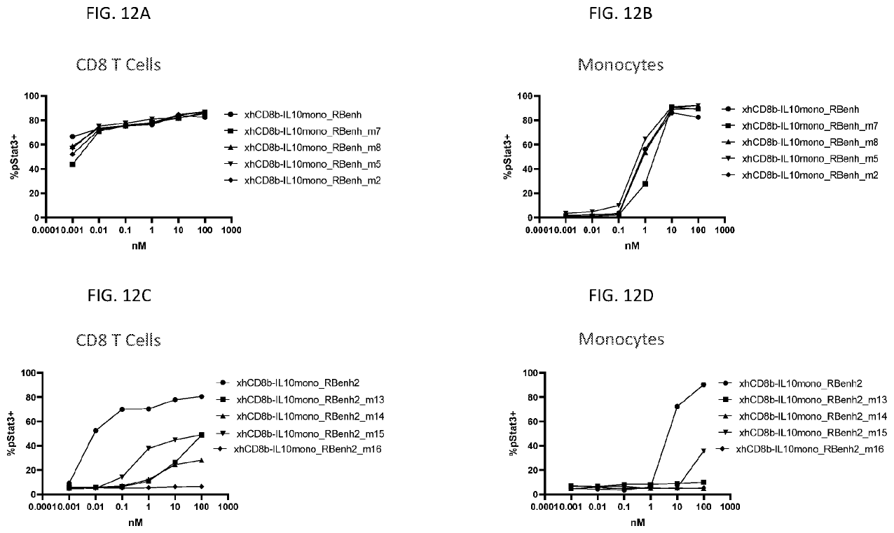

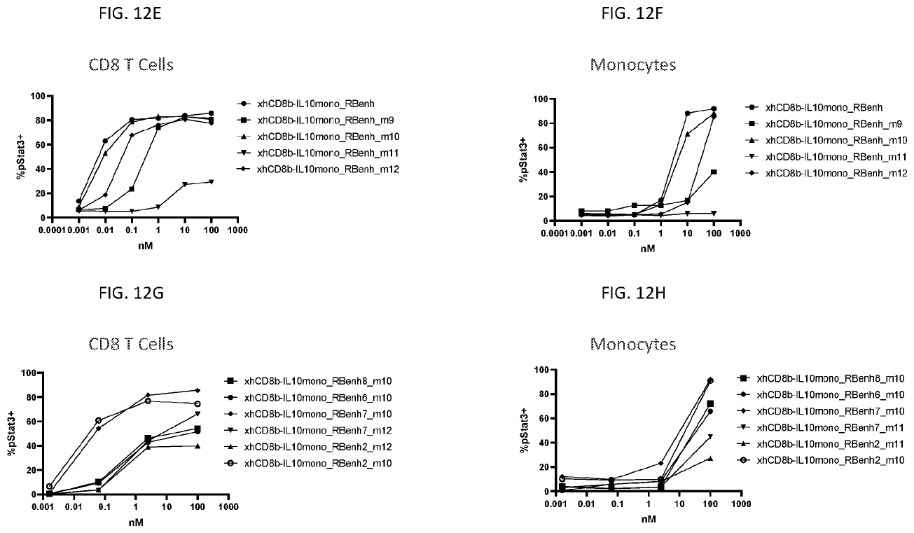

Selectivity and potency of STAT3 activation in hPBMCs by fusion proteins of xhCD8b antibody to a panel of IL-10RA-attenuated constructs on either the IL10mono_RBenh or IL10mono_RBenh2 background are shown in FIGS. 12A through 12F. All fusion proteins were constructed in format F, and STAT3 activation in human PBMCs was measured as described in Example 1. FIGS. 12A and 12B show STAT3 activation in CD8 T cells and monocytes, respectively, of fusion proteins that are insufficiently attenuated for binding to IL-10RA. As shown in FIG. 12B, activity on monocytes remains largely unchanged. FIGS. 12C, 12D, 12E and 12F show STAT3 activation in CD8 T cells and monocytes, respectively, of fusion proteins that are attenuated to varying degrees for binding to IL-10RA. Results in FIGS. 12D and 12F show that potency reductions in monocytes range from about 2-fold attenuation for IL10mono_RBenh_m10 to greater than 100-fold attenuation for IL10mono_RBenh_m11. Similar level of potency reduction was observed in both monocytes and CD8 T cells for each of these IL-10RA muteins compared to IL-10mono_RBenh, which does not contain any IL-10RA mutation. The affinities of selected fusions to IL-10RA were also reported in Table 7.

Based on these results, selected IL-10RA-attenuating mutations were combined with various IL-10RB-enhancing mutations and evaluated in a STAT3 assay in human whole blood. Amino acid sequences of representative mutant monomer IL-10 polypeptides that were evaluated are shown in Table 8. FIGS. 12G and 12H show STAT3 activation in CD8 T cells and monocytes, respectively, of these representative fusion proteins. In all constructs tested, there is selectivity of CD8 T cell activation over monocytes, with the highest potency and selectivity observed with fusion proteins of IL10mono_RBenh2_m10 or IL10mono_RBenh7 m10.

TABLE 7
BIAcore data for binding of selected
IL-10 polypeptides with IL-10RA.

| | Antibody binding | IL-10RA |
|---|---|---|
| | domain used in | Affinity at |
| IL-10 Construct | fusion protein (Format) | 37° C. (nM) |
| Wild type IL-10 dimer | xhCD8b (E) | 4.5 |
| IL10mono | xhCD8b (F) | 9.1 |
| IL10mono_RBenh | xhCD8b (F) | 4.5 |
| IL10mono_RBenh_m1 | xhCD8b (F) | 106 |
| IL10mono_RBenh_m2 | xhCD8b (F) | 52.3 |
| IL10mono_RBenh_m3 | xhCD8b (F) | ND |
| IL10mono_RBenh_m4 | xhCD8b (F) | 57.5 |
| IL10mono_RBenh_m5 | xhCD8b (F) | 1100 |
| IL10mono_RBenh_m6 | xhCD8b (F) | 25 |
| IL10mono_RBenh_m7 | xhCD8b (F) | 3310 |
| IL10mono_RBenh_m8 | xhCD8b (F) | 5144 |
| IL10mono_RBenh_m9 | xhCD8b (F) | ND* |
| IL10mono_RBenh_m10 | xhCD8b (F) | ND |
| IL10mono_RBenh_m11 | xhCD8b (F) | ND |
| IL10mono_RBenh_m12 | xhCD8b (F) | ND |
| IL10mono_RBenh2_m13 | xhCD8b (F) | NT** |
| IL10mono_RBenh2_m14 | xhCD8b (F) | NT |
| IL10mono_RBenh2_m15 | xhCD8b (F) | NT |
| IL10mono_RBenh2_m16 | xhCD8b (F) | NT |

*ND indicates that binding was detected, but the affinity was too low to be calculated.
**NT indicates that affinity was not tested

TABLE 8

| IL10 Mutein | Sequence | SEQ ID NO |
|---|---|---|
| IL10mono_RBenh2_ | SPGQGTQSENSCTHFPGNLPNMLRDLRDAFSRVKTFFQMKDQLDNLLLKE | 310 |
| m10 | SLLEDFKGYLGCQALSEMIQFYLEEVMPQAENQDPDIKAHVISLGENLKTLR | |
| | LRLRRCHRFLPCENGGGSGGKSKAVEQVKNAFNKLQEKGIYKAMSEFAIFIN | |
| | YIEAYMTMKIRN | |
| IL10mono_RBenh2_ | SPGQGTQSENSCTHFPGNLPNMLRDLRDAFSRVKTFFQMKDQLDNLLLKE | 311 |
| m12 | SLLEDFKGYLGCQALSEMIQFYLEEVMPQAENQDPDIKAHVISLGENLKTLR | |
| | LRLRRCHRFLPCENGGGSGGKSKAVEQVKNAFNKLQEKGIYAAMSAFDIFI | |
| | NYIEAYMTMKIRN | |
| IL10mono_RBenh7_ | SPGQGTQSENSCTHFPGNLPNMLRDLRDAFSRVKTFFQMKDQLDNLLLKE | 312 |
| m12 | SLLEDFKGYLGCQALSEMIQFYLEEVMPQAENQDPDIKAHVLSLGENLKTLR | |
| | LRLRRCHRFLPCENGGGSGGKSKAVEQVKNAFNKLQEKGIYAAMSAFDIFI | |
| | NYIEAYMTMKIRN | |
| IL10mono_RBenh7_ | SPGQGTQSENSCTHFPGNLPNMLRDLRDAFSRVKTFFQMKDQLDNLLLKE | 313 |
| m10 | SLLEDFKGYLGCQALSEMIQFYLEEVMPQAENQDPDIKAHVLSLGENLKTLR | |
| | LRLRRCHRFLPCENGGGSGGKSKAVEQVKNAFNKLQEKGIYKAMSEFAIFIN | |
| | YIEAYMTMKIRN | |
| IL10mono_RBenh6_ | SPGQGTQSENSCTHFPGNLPNMLRDLRDAFSRVKTFFQMKDQLDNLLLKE | 314 |
| m10 | SLLEDFKGYLGCQALSEMIQFYLEEVMPQAENQDPDIKAHVVSLGENLKTL | |
| | RLRLRRCHRFLPCENGGGSGGKSKAVEQVKNAFNKLQEKGIYKAMSEFAIFI | |
| | NYIEAYMTMKIRN | |
| IL10mono_RBenh8_ | SPGQGTQSENSCTHFPGNLPNMLRDLRDAFSRVKTFFQMKDQLDNLLLKE | 315 |
| m10 | SLLEDFKGYLGCQALSEMIQFYLEEVMPQAENQDPDIKAHVRSLGENLKTL | |
| | RLRLRRCHRFLPCENGGGSGGKSKAVEQVKNAFNKLQEKGIYKAMSEFAIFI | |
| | NYIEAYMTMKIRN | |
| IL10mono_RBenh2. | SPGQGTQSENSCTHFPGNLPNMLRDLRDAFSRVKTFFQMKD | 316 |
| 1-m10 | QLDNLLLKESLLEDFKGYLGCQALSEMIQFYLEEVMPQAEN | |
| | QDPDIKAHVISLGENLKTLRLRLRRCHRFLPCENKGGGSGGS | |
| | KAVEQVKNAFNKLQEKGIYKAMSEFAIFINYIEAYMTMKIRN | |
| IL10mono_RBenh7. | SPGQGTQSENSCTHFPGNLPNMLRDLRDAFSRVKTFFQMKD | 317 |
| 1-m10 | QLDNLLLKESLLEDFKGYLGCQALSEMIQFYLEEVMPQAEN | |
| | QDPDIKAHVLSLGENLKTLRLRLRRCHRFLPCENKGGGSGGS | |
| | KAVEQVKNAFNKLQEKGIYKAMSEFAIFINYIEAYMTMKIRN | |
| IL10mono_RBenh2. | SPGQGTQSENSCTHFPGNLPNMLADLADAFSRVKTFFQMKDQLDN | 318 |
| 1-m15 | LLLKESLLEDFKGYLGCQALSEMIQFYLEEVMPQAENQDPDIKAHVIS | |
| | LGENLKTLRLRLRRCHRFLPCENKGGGSGGSKAVEQVKNAFNKLQEK | |
| | GIYKAMSEFDIFINYIEAYMTMKIRN | |

### Example 6: Additional Screening of Constructs with Attenuated Binding Affinity to IL-10RA

Putative mutations for attenuation of IL-1 RA binding were designed and incorporated on the IL10mono_RBenh6 background. Amino acid sequences of representative mutant monomer IL-10 polypeptides that were evaluated are shown in Table 9.

Screening of muteins that attenuate binding affinity to IL-10RA was performed by BIAcore, as described in Example 1. Affinity data are shown in Table 10. Constructs with weak or undetectable binding to IL-10RA were evaluated for binding to Protein G, as well as for integrity by SDS-PAGE, in order to rule out the possibility of degraded protein. “ND” indicates that binding was detected, but the affinity was too low for a reliable calculation of Kd. “NB” indicates that no binding was detected above a buffer-only control.

TABLE 9

| | | SEQ |
|---|---|---|
| IL10 Mutein | Sequence | ID NO |
| IL10mono_RBenh6- | SPGQGTQSENSCTHFPGNLPNMLRDLRDAFSRVKTFFQMKDQLDNLL | 319 |
| m17 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFRIFINYIEAYMTMKIRN | |
| IL10mono_RBenh6- | SPGQGTQSENSCTHFPGNLPNMLRDLRDAFSRVKTFFQMKDQLDNLL | 320 |
| m18 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFKIFINYIEAYMTMKIRN | |
| IL10mono_RBenh6- | SPGQGTQSENSCTHFPGNLPNMLRDLRDAFSRVKTFFQMKDQLDNLL | 321 |
| m19 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSNFDIFINYIEAYMTMKIRN | |
| IL10mono_RBenh6- | SPGQGTQSENSCTHFPGNLPNMLRDLRDAFSRVKTFFQMKDQLDNLL | 322 |
| m20 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSFFDIFINYIEAYMTMKIRN | |
| IL10mono_RBenh6- | SPGQGTQSENSCTHFPGNLPNMLRDLRDAFSRVKTFFQMKDQLDNLL | 323 |
| m21 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIPAYMTMKIRN | |
| IL10mono_RBenh6- | SPGQGTQSENSCTHFPGNLPNMLRDLRDAFSRVKTFFEMKDQLDNLLL | 324 |
| m22 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFSMKDQLDNLLL | 325 |
| m23 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVGTFFQMKDQLDNLL | 326 |
| m24 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVTTFFQMKDQLDNLL | 327 |
| m25 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLGNLL | 328 |
| m26 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLTNLLL | 329 |
| m27 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLDNLL | 330 |
| m28 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFNIFINYIEAYMTMKIRN | |
| IL10mono_RBenh6- | SPGQGTQSENSCTHFPGNLPNMLRDLRDAFSRVKTFFQMKDQLDNLL | 331 |
| m29 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSPFDIFINYIEAYMTMKIRN | |
| IL10mono_RBenh6- | SPGQGTQSENSCTHFPGNLPNMLRDLRDAFSRVKTFFQMKDQLDNLL | 332 |
| m30 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIQAYMTMKIRN | |
| IL10mono_RBenh6- | SPGQGTQSENSCTHFPGNLPNMLRDLRDAFSRVKTFFQMKDQLDNLL | 333 |
| m31 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYISAYMTMKIRN | |
| IL10mono_RBenh6- | SPGQGTQSENSCTHFPGNLPNMLRDLRDAFSRVKTFFGMKDQLDNLL | 334 |
| m32 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFTMKDQLDNLLL | 335 |
| m33 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVHTFFQMKDQLDNLL | 336 |
| m34 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVYTFFQMKDQLDNLL | 337 |
| m35 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLHNLL | 338 |
| m36 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLDNLL | 339 |
| m37 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFQIFINYIEAYMTMKIRN | |
| IL10mono_RBenh6- | SPGQGTQSENSCTHFPGNLPNMLRDLRDAFSRVKTFFQMKDQLDNLL | 340 |
| m38 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFPIFINYIEAYMTMKIRN | |
| IL10mono_RBenh6- | SPGQGTQSENSCTHFPGNLPNMLRDLRDAFSRVKTFFQMKDQLDNLL | 341 |
| m39 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSQFDIFINYIEAYMTMKIRN | |
| IL10mono_RBenh6- | SPGQGTQSENSCTHFPGNLPNMLRDLRDAFSRVKTFFQMKDQLDNLL | 342 |
| m40 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSSFDIFINYIEAYMTMKIRN | |
| IL10mono_RBenh6- | SPGQGTQSENSCTHFPGNLPNMLRDLRDAFSRVKTFFQMKDQLDNLL | 343 |
| m41 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIGAYMTMKIRN | |
| IL10mono_RBenh6- | SPGQGTQSENSCTHFPGNLPNMLRDLRDAFSRVKTFFQMKDQLDNLL | 344 |
| m42 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYITAYMTMKIRN | |
| IL10mono_RBenh6- | SPGQGTQSENSCTHFPGNLPNMLRDLRDAFSRVKTFFHMKDQLDNLL | 345 |
| m43 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFYMKDQLDNLLL | 346 |
| m44 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVVTFFQMKDQLDNLL | 347 |
| m45 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLINLLL | 348 |
| m46 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLDNLL | 349 |
| m47 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFEIFINYIEAYMTMKIRN | |
| IL10mono_RBenh6- | SPGQGTQSENSCTHFPGNLPNMLRDLRDAFSRVKTFFQMKDQLDNLL | 350 |
| m48 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFSIFINYIEAYMTMKIRN | |
| IL10mono_RBenh6- | SPGQGTQSENSCTHFPGNLPNMLRDLRDAFSRVKTFFQMKDQLDNLL | 351 |
| m49 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSGFDIFINYIEAYMTMKIRN | |
| IL10mono_RBenh6- | SPGQGTQSENSCTHFPGNLPNMLRDLRDAFSRVKTFFQMKDQLDNLL | 352 |
| m50 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSTFDIFINYIEAYMTMKIRN | |
| IL10mono_RBenh6- | SPGQGTQSENSCTHFPGNLPNMLRDLRDAFSRVKTFFQMKDQLDNLL | 353 |
| m51 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIHAYMTMKIRN | |
| IL10mono_RBenh6- | SPGQGTQSENSCTHFPGNLPNMLRDLRDAFSRVKTFFQMKDQLDNLL | 354 |
| m52 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIYAYMTMKIRN | |
| IL10mono_RBenh6- | SPGQGTQSENSCTHFPGNLPNMLRDLRDAFSRVKTFFIMKDQLDNLLL | 355 |
| m53 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFVMKDQLDNLL | 356 |
| m54 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVLTFFQMKDQLDNLL | 357 |
| m55 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVATFFQMKDQLDNLL | 358 |
| m56 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLLNLLL | 359 |
| m57 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLDNLL | 360 |
| m58 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFGIFINYIEAYMTMKIRN | |
| IL10mono_RBenh6- | SPGQGTQSENSCTHFPGNLPNMLRDLRDAFSRVKTFFQMKDQLDNLL | 361 |
| m59 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFTIFINYIEAYMTMKIRN | |
| IL10mono_RBenh6- | SPGQGTQSENSCTHFPGNLPNMLRDLRDAFSRVKTFFQMKDQLDNLL | 362 |
| m60 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSYFDIFINYIEAYMTMKIRN | |
| IL10mono_RBenh6- | SPGQGTQSENSCTHFPGNLPNMLRDLRDAFSRVKTFFQMKDQLDNLL | 363 |
| m61 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIIAYMTMKIRN | |
| IL10mono_RBenh6- | SPGQGTQSENSCTHFPGNLPNMLRDLRDAFSRVKTFFQMKDQLDNLL | 364 |
| m62 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIVAYMTMKIRN | |
| IL10mono_RBenh6- | SPGQGTQSENSCTHFPGNLPNMLRDLRDAFSRVKTFFLMKDQLDNLLL | 365 |
| m63 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVRTFFQMKDQLDNLL | 366 |
| m64 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVQTFFQMKDQLDNLL | 367 |
| m65 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLRNLL | 368 |
| m66 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLQNLL | 369 |
| m67 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLDNLL | 370 |
| m68 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFHIFINYIEAYMTMKIRN | |
| IL10mono_RBenh6- | SPGQGTQSENSCTHFPGNLPNMLRDLRDAFSRVKTFFQMKDQLDNLL | 371 |
| m69 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFYIFINYIEAYMTMKIRN | |
| IL10mono_RBenh6- | SPGQGTQSENSCTHFPGNLPNMLRDLRDAFSRVKTFFQMKDQLDNLL | 372 |
| m70 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSIFDIFINYIEAYMTMKIRN | |
| IL10mono_RBenh6- | SPGQGTQSENSCTHFPGNLPNMLRDLRDAFSRVKTFFQMKDQLDNLL | 373 |
| m71 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSVFDIFINYIEAYMTMKIRN | |
| IL10mono_RBenh6- | SPGQGTQSENSCTHFPGNLPNMLRDLRDAFSRVKTFFQMKDQLDNLL | 374 |
| m72 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYILAYMTMKIRN | |
| IL10mono_RBenh6- | SPGQGTQSENSCTHFPGNLPNMLRDLRDAFSRVKTFFRMKDQLDNLLL | 375 |
| m73 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFKMKDQLDNLLL | 376 |
| m74 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVNTFFQMKDQLDNLL | 377 |
| m75 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVFTFFQMKDQLDNLL | 378 |
| m76 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLNNLL | 379 |
| m77 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLFNLLL | 380 |
| m78 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLDNLL | 381 |
| m79 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFIIFINYIEAYMTMKIRN | |
| IL10mono_RBenh6- | SPGQGTQSENSCTHFPGNLPNMLRDLRDAFSRVKTFFQMKDQLDNLL | 382 |
| m80 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFVIFINYIEAYMTMKIRN | |
| IL10mono_RBenh6- | SPGQGTQSENSCTHFPGNLPNMLRDLRDAFSRVKTFFQMKDQLDNLL | 383 |
| m81 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSLFDIFINYIEAYMTMKIRN | |
| IL10mono_RBenh6- | SPGQGTQSENSCTHFPGNLPNMLRDLRDAFSRVKTFFQMKDQLDNLL | 384 |
| m82 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIRAYMTMKIRN | |
| IL10mono_RBenh6- | SPGQGTQSENSCTHFPGNLPNMLRDLRDAFSRVKTFFQMKDQLDNLL | 385 |
| m83 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIKAYMTMKIRN | |
| IL10mono_RBenh6- | SPGQGTQSENSCTHFPGNLPNMLRDLRDAFSRVKTFFNMKDQLDNLL | 389 |
| m84 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFFMKDQLDNLLL | 390 |
| m85 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVDTFFQMKDQLDNLL | 391 |
| m86 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVPTFFQMKDQLDNLL | 392 |
| m87 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLKNLL | 393 |
| m88 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLPNLL | 394 |
| m89 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLDNLL | 395 |
| m90 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFLIFINYIEAYMTMKIRN | |
| IL10mono_RBenh6- | SPGQGTQSENSCTHFPGNLPNMLRDLRDAFSRVKTFFQMKDQLDNLL | 396 |
| m91 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSRFDIFINYIEAYMTMKIRN | |
| IL10mono_RBenh6- | SPGQGTQSENSCTHFPGNLPNMLRDLRDAFSRVKTFFQMKDQLDNLL | 397 |
| m92 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSKFDIFINYIEAYMTMKIRN | |
| IL10mono_RBenh6- | SPGQGTQSENSCTHFPGNLPNMLRDLRDAFSRVKTFFQMKDQLDNLL | 398 |
| m93 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYINAYMTMKIRN | |
| IL10mono_RBenh6- | SPGQGTQSENSCTHFPGNLPNMLRDLRDAFSRVKTFFQMKDQLDNLL | 399 |
| m94 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIFAYMTMKIRN | |
| IL10mono_RBenh6- | SPGQGTQSENSCTHFPGNLPNMLRDLRDAFSRVKTFFDMKDQLDNLL | 400 |
| m95 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFPMKDQLDNLLL | 401 |
| m96 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVETFFQMKDQLDNLL | 402 |
| m97 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVSTFFQMKDQLDNLL | 403 |
| m98 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLENLLL | 404 |
| m99 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLSNLLL | 405 |
| m100 | KESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLVNLL | 406 |
| m101 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLDNLL | 407 |
| m102 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFIPYIEAYMTMKIRN | |
| IL10mono_RBenh6- | SPGQGTQSENSCTHFPGNLPNMLRDLRDAFSRVKTFFQMKDQLANLL | 408 |
| m103 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |
| IL10mono_RBenh6- | SPGQGTQSENSCTHFPGNLPNMLRDLRDAFSRVKTFFQMKDQLDNLL | 409 |
| m104 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFISYIEAYMTMKIRN | |
| IL10mono_RBenh6- | SPGQGTQSENSCTHFPGNLPNMLRDLRDAFSRVKTFFQMKDQLDNLL | 410 |
| m105 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFIDYIEAYMTMKIRN | |
| IL10mono_RBenh6- | SPGQGTQSENSCTHFPGNLPNMLRDLRDAFSRVKTFFQMKDQLDNLL | 411 |
| m106 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFITYIEAYMTMKIRN | |
| IL10mono_RBenh6- | SPGQGTQSENSCTHFPGNLPNMLRDLRDAFSRVKTFFQMKDQLDNLL | 412 |
| m107 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFIKYIEAYMTMKIRN | |
| IL10mono_RBenh6- | SPGQGTQSENSCTHFPGNLPNMLRDLRDAFSRVKTFFQMKDQLDNLL | 413 |
| m108 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFIVYIEAYMTMKIRN | |
| IL10mono_RBenh6- | SPGQGTQSENSCTHFPGNLPNMLRDLRDAFSRVKTFFQMKDQLDNLL | 414 |
| m109 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFIEYIEAYMTMKIRN | |
| IL10mono_RBenh6- | SPGQGTQSENSCTHFPGNLPNMLRDLRDAFSRVKTFFQMKDQLDNLL | 415 |
| m110 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYAA | |
| | MSEFDIFINYIAAYMTMKIRN | |
| IL10mono_RBenh6- | SPGQGTQSENSCTHFPGNLPNMLRDLRDAFSRVKTFFQMKDQLDNLL | 416 |
| m111 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFIGYIEAYMTMKIRN | |
| IL10mono_RBenh6- | SPGQGTQSENSCTHFPGNLPNMLRDLRDAFSRVKTFFQMKDQLDNLL | 417 |
| m112 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFAIFINYIAAYMTMKIRN | |
| IL10mono_RBenh6- | SPGQGTQSENSCTHFPGNLPNMLRDLRDAFSRVKTFFQMKDQLDNLL | 418 |
| m113 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFIIYIEAYMTMKIRN | |
| IL10mono_RBenh6- | SPGQGTQSENSCTHFPGNLPNMLRDLADAFSRVKTFFQMKDQLDNLL | 419 |
| m114 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIAAYMTMKIRN | |
| IL10mono_RBenh6- | SPGQGTQSENSCTHFPGNLPNMLRDLRDAFSRVKTFFQMKDQLDNLL | 420 |
| m115 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFIFYIEAYMTMKIRN | |
| IL10mono_RBenh6- | SPGQGTQSENSCTHFPGNLPNMLRDLADAFSRVKTFFAMKDQLDNLL | 421 |
| m116 | LKESLLEDFKGYLGCQALSEMIQFYLEEVMPQAENQDPDIKAHVVSLGE | |
| | NLKTLRLRLRRCHRFLPCENGGGSGGKSKAVEQVKNAFNKLQEKGIYKA | |
| | MSEFDIFINYIEAYMTMKIRN | |

TABLE 10

| IL-10 Mutein | IL-10RA binding affinity (Kd, nM) |
|---|---|
| IL10mono | 2.7 |
| IL10mono_RBenh6 | 2.8 |
| IL10mono_RBenh6-m17 | >2500 |
| IL10mono_RBenh6-m18 | NB |
| IL10mono_RBenh6-m19 | >2500 |
| IL10mono_RBenh6-m20 | NB |
| IL10mono_RBenh6-m21 | 1013.7 |
| IL10mono_RBenh6-m22 | 585.6 |
| IL10mono_RBenh6-m23 | 37.7 |
| IL10mono_RBenh6-m24 | 123.2 |
| IL10mono_RBenh6-m25 | 99.3 |
| IL10mono_RBenh6-m26 | 2.7 |
| IL10mono_RBenh6-m27 | 2.3 |
| IL10mono_RBenh6-m28 | 161.1 |
| IL10mono_RBenh6-m29 | NB |
| IL10mono_RBenh6-m30 | 37.3 |
| IL10mono_RBenh6-m31 | 38.5 |
| IL10mono_RBenh6-m32 | 21.9 |
| IL10mono_RBenh6-m33 | 99.4 |
| IL10mono_RBenh6-m34 | 114.2 |
| IL10mono_RBenh6-m35 | 34.2 |
| IL10mono_RBenh6-m36 | 2.0 |
| IL10mono_RBenh6-m37 | 3076.9 |
| IL10mono_RBenh6-m38 | 1460.1 |
| IL10mono_RBenh6-m39 | >2500 |
| IL10mono_RBenh6-m40 | ND |
| IL10mono_RBenh6-m41 | 52.8 |
| IL10mono_RBenh6-m42 | 147.7 |
| IL10mono_RBenh6-m43 | 9.5 |
| IL10mono_RBenh6-m44 | 325.6 |
| IL10mono_RBenh6-m45 | 25.4 |
| IL10mono_RBenh6-m46 | 2.2 |
| IL10mono_RBenh6-m47 | 783.0 |
| IL10mono_RBenh6-m48 | 433.1 |
| IL10mono_RBenh6-m49 | ND |
| IL10mono_RBenh6-m50 | NB |
| IL10mono_RBenh6-m51 | 107.1 |
| IL10mono_RBenh6-m52 | 236.0 |
| IL10mono_RBenh6-m53 | 345.2 |
| IL10mono_RBenh6-m54 | 269.7 |
| IL10mono_RBenh6-m55 | 66.5 |
| IL10mono_RBenh6-m56 | 57.3 |
| IL10mono_RBenh6-m57 | 1.9 |
| IL10mono_RBenh6-m58 | 538.1 |
| IL10mono_RBenh6-m59 | 1950.3 |
| IL10mono_RBenh6-m60 | ND |
| IL10mono_RBenh6-m61 | 130.9 |
| IL10mono_RBenh6-m62 | 42.9 |
| IL10mono_RBenh6-m63 | 431.7 |
| IL10mono_RBenh6-m64 | 4.1 |
| IL10mono_RBenh6-m65 | 14.2 |
| IL10mono_RBenh6-m66 | 12.2 |
| IL10mono_RBenh6-m67 | 3.1 |
| IL10mono_RBenh6-m68 | 2618.8 |
| IL10mono_RBenh6-m69 | 469.5 |
| IL10mono_RBenh6-m70 | NB |
| IL10mono_RBenh6-m71 | NB |
| IL10mono_RBenh6-m72 | 663.9 |
| IL10mono_RBenh6-m73 | 1870.0 |
| IL10mono_RBenh6-m74 | 3143.0 |
| IL10mono_RBenh6-m75 | 43.9 |
| IL10mono_RBenh6-m76 | 288.0 |
| IL10mono_RBenh6-m77 | 2.4 |
| IL10mono_RBenh6-m78 | 1.4 |
| IL10mono_RBenh6-m79 | >2500 |
| IL10mono_RBenh6-m80 | 3432.0 |
| IL10mono_RBenh6-m81 | NB |
| IL10mono_RBenh6-m82 | 472.1 |
| IL10mono_RBenh6-m83 | 288.9 |
| IL10mono_RBenh6-m84 | 34.0 |
| IL10mono_RBenh6-m85 | 232.2 |
| IL10mono_RBenh6-m86 | 555.4 |
| IL10mono_RBenh6-m87 | 2668.0 |
| IL10mono_RBenh6-m88 | 16.2 |
| IL10mono_RBenh6-m89 | 2.1 |
| IL10mono_RBenh6-m90 | 876.8 |
| IL10mono_RBenh6-m91 | NB |
| IL10mono_RBenh6-m92 | NB |
| IL10mono_RBenh6-m93 | 79.9 |
| IL10mono_RBenh6-m94 | 307.4 |
| IL10mono_RBenh6-m95 | 20.2 |
| IL10mono_RBenh6-m96 | NB |
| IL10mono_RBenh6-m97 | 626.6 |
| IL10mono_RBenh6-m98 | 62.4 |
| IL10mono_RBenh6-m99 | 3.1 |
| IL10mono_RBenh6-m100 | 2.4 |
| IL10mono_RBenh6-m101 | 2.6 |
| IL10mono_RBenh6-m102 | 1917.9 |
| IL10mono_RBenh6-m103 | 1.8 |
| IL10mono_RBenh6-m104 | 8.5 |
| IL10mono_RBenh6-m105 | 25.9 |
| IL10mono_RBenh6-m106 | 7.8 |
| IL10mono_RBenh6-m107 | 12.1 |
| IL10mono_RBenh6-m108 | 11.8 |
| IL10mono_RBenh6-m109 | 8.8 |
| IL10mono_RBenh6-m110 | >2500 |
| IL10mono_RBenh6-m111 | 11.0 |
| IL10mono_RBenh6-m112 | 3245.0 |
| IL10mono_RBenh6-m113 | 19.1 |
| IL10mono_RBenh6-m114 | 590.4 |
| IL10mono_RBenh6-m115 | 11.1 |
| IL10mono_RBenh6-m116 | 2536.0 |

### Example 7: Removal of a Glycosaminoglycan Binding Site on IL-10

IL-10 contains a positively charged patch that has been shown to bind glycosaminoglycans, with heparin as the strongest binder to IL-10 (Kunze et al. J Biol Chem, 2016). This property of IL-10 is speculated to help modulate the function of IL-10 but it may also limit therapeutic efficacy of IL-10 through reduced exposure. R107 in particular has been identified as the most important residue that interacts with gly cos ammnogly cans, and based on molecular modeling, no other residue is able to compensate for the loss of R107 (Gehrcke et al. J Mol Graph Model, 2015).

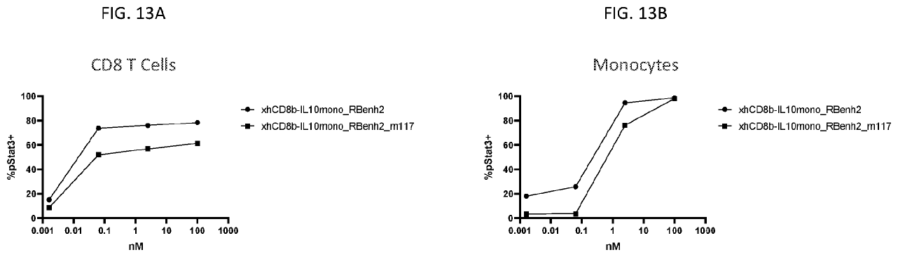

A mutation at R107A, designated m117, was introduced on the IL10mono_RBenh2 background as a fusion to xhCD8b antibody in format F, and tested in a STAT3 assay in human whole blood. The sequence of this construct is shown in Table 11. STAT3 activation data for CD8 T cells and monocytes are shown in FIGS. 13A and 13B, respectively. The STAT3 activation on both CD8 T cells and monocytes are comparable between IL10mono_RBenh2 and IL10mono_RBenh2_m117, indicating that the addition of the m117 mutation does not significantly affect activity.

The results of a polyreactivity ELISA assay that measures binding to a panel of irrelevant proteins are shown in Table 12. Bococizumab, which has been shown to be polyreactive, is also included as a positive control. When comparing IL10mono_RBenh2 to IL10mono_RBenh2_M117, the non-specific binding to many irrelevant targets is reduced. This is also the case for heparin Altogether, this indicates that the introduction of m117 reduces reactivity to non-specific irrelevant proteins which in turn can lead to improved exposure.

TABLE 11

| | | SEQ ID |
|---|---|---|
| IL10 Mutein | Sequence | NO |
| IL10mono_RBenh2_ | SPGQGTQSENSCTHFPGNLPNMLRDLRDAFSRVKTFFQMKDQLDNLLLKES | 422 |
| m117 | LLEDFKGYLGCQALSEMIQFYLEEVMPQAENQDPDIKAHVISLGENLKTLRLR | |
| | LRACHRFLPCENGGGSGGKSKAVEQVKNAFNKLQEKGIYKAMSEFDIFINYIE | |
| | AYMTMKIRN | |
| IL-10mono_RBenh2- | SPGQGTQSENSCTHFPGNLPNMLRDLRDAFSRVKTFFQMKDQLDNL | 423 |
| m10 m117 | LLKESLLEDFKGYLGCQALSEMIQFYLEEVMPQAENQDPDIKAHVISLG | |
| | ENLKTLRLRLRACHRFLPCENGGGSGGKSKAVEQVKNAFNKLQEKGIY | |
| | KAMSEFAIFINYIEAYMTMKIRN | |
| IL-10mono_RBenh7- | SPGQGTQSENSCTHFPGNLPNMLRDLRDAFSRVKTFFQMKDQLDNL | 424 |
| m10 m117 | LLKESLLEDFKGYLGCQALSEMIQFYLEEVMPQAENQDPDIKAHVLSL | |
| | GENLKTLRLRLRACHRFLPCENGGGSGGKSKAVEQVKNAFNKLQEKG | |
| | IYKAMSEFAIFINYIEAYMTMKIRN | |
| IL-10mono_RBenh2- | SPGQGTQSENSCTHFPGNLPNMLADLADAFSRVKTFFQMKDQLDNL | 425 |
| m15 m117 | LLKESLLEDFKGYLGCQALSEMIQFYLEEVMPQAENQDPDIKAHVISLG | |
| | ENLKTLRLRLRACHRFLPCENGGGSGGKSKAVEQVKNAFNKLQEKGIY | |
| | KAMSEFDIFINYIEAYMTMKIRN | |
| IL-10mono_RBenh2.1- | SPGQGTQSENSCTHFPGNLPNMLRDLRDAFSRVKTFFQMKDQLDNL | 426 |
| m10 m117 | LLKESLLEDFKGYLGCQALSEMIQFYLEEVMPQAENQDPDIKAHVISLG | |
| | ENLKTLRLRLRACHRFLPCENKGGGSGGSKAVEQVKNAFNKLQEKGIY | |
| | KAMSEFAIFINYIEAYMTMKIRN | |
| IL-10mono_RBenh7.1- | SPGQGTQSENSCTHFPGNLPNMLRDLRDAFSRVKTFFQMKDQLDNL | 427 |
| m10 m117 | LLKESLLEDFKGYLGCQALSEMIQFYLEEVMPQAENQDPDIKAHVLSL | |
| | GENLKTLRLRLRACHRFLPCENKGGGSGGSKAVEQVKNAFNKLQEKG | |
| | IYKAMSEFAIFINYIEAYMTMKIRN | |
| IL-10mono_RBenh2.1- | SPGQGTQSENSCTHFPGNLPNMLADLADAFSRVKTFFQMKDQLDNL | 428 |
| m15 m117 | LLKESLLEDFKGYLGCQALSEMIQFYLEEVMPQAENQDPDIKAHVISLG | |
| | ENLKTLRLRLRACHRFLPCENKGGGSGGSKAVEQVKNAFNKLQEKGIY | |
| | KAMSEFDIFINYIEAYMTMKIRN | |

TABLE 12

| | xhCD8b- | xhCD8b- | |
|---|---|---|---|
| Protein target | IL10mono_Rbenh2 | IL10mono_Rbenh2_m117 | Bococizumab |
| dsDNA salmon sperm | 3.99 | 3.81 | 17.85 |
| Human serum Albumin | 3.12 | 2.10 | 16.61 |
| KLH | 7.14 | 5.32 | 38.80 |
| LPS | 2.81 | 1.64 | 9.60 |
| Insulin | 3.86 | 2.36 | 21.39 |
| Heparin | 3.49 | 1.92 | 11.81 |

What is claimed is:

1. A mutant IL-10 polypeptide, wherein the mutant IL-10 polypeptide comprises an amino acid sequence having at least 80% amino acid sequence identity to the amino acid sequence of SEQ ID NO:1 with one or more amino acid substitutions relative to the amino acid sequence of SEQ ID NO:1, wherein the one or more amino acid substitutions are at position(s) selected from the group consisting of: N18, N21, M22, R24, D25, D28, S31, R32, D55, M68, I69, L73, E74, M77, P78, Q79, E81, N82, K88, A89, H90, N92, S93, G95, E96, N97, K99, T100, L101, L103, R104, R107, R110 and F111, numbering according to SEQ ID NO:1.

2. The mutant IL-10 polypeptide of claim 1, wherein the one or more amino acid substitutions are at position(s) selected from the group consisting of: N18, D28, N92, K99, and L103, numbering according to SEQ ID NO: 1.

3. The mutant IL-10 polypeptide of claim 2, wherein the one or more amino acid substitutions are selected from the group consisting of: N18F, N18L, N18Y, D28Q, D28R, N92F, N92H, N92I, N92K, N92L, N92R, N92S, N92T, N92V, N92Y, K99N, L103N, and L103Q, numbering according to SEQ ID NO: 1.

4. The mutant IL-10 polypeptide of any one of claims 1-3, wherein the mutant IL-10 polypeptide exhibits increased binding affinity to an IL-10RB polypeptide comprising the amino acid sequence of SEQ ID NO:3, as compared to binding affinity of the wild-type mature IL-10 polypeptide comprising the amino acid sequence of SEQ ID NO:1 to the IL-10RB polypeptide comprising the amino acid sequence of SEQ ID NO:3.

5. The mutant IL-10 polypeptide of claim 4, wherein said polypeptide exhibits increased binding affinity by 50% or more to an IL-10RB polypeptide comprising the amino acid sequence of SEQ ID NO:3, as compared to binding affinity of the wild-type mature IL-10 polypeptide comprising the amino acid sequence of SEQ ID NO:1 to the IL-10RB polypeptide comprising the amino acid sequence of SEQ ID NO:3.

6. The mutant IL-10 polypeptide of claim 4, wherein said polypeptide exhibits increased binding affinity by 150% or more to an IL-10RB polypeptide comprising the amino acid sequence of SEQ ID NO:3, as compared to binding affinity of the wild-type mature IL-10 polypeptide comprising the amino acid sequence of SEQ ID NO:1 to the IL-10RB polypeptide comprising the amino acid sequence of SEQ ID NO:3.

7. The mutant IL-10 polypeptide of any one of claims 1-6, wherein the mutant IL-10 polypeptide comprises one or more further amino acid substitutions relative to the amino acid sequence of SEQ ID NO: 1, wherein the one or more further amino acid substitutions are at position(s) selected from the group consisting of: P20, L23, R24, R27, D28, K34, T35, Q38, M39, D41, L43, D44, N45, L46, K49, I87, V91, L94, L98, K138, S141, E142, D144, N148, E151, and I158.

8. The mutant IL-10 polypeptide of claim 7, wherein the mutant IL-10 polypeptide comprises one or more further amino acid substitutions relative to the amino acid sequence of SEQ ID NO:1, wherein the one or more further amino acid substitutions are at position(s) selected from the group consisting of R24, R27, K34, Q38, D44, I87, K138, E142, D144, N148, and E151.

9. The mutant IL-10 polypeptide of claim 8, wherein the one or more further amino acid substitutions are selected from the group consisting of: R24A, R27A, K34A, K34D, K34E, K34S, K34P, K34G, K34T, K34H, K34L, K34N, K34F, K34R, K34Q, K34V, K34Y, Q38A, Q38D, Q38P, Q38G, Q38H, Q38I, Q38L, Q38R, Q38K, Q38N, Q38F, Q38T, Q38E, Q38S, Q38V, Q38Y, D44A, D44E, D44S, D44V, D44G, D44H, D44I, D44K, D44P, D44L, D44N, D44F, D44T, D44R, D44Q, I87A, K138A, E142A, E142G, E142N, E142L, E142F, E142I, E142V, E142K, E142R, E142P, E142Q, E142T, E142S, E142Y, D144A, D144E, D144G, D144H, D144R, D144I, D144K, D144N, D144Q, D144P, D144S, D144L, D144T, D144V, D144Y, N148G, N148P, N148S, N148D, N148T, N148K, N148V, N148I, N148E, N148F, E151A, E151G, E151H, E151I, E151N, E151F, E151L, E151V, E151R, E151K, E151P, E151Q, E151S, E151T, and E151Y.

10. The mutant IL-10 polypeptide of claim 9, wherein the one or more further amino acid substitutions are selected from the group consisting of: R24A, R27A, K34A, K34D, K34E, K34S, K34P, K34G, K34T, K34H, K34L, K34N, K34F, K34V, K34Y, Q38A, Q38D, Q38P, Q38G, Q38I, Q38L, Q38R, Q38K, Q38F, Q38T, Q38E, Q38S, Q38V, Q38Y, I87A, K138A, E142A, E142G, E142N, E142L, E142F, E142I, E142V, E142K, E142R, E142P, E142Q, E142T, E142S, E142Y, D144A, D144E, D144G, D144H, D144R, D144I, D144K, D144N, D144Q, D144P, D144S, D144L, D144T, D144V, D144Y, N148P, N148D, N148I, E151A, E151G, E151H, E151I, E151N, E151F, E151L, E151V, E151R, E151K, E151P, E151Q, E151S, E151T, and E151Y.

11. The mutant IL-10 polypeptide of any one of claims 1-10, wherein the mutant IL-10 polypeptide comprises an amino acid sequence selected from the group consisting of SEQ ID Nos: 87-89, 188-201, and 310-318.

12. A mutant IL-10 polypeptide, wherein the mutant IL-10 polypeptide comprises an amino acid sequence having at least 80% amino acid sequence identity to the amino acid sequence of SEQ ID NO:1 with one or more amino acid substitutions relative to the amino acid sequence of SEQ ID NO:1, wherein the one or more amino acid substitutions are at position(s) selected from the group consisting of: P20, L23, R24, R27, D28, K34, T35, Q38, M39, D41, L43, D44, N45, L46, K49, I87, V91, L94, L98, K138, S141, E142, D144, N148, E151, and I158, numbering according to SEQ ID NO:1.

13. The mutant IL-10 polypeptide of claim 12, wherein the one or more amino acid substitutions are at position(s) selected from the group consisting of: R24, R27, K34, Q38, D44, I87, K138, E142, D144, N148, and E151.

14. The mutant IL-10 polypeptide of claim 13, wherein the one or more amino acid substitutions are selected from the group consisting of: R24A, R27A, K34A, K34D, K34E, K34S, K34P, K34G, K34T, K34H, K34L, K34N, K34F, K34R, K34Q, K34V, K34Y, Q38A, Q38D, Q38P, Q38G, Q38H, Q38I, Q38L, Q38R, Q38K, Q38N, Q38F, Q38T, Q38E, Q38S, Q38V, Q38Y, D44A, D44E, D44S, D44V, D44G, D44H, D44I, D44K, D44P, D44L, D44N, D44F, D44T, D44R, D44Q, I87A, K138A, E142A, E142G, E142N, E142L, E142F, E142I, E142V, E142K, E142R, E142P, E142Q, E142T, E142S, E142Y, D144A, D144E, D144G, D144H, D144R, D144I, D144K, D144N, D144Q, D144P, D144S, D144L, D144T, D144V, D144Y, N148G, N148P, N148S, N148D, N148T, N148K, N148V, N148I, N148E, N148F, E151A, E151G, E151H, E151I, E151N, E151F, E151L, E151V, E151R, E151K, E151P, E151Q, E151S, E151T, and E151Y.

15. The mutant IL-10 polypeptide of claim 14, wherein the one or more amino acid substitutions are selected from the group consisting of: R24A, R27A, K34A, K34D, K34E, K34S, K34P, K34G, K34T, K34H, K34L, K34N, K34F, K34V, K34Y, Q38A, Q38D, Q38P, Q38G, Q38I, Q38L, Q38R, Q38K, Q38F, Q38T, Q38E, Q38S, Q38V, Q38Y, I87A, K138A, E142A, E142G, E142N, E142L, E142F, E142I, E142V, E142K, E142R, E142P, E142Q, E142T, E142S, E142Y, D144A, D144E, D144G, D144H, D144R, D144I, D144K, D144N, D144Q, D144P, D144S, D144L, D144T, D144V, D144Y, N148P, N148D, N148I, E151A, E151G, E151H, E151I, E151N, E151F, E151L, E151V, E151R, E151K, E151P, E151Q, E151S, E151T, and E151Y.

16. The mutant IL-10 polypeptide of any one of claims 7-15, wherein the mutant IL-10 polypeptide exhibits reduced binding affinity to an IL-10RA polypeptide comprising the amino acid sequence of SEQ ID NO:2, as compared to binding affinity of the wild-type mature IL-10 polypeptide comprising the amino acid sequence of SEQ ID NO:1 to the IL-10RA polypeptide comprising the amino acid sequence of SEQ ID NO:2.

17. The mutant IL-10 polypeptide of claim 16, wherein said polypeptide exhibits reduced binding affinity by 50% or more to an IL-10RA polypeptide comprising the amino acid sequence of SEQ ID NO:2, as compared to binding affinity of the wild-type mature IL-10 polypeptide comprising the amino acid sequence of SEQ ID NO:1 to the IL-10RA polypeptide comprising the amino acid sequence of SEQ ID NO:2.

18. The mutant IL-10 polypeptide of claim 16, wherein said polypeptide exhibits reduced binding affinity by 150% or more to an IL-10RA polypeptide comprising the amino acid sequence of SEQ ID NO:2, as compared to binding affinity of the wild-type mature IL-10 polypeptide comprising the amino acid sequence of SEQ ID NO:1 to the IL-10RA polypeptide comprising the amino acid sequence of SEQ ID NO:2.

19. The mutant IL-10 polypeptide of claim 16, wherein said polypeptide exhibits reduced binding affinity by two-fold or more to an IL-10RA polypeptide comprising the amino acid sequence of SEQ ID NO:2, as compared to binding affinity of the wild-type mature IL-10 polypeptide comprising the amino acid sequence of SEQ ID NO:1 to the IL-10RA polypeptide comprising the amino acid sequence of SEQ ID NO:2.

20. The mutant IL-10 polypeptide of claim 16, wherein said polypeptide exhibits reduced binding affinity by ten-fold or more to an IL-10RA polypeptide comprising the amino acid sequence of SEQ ID NO:2, as compared to binding affinity of the wild-type mature IL-10 polypeptide comprising the amino acid sequence of SEQ ID NO:1 to the IL-10RA polypeptide comprising the amino acid sequence of SEQ ID NO:2.

21. The mutant IL-10 polypeptide of any one of claims 1-20, wherein the mutant IL-10 polypeptide further comprises an amino acid substitution relative to the amino acid sequence of SEQ ID NO:1 at position R107.

22. The mutant IL-10 polypeptide of claim 21, wherein the mutant IL-10 polypeptide further comprises an R107A mutation, numbering according to SEQ ID NO:1.

23. The mutant IL-10 polypeptide of any one of claims 1-20, wherein the mutant IL-10 polypeptide comprises an amino acid sequence selected from the group consisting of SEQ ID Nos:422-428.

24. The mutant IL-10 polypeptide of any one of claims 1-23, wherein the mutant IL-10 polypeptide is a dimer.

25. The mutant IL-10 polypeptide of claim 24, wherein the mutant IL-10 polypeptide is a homodimer.

26. The mutant IL-10 polypeptide of claim 24, wherein the mutant IL-10 polypeptide is a heterodimer.

27. The mutant IL-10 polypeptide of any one of claims 1-23, wherein the mutant IL-10 polypeptide is a monomer.

28. The mutant IL-10 polypeptide of claim 27, wherein the mutant IL-10 monomer polypeptide comprises the amino acid sequence of SEQ ID NO:1 with a peptide insertion of between 1 and 15 amino acids immediately following residue C114, E15, N116, K117, S118, K119, or A120, numbering based on SEQ ID NO:1.

29. The mutant IL-10 polypeptide of claim 27 or claim 28, wherein the mutant IL-10 monomer polypeptide comprises an amino acid substitution at position N92, numbering based on SEQ ID NO:1.

30. The mutant IL-10 polypeptide of claim 29, wherein the mutant IL-10 monomer polypeptide comprises amino acid substitution N92I.

31. The mutant IL-10 polypeptide of claim 29, wherein the mutant IL-10 monomer polypeptide comprises amino acid substitution N92F, N92H, N92K, N92L, N92R, N92S, N92T, N92V, or N92Y.

32. The mutant IL-10 polypeptide of any one of claims 29-31, wherein the mutant IL-10 monomer polypeptide further comprises one or more of amino acid substitutions N18I, K99N and F111L, numbering based on SEQ ID NO:1.

33. The mutant IL-10 polypeptide of any one of claims 29-32, wherein the mutant IL-10 monomer polypeptide further comprises one or more amino acid substitutions at position(s) R24, R27, Q38, I87, K138, E142, D144, and/or E151, numbering based on SEQ ID NO:1.

34. The mutant IL-10 polypeptide of claim 33, wherein the mutant IL-10 monomer polypeptide comprises an amino acid sequence selected from the group consisting of SEQ ID Nos: 87-89, 188-201, 310-318, and 422-428.

35. A fusion protein comprising the mutant IL-10 polypeptide of any one of claims 1 to 34 and an antigen binding molecule that binds to an antigen on T cells.

36. The fusion protein of claim 35, wherein said fusion protein selectively stimulates T cells over monocytes.

37. The fusion protein of claim 35 or claim 36, wherein the antigen binding molecule binds to CD8.

38. The fusion protein of claim 37, wherein the antigen binding molecule binds to CD8ab, CD8a, or CD8aa.

39. The fusion protein of claim 37, wherein the antigen binding molecule binds to CD8b and/or CD8ab.

40. The fusion protein of claim 37, wherein the antigen binding molecule comprises a heavy chain variable (VH) domain and a light chain variable (VL) domain; and wherein:
(a) the VH domain comprises a CDR-H1 comprising the amino acid sequence of SEQ ID NO:110, a CDR-H2 comprising the amino acid sequence of SEQ ID NO:111, and a CDR-H3 comprising the amino acid sequence of SEQ ID NO:112; and wherein the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6;
(b) the VH domain comprises a CDR-H1 comprising the amino acid sequence of SEQ ID NO:13, a CDR-H2 comprising the amino acid sequence of SEQ ID NO:14, and a CDR-H3 comprising the amino acid sequence of SEQ ID NO:15; and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:18;
(c) the VH domain comprises a CDR-H1 comprising the amino acid sequence of SEQ ID NO:19, a CDR-H2 comprising the amino acid sequence of SEQ ID NO:20, and a CDR-H3 comprising the amino acid sequence of SEQ ID NO:21; and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24;
(d) the VH domain comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; and wherein the VL domain comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30;
(e) the VH domain comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; and wherein the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36;
(f) the VH domain comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39; and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42;
(g) the VH domain comprises a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45; and wherein the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48;
(h) the VH domain comprises a CDR-H1 comprising the amino acid sequence of SEQ ID NO:177, a CDR-H2 comprising the amino acid sequence of SEQ ID NO:178, and a CDR-H3 comprising the amino acid sequence of SEQ ID NO:179; and wherein the VL domain comprises a CDR-L1 comprising the amino acid sequence of SEQ ID NO:180, a CDR-L2 comprising the amino acid sequence of SEQ ID NO:181, and a CDR-L3 comprising the amino acid sequence of SEQ ID NO:182;
(i) the VH domain comprises a CDR-H1 comprising the amino acid sequence of X₁X₂AIS, wherein X₁ is S, K, G, N, R, D, T, or G, and wherein X₂ is Y, L, H, or F (SEQ ID NO:259), a CDR-H2 comprising the amino acid sequence of X₁X₂X₃PX₄X₅X₆X₇X₈X₉YX₁₀QKFX₁₁G, wherein X₁ is G or H, X₂ is I or F, X₃ is I, N, or M, X₄ is G, N, H, S, R, I, or A, X₅ is A, N, H, S, T, F, or Y, X₆ is A, D, or G, X₇ is T, E, K, V, Q, or A, X₈ is A or T, X₉ is N or K, X₁₀ is A or N, and X₁₁ is Q or T (SEQ ID NO:260), and a CDR-H3 comprising the amino acid sequence of X₁X₂X₃GX₄X₅LFX₆X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO:261); and wherein the VL domain comprises a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO:262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO:263), and a CDR-L3 comprising the amino acid sequence of QX₁X₂X₃X₄X₅PWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO:264);
(j) the VH domain comprises a CDR-H1 comprising the amino acid sequence of SEQ ID NO:225, a CDR-H2 comprising the amino acid sequence of SEQ ID NO:226, and a CDR-H3 comprising the amino acid sequence of SEQ ID NO:227; and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228;
(k) the VH domain comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233; and wherein the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236;
(l) the VH domain comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233; and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228;
(m) the VH domain comprises a CDR-H1 comprising the amino acid sequence of X₁YX₂MS, wherein X₁ is S, D, E, A, or Q and X₂ is A, G, or T (SEQ ID NO:268), a CDR-H2 comprising the amino acid sequence of DIX₁X₂X₃GX₄X₅TX₆YADSVKG, wherein X₁ is T, N, S, Q, E, H, R, or A, X₂ is Y, W, F, or H, X₃ is A, S, Q, E, or T, X₄ is G or E, X₅ is S or I, and X₆ is A or G (SEQ ID NO:269), and a CDR-H3 comprising the amino acid sequence of X₁X₂X₃YX₄WX₅X₆AX₇DX₈, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO:270); and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42);
(n) the VH domain comprises a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231; and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42); or
(o) the VH domain comprises a CDR-H1 comprising the amino acid sequence of SEQ ID NO:229, a CDR-H2 comprising the amino acid sequence of SEQ ID NO:237, and a CDR-H3 comprising the amino acid sequence of SEQ ID NO:231; and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42).

41. The fusion protein of claim 37, wherein the antigen binding molecule comprises a heavy chain variable (VH) domain and a light chain variable (VL) domain; and wherein:
(a) the VH domain comprises a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:15; and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:18;
(b) the VH domain comprises a CDR-H1 comprising the amino acid sequence of SEQ ID NO:53, a CDR-H2 comprising the amino acid sequence of SEQ ID NO:52, and a CDR-H3 comprising the amino acid sequence of SEQ ID NO:21; and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24;
(c) the VH domain comprises a CDR-H1 comprising the amino acid sequence of SEQ ID NO:49, a CDR-H2 comprising the amino acid sequence of SEQ ID NO:52, and a CDR-H3 comprising the amino acid sequence of SEQ ID NO:27; and wherein the VL domain comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30;
(d) the VH domain comprises a CDR-H1 comprising the amino acid sequence of SEQ ID NO:54, a CDR-H2 comprising the amino acid sequence of SEQ ID NO:52, and a CDR-H3 comprising the amino acid sequence of SEQ ID NO:33; and wherein the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36;
(e) the VH domain comprises a CDR-H1 comprising the amino acid sequence of SEQ ID NO:55, a CDR-H2 comprising the amino acid sequence of SEQ ID NO:56, and a CDR-H3 comprising the amino acid sequence of SEQ ID NO:39; and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42;
(f) the VH domain comprises a CDR-H1 comprising the amino acid sequence of SEQ ID NO:55, a CDR-H2 comprising the amino acid sequence of SEQ ID NO:57, and a CDR-H3 comprising the amino acid sequence of SEQ ID NO:45; and wherein the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48;
(g) the VH domain comprises a CDR-H1 comprising the amino acid sequence of SEQ ID NO:49, a CDR-H2 comprising the amino acid sequence of SEQ ID NO:50, and a CDR-H3 comprising the amino acid sequence of SEQ ID NO:3; and wherein the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6;
(h) the VH domain comprises a CDR-H1 comprising the amino acid sequence of SEQ ID NO:183, a CDR-H2 comprising the amino acid sequence of SEQ ID NO:184, and a CDR-H3 comprising the amino acid sequence of SEQ ID NO:179; and wherein the VL domain comprises a CDR-L1 comprising the amino acid sequence of SEQ ID NO:180, a CDR-L2 comprising the amino acid sequence of SEQ ID NO:181, and a CDR-L3 comprising the amino acid sequence of SEQ ID NO:182;
(i) the VH domain comprises a CDR-H1 comprising the amino acid sequence of GX₁X₂FX₃X₄X₅, wherein X₁ is G, Y, S, or A, X₂ is T, S, G, R, N, or H, X₃ is S, T, R, H, Y, G, or P, X₄ is S, K, G, N, R, D, T, or G, and X₅ is Y, L, H, or F (SEQ ID NO:265), a CDR-H2 comprising the amino acid sequence of X₁PX₂X₃X₄X₅, wherein X₁ is I, N, or M, X₂ is G, N, H, S, R, I, or A, X₃ is A, N, H, S, T, F, or Y, X₄ is A, D, or G, and X₅ is T, E, K, V, Q, or A (SEQ ID NO:266), and a CDR-H3 comprising the amino acid sequence of X₁X₂X₃GX₄X₅LFX₆X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO:267); and wherein the VL domain comprises a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO:262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO:263), and a CDR-L3 comprising the amino acid sequence of QX₁X₂X₃X₄X₅PWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO:264);
(j) the VH domain comprises a CDR-H1 comprising the amino acid sequence of SEQ ID NO:238, a CDR-H2 comprising the amino acid sequence of SEQ ID NO:239, and a CDR-H3 comprising the amino acid sequence of SEQ ID NO:233; and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228;
(k) the VH domain comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233; and wherein the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236;
(l) the VH domain comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233; and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228;
(m) the VH domain comprises a CDR-H1 comprising the amino acid sequence of GFTFX₁X₂Y, wherein X₁ is S, D, E, Q, S, or A and X₂ is S, D, E, A, or Q (SEQ ID NO:271), a CDR-H2 comprising the amino acid sequence of X₁X₂X₃GX₄X₅, wherein X₁ is T, N, S, Q, E, H, R or A, X₂ is Y, W, F, or H, X₃ is A, S, Q, E, or T, X₄ is G or E, and X₅ is S or I (SEQ ID NO:272), and a CDR-H3 comprising the amino acid sequence of X₁X₂X₃YX₄WX₅X₆AX₇DX₈, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO:273); and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42);
(n) the VH domain comprises a CDR-H1 comprising the amino acid sequence of SEQ ID NO:240, a CDR-H2 comprising the amino acid sequence of SEQ ID NO:241, and a CDR-H3 comprising the amino acid sequence of SEQ ID NO:242; and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42); or
(o) the VH domain comprises a CDR-H1 comprising the amino acid sequence of SEQ ID NO:240, a CDR-H2 comprising the amino acid sequence of SEQ ID NO:244, and a CDR-H3 comprising the amino acid sequence of SEQ ID NO:242; and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42).

42. The fusion protein of claim 40 or claim 41, wherein:
(a) the VH domain comprises an amino acid sequence that is at least 90%, at least 95%, at least 99%, or 100% identical to the sequence of SEQ ID NO:62, and wherein the VL domain comprises an amino acid sequence that is at least 90%, at least 95%, at least 99%, or 100% identical to the sequence of SEQ ID NO:63;
(b) the VH domain comprises an amino acid sequence that is at least 90%, at least 95%, at least 99%, or 100% identical to the sequence of SEQ ID NO:64, and wherein the VL domain comprises an amino acid sequence that is at least 90%, at least 95%, at least 99%, or 100% identical to the sequence of SEQ ID NO:65;
(c) the VH domain comprises an amino acid sequence that is at least 90%, at least 95%, at least 99%, or 100% identical to the sequence of SEQ ID NO:66, and wherein the VL domain comprises an amino acid sequence that is at least 90%, at least 95%, at least 99%, or 100% identical to the sequence of SEQ ID NO:67;
(d) the VH domain comprises an amino acid sequence that is at least 90%, at least 95%, at least 99%, or 100% identical to the sequence of SEQ ID NO:68, and wherein the VL domain comprises an amino acid sequence that is at least 90%, at least 95%, at least 99%, or 100% identical to the sequence of SEQ ID NO:69;
(e) the VH domain comprises an amino acid sequence that is at least 90%, at least 95%, at least 99%, or 100% identical to the sequence of SEQ ID NO:70, and wherein the VL domain comprises an amino acid sequence that is at least 90%, at least 95%, at least 99%, or 100% identical to the sequence of SEQ ID NO:71;
(f) the VH domain comprises an amino acid sequence that is at least 90%, at least 95%, at least 99%, or 100% identical to the sequence of SEQ ID NO:72, and wherein the VL domain comprises an amino acid sequence that is at least 90%, at least 95%, at least 99%, or 100% identical to the sequence of SEQ ID NO:73;
(g) the VH domain comprises an amino acid sequence that is at least 90%, at least 95%, at least 99%, or 100% identical to the sequence of SEQ ID NO:245; and wherein the VL domain comprises an amino acid sequence that is at least 90%, at least 95%, at least 99%, or 100% identical to the sequence of SEQ ID NO:246;
(h) the VH domain comprises an amino acid sequence that is at least 90%, at least 95%, at least 99%, or 100% identical to the sequence of SEQ ID NO:251, and wherein the VL domain comprises an amino acid sequence that is at least 90%, at least 95%, at least 99%, or 100% identical to the sequence of SEQ ID NO:252;
(i) the VH domain comprises an amino acid sequence that is at least 90%, at least 95%, at least 99%, or 100% identical to the sequence of SEQ ID NO:253; and wherein the VL domain comprises an amino acid sequence that is at least 90%, at least 95%, at least 99%, or 100% identical to the sequence of SEQ ID NO:254;
(j) the VH domain comprises an amino acid sequence that is at least 90%, at least 95%, at least 99%, or 100% identical to the sequence of SEQ ID NO:247; and wherein the VL domain comprises an amino acid sequence that is at least 90%, at least 95%, at least 99%, or 100% identical to the sequence of SEQ ID NO:248;
(k) the VH domain comprises an amino acid sequence that is at least 90%, at least 95%, at least 99%, or 100% identical to the sequence of SEQ ID NO:249, and wherein the VL domain comprises an amino acid sequence that is at least 90%, at least 95%, at least 99%, or 100% identical to the sequence of SEQ ID NO:250;
(l) the VH domain comprises an amino acid sequence that is at least 90%, at least 95%, at least 99%, or 100% identical to the sequence of SEQ ID NO:255; and wherein the VL domain comprises an amino acid sequence that is at least 90%, at least 95%, at least 99%, or 100% identical to the sequence of SEQ ID NO:256;
(m) the VH domain comprises an amino acid sequence that is at least 90%, at least 95%, at least 99%, or 100% identical to the sequence of SEQ ID NO:257; and wherein the VL domain comprises an amino acid sequence that is at least 90%, at least 95%, at least 99%, or 100% identical to the sequence of SEQ ID NO:258;
(n) the VH domain comprises an amino acid sequence that is at least 90%, at least 95%, at least 99%, or 100% identical to the sequence of SEQ ID NO:58; and wherein the VL domain comprises an amino acid sequence that is at least 90%, at least 95%, at least 99%, or 100% identical to the sequence of SEQ ID NO:59; or
(o) the VH domain comprises an amino acid sequence that is at least 90%, at least 95%, at least 99%, or 100% identical to the sequence of SEQ ID NO: 185; and wherein the VL domain comprises an amino acid sequence that is at least 90%, at least 95%, at least 99%, or 100% identical to the sequence of SEQ ID NO:186.

43. The fusion protein of claim 35 or claim 36, wherein the antigen binding molecule binds to CD4.

44. The fusion protein of claim 35 or claim 36, wherein the antigen binding molecule binds to PD-1.

45. The fusion protein of any one of claims 35-44, wherein the T cells are human T cells.

46. The fusion protein of any one of claims 35-45, wherein the fusion protein comprises a dimer of two mutant IL-10 polypeptides, and wherein one of the two mutant IL-10 polypeptides is fused to the antigen binding molecule.

47. The fusion protein of any one of claims 35-45, wherein the fusion protein comprises two polypeptides, each comprising an antigen binding site, and wherein one mutant IL-10 polypeptide is fused to each of the polypeptides.

48. The fusion protein of any one of claims 35-45, wherein the fusion protein comprises a mutant IL-10 monomer polypeptide, and wherein the mutant IL-10 monomer polypeptide is fused to the antigen binding molecule.

49. The fusion protein of any one of claims 35-48, wherein the mutant IL-10 polypeptide is fused to the antigen binding molecule directly or via linker.

50. The fusion protein of any one of claims 35-45 and 49, wherein the antigen binding molecule comprises two antibody heavy chain polypeptides comprising a structure according to formula [I], from N-terminus to C-terminus:
VH-CH1-hinge-CH2-CH3 [I]
and two antibody light chain polypeptides comprising a structure according to formula [II], from N-terminus to C-terminus:
VL-CL [II]
wherein VH is an antibody heavy chain variable (VH) domain, wherein CH1 is an antibody CH1 domain, wherein hinge is an antibody hinge domain, wherein CH2 is an antibody CH2 domain, wherein CH3 is an antibody CH3 domain, wherein VL is an antibody light chain variable (VL) domain, wherein CL is an antibody constant light chain domain, and wherein VH/VL forms an antigen binding site.

51. The fusion protein of claim 50, wherein the fusion protein comprises two mutant IL-10 polypeptides associated in a dimer; and wherein the N-terminus of one of the two mutant IL-10 polypeptides is fused to the C-terminus of one of the two CH3 domains directly or via linker.

52. The fusion protein of claim 50, wherein the fusion protein comprises two mutant IL-10 polypeptides associated in a dimer; and wherein the N-terminus of a first of the two mutant IL-10 polypeptides is fused to the C-terminus of a first of the two CH3 domains directly or via linker, and the N-terminus of the second of the two mutant IL-10 polypeptides is fused to the C-terminus of the second of the two CH3 domains directly or via linker.

53. The fusion protein of claim 50, wherein the fusion protein comprises one mutant IL-10 monomer polypeptide; and wherein the N-terminus of the mutant IL-10 monomer polypeptide is fused to the C-terminus of one of the two CH3 domains directly or via linker.

54. The fusion protein of any one of claims 35-45 and 49, wherein the antigen binding molecule comprises a first antibody heavy chain polypeptide comprising a structure according to formula [I], from N-terminus to C-terminus:
VH-CH1-hinge-CH2-CH3 [I],
an antibody light chain polypeptide comprising a structure according to formula [II], from N-terminus to C-terminus:
VL-CL [II],
and a second antibody heavy chain polypeptide comprising a structure according to formula [III], from N-terminus to C-terminus:
hinge-CH2-CH3 [III],
wherein VH is an antibody heavy chain variable (VH) domain, wherein CH1 is an antibody CH1 domain, wherein hinge is an antibody hinge domain, wherein CH2 is an antibody CH2 domain, wherein CH3 is an antibody CH3 domain, wherein VL is an antibody light chain variable (VL) domain, wherein CL is an antibody constant light chain domain, and wherein VH/VL forms an antigen binding site.

55. The fusion protein of claim 54, wherein the fusion protein comprises two mutant IL-10 polypeptides associated in a dimer; and wherein the N-terminus of one of the two mutant IL-10 polypeptides is fused, directly or via linker, to one of: the C-terminus of the CH3 domain of the second antibody heavy chain polypeptide or the C-terminus of the CH3 domain of the first antibody heavy chain polypeptide.

56. The fusion protein of claim 54, wherein the fusion protein comprises two mutant IL-10 polypeptides associated in a dimer; and wherein the N-terminus of a first of the two mutant IL-10 polypeptides is fused to the C-terminus of the CH3 domain of the first antibody heavy chain polypeptide directly or via linker, and the N-terminus of the second of the two mutant IL-10 polypeptides is fused to the C-terminus of the CH3 domain of the second antibody heavy chain polypeptide directly or via linker.

57. The fusion protein of claim 54, wherein the fusion protein comprises one mutant IL-10 monomer polypeptide; and wherein the N-terminus of the mutant IL-10 monomer polypeptide is fused, directly or via linker, to one of: the C-terminus of the CH3 domain of the second antibody heavy chain polypeptide or the C-terminus of the CH3 domain of the first antibody heavy chain polypeptide.

58. The fusion protein of any one of claims 50-57, wherein one or both of the antibody heavy chain polypeptides comprise(s) the following amino acid substitutions: L234A, L235A, and G237A, numbering according to EU index.

59. The fusion protein of any one of claims 50-58, wherein a first of the two Fc domains comprises amino acid substitutions Y349C and T366W, and a second of the two Fc domain comprises amino acid substitutions S354C, T366S, L368A and Y407V, numbering according to EU index.

60. The fusion protein of any one of claims 50-59, wherein the linker comprises the sequence (GGGS)xGn (SEQ ID NO:74), (GGGGS)xGn (SEQ ID NO:75), (GGGGGS)xGn (SEQ ID NO:76), S(GGGS)xGn (SEQ ID NO:386), S(GGGGS)xGn (SEQ ID NO:387), or S(GGGGGS)xGn (SEQ ID NO:388), wherein x=1, 2, 3, 4, 5, 6, 7, 8, 9, 10, 11, or 12, and wherein n=0, 1, 2 or 3.

61. The fusion protein of claim 60, wherein the linker comprises the sequence GGGGSGGGGSGGGGS (SEQ ID NO:79) or SGGGGSGGGGSGGGGS (SEQ ID NO:77).

62. The fusion protein of claim 1, wherein the fusion protein comprises four polypeptide chains, wherein:
the first polypeptide chain comprises the amino acid sequence of SEQ ID NO:113, the second polypeptide chain comprises the amino acid sequence of SEQ ID NO:114, the third polypeptide chain comprises the amino acid sequence of SEQ ID NO:115, and the fourth polypeptide chain comprises the amino acid sequence of SEQ ID NO:113;
the first polypeptide chain comprises the amino acid sequence of SEQ ID NO: 113, the second polypeptide chain comprises the amino acid sequence of SEQ ID NO: 114, the third polypeptide chain comprises the amino acid sequence of SEQ ID NO: 116, and the fourth polypeptide chain comprises the amino acid sequence of SEQ ID NO:113;
the first polypeptide chain comprises the amino acid sequence of SEQ ID NO: 117, the second polypeptide chain comprises the amino acid sequence of SEQ ID NO:118, the third polypeptide chain comprises the amino acid sequence of SEQ ID NO:119, and the fourth polypeptide chain comprises the amino acid sequence of SEQ ID NO: 117;
the first polypeptide chain comprises the amino acid sequence of SEQ ID NO: 117, the second polypeptide chain comprises the amino acid sequence of SEQ ID NO:118, the third polypeptide chain comprises the amino acid sequence of SEQ ID NO:120, and the fourth polypeptide chain comprises the amino acid sequence of SEQ ID NO: 117;
the first polypeptide chain comprises the amino acid sequence of SEQ ID NO:121, the second polypeptide chain comprises the amino acid sequence of SEQ ID NO:122, the third polypeptide chain comprises the amino acid sequence of SEQ ID NO:123, and the fourth polypeptide chain comprises the amino acid sequence of SEQ ID NO: 121;
the first polypeptide chain comprises the amino acid sequence of SEQ ID NO: 121, the second polypeptide chain comprises the amino acid sequence of SEQ ID NO:122, the third polypeptide chain comprises the amino acid sequence of SEQ ID NO:124, and the fourth polypeptide chain comprises the amino acid sequence of SEQ ID NO: 121;
the first polypeptide chain comprises the amino acid sequence of SEQ ID NO:125, the second polypeptide chain comprises the amino acid sequence of SEQ ID NO:126, the third polypeptide chain comprises the amino acid sequence of SEQ ID NO:127, and the fourth polypeptide chain comprises the amino acid sequence of SEQ ID NO: 125;
the first polypeptide chain comprises the amino acid sequence of SEQ ID NO: 125, the second polypeptide chain comprises the amino acid sequence of SEQ ID NO:126, the third polypeptide chain comprises the amino acid sequence of SEQ ID NO:128, and the fourth polypeptide chain comprises the amino acid sequence of SEQ ID NO: 125;
the first polypeptide chain comprises the amino acid sequence of SEQ ID NO:129, the second polypeptide chain comprises the amino acid sequence of SEQ ID NO:130, the third polypeptide chain comprises the amino acid sequence of SEQ ID NO:131, and the fourth polypeptide chain comprises the amino acid sequence of SEQ ID NO: 129;
the first polypeptide chain comprises the amino acid sequence of SEQ ID NO: 129, the second polypeptide chain comprises the amino acid sequence of SEQ ID NO:130, the third polypeptide chain comprises the amino acid sequence of SEQ ID NO:132, and the fourth polypeptide chain comprises the amino acid sequence of SEQ ID NO: 129;
the first polypeptide chain comprises the amino acid sequence of SEQ ID NO:133, the second polypeptide chain comprises the amino acid sequence of SEQ ID NO:134, the third polypeptide chain comprises the amino acid sequence of SEQ ID NO:135, and the fourth polypeptide chain comprises the amino acid sequence of SEQ ID NO: 133;
the first polypeptide chain comprises the amino acid sequence of SEQ ID NO: 133, the second polypeptide chain comprises the amino acid sequence of SEQ ID NO:134, the third polypeptide chain comprises the amino acid sequence of SEQ ID NO:136, and the fourth polypeptide chain comprises the amino acid sequence of SEQ ID NO: 133;
the first polypeptide chain comprises the amino acid sequence of SEQ ID NO:137, the second polypeptide chain comprises the amino acid sequence of SEQ ID NO:138, the third polypeptide chain comprises the amino acid sequence of SEQ ID NO:139, and the fourth polypeptide chain comprises the amino acid sequence of SEQ ID NO: 137;
the first polypeptide chain comprises the amino acid sequence of SEQ ID NO: 137, the second polypeptide chain comprises the amino acid sequence of SEQ ID NO:138, the third polypeptide chain comprises the amino acid sequence of SEQ ID NO:140, and the fourth polypeptide chain comprises the amino acid sequence of SEQ ID NO: 137;
the first polypeptide chain comprises the amino acid sequence of SEQ ID NO:141, the second polypeptide chain comprises the amino acid sequence of SEQ ID NO:142, the third polypeptide chain comprises the amino acid sequence of SEQ ID NO:143, and the fourth polypeptide chain comprises the amino acid sequence of SEQ ID NO: 141;
the first polypeptide chain comprises the amino acid sequence of SEQ ID NO: 141, the second polypeptide chain comprises the amino acid sequence of SEQ ID NO:142, the third polypeptide chain comprises the amino acid sequence of SEQ ID NO:144, and the fourth polypeptide chain comprises the amino acid sequence of SEQ ID NO: 141;
the first polypeptide chain comprises the amino acid sequence of SEQ ID NO:145, the second polypeptide chain comprises the amino acid sequence of SEQ ID NO:146, the third polypeptide chain comprises the amino acid sequence of SEQ ID NO:147, and the fourth polypeptide chain comprises the amino acid sequence of SEQ ID NO: 145;
the first polypeptide chain comprises the amino acid sequence of SEQ ID NO: 145, the second polypeptide chain comprises the amino acid sequence of SEQ ID NO:146, the third polypeptide chain comprises the amino acid sequence of SEQ ID NO:148, and the fourth polypeptide chain comprises the amino acid sequence of SEQ ID NO: 145;
the first polypeptide chain comprises the amino acid sequence of SEQ ID NO:149, the second polypeptide chain comprises the amino acid sequence of SEQ ID NO: 150, the third polypeptide chain comprises the amino acid sequence of SEQ ID NO:151, and the fourth polypeptide chain comprises the amino acid sequence of SEQ ID NO: 149;
the first polypeptide chain comprises the amino acid sequence of SEQ ID NO: 149, the second polypeptide chain comprises the amino acid sequence of SEQ ID NO:150, the third polypeptide chain comprises the amino acid sequence of SEQ ID NO:152, and the fourth polypeptide chain comprises the amino acid sequence of SEQ ID NO: 149;
the first polypeptide chain comprises the amino acid sequence of SEQ ID NO:153, the second polypeptide chain comprises the amino acid sequence of SEQ ID NO:154, the third polypeptide chain comprises the amino acid sequence of SEQ ID NO:155, and the fourth polypeptide chain comprises the amino acid sequence of SEQ ID NO: 153;
the first polypeptide chain comprises the amino acid sequence of SEQ ID NO: 153, the second polypeptide chain comprises the amino acid sequence of SEQ ID NO:154, the third polypeptide chain comprises the amino acid sequence of SEQ ID NO:156, and the fourth polypeptide chain comprises the amino acid sequence of SEQ ID NO: 153;
the first polypeptide chain comprises the amino acid sequence of SEQ ID NO:157, the second polypeptide chain comprises the amino acid sequence of SEQ ID NO:158, the third polypeptide chain comprises the amino acid sequence of SEQ ID NO:159, and the fourth polypeptide chain comprises the amino acid sequence of SEQ ID NO: 157; or
the first polypeptide chain comprises the amino acid sequence of SEQ ID NO: 157, the second polypeptide chain comprises the amino acid sequence of SEQ ID NO:158, the third polypeptide chain comprises the amino acid sequence of SEQ ID NO:160, and the fourth polypeptide chain comprises the amino acid sequence of SEQ ID NO: 157.

63. One or more isolated polynucleotides encoding the mutant IL-10 polypeptide or fusion protein of any one of claims 1-62.

64. One or more vectors comprising the one or more polynucleotides of claim 63.

65. The one or more vectors of claim 64, wherein the vector(s) are expression vector(s).

66. A host cell comprising the one or more polynucleotides of claim 62 or the one or more vectors of claim 64 or claim 65.

67. A method of producing a mutant IL-10 polypeptide or fusion protein, comprising culturing the host cell of claim 66 under conditions suitable for production of the polypeptide or fusion protein.

68. The method of claim 67, further comprising recovering the polypeptide or fusion protein from the host cell.

69. A pharmaceutical composition comprising the mutant IL-10 polypeptide or fusion protein according to any one of claims 1-62 and a pharmaceutically acceptable carrier.

70. The mutant IL-10 polypeptide or fusion protein according to any one of claims 1-62 for use as a medicament.

71. A method of treating cancer comprising administering to an individual with cancer an effective amount of the mutant IL-10 polypeptide or fusion protein according to any one of claims 1-62 or the composition of claim 69.

72. The method of claim 71, further comprising administering to the individual a T cell therapy, cancer vaccine, chemotherapeutic agent, IL-2 polypeptide, or immune checkpoint inhibitor (ICI).

73. The method of claim 72, wherein the ICI is an inhibitor of PD-1, PD-L1, or CTLA-4.

74. The method of claim 72, wherein the T cell therapy comprises a chimeric antigen receptor (CAR)-based T cell therapy, a tumor-infiltrating lymphocyte (TIL)-based therapy, or a therapy with T cells bearing a transduced TCR.

75. The mutant IL-10 polypeptide or fusion protein according to any one of claims 1-62 for use in a method of treating cancer, said method comprising administering to an individual with cancer an effective amount of the polypeptide or fusion protein.

76. A method of treating infection comprising administering to an individual in need thereof an effective amount of the mutant IL-10 polypeptide or fusion protein according to any one of claims 1-62 or the composition of claim 69.

77. The method of claim 76, wherein the infection is a viral infection.

78. Use of the mutant IL-10 polypeptide or fusion protein according to any one of claims 1-62 for the manufacture of a medicament for treating cancer or chronic infection.

79. A method of expanding T cells ex vivo comprising contacting one or more T cells ex vivo with an effective amount of the mutant IL-10 polypeptide or fusion protein according to any one of claims 1-62 or the composition of claim 69.

80. The method of claim 79, wherein the one or more T cells are tumor infiltrating lymphocytes (TILs).


SEQUENCE LISTING (428 sequences)

SEQ ID NO: 1 - protein, 160 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 557 aa, Artificial Sequence
  Synthetic Construct
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 3 - protein, 306 aa, Artificial Sequence
  Synthetic Construct
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 4 - protein, 11 aa, Artificial Sequence
  Synthetic Construct
GASENIYGALN

SEQ ID NO: 5 - protein, 7 aa, Artificial Sequence
  Synthetic Construct
GATNLAD

SEQ ID NO: 6 - protein, 9 aa, Artificial Sequence
  Synthetic Construct
QNILDTPWT

SEQ ID NO: 7 - protein, 5 aa, Artificial Sequence
  Synthetic Construct
KYAIS

SEQ ID NO: 8 - protein, 17 aa, Artificial Sequence
  Synthetic Construct
HFNPNNDETKYNQKFQG

SEQ ID NO: 9 - protein, 10 aa, Artificial Sequence
  Synthetic Construct
DGLGLRLFAD

SEQ ID NO: 10 - protein, 11 aa, Artificial Sequence
  Synthetic Construct
RASENIYGALN

SEQ ID NO: 11 - protein, 7 aa, Artificial Sequence
  Synthetic Construct
GATNLAD

SEQ ID NO: 12 - protein, 9 aa, Artificial Sequence
  Synthetic Construct
QNILDTPWT

SEQ ID NO: 13 - protein, 5 aa, Artificial Sequence
  Synthetic Construct
NFAIS

SEQ ID NO: 14 - protein, 17 aa, Artificial Sequence
  Synthetic Construct
GIIPGHAKANYAQKFQG

SEQ ID NO: 15 - protein, 10 aa, Artificial Sequence
  Synthetic Construct
DGLGIRLFAD

SEQ ID NO: 16 - protein, 11 aa, Artificial Sequence
  Synthetic Construct
RASQEIYGALN

SEQ ID NO: 17 - protein, 7 aa, Artificial Sequence
  Synthetic Construct
GATNLQS

SEQ ID NO: 18 - protein, 9 aa, Artificial Sequence
  Synthetic Construct
QDIYDAPWT

SEQ ID NO: 19 - protein, 5 aa, Artificial Sequence
  Synthetic Construct
KFAIS

SEQ ID NO: 20 - protein, 17 aa, Artificial Sequence
  Synthetic Construct
GIIPGHAKANYAQKFQG

SEQ ID NO: 21 - protein, 10 aa, Artificial Sequence
  Synthetic Construct
DGLGIRLFAD

SEQ ID NO: 22 - protein, 11 aa, Artificial Sequence
  Synthetic Construct
RASQEIYGALN

SEQ ID NO: 23 - protein, 7 aa, Artificial Sequence
  Synthetic Construct
GATNLQS

SEQ ID NO: 24 - protein, 9 aa, Artificial Sequence
  Synthetic Construct
QDIYDAPWT

SEQ ID NO: 25 - protein, 5 aa, Artificial Sequence
  Synthetic Construct
KYAIS

SEQ ID NO: 26 - protein, 17 aa, Artificial Sequence
  Synthetic Construct
GIIPGHAKANYAQKFQG

SEQ ID NO: 27 - protein, 10 aa, Artificial Sequence
  Synthetic Construct
DGLGIRLFAD

SEQ ID NO: 28 - protein, 11 aa, Artificial Sequence
  Synthetic Construct
RASQKIYGALN

SEQ ID NO: 29 - protein, 7 aa, Artificial Sequence
  Synthetic Construct
GATNLQS

SEQ ID NO: 30 - protein, 9 aa, Artificial Sequence
  Synthetic Construct
QNTYDTPWT

SEQ ID NO: 31 - protein, 5 aa, Artificial Sequence
  Synthetic Construct
GHAIS

SEQ ID NO: 32 - protein, 17 aa, Artificial Sequence
  Synthetic Construct
GIIPGHAKANYAQKFQG

SEQ ID NO: 33 - protein, 10 aa, Artificial Sequence
  Synthetic Construct
DGLGIRLFAD

SEQ ID NO: 34 - protein, 11 aa, Artificial Sequence
  Synthetic Construct
RASQKIYGALN

SEQ ID NO: 35 - protein, 7 aa, Artificial Sequence
  Synthetic Construct
GATNLQS

SEQ ID NO: 36 - protein, 9 aa, Artificial Sequence
  Synthetic Construct
QNTYDTPWT

SEQ ID NO: 37 - protein, 5 aa, Artificial Sequence
  Synthetic Construct
DYGMS

SEQ ID NO: 38 - protein, 17 aa, Artificial Sequence
  Synthetic Construct
DINWSGEITAYADSVKG

SEQ ID NO: 39 - protein, 12 aa, Artificial Sequence
  Synthetic Construct
SNSYRWDDALDI

SEQ ID NO: 40 - protein, 11 aa, Artificial Sequence
  Synthetic Construct
RASQSVSSNLA

SEQ ID NO: 41 - protein, 7 aa, Artificial Sequence
  Synthetic Construct
GASSRAT

SEQ ID NO: 42 - protein, 10 aa, Artificial Sequence
  Synthetic Construct
QQYGSSPPVT

SEQ ID NO: 43 - protein, 5 aa, Artificial Sequence
  Synthetic Construct
DYAMH

SEQ ID NO: 44 - protein, 17 aa, Artificial Sequence
  Synthetic Construct
VISYDGSNKYYADSVKG

SEQ ID NO: 45 - protein, 13 aa, Artificial Sequence
  Synthetic Construct
DRIGWYDYDAFDI

SEQ ID NO: 46 - protein, 11 aa, Artificial Sequence
  Synthetic Construct
RASHSVGSNLA

SEQ ID NO: 47 - protein, 7 aa, Artificial Sequence
  Synthetic Construct
DASNRAT

SEQ ID NO: 48 - protein, 9 aa, Artificial Sequence
  Synthetic Construct
QQRSNWPPT

SEQ ID NO: 49 - protein, 7 aa, Artificial Sequence
  Synthetic Construct
GYTFTKY

SEQ ID NO: 50 - protein, 6 aa, Artificial Sequence
  Synthetic Construct
NPNNDE

SEQ ID NO: 51 - protein, 7 aa, Artificial Sequence
  Synthetic Construct
GYRFHNF

SEQ ID NO: 52 - protein, 6 aa, Artificial Sequence
  Synthetic Construct
IPGHAK

SEQ ID NO: 53 - protein, 7 aa, Artificial Sequence
  Synthetic Construct
GSRFYKF

SEQ ID NO: 54 - protein, 7 aa, Artificial Sequence
  Synthetic Construct
GSGFRGH

SEQ ID NO: 55 - protein, 7 aa, Artificial Sequence
  Synthetic Construct
GFTFDDY

SEQ ID NO: 56 - protein, 6 aa, Artificial Sequence
  Synthetic Construct
NWSGEI

SEQ ID NO: 57 - protein, 6 aa, Artificial Sequence
  Synthetic Construct
SYDGSN

SEQ ID NO: 58 - protein, 119 aa, Artificial Sequence
  Synthetic Construct
QVHLQQSGPELVKPGASVKMSCKTSGYTFTKYTMHWVKQGHEESLEWIGHFNPNNDETKYNQKFTGKATLTVDKSSTTAYMELRSLTSDDSALYYCARDGLGLRLFADWGQGTLITVSA

SEQ ID NO: 59 - protein, 107 aa, Artificial Sequence
  Synthetic Construct
DIQMTQSPASLSASVGETVTITCGASENIYGALNWYQRKQGKSPQLLIFGATNLADGVSSRFSGSGSDRQYSLKISSLHPDDVATYYCQNILDTPWTFGGGTKLEIK

SEQ ID NO: 60 - protein, 119 aa, Artificial Sequence
  Synthetic Construct
QVQLVQSGAEVKKPGSSVKVSCKASGYTFTKYAISWVRQAPGQGLEWMGHFNPNNDETKYNQKFQGRVTITADESTSTAYMELSSLRSEDTAVYYCARDGLGLRLFADWGQGTLVTVSS

SEQ ID NO: 61 - protein, 107 aa, Artificial Sequence
  Synthetic Construct
DIQMTQSPSSLSASVGDRVTITCRASENIYGALNWYQQKPGKAPKLLIYGATNLADGVPSRFSGSGSGTDFTLTISSLQPEDFATYYCQNILDTPWTFGGGTKLEIK

SEQ ID NO: 62 - protein, 119 aa, Artificial Sequence
  Synthetic Construct
QVQLVQSGAEVKKPGSSVKVSCKASGYRFHNFAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 63 - protein, 107 aa, Artificial Sequence
  Synthetic Construct
DIQMTQSPSSLSASVGDRVTITCRASQEIYGALNWYQQKPGKAPKLLIYGATNLQSGVPSRFSGSGSGTDFTLTISSLQPEDFATYYCQDIYDAPWTFGGGTKVEIK

SEQ ID NO: 64 - protein, 119 aa, Artificial Sequence
  Synthetic Construct
QVQLVQSGAEVKKPGSSVKVSCKASGSRFYKFAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 65 - protein, 107 aa, Artificial Sequence; identical to SEQ ID NO: 63
  Synthetic Construct

SEQ ID NO: 66 - protein, 119 aa, Artificial Sequence
  Synthetic Construct
QVQLVQSGAEVKKPGSSVKVSCKASGYTFTKYAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 67 - protein, 107 aa, Artificial Sequence
  Synthetic Construct
DIQMTQSPSSLSASVGDRVTITCRASQKIYGALNWYQQKPGKAPKLLIYGATNLQSGVPSRFSGSGSGTDFTLTISSLQPEDFATYYCQNTYDTPWTFGGGTKVEIK

SEQ ID NO: 68 - protein, 119 aa, Artificial Sequence
  Synthetic Construct
QVQLVQSGAEVKKPGSSVKVSCKASGSGFRGHAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 69 - protein, 107 aa, Artificial Sequence; identical to SEQ ID NO: 67
  Synthetic Construct

SEQ ID NO: 70 - protein, 121 aa, Artificial Sequence
  Synthetic Construct
EVQLVESGGGAVRPGGSLRLSCAASGFTFDDYGMSWVRQAPGKGLEWVSDINWSGEITAYADSVKGRFTISRDNAKNSLYLQMNSLRAEDTAVYYCARSNSYRWDDALDIWGQGTMVTVSS

SEQ ID NO: 71 - protein, 108 aa, Artificial Sequence
  Synthetic Construct
EIVLTQSPATLSVSPGERATLSCRASQSVSSNLAWYQQKPGQAPRLLIYGASSRATGIPDRFSGSGSGTDFTLTISRLEPEDFAVYYCQQYGSSPPVTFGQGTKVEIK

SEQ ID NO: 72 - protein, 122 aa, Artificial Sequence
  Synthetic Construct
EVQLVESGGGLVQPGRSLRLSCAASGFTFDDYAMHWVRQAPGKGLEWVAVISYDGSNKYYADSVKGRFTISRDNSKNTLYLQMNSLRAEDTAVYYCAKDRIGWYDYDAFDIWGQGTMVTVSS

SEQ ID NO: 73 - protein, 107 aa, Artificial Sequence
  Synthetic Construct
EIVLTQSPATLSVTPGEGATLSCRASHSVGSNLAWYQQKPGQAPRLLIYDASNRATGIPARFSGSGSGTDFTLTISSLEPEDLAVYYCQQRSNWPPTFGQGTRLEIK

SEQ ID NO: 74 - protein, 51 aa, Artificial Sequence
  Synthetic Construct
  VARIANT (5)..(8), (9)..(12), (13)..(16), (17)..(20), (21)..(24),(25)..(28), (29)..(32), (33)..(36), (37)..(40), (41)..(44),(45)..(48): Can be present or absent
  VARIANT 49, 50, 51: Can be present or absent
GGGSGGGSGGGSGGGSGGGSGGGSGGGSGGGSGGGSGGGSGGGSGGGSGGG

SEQ ID NO: 75 - protein, 63 aa, Artificial Sequence
  Synthetic Construct
  VARIANT (6)..(10), (1)..(15), (16)..(20), (21)..(25),(26)..(30), (31)..(35), (36)..(40), (41)..(45), (46)..(50),(51)..(55), (56)..(60): Can be present or absent
  VARIANT 61, 62, 63: Can be present or absent
GGGGSGGGGSGGGGSGGGGSGGGGSGGGGSGGGGSGGGGSGGGGSGGGGSGGGGSGGGGSGGG

SEQ ID NO: 76 - protein, 75 aa, Artificial Sequence
  Synthetic Construct
  VARIANT (7)..(12), (13)..(18), (19)..(24), (25)..(30),(31)..(36), (37)..(42), (43)..(48), (49)..(54), (55)..(60),(61)..(66), (67)..(72): Can be present or absent
  VARIANT 73, 74, 75: Can be present or absent
GGGGGSGGGGGSGGGGGSGGGGGSGGGGGSGGGGGSGGGGGSGGGGGSGGGGGSGGGGGSGGGGGSGGGGGSGGG

SEQ ID NO: 77 - protein, 16 aa, Artificial Sequence
  Synthetic Construct
SGGGGSGGGGSGGGGS

SEQ ID NO: 78 - protein, 15 aa, Artificial Sequence
  Synthetic Construct
SGGGGSGGGGSGGGG

SEQ ID NO: 79 - protein, 15 aa, Artificial Sequence
  Synthetic Construct
GGGGSGGGGSGGGGS

SEQ ID NO: 80 - protein, 4 aa, Artificial Sequence
  Synthetic Construct
GGGG

SEQ ID NO: 81 - protein, 5 aa, Artificial Sequence
  Synthetic Construct
GGGSG

SEQ ID NO: 82 - protein, 5 aa, Artificial Sequence
  Synthetic Construct
GGGGG

SEQ ID NO: 83 - protein, 6 aa, Artificial Sequence
  Synthetic Construct
GGGGGG

SEQ ID NO: 84 - protein, 6 aa, Artificial Sequence
  Synthetic Construct
GGGSGG

SEQ ID NO: 85 - protein, 20 aa, Artificial Sequence
  Synthetic Construct
  VARIANT (10)..(15), (16)..(20): Can be present or absent
GGGGSGGGGSGGGGSGGGGS

SEQ ID NO: 86 - protein, 15 aa, Artificial Sequence
  Synthetic Construct
GGGGSGGGGSGGGGS

SEQ ID NO: 87 - protein, 166 aa, Artificial Sequence
  Synthetic Construct
SPGQGTQSENSCTHFPGNLPNMLRDLADAFSRVKTFFQMKDQLDNLLLKESLLEDFKGYLGCQALSEMIQFYLEEVMPQAENQDPDIKAHVISLGENLKTLRLRLRRCHRFLPCENGGGSGGKSKAVEQVKNAFNKLQEKGIYAAMSEFDIFINYIEAYMTMKIRN

SEQ ID NO: 88 - protein, 166 aa, Artificial Sequence
  Synthetic Construct
SPGQGTQSENSCTHFPGNLPNMLADLRDAFSRVKTFFQMKDQLDNLLLKESLLEDFKGYLGCQALSEMIQFYLEEVMPQAENQDPDIKAHVISLGENLKTLRLRLRRCHRFLPCENGGGSGGKSKAVEQVKNAFNKLQEKGIYAAMSEFDIFINYIEAYMTMKIRN

SEQ ID NO: 89 - protein, 166 aa, Artificial Sequence
  Synthetic Construct
SPGQGTQSENSCTHFPGNLPNMLADLADAFSRVKTFFQMKDQLDNLLLKESLLEDFKGYLGCQALSEMIQFYLEEVMPQAENQDPDIKAHVISLGENLKTLRLRLRRCHRFLPCENGGGSGGKSKAVEQVKNAFNKLQEKGIYKAMSEFDIFINYIEAYMTMKIRN

SEQ ID NO: 90 - protein, 166 aa, Artificial Sequence
  Synthetic Construct
SPGQGTQSENSCTHFPGNLPNMLADLADAFSRVKTFFQMKDQLDNLLLKESLLEDFKGYLGCQALSEMIQFYLEEVMPQAENQDPDIKAHVISLGENLKTLRLRLRRCHRFLPCENGGGSGGKSKAVEQVKNAFNKLQEKGIYAAMSEFDIFINYIEAYMTMKIRN

SEQ ID NO: 91 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCGGGSGGENKSKAVEQVKNAFNKLQEKGIYKAMSEFDIFINYIEAYMTMKIRN

SEQ ID NO: 92 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEGGGSGGNKSKAVEQVKNAFNKLQEKGIYKAMSEFDIFINYIEAYMTMKIRN

SEQ ID NO: 93 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKGGGSGGSKAVEQVKNAFNKLQEKGIYKAMSEFDIFINYIEAYMTMKIRN

SEQ ID NO: 94 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSGGGSGGKAVEQVKNAFNKLQEKGIYKAMSEFDIFINYIEAYMTMKIRN

SEQ ID NO: 95 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKGGGSGGAVEQVKNAFNKLQEKGIYKAMSEFDIFINYIEAYMTMKIRN

SEQ ID NO: 96 - protein, 161 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKGSKAVEQVKNAFNKLQEKGIYKAMSEFDIFINYIEAYMTMKIRN

SEQ ID NO: 97 - protein, 162 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKGGSKAVEQVKNAFNKLQEKGIYKAMSEFDIFINYIEAYMTMKIRN

SEQ ID NO: 98 - protein, 163 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKGGGSKAVEQVKNAFNKLQEKGIYKAMSEFDIFINYIEAYMTMKIRN

SEQ ID NO: 99 - protein, 164 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKGGGGSKAVEQVKNAFNKLQEKGIYKAMSEFDIFINYIEAYMTMKIRN

SEQ ID NO: 100 - protein, 165 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKGGGGGSKAVEQVKNAFNKLQEKGIYKAMSEFDIFINYIEAYMTMKIRN

SEQ ID NO: 101 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKGGGGGGSKAVEQVKNAFNKLQEKGIYKAMSEFDIFINYIEAYMTMKIRN

SEQ ID NO: 102 - protein, 117 aa, Artificial Sequence
  Synthetic Construct
EVQLVESGGGLVQPGRSLKLSCAASGFTFSNYYMAWVRQAPTKGLEWVAYINTGGGTTYYRDSVKGRFTISRDDAKSTLYLQMDSLRSEDTATYYCTTAIGYYFDYWGQGVMVTVSS

SEQ ID NO: 103 - protein, 107 aa, Artificial Sequence
  Synthetic Construct
DIQLTQSPASLSASLGETVSIECLASEDIYSYLAWYQQKPGKSPQVLIYAANRLQDGVPSRFSGSGSGTQYSLKISGMQPEDEGDYFCLQGSKFPYTFGAGTKLELK

SEQ ID NO: 104 - protein, 117 aa, Artificial Sequence
  Synthetic Construct
EVKLQESGPSLVQPSQTLSLTCSVSGFSLISDSVHWVRQPPGKGLEWMGGIWADGSTDYNSALKSRLSISRDTSKSQGFLKMNSLQTDDTAIYFCTSNRESYYFDYWGQGTMVTVSS

SEQ ID NO: 105 - protein, 106 aa, Artificial Sequence
  Synthetic Construct
DIQMTQSPASLSASLGDKVTITCQASQNIDKYIAWYQQKPGKAPRQLIHYTSTLVSGTPSRFSGSGSGRDYSFSISSVESEDIASYYCLQYDTLYTFGAGTKLELK

SEQ ID NO: 106 - protein, 117 aa, Artificial Sequence
  Synthetic Construct
EVKLQESGPSLVQPSQTLSLTCSVSGFSLISDSVHWVRQPPGKGLEWMGGIWADGSTDYNSALKSRLSISRDTSKSQGFLKMNSLQTDDTAIYFCTSARESYYFDYWGQGTMVTVSS

SEQ ID NO: 107 - protein, 106 aa, Artificial Sequence
  Synthetic Construct
DIQMTQSPASLSASLGDKVTITCQASQNIDKYIAWYQQKPGKAPRQLIHYTSTLVSGTPSRFSGSGSGRDYSFSISSVESEDIASYYCLQYATLYTFGAGTKLELK

SEQ ID NO: 108 - protein, 121 aa, Artificial Sequence
  Synthetic Construct
EVQLVESGGALVQPGRSLKLSCAASGLTFSDCYMAWVRQTPTKGLEWVSYISSDGGSTYYGDSVKGRFTISRDNAKSTLYLQMNSLRSEDMATYYCACATDLSSYWSFDFWGPGTMVTVSS

SEQ ID NO: 109 - protein, 107 aa, Artificial Sequence
  Synthetic Construct
DIQMTQSPSSLPVSLGERVTISCRASQGISNNLNWYQQKPDGTIKPLIYHTSNLQSGVPSRFSGSGSGTDYSLTISSLEPEDFAMYYCQQDATFPLTFGSGTKLEIK

SEQ ID NO: 110 - protein, 5 aa, Artificial Sequence
  Synthetic Construct
KYTMH

SEQ ID NO: 111 - protein, 17 aa, Artificial Sequence
  Synthetic Construct
HFNPNNDETKYNQKFTG

SEQ ID NO: 112 - protein, 10 aa, Artificial Sequence
  Synthetic Construct
DGLGLRLFAD

SEQ ID NO: 113 - protein, 214 aa, Artificial Sequence
  Synthetic Construct
DIQMTQSPSSLSASVGDRVTITCRASQSIYGALNWYQQKPGKAPKLLIYGASNLQSGVPSRFSGSGSGTDFTLTISSLQPEDFATYYCQSTYTAPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 630 aa, Artificial Sequence
  Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVISLGENLKTLRLRLRRCHRFLPCENGGGSGGKSKAVEQVKNAFNKLQEKGIYKAMSEFAIFINYIEAYMTMKIRN

SEQ ID NO: 115 - protein, 448 aa, Artificial Sequence
  Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 449 aa, Artificial Sequence
  Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 214 aa, Artificial Sequence; identical to SEQ ID NO: 113
  Synthetic Construct

SEQ ID NO: 118 - protein, 630 aa, Artificial Sequence
  Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVLSLGENLKTLRLRLRRCHRFLPCENGGGSGGKSKAVEQVKNAFNKLQEKGIYKAMSEFAIFINYIEAYMTMKIRN

SEQ ID NO: 119 - protein, 448 aa, Artificial Sequence; identical to SEQ ID NO: 115
  Synthetic Construct

SEQ ID NO: 120 - protein, 449 aa, Artificial Sequence; identical to SEQ ID NO: 116
  Synthetic Construct

SEQ ID NO: 121 - protein, 214 aa, Artificial Sequence; identical to SEQ ID NO: 113
  Synthetic Construct

SEQ ID NO: 122 - protein, 630 aa, Artificial Sequence
  Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSSPGQGTQSENSCTHFPGNLPNMLADLADAFSRVKTFFQMKDQLDNLLLKESLLEDFKGYLGCQALSEMIQFYLEEVMPQAENQDPDIKAHVISLGENLKTLRLRLRRCHRFLPCENGGGSGGKSKAVEQVKNAFNKLQEKGIYKAMSEFDIFINYIEAYMTMKIRN

SEQ ID NO: 123 - protein, 448 aa, Artificial Sequence; identical to SEQ ID NO: 115
  Synthetic Construct

SEQ ID NO: 124 - protein, 449 aa, Artificial Sequence; identical to SEQ ID NO: 116
  Synthetic Construct

SEQ ID NO: 125 - protein, 214 aa, Artificial Sequence; identical to SEQ ID NO: 113
  Synthetic Construct

SEQ ID NO: 126 - protein, 630 aa, Artificial Sequence
  Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVISLGENLKTLRLRLRACHRFLPCENGGGSGGKSKAVEQVKNAFNKLQEKGIYKAMSEFAIFINYIEAYMTMKIRN

SEQ ID NO: 127 - protein, 448 aa, Artificial Sequence; identical to SEQ ID NO: 115
  Synthetic Construct

SEQ ID NO: 128 - protein, 449 aa, Artificial Sequence; identical to SEQ ID NO: 116
  Synthetic Construct

SEQ ID NO: 129 - protein, 214 aa, Artificial Sequence; identical to SEQ ID NO: 113
  Synthetic Construct

SEQ ID NO: 130 - protein, 630 aa, Artificial Sequence
  Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVLSLGENLKTLRLRLRACHRFLPCENGGGSGGKSKAVEQVKNAFNKLQEKGIYKAMSEFAIFINYIEAYMTMKIRN

SEQ ID NO: 131 - protein, 448 aa, Artificial Sequence; identical to SEQ ID NO: 115
  Synthetic Construct

SEQ ID NO: 132 - protein, 449 aa, Artificial Sequence; identical to SEQ ID NO: 116
  Synthetic Construct

SEQ ID NO: 133 - protein, 214 aa, Artificial Sequence; identical to SEQ ID NO: 113
  Synthetic Construct

SEQ ID NO: 134 - protein, 630 aa, Artificial Sequence
  Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSSPGQGTQSENSCTHFPGNLPNMLADLADAFSRVKTFFQMKDQLDNLLLKESLLEDFKGYLGCQALSEMIQFYLEEVMPQAENQDPDIKAHVISLGENLKTLRLRLRACHRFLPCENGGGSGGKSKAVEQVKNAFNKLQEKGIYKAMSEFDIFINYIEAYMTMKIRN

SEQ ID NO: 135 - protein, 448 aa, Artificial Sequence; identical to SEQ ID NO: 115
  Synthetic Construct

SEQ ID NO: 136 - protein, 449 aa, Artificial Sequence; identical to SEQ ID NO: 116
  Synthetic Construct

SEQ ID NO: 137 - protein, 214 aa, Artificial Sequence; identical to SEQ ID NO: 113
  Synthetic Construct

SEQ ID NO: 138 - protein, 630 aa, Artificial Sequence
  Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVISLGENLKTLRLRLRRCHRFLPCENKGGGSGGSKAVEQVKNAFNKLQEKGIYKAMSEFAIFINYIEAYMTMKIRN

SEQ ID NO: 139 - protein, 448 aa, Artificial Sequence; identical to SEQ ID NO: 115
  Synthetic Construct

SEQ ID NO: 140 - protein, 449 aa, Artificial Sequence; identical to SEQ ID NO: 116
  Synthetic Construct

SEQ ID NO: 141 - protein, 214 aa, Artificial Sequence; identical to SEQ ID NO: 113
  Synthetic Construct

SEQ ID NO: 142 - protein, 630 aa, Artificial Sequence
  Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVLSLGENLKTLRLRLRRCHRFLPCENKGGGSGGSKAVEQVKNAFNKLQEKGIYKAMSEFAIFINYIEAYMTMKIRN

SEQ ID NO: 143 - protein, 448 aa, Artificial Sequence; identical to SEQ ID NO: 115
  Synthetic Construct

SEQ ID NO: 144 - protein, 449 aa, Artificial Sequence; identical to SEQ ID NO: 116
  Synthetic Construct

SEQ ID NO: 145 - protein, 214 aa, Artificial Sequence; identical to SEQ ID NO: 113
  Synthetic Construct

SEQ ID NO: 146 - protein, 630 aa, Artificial Sequence
  Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSSPGQGTQSENSCTHFPGNLPNMLADLADAFSRVKTFFQMKDQLDNLLLKESLLEDFKGYLGCQALSEMIQFYLEEVMPQAENQDPDIKAHVISLGENLKTLRLRLRRCHRFLPCENKGGGSGGSKAVEQVKNAFNKLQEKGIYKAMSEFDIFINYIEAYMTMKIRN

SEQ ID NO: 147 - protein, 448 aa, Artificial Sequence; identical to SEQ ID NO: 115
  Synthetic Construct

SEQ ID NO: 148 - protein, 449 aa, Artificial Sequence; identical to SEQ ID NO: 116
  Synthetic Construct

SEQ ID NO: 149 - protein, 214 aa, Artificial Sequence; identical to SEQ ID NO: 113
  Synthetic Construct

SEQ ID NO: 150 - protein, 630 aa, Artificial Sequence
  Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVISLGENLKTLRLRLRACHRFLPCENKGGGSGGSKAVEQVKNAFNKLQEKGIYKAMSEFAIFINYIEAYMTMKIRN

SEQ ID NO: 151 - protein, 448 aa, Artificial Sequence; identical to SEQ ID NO: 115
  Synthetic Construct

SEQ ID NO: 152 - protein, 449 aa, Artificial Sequence; identical to SEQ ID NO: 116
  Synthetic Construct

SEQ ID NO: 153 - protein, 214 aa, Artificial Sequence; identical to SEQ ID NO: 113
  Synthetic Construct

SEQ ID NO: 154 - protein, 630 aa, Artificial Sequence
  Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVLSLGENLKTLRLRLRACHRFLPCENKGGGSGGSKAVEQVKNAFNKLQEKGIYKAMSEFAIFINYIEAYMTMKIRN

SEQ ID NO: 155 - protein, 448 aa, Artificial Sequence; identical to SEQ ID NO: 115
  Synthetic Construct

SEQ ID NO: 156 - protein, 449 aa, Artificial Sequence; identical to SEQ ID NO: 116
  Synthetic Construct

SEQ ID NO: 157 - protein, 214 aa, Artificial Sequence; identical to SEQ ID NO: 113
  Synthetic Construct

SEQ ID NO: 158 - protein, 630 aa, Artificial Sequence
  Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSSPGQGTQSENSCTHFPGNLPNMLADLADAFSRVKTFFQMKDQLDNLLLKESLLEDFKGYLGCQALSEMIQFYLEEVMPQAENQDPDIKAHVISLGENLKTLRLRLRACHRFLPCENKGGGSGGSKAVEQVKNAFNKLQEKGIYKAMSEFDIFINYIEAYMTMKIRN

SEQ ID NO: 159 - protein, 448 aa, Artificial Sequence; identical to SEQ ID NO: 115
  Synthetic Construct

SEQ ID NO: 160 - protein, 449 aa, Artificial Sequence; identical to SEQ ID NO: 116
  Synthetic Construct

SEQ ID NO: 161 - protein, 166 aa, Artificial Sequence
  Synthetic Construct
SPGQGTQSENSCTHFPGYLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 162 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLNTLRLRLRRCHRFLPCENGGGSGGKSKAVEQVKNAFNKLQEKGIYKAMSEFDIFINYIEAYMTMKIRN

SEQ ID NO: 163 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVSSLGENLKTLRLRLRRCHRFLPCENGGGSGGKSKAVEQVKNAFNKLQEKGIYKAMSEFDIFINYIEAYMTMKIRN

SEQ ID NO: 164 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 165 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVLSLGENLKTLRLRLRRCHRFLPCENGGGSGGKSKAVEQVKNAFNKLQEKGIYKAMSEFDIFINYIEAYMTMKIRN

SEQ ID NO: 166 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVRSLGENLKTLRLRLRRCHRFLPCENGGGSGGKSKAVEQVKNAFNKLQEKGIYKAMSEFDIFINYIEAYMTMKIRN

SEQ ID NO: 167 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVFSLGENLKTLRLRLRRCHRFLPCENGGGSGGKSKAVEQVKNAFNKLQEKGIYKAMSEFDIFINYIEAYMTMKIRN

SEQ ID NO: 168 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVHSLGENLKTLRLRLRRCHRFLPCENGGGSGGKSKAVEQVKNAFNKLQEKGIYKAMSEFDIFINYIEAYMTMKIRN

SEQ ID NO: 169 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVYSLGENLKTLRLRLRRCHRFLPCENGGGSGGKSKAVEQVKNAFNKLQEKGIYKAMSEFDIFINYIEAYMTMKIRN

SEQ ID NO: 170 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVKSLGENLKTLRLRLRRCHRFLPCENGGGSGGKSKAVEQVKNAFNKLQEKGIYKAMSEFDIFINYIEAYMTMKIRN

SEQ ID NO: 171 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVTSLGENLKTLRLRLRRCHRFLPCENGGGSGGKSKAVEQVKNAFNKLQEKGIYKAMSEFDIFINYIEAYMTMKIRN

SEQ ID NO: 172 - protein, 166 aa, Artificial Sequence
  Synthetic Construct
SPGQGTQSENSCTHFPGNLPNMLRDLRDAFSKVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 173 - protein, 166 aa, Artificial Sequence
  Synthetic Construct
SPGQGTQSENSCTHFPGNLPNMLRDLRDAFSQVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 174 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAQNQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 175 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 176 - protein, 166 aa, Artificial Sequence
  Synthetic Construct
SPGQGTQSEQSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 177 - protein, 5 aa, Artificial Sequence
  Synthetic Construct
SYWMN

SEQ ID NO: 178 - protein, 17 aa, Artificial Sequence
  Synthetic Construct
QIYPGDGDTNYNGKFKG

SEQ ID NO: 179 - protein, 13 aa, Artificial Sequence
  Synthetic Construct
SGAAFSSYYAMDY

SEQ ID NO: 180 - protein, 11 aa, Artificial Sequence
  Synthetic Construct
RASENIYSNLA

SEQ ID NO: 181 - protein, 7 aa, Artificial Sequence
  Synthetic Construct
AATNLAD

SEQ ID NO: 182 - protein, 9 aa, Artificial Sequence
  Synthetic Construct
QHFWGTPWT

SEQ ID NO: 183 - protein, 7 aa, Artificial Sequence
  Synthetic Construct
GYAFSSY

SEQ ID NO: 184 - protein, 6 aa, Artificial Sequence
  Synthetic Construct
YPGDGD

SEQ ID NO: 185 - protein, 122 aa, Artificial Sequence
  Synthetic Construct
QVQLQQSGAELVRPGSSVKISCKASGYAFSSYWMNWVKQRPGQGLEWIGQIYPGDGDTNYNGKFKGKATLTADKSSSTAYMQLSSLTSEDSAVYFCARSGAAFSSYYAMDYWGQGTSVTVSS

SEQ ID NO: 186 - protein, 107 aa, Artificial Sequence
  Synthetic Construct
DIQMTQSPASLSVSVGETVTITCRASENIYSNLAWYQQKQGKSPQLLVYAATNLADGVPSRFSGSGSGTQYSLKINSLQSEDFGSYYCQHFWGTPWTFGGGTKLEIK

SEQ ID NO: 187 - protein, 166 aa, Artificial Sequence; identical to SEQ ID NO: 175
  Synthetic Construct

SEQ ID NO: 188 - protein, 166 aa, Artificial Sequence
  Synthetic Construct
SPGQGTQSENSCTHFPGILPNMLRDLRDAFSRVKTFFQMKDQLDNLLLKESLLEDFKGYLGCQALSEMIQFYLEEVMPQAENQDPDIKAHVISLGENLNTLRLRLRRCHRLLPCENGGGSGGKSKAVEQVKNAFNKLQEKGIYKAMSEFDIFINYIEAYMTMKIRN

SEQ ID NO: 189 - protein, 166 aa, Artificial Sequence
  Synthetic Construct
SPGQGTQSENSCTHFPGILPNMLRDLRDAFSRVKTFFAMKDQLDNLLLKESLLEDFKGYLGCQALSEMIQFYLEEVMPQAENQDPDIKAHVISLGENLNTLRLRLRRCHRLLPCENGGGSGGKSKAVEQVKNAFNKLQEKGIYKAMSEFDIFINYIEAYMTMKIRN

SEQ ID NO: 190 - protein, 166 aa, Artificial Sequence
  Synthetic Construct
SPGQGTQSENSCTHFPGILPNMLADLRDAFSRVKTFFQMKDQLDNLLLKESLLEDFKGYLGCQALSEMIQFYLEEVMPQAENQDPDIKAHVISLGENLNTLRLRLRRCHRLLPCENGGGSGGKSKAVEQVKNAFNKLQEKGIYKAMSEFDIFINYIEAYMTMKIRN

SEQ ID NO: 191 - protein, 166 aa, Artificial Sequence
  Synthetic Construct
SPGQGTQSENSCTHFPGILPNMLADLRDAFSRVKTFFAMKDQLDNLLLKESLLEDFKGYLGCQALSEMIQFYLEEVMPQAENQDPDIKAHVISLGENLNTLRLRLRRCHRLLPCENGGGSGGKSKAVEQVKNAFNKLQEKGIYKAMSEFDIFINYIEAYMTMKIRN

SEQ ID NO: 192 - protein, 166 aa, Artificial Sequence
  Synthetic Construct
SPGQGTQSENSCTHFPGILPNMLRDLRDAFSRVKTFFQMKDQLDNLLLKESLLEDFKGYLGCQALSEMIQFYLEEVMPQAENQDPDIKAHVISLGENLNTLRLRLRRCHRLLPCENGGGSGGKSKAVEQVKNAFNKLQEKGIYKAMSEFDIFINYIAAYMTMKIRN

SEQ ID NO: 193 - protein, 166 aa, Artificial Sequence
  Synthetic Construct
SPGQGTQSENSCTHFPGILPNMLADLRDAFSRVKTFFQMKDQLDNLLLKESLLEDFKGYLGCQALSEMIQFYLEEVMPQAENQDPDIKAHVISLGENLNTLRLRLRRCHRLLPCENGGGSGGKSKAVEQVKNAFNKLQEKGIYKAMSEFDIFINYIAAYMTMKIRN

SEQ ID NO: 194 - protein, 166 aa, Artificial Sequence
  Synthetic Construct
SPGQGTQSENSCTHFPGILPNMLRDLRDAFSRVKTFFQMKDQLDNLLLKESLLEDFKGYLGCQALSEMIQFYLEEVMPQAENQDPDAKAHVISLGENLNTLRLRLRRCHRLLPCENGGGSGGKSKAVEQVKNAFNKLQEKGIYKAMSEFDIFINYIEAYMTMKIRN

SEQ ID NO: 195 - protein, 166 aa, Artificial Sequence
  Synthetic Construct
SPGQGTQSENSCTHFPGILPNMLRDLRDAFSRVKTFFQMKDQLDNLLLKESLLEDFKGYLGCQALSEMIQFYLEEVMPQAENQDPDIKAHVISLGENLNTLRLRLRRCHRLLPCENGGGSGGKSKAVEQVKNAFNKLQEKGIYAAMSEFDIFINYIEAYMTMKIRN

SEQ ID NO: 196 - protein, 166 aa, Artificial Sequence
  Synthetic Construct
SPGQGTQSENSCTHFPGILPNMLRDLADAFSRVKTFFQMKDQLDNLLLKESLLEDFKGYLGCQALSEMIQFYLEEVMPQAENQDPDIKAHVISLGENLNTLRLRLRRCHRLLPCENGGGSGGKSKAVEQVKNAFNKLQEKGIYKAMSEFDIFINYIEAYMTMKIRN

SEQ ID NO: 197 - protein, 166 aa, Artificial Sequence
  Synthetic Construct
SPGQGTQSENSCTHFPGILPNMLRDLRDAFSRVKTFFQMKDQLDNLLLKESLLEDFKGYLGCQALSEMIQFYLEEVMPQAENQDPDIKAHVISLGENLNTLRLRLRRCHRLLPCENGGGSGGKSKAVEQVKNAFNKLQEKGIYKAMSAFDIFINYIEAYMTMKIRN

SEQ ID NO: 198 - protein, 166 aa, Artificial Sequence
  Synthetic Construct
SPGQGTQSENSCTHFPGILPNMLRDLRDAFSRVKTFFQMKDQLDNLLLKESLLEDFKGYLGCQALSEMIQFYLEEVMPQAENQDPDIKAHVISLGENLNTLRLRLRRCHRLLPCENGGGSGGKSKAVEQVKNAFNKLQEKGIYKAMSEFAIFINYIEAYMTMKIRN

SEQ ID NO: 199 - protein, 166 aa, Artificial Sequence
  Synthetic Construct
SPGQGTQSENSCTHFPGILPNMLRDLRDAFSRVKTFFAMKDQLDNLLLKESLLEDFKGYLGCQALSEMIQFYLEEVMPQAENQDPDIKAHVISLGENLNTLRLRLRRCHRLLPCENGGGSGGKSKAVEQVKNAFNKLQEKGIYKAMSAFDIFINYIEAYMTMKIRN

SEQ ID NO: 200 - protein, 166 aa, Artificial Sequence
  Synthetic Construct
SPGQGTQSENSCTHFPGILPNMLRDLRDAFSRVKTFFQMKDQLDNLLLKESLLEDFKGYLGCQALSEMIQFYLEEVMPQAENQDPDIKAHVISLGENLNTLRLRLRRCHRLLPCENGGGSGGKSKAVEQVKNAFNKLQEKGIYAAMSAFDIFINYIEAYMTMKIRN

SEQ ID NO: 201 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVISLGENLKTLRLRLRRCHRFLPCENGGGSGGKSKAVEQVKNAFNKLQEKGIYKAMSEFDIFINYIEAYMTMKIRN

SEQ ID NO: 202 - protein, 166 aa, Artificial Sequence
  Synthetic Construct
SPGQGTQSENSCTHFPGNLPNMLRDLRRAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 203 - protein, 166 aa, Artificial Sequence
  Synthetic Construct
SPGQGTQSENSCTHFPGNLPNMLRDLRQAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 204 - protein, 166 aa, Artificial Sequence
  Synthetic Construct
SPGQGTQSENSCTHFPGNLPNMLRDLRDAFSHVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 205 - protein, 166 aa, Artificial Sequence
  Synthetic Construct
SPGQGTQSENSCTHFPGNLPNMLRDLRDAFSYVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 206 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAYNQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 207 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQANNQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 208 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQASNQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 209 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAINQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 210 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAVNQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 211 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRHRLRRCHRFLPCENGGGSGGKSKAVEQVKNAFNKLQEKGIYKAMSEFDIFINYIEAYMTMKIRN

SEQ ID NO: 212 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRRRLRRCHRFLPCENGGGSGGKSKAVEQVKNAFNKLQEKGIYKAMSEFDIFINYIEAYMTMKIRN

SEQ ID NO: 213 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRKRLRRCHRFLPCENGGGSGGKSKAVEQVKNAFNKLQEKGIYKAMSEFDIFINYIEAYMTMKIRN

SEQ ID NO: 214 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRTRLRRCHRFLPCENGGGSGGKSKAVEQVKNAFNKLQEKGIYKAMSEFDIFINYIEAYMTMKIRN

SEQ ID NO: 215 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRSRLRRCHRFLPCENGGGSGGKSKAVEQVKNAFNKLQEKGIYKAMSEFDIFINYIEAYMTMKIRN

SEQ ID NO: 216 - protein, 166 aa, Artificial Sequence
  Synthetic Construct
SPGQGTQSENSCTHFPGNLPNMLRDLRKAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 217 - protein, 166 aa, Artificial Sequence
  Synthetic Construct
SPGQGTQSENSCTHFPGNLPNMLRDLRHAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 218 - protein, 166 aa, Artificial Sequence
  Synthetic Construct
SPGQGTQSENSCTHFPGELPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 219 - protein, 166 aa, Artificial Sequence
  Synthetic Construct
SPGQGTQSENSCTHFPGSLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 220 - protein, 166 aa, Artificial Sequence
  Synthetic Construct
SPGQGTQSENSCTHFPGTLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 221 - protein, 166 aa, Artificial Sequence
  Synthetic Construct
SPGQGTQSENSCTHFPGLLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 222 - protein, 166 aa, Artificial Sequence
  Synthetic Construct
SPGQGTQSENSCTHFPGVLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 223 - protein, 166 aa, Artificial Sequence
  Synthetic Construct
SPGQGTQSENSCTHFPGFLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 224 - protein, 166 aa, Artificial Sequence
  Synthetic Construct
SPGQGTQSENSCTEFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 225 - protein, 5 aa, Artificial Sequence
  Synthetic Construct
SYAIS

SEQ ID NO: 226 - protein, 17 aa, Artificial Sequence
  Synthetic Construct
GIIPGAATANYAQKFQG

SEQ ID NO: 227 - protein, 10 aa, Artificial Sequence
  Synthetic Construct
DAAGIRLFAD

SEQ ID NO: 228 - protein, 9 aa, Artificial Sequence
  Synthetic Construct
QSTYDAPWT

SEQ ID NO: 229 - protein, 5 aa, Artificial Sequence
  Synthetic Construct
SYAMS

SEQ ID NO: 230 - protein, 17 aa, Artificial Sequence
  Synthetic Construct
DITYAGGSTAYADSVKG

SEQ ID NO: 231 - protein, 12 aa, Artificial Sequence
  Synthetic Construct
SNAYAWDDALDI

SEQ ID NO: 232 - protein, 17 aa, Artificial Sequence
  Synthetic Construct
GIIPGYATANYAQKFQG

SEQ ID NO: 233 - protein, 10 aa, Artificial Sequence
  Synthetic Construct
DAAGIRLFAD

SEQ ID NO: 234 - protein, 11 aa, Artificial Sequence
  Synthetic Construct
RASQSIYGALN

SEQ ID NO: 235 - protein, 7 aa, Artificial Sequence
  Synthetic Construct
GASNLQS

SEQ ID NO: 236 - protein, 9 aa, Artificial Sequence
  Synthetic Construct
QSTYTAPWT

SEQ ID NO: 237 - protein, 17 aa, Artificial Sequence
  Synthetic Construct
DISYAGGSTAYADSVKG

SEQ ID NO: 238 - protein, 7 aa, Artificial Sequence
  Synthetic Construct
GGTFSSY

SEQ ID NO: 239 - protein, 6 aa, Artificial Sequence
  Synthetic Construct
IPGAAT

SEQ ID NO: 240 - protein, 7 aa, Artificial Sequence
  Synthetic Construct
GFTFSSY

SEQ ID NO: 241 - protein, 6 aa, Artificial Sequence
  Synthetic Construct
TYAGGS

SEQ ID NO: 242 - protein, 12 aa, Artificial Sequence
  Synthetic Construct
SNAYAWDDALDI

SEQ ID NO: 243 - protein, 6 aa, Artificial Sequence
  Synthetic Construct
IPGYAT

SEQ ID NO: 244 - protein, 6 aa, Artificial Sequence
  Synthetic Construct
SYAGGS

SEQ ID NO: 245 - protein, 119 aa, Artificial Sequence
  Synthetic Construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSS

SEQ ID NO: 246 - protein, 107 aa, Artificial Sequence
  Synthetic Construct
DIQMTQSPSSLSASVGDRVTITCRASQEIYGALNWYQQKPGKAPKLLIYGATNLQSGVPSRFSGSGSGTDFTLTISSLQPEDFATYYCQSTYDAPWTFGGGTKVEIK

SEQ ID NO: 247 - protein, 121 aa, Artificial Sequence
  Synthetic Construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSS

SEQ ID NO: 248 - protein, 108 aa, Artificial Sequence
  Synthetic Construct
EIVLTQSPGTLSLSPGERATLSCRASQSVSSNLAWYQQKPGQAPRLLIYGASSRATGIPDRFSGSGSGTDFTLTISRLEPEDFAVYYCQQYGSSPPVTFGQGTKVEIK

SEQ ID NO: 249 - protein, 121 aa, Artificial Sequence
  Synthetic Construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSS

SEQ ID NO: 250 - protein, 108 aa, Artificial Sequence; identical to SEQ ID NO: 248
  Synthetic Construct

SEQ ID NO: 251 - protein, 119 aa, Artificial Sequence
  Synthetic Construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSS

SEQ ID NO: 252 - protein, 107 aa, Artificial Sequence
  Synthetic Construct
DIQMTQSPSSLSASVGDRVTITCRASQSIYGALNWYQQKPGKAPKLLIYGASNLQSGVPSRFSGSGSGTDFTLTISSLQPEDFATYYCQSTYTAPWTFGGGTKVEIK

SEQ ID NO: 253 - protein, 119 aa, Artificial Sequence; identical to SEQ ID NO: 251
  Synthetic Construct

SEQ ID NO: 254 - protein, 107 aa, Artificial Sequence; identical to SEQ ID NO: 246
  Synthetic Construct

SEQ ID NO: 255 - protein, 121 aa, Artificial Sequence
  Synthetic Construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSS

SEQ ID NO: 256 - protein, 108 aa, Artificial Sequence; identical to SEQ ID NO: 248
  Synthetic Construct

SEQ ID NO: 257 - protein, 121 aa, Artificial Sequence
  Synthetic Construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSS

SEQ ID NO: 258 - protein, 108 aa, Artificial Sequence; identical to SEQ ID NO: 248
  Synthetic Construct

SEQ ID NO: 259 - protein, 5 aa, Artificial Sequence
  Synthetic Construct
  VARIANT 1: Xaa = S, K, G, N, R, D, T, or G
  VARIANT 2: Xaa = Y, L, H, or F
XXAIS

SEQ ID NO: 260 - protein, 17 aa, Artificial Sequence
  Synthetic Construct
  VARIANT 1: Xaa = G or H
  VARIANT 2: Xaa = I or F
  VARIANT 3: Xaa = I, N, or M
  VARIANT 5: Xaa = G, N, H, S, R, I, or A
  VARIANT 6: Xaa = A, N, H, S, T, F, or Y
  VARIANT 7: Xaa = A, D, or G
  VARIANT 8: Xaa = T, E, K, V, Q, or A
  VARIANT 9: Xaa = A or T
  VARIANT 10: Xaa = N or K
  VARIANT 12: Xaa = A or N
  VARIANT 16: Xaa = Q or T
XXXPXXXXXXYXQKFXG

SEQ ID NO: 261 - protein, 10 aa, Artificial Sequence
  Synthetic Construct
  VARIANT 1: Xaa = D or A
  VARIANT 2: Xaa = A, G, E, R, Y, K, N, Q, L, or F
  VARIANT 3: Xaa = A, L, P, or Y
  VARIANT 5: Xaa = I or L
  VARIANT 6: Xaa = R, A, Q, or S
  VARIANT 9: Xaa = A or D
  VARIANT 10: Xaa = D, E, A, or S
XXXGXXLFXX

SEQ ID NO: 262 - protein, 11 aa, Artificial Sequence
  Synthetic Construct
  VARIANT 1: Xaa = R or G
  VARIANT 2: Xaa = A or T
  VARIANT 4: Xaa = Q or E
  VARIANT 5: Xaa = E, N, T, S, A, K, D, G, R, or Q
  VARIANT 7: Xaa = Y or S
  VARIANT 9: Xaa = A or V
XXSXXIXGXLN

SEQ ID NO: 263 - protein, 7 aa, Artificial Sequence
  Synthetic Construct
  VARIANT 2: Xaa = A or S
  VARIANT 3: Xaa = T, S, E, Q, or D
  VARIANT 4: Xaa = N, R, A, E, or H
  VARIANT 6: Xaa = Q or A
  VARIANT 7: Xaa = S or D
GXXXLXX

SEQ ID NO: 264 - protein, 9 aa, Artificial Sequence
  Synthetic Construct
  VARIANT 2: Xaa = S, N, D, Q, A, or E
  VARIANT 3: Xaa = T, I, or S
  VARIANT 4: Xaa = Y, L, or F
  VARIANT 5: Xaa = D, G, T, E, Q, A, or Y
  VARIANT 6: Xaa = A, T, R, S, K, or Y
QXXXXXPWT

SEQ ID NO: 265 - protein, 7 aa, Artificial Sequence
  Synthetic Construct
  VARIANT 2: Xaa = G, Y, S, or A
  VARIANT 3: Xaa = T, S, G, R, N, or H
  VARIANT 5: Xaa = S, T, R, H, Y, G, or P
  VARIANT 6: Xaa = S, K, G, N, R, D, T, or G
  VARIANT 7: Xaa = Y, L, H, or F
GXXFXXX

SEQ ID NO: 266 - protein, 6 aa, Artificial Sequence
  Synthetic Construct
  VARIANT 1: Xaa = I, N, or M
  VARIANT 3: Xaa = G, N, H, S, R, I, or A
  VARIANT 4: Xaa = A, N, H, S, T, F, or Y
  VARIANT 5: Xaa = A, D, or G
  VARIANT 6: Xaa = T, E, K, V, Q, or A
XPXXXX

SEQ ID NO: 267 - protein, 10 aa, Artificial Sequence
  Synthetic Construct
  VARIANT 1: Xaa = D or A
  VARIANT 2: Xaa = A, G, E, R, Y, K, N, Q, L, or F
  VARIANT 3: Xaa = A, L, P, or Y
  VARIANT 5: Xaa = I or L
  VARIANT 6: Xaa = R, A, Q, or S
  VARIANT 9: Xaa = A or D
  VARIANT 10: Xaa = D, E, A, or S
XXXGXXLFXX

SEQ ID NO: 268 - protein, 5 aa, Artificial Sequence
  Synthetic Construct
  VARIANT 1: Xaa = S, D, E, A, or Q
  VARIANT 3: Xaa = A, G, or T
XYXMS

SEQ ID NO: 269 - protein, 17 aa, Artificial Sequence
  Synthetic Construct
  VARIANT 3: Xaa = T, N, S, Q, E, H, R, or A
  VARIANT 4: Xaa = Y, W, F, or H
  VARIANT 5: Xaa = A, S, Q, E, or T
  VARIANT 7: Xaa = G or E
  VARIANT 8: Xaa = S or I
  VARIANT 10: Xaa = A or G
DIXXXGXXTXYADSVKG

SEQ ID NO: 270 - protein, 12 aa, Artificial Sequence
  Synthetic Construct
  VARIANT 1: Xaa = S or A
  VARIANT 2: Xaa = N, H, A, D, L, Q, Y, or R
  VARIANT 3: Xaa = A, N, S, or G
  VARIANT 5: Xaa = A, V, R, E, or S
  VARIANT 7: Xaa = D or S
  VARIANT 8: Xaa = D, N, Q, E, S, T, or L
  VARIANT 10: Xaa = L, F, or M,
  VARIANT 12: Xaa = I, Y, or V
XXXYXWXXAXDX

SEQ ID NO: 271 - protein, 7 aa, Artificial Sequence
  Synthetic Construct
  VARIANT 5: Xaa = S, D, E, Q, S, or A
  VARIANT 6: Xaa = S, D, E, A, or Q
GFTFXXY

SEQ ID NO: 272 - protein, 6 aa, Artificial Sequence
  Synthetic Construct
  VARIANT 1: Xaa = T, N, S, Q, E, H, R or A
  VARIANT 2: Xaa = Y, W, F, or H
  VARIANT 3: Xaa = A, S, Q, E, or T,
  VARIANT 5: Xaa = G or E
  VARIANT 6: Xaa = S or I
XXXGXX

SEQ ID NO: 273 - protein, 12 aa, Artificial Sequence
  Synthetic Construct
  VARIANT 1: Xaa = S or A
  VARIANT 2: Xaa = N, H, A, D, L, Q, Y, or R
  VARIANT 3: Xaa = A, N, S, or G
  VARIANT 5: Xaa = A, V, R, E, or S
  VARIANT 7: Xaa = D or S
  VARIANT 8: Xaa = D, N, Q, E, S, T, or L
  VARIANT 10: Xaa = L, F, or M
  VARIANT 12: Xaa = I, Y, or V
XXXYXWXXAXDX

SEQ ID NO: 274 - protein, 166 aa, Artificial Sequence
  Synthetic Construct
SPGQGTQSENSCTQFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 275 - protein, 166 aa, Artificial Sequence
  Synthetic Construct
SPGQGTQSENSCTRFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 276 - protein, 166 aa, Artificial Sequence
  Synthetic Construct
SPGQGTQSENSCTKFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 277 - protein, 11 aa, Artificial Sequence
  Synthetic Construct
WGQGTLVTVSS

SEQ ID NO: 278 - protein, 25 aa, Artificial Sequence [sequence omitted]

SEQ ID NO: 279 - protein, 19 aa, Artificial Sequence
  Synthetic Construct
AISWVRQAPGQGLEWMGGI

SEQ ID NO: 280 - protein, 41 aa, Artificial Sequence
  Synthetic Construct
ANYAQKFQGRVTITADESTSTAYMELSSLRSEDTAVYYCAR

SEQ ID NO: 281 - protein, 166 aa, Artificial Sequence
  Synthetic Construct
SPGQGTQSENSCTSFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 282 - protein, 166 aa, Artificial Sequence
  Synthetic Construct
SPGQGTQSENSCTTFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 283 - protein, 166 aa, Artificial Sequence
  Synthetic Construct
SPGQGTQSENSCTIFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 284 - protein, 11 aa, Artificial Sequence
  Synthetic Construct
WGQGTMVTVSS

SEQ ID NO: 285 - protein, 11 aa, Artificial Sequence
  Synthetic Construct
WGQGTLVTVSS

SEQ ID NO: 286 - protein, 25 aa, Artificial Sequence [sequence omitted]

SEQ ID NO: 287 - protein, 19 aa, Artificial Sequence
  Synthetic Construct
AMSWVRQAPGKGLEWVSDI

SEQ ID NO: 288 - protein, 41 aa, Artificial Sequence
  Synthetic Construct
TAYADSVKGRFTISRDNAKNSLYLQMNSLRAEDTAVYYCAR

SEQ ID NO: 289 - protein, 23 aa, Artificial Sequence
  Synthetic Construct
DIQMTQSPSSLSASVGDRVTITC

SEQ ID NO: 290 - protein, 15 aa, Artificial Sequence
  Synthetic Construct
WYQQKPGKAPKLLIY

SEQ ID NO: 291 - protein, 32 aa, Artificial Sequence
  Synthetic Construct
GVPSRFSGSGSGTDFTLTISSLQPEDFATYYC

SEQ ID NO: 292 - protein, 10 aa, Artificial Sequence
  Synthetic Construct
FGGGTKVEIK

SEQ ID NO: 293 - protein, 23 aa, Artificial Sequence
  Synthetic Construct
EIVLTQSPGTLSLSPGERATLSC

SEQ ID NO: 294 - protein, 15 aa, Artificial Sequence
  Synthetic Construct
WYQQKPGQAPRLLIY

SEQ ID NO: 295 - protein, 32 aa, Artificial Sequence
  Synthetic Construct
GIPDRFSGSGSGTDFTLTISRLEPEDFAVYYC

SEQ ID NO: 296 - protein, 10 aa, Artificial Sequence
  Synthetic Construct
FGQGTKVEIK

SEQ ID NO: 297 - protein, 166 aa, Artificial Sequence
  Synthetic Construct
SPGQGTQSENSCTVFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 298 - protein, 166 aa, Artificial Sequence
  Synthetic Construct
SPGQGTQSENSCTYFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 299 - protein, 166 aa, Artificial Sequence
  Synthetic Construct
SPGQGTQSENSCTHFPGNLPEMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 300 - protein, 166 aa, Artificial Sequence
  Synthetic Construct
SPGQGTQSENSCTHFPGNLPRMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 301 - protein, 166 aa, Artificial Sequence
  Synthetic Construct
SPGQGTQSENSCTHFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 302 - protein, 166 aa, Artificial Sequence
  Synthetic Construct
SPGQGTQSENSCTHFPGNLPLMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 303 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMLQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 304 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMIQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 305 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMFQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 306 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMRQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 307 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKELRLRLRRCHRFLPCENGGGSGGKSKAVEQVKNAFNKLQEKGIYKAMSEFDIFINYIEAYMTMKIRN

SEQ ID NO: 308 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENGGGSGGKSKAVEQVKNAFNKLQEKGIYKAMSEFDIFINYIEAYMTMKIRN

SEQ ID NO: 309 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKILRLRLRRCHRFLPCENGGGSGGKSKAVEQVKNAFNKLQEKGIYKAMSEFDIFINYIEAYMTMKIRN

SEQ ID NO: 310 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVISLGENLKTLRLRLRRCHRFLPCENGGGSGGKSKAVEQVKNAFNKLQEKGIYKAMSEFAIFINYIEAYMTMKIRN

SEQ ID NO: 311 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVISLGENLKTLRLRLRRCHRFLPCENGGGSGGKSKAVEQVKNAFNKLQEKGIYAAMSAFDIFINYIEAYMTMKIRN

SEQ ID NO: 312 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVLSLGENLKTLRLRLRRCHRFLPCENGGGSGGKSKAVEQVKNAFNKLQEKGIYAAMSAFDIFINYIEAYMTMKIRN

SEQ ID NO: 313 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVLSLGENLKTLRLRLRRCHRFLPCENGGGSGGKSKAVEQVKNAFNKLQEKGIYKAMSEFAIFINYIEAYMTMKIRN

SEQ ID NO: 314 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFAIFINYIEAYMTMKIRN

SEQ ID NO: 315 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVRSLGENLKTLRLRLRRCHRFLPCENGGGSGGKSKAVEQVKNAFNKLQEKGIYKAMSEFAIFINYIEAYMTMKIRN

SEQ ID NO: 316 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVISLGENLKTLRLRLRRCHRFLPCENKGGGSGGSKAVEQVKNAFNKLQEKGIYKAMSEFAIFINYIEAYMTMKIRN

SEQ ID NO: 317 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVLSLGENLKTLRLRLRRCHRFLPCENKGGGSGGSKAVEQVKNAFNKLQEKGIYKAMSEFAIFINYIEAYMTMKIRN

SEQ ID NO: 318 - protein, 166 aa, Artificial Sequence
  Synthetic Construct
SPGQGTQSENSCTHFPGNLPNMLADLADAFSRVKTFFQMKDQLDNLLLKESLLEDFKGYLGCQALSEMIQFYLEEVMPQAENQDPDIKAHVISLGENLKTLRLRLRRCHRFLPCENKGGGSGGSKAVEQVKNAFNKLQEKGIYKAMSEFDIFINYIEAYMTMKIRN

SEQ ID NO: 319 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFRIFINYIEAYMTMKIRN

SEQ ID NO: 320 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFKIFINYIEAYMTMKIRN

SEQ ID NO: 321 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSNFDIFINYIEAYMTMKIRN

SEQ ID NO: 322 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSFFDIFINYIEAYMTMKIRN

SEQ ID NO: 323 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIPAYMTMKIRN

SEQ ID NO: 324 - protein, 166 aa, Artificial Sequence
  Synthetic Construct
SPGQGTQSENSCTHFPGNLPNMLRDLRDAFSRVKTFFEMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 325 - protein, 166 aa, Artificial Sequence
  Synthetic Construct
SPGQGTQSENSCTHFPGNLPNMLRDLRDAFSRVKTFFSMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 326 - protein, 166 aa, Artificial Sequence
  Synthetic Construct
SPGQGTQSENSCTHFPGNLPNMLRDLRDAFSRVGTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 327 - protein, 166 aa, Artificial Sequence
  Synthetic Construct
SPGQGTQSENSCTHFPGNLPNMLRDLRDAFSRVTTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 328 - protein, 166 aa, Artificial Sequence
  Synthetic Construct
SPGQGTQSENSCTHFPGNLPNMLRDLRDAFSRVKTFFQMKDQLGNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 329 - protein, 166 aa, Artificial Sequence
  Synthetic Construct
SPGQGTQSENSCTHFPGNLPNMLRDLRDAFSRVKTFFQMKDQLTNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 330 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFNIFINYIEAYMTMKIRN

SEQ ID NO: 331 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSPFDIFINYIEAYMTMKIRN

SEQ ID NO: 332 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIQAYMTMKIRN

SEQ ID NO: 333 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYISAYMTMKIRN

SEQ ID NO: 334 - protein, 166 aa, Artificial Sequence
  Synthetic Construct
SPGQGTQSENSCTHFPGNLPNMLRDLRDAFSRVKTFFGMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 335 - protein, 166 aa, Artificial Sequence
  Synthetic Construct
SPGQGTQSENSCTHFPGNLPNMLRDLRDAFSRVKTFFTMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 336 - protein, 166 aa, Artificial Sequence
  Synthetic Construct
SPGQGTQSENSCTHFPGNLPNMLRDLRDAFSRVHTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 337 - protein, 166 aa, Artificial Sequence
  Synthetic Construct
SPGQGTQSENSCTHFPGNLPNMLRDLRDAFSRVYTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 338 - protein, 166 aa, Artificial Sequence
  Synthetic Construct
SPGQGTQSENSCTHFPGNLPNMLRDLRDAFSRVKTFFQMKDQLHNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 339 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFQIFINYIEAYMTMKIRN

SEQ ID NO: 340 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFPIFINYIEAYMTMKIRN

SEQ ID NO: 341 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSQFDIFINYIEAYMTMKIRN

SEQ ID NO: 342 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSSFDIFINYIEAYMTMKIRN

SEQ ID NO: 343 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIGAYMTMKIRN

SEQ ID NO: 344 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYITAYMTMKIRN

SEQ ID NO: 345 - protein, 166 aa, Artificial Sequence
  Synthetic Construct
SPGQGTQSENSCTHFPGNLPNMLRDLRDAFSRVKTFFHMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 346 - protein, 166 aa, Artificial Sequence
  Synthetic Construct
SPGQGTQSENSCTHFPGNLPNMLRDLRDAFSRVKTFFYMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 347 - protein, 166 aa, Artificial Sequence
  Synthetic Construct
SPGQGTQSENSCTHFPGNLPNMLRDLRDAFSRVVTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 348 - protein, 166 aa, Artificial Sequence
  Synthetic Construct
SPGQGTQSENSCTHFPGNLPNMLRDLRDAFSRVKTFFQMKDQLINLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 349 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFEIFINYIEAYMTMKIRN

SEQ ID NO: 350 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFSIFINYIEAYMTMKIRN

SEQ ID NO: 351 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSGFDIFINYIEAYMTMKIRN

SEQ ID NO: 352 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSTFDIFINYIEAYMTMKIRN

SEQ ID NO: 353 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIHAYMTMKIRN

SEQ ID NO: 354 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIYAYMTMKIRN

SEQ ID NO: 355 - protein, 166 aa, Artificial Sequence
  Synthetic Construct
SPGQGTQSENSCTHFPGNLPNMLRDLRDAFSRVKTFFIMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 356 - protein, 166 aa, Artificial Sequence
  Synthetic Construct
SPGQGTQSENSCTHFPGNLPNMLRDLRDAFSRVKTFFVMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 357 - protein, 166 aa, Artificial Sequence
  Synthetic Construct
SPGQGTQSENSCTHFPGNLPNMLRDLRDAFSRVLTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 358 - protein, 166 aa, Artificial Sequence
  Synthetic Construct
SPGQGTQSENSCTHFPGNLPNMLRDLRDAFSRVATFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 359 - protein, 166 aa, Artificial Sequence
  Synthetic Construct
SPGQGTQSENSCTHFPGNLPNMLRDLRDAFSRVKTFFQMKDQLLNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 360 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFGIFINYIEAYMTMKIRN

SEQ ID NO: 361 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFTIFINYIEAYMTMKIRN

SEQ ID NO: 362 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSYFDIFINYIEAYMTMKIRN

SEQ ID NO: 363 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIIAYMTMKIRN

SEQ ID NO: 364 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIVAYMTMKIRN

SEQ ID NO: 365 - protein, 166 aa, Artificial Sequence
  Synthetic Construct
SPGQGTQSENSCTHFPGNLPNMLRDLRDAFSRVKTFFLMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 366 - protein, 166 aa, Artificial Sequence
  Synthetic Construct
SPGQGTQSENSCTHFPGNLPNMLRDLRDAFSRVRTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 367 - protein, 166 aa, Artificial Sequence
  Synthetic Construct
SPGQGTQSENSCTHFPGNLPNMLRDLRDAFSRVQTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 368 - protein, 166 aa, Artificial Sequence
  Synthetic Construct
SPGQGTQSENSCTHFPGNLPNMLRDLRDAFSRVKTFFQMKDQLRNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 369 - protein, 166 aa, Artificial Sequence
  Synthetic Construct
SPGQGTQSENSCTHFPGNLPNMLRDLRDAFSRVKTFFQMKDQLQNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 370 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFHIFINYIEAYMTMKIRN

SEQ ID NO: 371 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFYIFINYIEAYMTMKIRN

SEQ ID NO: 372 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSIFDIFINYIEAYMTMKIRN

SEQ ID NO: 373 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSVFDIFINYIEAYMTMKIRN

SEQ ID NO: 374 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYILAYMTMKIRN

SEQ ID NO: 375 - protein, 166 aa, Artificial Sequence
  Synthetic Construct
SPGQGTQSENSCTHFPGNLPNMLRDLRDAFSRVKTFFRMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 376 - protein, 166 aa, Artificial Sequence
  Synthetic Construct
SPGQGTQSENSCTHFPGNLPNMLRDLRDAFSRVKTFFKMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 377 - protein, 166 aa, Artificial Sequence
  Synthetic Construct
SPGQGTQSENSCTHFPGNLPNMLRDLRDAFSRVNTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 378 - protein, 166 aa, Artificial Sequence
  Synthetic Construct
SPGQGTQSENSCTHFPGNLPNMLRDLRDAFSRVFTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 379 - protein, 166 aa, Artificial Sequence
  Synthetic Construct
SPGQGTQSENSCTHFPGNLPNMLRDLRDAFSRVKTFFQMKDQLNNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 380 - protein, 166 aa, Artificial Sequence
  Synthetic Construct
SPGQGTQSENSCTHFPGNLPNMLRDLRDAFSRVKTFFQMKDQLFNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 381 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFIIFINYIEAYMTMKIRN

SEQ ID NO: 382 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFVIFINYIEAYMTMKIRN

SEQ ID NO: 383 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSLFDIFINYIEAYMTMKIRN

SEQ ID NO: 384 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIRAYMTMKIRN

SEQ ID NO: 385 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIKAYMTMKIRN

SEQ ID NO: 386 - protein, 52 aa, Artificial Sequence
  Synthetic Construct
  VARIANT (6)..(9), (10)..(13), (14)..(17), (18)..(21),(22)..(25), (26)..(29), (30)..(33), (34)..(37), (38)..(41),(42)..(45), (46)..(49): Can be present or absent
  VARIANT 50, 51, 52: Can be present or absent
SGGGSGGGSGGGSGGGSGGGSGGGSGGGSGGGSGGGSGGGSGGGSGGGSGGG

SEQ ID NO: 387 - protein, 64 aa, Artificial Sequence
  Synthetic Construct
  VARIANT (7)..(11), (12)..(16), (17)..(21), (22)..(26),(27)..(31), (32)..(36), (37)..(41), (42)..(46), (47)..(51),(52)..(56), (57)..(61): Can be present or absent
  VARIANT 62, 63, 64: Can be present or absent
SGGGGSGGGGSGGGGSGGGGSGGGGSGGGGSGGGGSGGGGSGGGGSGGGGSGGGGSGGGGSGGG

SEQ ID NO: 388 - protein, 76 aa, Artificial Sequence
  Synthetic Construct
  VARIANT (8)..(13), (14)..(19), (20)..(25), (26)..(31),(32)..(37), (38)..(43), (44)..(49), (50)..(55), (56)..(61),(62)..(67), (68)..(73): Can be present or absent
  VARIANT 74, 75, 76: Can be present or absent
SGGGGGSGGGGGSGGGGGSGGGGGSGGGGGSGGGGGSGGGGGSGGGGGSGGGGGSGGGGGSGGGGGSGGGGGSGGG

SEQ ID NO: 389 - protein, 166 aa, Artificial Sequence
  Synthetic Construct
SPGQGTQSENSCTHFPGNLPNMLRDLRDAFSRVKTFFNMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 390 - protein, 166 aa, Artificial Sequence
  Synthetic Construct
SPGQGTQSENSCTHFPGNLPNMLRDLRDAFSRVKTFFFMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 391 - protein, 166 aa, Artificial Sequence
  Synthetic Construct
SPGQGTQSENSCTHFPGNLPNMLRDLRDAFSRVDTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 392 - protein, 166 aa, Artificial Sequence
  Synthetic Construct
SPGQGTQSENSCTHFPGNLPNMLRDLRDAFSRVPTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 393 - protein, 166 aa, Artificial Sequence
  Synthetic Construct
SPGQGTQSENSCTHFPGNLPNMLRDLRDAFSRVKTFFQMKDQLKNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 394 - protein, 166 aa, Artificial Sequence
  Synthetic Construct
SPGQGTQSENSCTHFPGNLPNMLRDLRDAFSRVKTFFQMKDQLPNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 395 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFLIFINYIEAYMTMKIRN

SEQ ID NO: 396 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSRFDIFINYIEAYMTMKIRN

SEQ ID NO: 397 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSKFDIFINYIEAYMTMKIRN

SEQ ID NO: 398 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYINAYMTMKIRN

SEQ ID NO: 399 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIFAYMTMKIRN

SEQ ID NO: 400 - protein, 166 aa, Artificial Sequence
  Synthetic Construct
SPGQGTQSENSCTHFPGNLPNMLRDLRDAFSRVKTFFDMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 401 - protein, 166 aa, Artificial Sequence
  Synthetic Construct
SPGQGTQSENSCTHFPGNLPNMLRDLRDAFSRVKTFFPMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 402 - protein, 166 aa, Artificial Sequence
  Synthetic Construct
SPGQGTQSENSCTHFPGNLPNMLRDLRDAFSRVETFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 403 - protein, 166 aa, Artificial Sequence
  Synthetic Construct
SPGQGTQSENSCTHFPGNLPNMLRDLRDAFSRVSTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 404 - protein, 166 aa, Artificial Sequence
  Synthetic Construct
SPGQGTQSENSCTHFPGNLPNMLRDLRDAFSRVKTFFQMKDQLENLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 405 - protein, 166 aa, Artificial Sequence
  Synthetic Construct
SPGQGTQSENSCTHFPGNLPNMLRDLRDAFSRVKTFFQMKDQLSNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 406 - protein, 166 aa, Artificial Sequence
  Synthetic Construct
SPGQGTQSENSCTHFPGNLPNMLRDLRDAFSRVKTFFQMKDQLVNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 407 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFIPYIEAYMTMKIRN

SEQ ID NO: 408 - protein, 166 aa, Artificial Sequence
  Synthetic Construct
SPGQGTQSENSCTHFPGNLPNMLRDLRDAFSRVKTFFQMKDQLANLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 409 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFISYIEAYMTMKIRN

SEQ ID NO: 410 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFIDYIEAYMTMKIRN

SEQ ID NO: 411 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFITYIEAYMTMKIRN

SEQ ID NO: 412 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFIKYIEAYMTMKIRN

SEQ ID NO: 413 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFIVYIEAYMTMKIRN

SEQ ID NO: 414 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFIEYIEAYMTMKIRN

SEQ ID NO: 415 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYAAMSEFDIFINYIAAYMTMKIRN

SEQ ID NO: 416 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFIGYIEAYMTMKIRN

SEQ ID NO: 417 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFAIFINYIAAYMTMKIRN

SEQ ID NO: 418 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFIIYIEAYMTMKIRN

SEQ ID NO: 419 - protein, 166 aa, Artificial Sequence
  Synthetic Construct
SPGQGTQSENSCTHFPGNLPNMLRDLADAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIAAYMTMKIRN

SEQ ID NO: 420 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFIFYIEAYMTMKIRN

SEQ ID NO: 421 - protein, 166 aa, Artificial Sequence
  Synthetic Construct
SPGQGTQSENSCTHFPGNLPNMLRDLADAFSRVKTFFAMKDQLDNLLLKESLLEDFKGYLGCQALSEMIQFYLEEVMPQAENQDPDIKAHVVSLGENLKTLRLRLRRCHRFLPCENGGGSGGKSKAVEQVKNAFNKLQEKGIYKAMSEFDIFINYIEAYMTMKIRN

SEQ ID NO: 422 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVISLGENLKTLRLRLRACHRFLPCENGGGSGGKSKAVEQVKNAFNKLQEKGIYKAMSEFDIFINYIEAYMTMKIRN

SEQ ID NO: 423 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVISLGENLKTLRLRLRACHRFLPCENGGGSGGKSKAVEQVKNAFNKLQEKGIYKAMSEFAIFINYIEAYMTMKIRN

SEQ ID NO: 424 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVLSLGENLKTLRLRLRACHRFLPCENGGGSGGKSKAVEQVKNAFNKLQEKGIYKAMSEFAIFINYIEAYMTMKIRN

SEQ ID NO: 425 - protein, 166 aa, Artificial Sequence
  Synthetic Construct
SPGQGTQSENSCTHFPGNLPNMLADLADAFSRVKTFFQMKDQLDNLLLKESLLEDFKGYLGCQALSEMIQFYLEEVMPQAENQDPDIKAHVISLGENLKTLRLRLRACHRFLPCENGGGSGGKSKAVEQVKNAFNKLQEKGIYKAMSEFDIFINYIEAYMTMKIRN

SEQ ID NO: 426 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVISLGENLKTLRLRLRACHRFLPCENKGGGSGGSKAVEQVKNAFNKLQEKGIYKAMSEFAIFINYIEAYMTMKIRN

SEQ ID NO: 427 - protein, 166 aa, Artificial Sequence
  Synthetic Construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVLSLGENLKTLRLRLRACHRFLPCENKGGGSGGSKAVEQVKNAFNKLQEKGIYKAMSEFAIFINYIEAYMTMKIRN

SEQ ID NO: 428 - protein, 166 aa, Artificial Sequence
  Synthetic Construct
SPGQGTQSENSCTHFPGNLPNMLADLADAFSRVKTFFQMKDQLDNLLLKESLLEDFKGYLGCQALSEMIQFYLEEVMPQAENQDPDIKAHVISLGENLKTLRLRLRACHRFLPCENKGGGSGGSKAVEQVKNAFNKLQEKGIYKAMSEFDIFINYIEAYMTMKIRN